(19) United States
(12) Patent Application Publication
(10) Pub. No.: US 2023/0125810 A1
(43) Pub. Date: Apr. 27, 2023

(54) DESIGNED BACTERIAL COMPOSITIONS AND USES THEREOF

(71) Applicant: Seres Therapeutics, Inc., Cambridge, MA (US)

(72) Inventors: Asuncion MARTINEZ, Arlington, MA (US); Edward J. O'BRIEN, Arlington, MA (US); Sheri Lynn SIMMONS, Brookline, MA (US); David COOK, Brookline, MA (US); Matthew R. HENN, Cambridge, MA (US); Christopher B. FORD, Swampscott, MA (US); Divya BALASUBRAMANIAN, Arlington, MA (US); Ambar PIÑA, Boston, MA (US); Liyang DIAO, Belmont, MA (US); Meghan CHAFEE, Acton, MA (US); Marin VULIC, Boston, MA (US); Madhumitha NANDAKUMAR, Belmont, CA (US); Sumon DATTA, Cambridge, MA (US); Lata JAYARAMAN, Cambridge, MA (US)

(73) Assignee: Seres Therapeutics, Inc., Cambridge, MA (US)

(21) Appl. No.: 17/780,831

(22) PCT Filed: Nov. 25, 2020
(86) PCT No.: PCT/US2020/062440

§ 371 Date: May 27, 2022

Related U.S. Application Data
(60) Provisional application No. 62/941,534, filed on Nov. 27, 2019.

(51) Int. Cl. A61K 35/741; A61P 35/00; A61P 37/00

(52) U.S. Cl. CPC A61K 35/741; A61P 35/00; A61P 37/00

(57) ABSTRACT

Provided herein are bacterial compositions that are useful for treating and preventing complications and side effects associated with a disease or disorder, such as those associated with dysbiosis of a gastrointestinal tract. The bacterial compositions disclosed herein are designed to exhibit one or more functional features that are useful for the treatment of such diseases and disorders.

### CROSS-REFERENCE TO RELATED APPLICATIONS

This PCT application claims the priority benefit of U.S. Provisional Application No. 62/941,534, filed Nov. 27, 2019, which is incorporated herein by reference in its entirety.

### REFERENCE TO SEQUENCE LISTING SUBMITTED ELECTRONICALLY VIA EFS-WEB

The content of the electronically submitted sequence listing in ASCII text file (Name: 4268.054PC01_SeqListing_ST25.txt; Size: 836,754 bytes; and Date of Creation: Nov. 25, 2019) filed with the application is herein incorporated by reference in its entirety.

### FIELD OF THE DISCLOSURE

The present disclosure relates to bacterial compositions designed to have certain functional features that are useful for treating and/or preventing a range of diseases and disorders, such as those associated with dysbiosis of the gastrointestinal microbiome (e.g., inflammatory bowel disease (IBD), for example, ulcerative colitis and certain cancers).

### BACKGROUND OF THE DISCLOSURE

A healthy gut microbiota is essential for the overall well-being of an individual. Accordingly, dysbiosis of the gut microbiota has been implicated in the pathogenesis of many diseases and disorders, such as inflammatory bowel disease (e.g., colitis), irritable bowel syndrome, coeliac disease, allergy, asthma, metabolic syndrome, cardiovascular disease, and obesity. Carding, S. et al., Micro Ecol Health Dis 26 (2015).

Methods of treating a dysbiosis-related condition have included fecal microbiome transplantation (FMT), which can provide microorganisms to the gastrointestinal tract (GI). However, fecal transplant presents a number of issues, including those related to safety and methods of delivery, such as naso-duodenal-, transcolonoscopic-, or enema-based methods that generally require in-clinic procedures and may introduce adverse events. Treatments using FMT have a likelihood of being inherently inconsistent because of the variability between individuals donating the feces for transplant. FMT methods also introduce a risk of infection by pathogenic organisms, including viruses, bacteria, fungi and protists in the source material. Furthermore, there can be issues related to the stability and storage of donated feces, for example, related to the survival of bacterial species. Some treatments using fecal bacteria delivered in capsules have required that patients take large numbers of capsules, which can be difficult for people with GI illnesses and may reduce compliance with complete treatment.

Accordingly, there is a need for compositions that deliver a consistent product containing cultured bacteria that are of sufficient complexity and that can exhibit key functional features that are useful for the treatment of a dysbiosis or dysbiosis-related condition.

### SUMMARY OF THE DISCLOSURE

Provided herein is a composition comprising a first purified bacterial population and a second purified bacterial population, wherein the first purified bacterial population comprises one or more bacteria selected having a 16S rDNA sequence that is at least 97%, at least 97.5%, at least 98%, at least 98.5%, at least 99%, at least 99.5%, or 100% identical to a 16S rDNA sequence set forth in SEQ ID NO: 215, SEQ ID NO: SEQ ID NO: 112, SEQ ID NO: 113, SEQ ID NO: 114, SEQ ID NO: 115, SEQ ID NO: 116, SEQ ID NO: 188, SEQ ID NO: 212, SEQ ID NO: 160, SEQ ID NO: 186, SEQ ID NO: 203, SEQ ID NO: 104, SEQ ID NO: 208, SEQ ID NO: 189, SEQ ID NO: 187, SEQ ID NO: 207, SEQ ID NO: 190, SEQ ID NO: 191, SEQ ID NO: 211, SEQ ID NO: 209, SEQ ID NO: 110, SEQ ID NO: 150, SEQ ID NO: 175, SEQ ID NO: 158, SEQ ID NO: 210, or SEQ ID NO: 106, and wherein the second purified bacterial population comprises one or more bacteria having one or more features selected from the group consisting of: (i) capable of engrafting when administered to a subject, (ii) capable of having anti-inflammatory activity, (iii) not capable of inducing pro-inflammatory activity, (iv) capable of producing a secondary bile acid, (v) capable of producing a tryptophan metabolite, (vi) capable of restoring epithelial integrity as determined by a primary epithelial cell monolayer barrier integrity assay, (vii) capable of being associated with remission of an inflammatory bowel disease, (viii) capable of producing a short-chain fatty acid, (ix) capable of inhibiting a HDAC activity, (x) capable of producing a medium-chain fatty acid, (xi) capable of expressing catalase activity, (xii) capable of having alpha-fucosidase activity, (xiii) capable of inducing Wnt activation, (xiv) capable of producing a B vitamin, (xv) capable of modulating host metabolism of endocannabinoid, (xvi) capable of producing a polyamine and/or modulating a host metabolism of a polyamine, (xvii) capable of reducing fecal levels of a sphingolipid, (xviii) capable of modulating host production of kynurenine, (xix) capable of reducing fecal calprotectin level, (xx) not capable of activating a toll-like receptor pathway (e.g., TLR4 or TLR5), (xxi) capable of activating a toll-like receptor pathway (e.g., TLR2), (xxii) not capable of producing ursodeoxycholic acid, (xxiii) capable of not being associated with clinical non-remission of an inflammatory bowel disease, (xxiv) capable of inhibiting apoptosis of intestinal epithelial cells, (xxv) capable of inducing an anti-inflammatory IL-10-skewed IL-10/IL-6 cytokine ratio in macrophages, (xxvi) capable of not inducing pro-inflammatory IL-6, TNFa, IL-1b, IL-23 or IL-12 production or gene expression in macrophages, (xxvii) capable of downmodulating one or more genes induced in IFN-γ treated colonic organoids (e.g., those associated with inflammatory chemokine signaling, NF-κB signaling, TNF family signaling, type I interferon signaling, type II interferon signaling, TLR signaling, lymphocyte trafficking, Th17 cell differentiation, Th1 differentiation, Th2 differentiation, apoptosis, inflammasomes, autophagy, oxidative stress, MHC class I and II antigen presentation, complement, mTor, nod-like receptor signaling, PI3K signaling, or combinations thereof), (xxix) capable of producing IL-18, (xxx) capable of inducing the activation of antigen presenting cells, (xxxi) capable of reducing the expression of one or more inhibitory receptors (e.g., TIGIT, TIM-3, or LAG-3) on T cells, (xxxii) capable of increasing expression of one or more genes/proteins associated with T cell activation and/or function (e.g., CD45RO, CD69, IL-24, TNF-α, perforin, or IFN-γ), (xxxiii) capable of enhancing the ability of CD8+ T cells to kill tumor cells, (xxxiv) capable of enhancing the efficacy of an immune checkpoint inhibitor therapy, (xxxv) capable of reducing colonic inflammation, (xxxvi) capable of promoting the recruitment of CD8+ T cells to tumors, and (xxxvii) any combination thereof.

Also provided herein is a composition comprising a first purified bacterial population and a second purified bacterial population, wherein the first bacterial population comprises one or more bacteria having a 16S rDNA sequence that is at least 97%, at least 97.5%, at least 98%, at least 98.5%, at least 99%, at least 99.5%, or 100% identical to a 16S rDNA sequence set forth in SEQ ID NO: 118, SEQ ID NO: SEQ ID NO: 166, SEQ ID NO: 167, SEQ ID NO: 168, SEQ ID NO: 169, SEQ ID NO: 176, SEQ ID NO: 177, SEQ ID NO: 178, or SEQ ID NO: 137, and wherein the second purified bacterial population comprises one or more bacteria having one or more features selected from the group consisting of: (i) capable of engrafting when administered to a subject, (ii) capable of having anti-inflammatory activity, (iii) not capable of inducing pro-inflammatory activity, (iv) capable of producing a secondary bile acid, (v) capable of producing a tryptophan metabolite, (vi) capable of restoring epithelial integrity as determined by a primary epithelial cell monolayer barrier integrity assay, (vii) capable of being associated with remission of an inflammatory bowel disease, (viii) capable of producing a short-chain fatty acid, (ix) capable of inhibiting a HDAC activity, (x) capable of producing a medium-chain fatty acid, (xi) capable of expressing catalase activity, (xii) capable of having alpha-fucosidase activity, (xiii) capable of inducing Wnt activation, (xiv) capable of producing a B vitamin, (xv) capable of modulating host metabolism of endocannabinoid, (xvi) capable of producing a polyamine and/or modulating a host metabolism of a polyamine, (xvii) capable of reducing fecal levels of a sphingolipid, (xviii) capable of modulating host production of kynurenine, (xix) capable of reducing fecal calprotectin level, (xx) not capable of activating a toll-like receptor pathway (e.g., TLR4 or TLR5), (xxi) capable of activating a toll-like receptor pathway (e.g., TLR2), (xxii) not capable of producing ursodeoxycholic acid, (xxiii) capable of not being associated with clinical non-remission of an inflammatory bowel disease, (xxiv) capable of inhibiting apoptosis of intestinal epithelial cells, (xxv) capable of inducing an anti-inflammatory IL-10-skewed IL-10/IL-6 cytokine ratio in macrophages, (xxvi) capable of not inducing pro-inflammatory IL-6, TNFa, IL-1b, IL-23 or IL-12 production or gene expression in macrophages, (xxvii) capable of downmodulating one or more genes induced in IFN-γ treated colonic organoids (e.g., those associated with inflammatory chemokine signaling, NF-κB signaling, TNF family signaling, type I interferon signaling, type II interferon signaling, TLR signaling, lymphocyte trafficking, Th17 cell differentiation, Th1 differentiation, Th2 differentiation, apoptosis, inflammasomes, autophagy, oxidative stress, MHC class I and II antigen presentation, complement, mTor, nod-like receptor signaling, PI3K signaling, or combinations thereof), (xxix) capable of producing IL-18, (xxx) capable of inducing the activation of antigen presenting cells, (xxxi) capable of reducing the expression of one or more inhibitory receptors (e.g., TIGIT, TIM-3, or LAG-3) on T cells, (xxxii) capable of increasing expression of one or more genes/proteins associated with T cell activation and/or function (e.g., CD45RO, CD69, IL-24, TNF-α, perforin, or IFN-γ), (xxxiii) capable of enhancing the ability of CD8+ T cells to kill tumor cells, (xxxiv) capable of enhancing the efficacy of an immune checkpoint inhibitor therapy, (xxxv) capable of reducing colonic inflammation, (xxxvi) capable of promoting the recruitment of CD8+ T cells to tumors, and (xxxvii) any combination thereof.

Provided herein is also composition comprising a first purified bacterial population and a second purified bacterial population, wherein the first bacterial population comprises one or more bacteria having a 16S rDNA sequence that is at least 97%, at least 97.5%, at least 98%, at least 98.5%, at least 99%, at least 99.5%, or 100% identical to a 16S rDNA sequence set forth in SEQ ID NO: 117, SEQ ID NO: 137, SEQ ID NO: 111, or SEQ ID NO: 103, and wherein the second purified bacterial population comprises one or more bacteria having one or more features selected from the group consisting of: (i) capable of engrafting when administered to a subject, (ii) capable of having anti-inflammatory activity, (iii) not capable of inducing pro-inflammatory activity, (iv) capable of producing a secondary bile acid, (v) capable of producing a tryptophan metabolite, (vi) capable of restoring epithelial integrity as determined by a primary epithelial cell monolayer barrier integrity assay, (vii) capable of being associated with remission of an inflammatory bowel disease, (viii) capable of producing a short-chain fatty acid, (ix) capable of inhibiting a HDAC activity, (x) capable of producing a medium-chain fatty acid, (xi) capable of expressing catalase activity, (xii) capable of having alpha-fucosidase activity, (xiii) capable of inducing Wnt activation, (xiv) capable of producing a B vitamin, (xv) capable of modulating host metabolism of endocannabinoid, (xvi) capable of producing a polyamine and/or modulating host metabolism of a polyamine, (xvii) capable of producing a sphingolipid, (xviii) capable of modulating host production of kynurenine, (xix) capable of reducing fecal calprotectin level, (xx) not capable of activating a toll-like receptor pathway (e.g., TLR4 or TLR5), (xxi) capable of activating a toll-like receptor pathway (e.g., TLR2), (xxii) not capable of producing ursodeoxycholic acid, (xxiii) capable of not being associated with clinical non-remission of an inflammatory bowel disease, (xxiv) capable of inhibiting apoptosis of intestinal epithelial cells, (xxv) capable of inducing an anti-inflammatory IL-10-skewed IL-10/IL-6 cytokine ratio in macrophages, (xxvi) capable of not inducing pro-inflammatory IL-6, TNFa, IL-1b, IL-23 or IL-12 production or gene expression in macrophages, (xxvii) capable of downmodulating one or more genes induced in IFN-γ treated colonic organoids (e.g., those associated with inflammatory chemokine signaling, NF-κB signaling, TNF family signaling, type I interferon signaling, type II interferon signaling, TLR signaling, lymphocyte trafficking, Th17 cell differentiation, Th1 differentiation, Th2 differentiation, apoptosis, inflammasomes, autophagy, oxidative stress, MHC class I and II antigen presentation, complement, mTor, nod-like receptor signaling, PI3K signaling, or combinations thereof), (xxix) capable of producing IL-18, (xxx) capable of inducing the activation of antigen presenting cells, (xxxi) capable of reducing the expression of one or more inhibitory receptors (e.g., TIGIT, TIM-3, or LAG-3) on T cells, (xxxii) capable of increasing expression of one or more genes/proteins associated with T cell activation and/or function (e.g., CD45RO, CD69, IL-24, TNF-α, perforin, or IFN-γ), (xxxiii) capable of enhancing the ability of CD8+ T cells to kill tumor cells, (xxxiv) capable of enhancing the efficacy of an immune checkpoint inhibitor therapy, (xxxv) capable of reducing colonic inflammation, (xxxvi) capable of promoting the recruitment of CD8+ T cells to tumors, and (xxxvii) any combination thereof.

In some aspects, the one or more features are selected from (i) capable of engrafting when administered to a subject; (ii) capable of having anti-inflammatory activity, (iii) not capable of inducing pro-inflammatory activity, (iv) capable of producing a secondary bile acid, (v) capable of producing a tryptophan metabolite, (vi) capable of restoring epithelial integrity as determined by a primary epithelial cell monolayer barrier integrity assay, (vii) capable of being associated with remission of an inflammatory bowel disease, (viii) capable of producing a short-chain fatty acid, (ix) capable of inhibiting a HDAC activity, (x) capable of producing a medium-chain fatty acid, or (xi) any combination thereof. In some aspects, the one or more features selected from (i) capable of inhibiting HDAC activity, (ii) capable of producing short-chain fatty acids, (iii) capable of producing tryptophan metabolites, (iv) capable of producing IL-18, (v) capable of inducing the activation of antigen presenting cells, (vi) capable of reducing the expression of one or more inhibitory receptors (e.g., TIGIT, TIM-3, or LAG-3) on T cells, (vii) capable of increasing expression of one or more genes/proteins associated with T cell activation and/or function (e.g., CD45RO, CD69, IL-24, TNF-α, perforin, or IFN-γ), (viii) capable of enhancing the ability of CD8+ T cells to kill tumor cells, (ix) capable of enhancing the efficacy of an immune checkpoint inhibitor therapy, (x) capable of reducing colonic inflammation, (xi) capable of promoting the recruitment of CD8+ T cells to tumors, or (xii) any combination thereof.

In some aspects, the second purified bacterial population comprises a long-term engrafter and/or a transient engrafter. In certain aspects, the second purified bacterial population comprises two, three, four, five, six, seven or more long-term engrafters. In further aspects, the second purified bacterial population comprises two, three or more transient engrafters. In certain aspects, a combination of the first purified bacterial population and the second purified bacterial population comprises three or more transient engrafters and/or seven or more long-term engrafters.

In some aspects, the second purified bacterial population comprises one or more bacteria that are capable of producing a tryptophan metabolite. In some aspects, the second purified bacterial population comprises one or more bacteria that are capable of producing a secondary bile acid. In some aspects, the second purified bacterial population comprises one or more bacteria that are capable of having anti-inflammatory activity. In certain aspects, the second purified bacterial population comprises one or more bacteria that are not capable of inducing pro-inflammatory activity. In some aspects, the second purified bacterial population comprises one or more bacteria that are capable of producing a short-chain fatty acid. In some aspects, the second purified bacterial population comprises one or more bacteria that are capable of producing a medium-chain fatty acid. In some aspects, the second purified bacterial population comprises one or more bacteria that are capable of inhibiting HDAC activity. In some aspects, the second purified bacterial population comprises one or more bacteria that are capable of reducing the expression of one or more inhibitory receptors (e.g., TIGIT, TIM-3, or LAG-3) on T cells. In some aspects, the second purified bacterial population comprises one or more bacteria that are capable of increasing expression of one or more genes/proteins associated with T cell activation and/or function (e.g., CD45RO, CD69, IL-24, TNF-α, perforin, or IFN-γ). In some aspects, the second purified bacterial population comprises one or more bacteria that are capable of enhancing the ability of CD8+ T cells to kill tumor cells. In some aspects, the second purified bacterial population comprises one or more bacteria that are capable of enhancing the efficacy of an immune checkpoint inhibitor therapy. In some aspects, the second purified bacterial population comprises one or more bacteria that are capable of promoting the recruitment of CD8+ T cells to tumors.

Also provided herein is a composition comprising a purified bacterial population, wherein the composition comprises one or more features selected from the group consisting of: (i) capable of engrafting when administered to a subject, (ii) capable of having anti-inflammatory activity, (iii) not capable of inducing pro-inflammatory activity, (iv) capable of producing a secondary bile acid, (v) capable of producing a tryptophan metabolite, (vi) capable of restoring epithelial integrity as determined by a primary epithelial cell monolayer barrier integrity assay, (vii) capable of being associated with remission of an inflammatory bowel disease, (viii) capable of producing a short-chain fatty acid, (ix) capable of inhibiting a HDAC activity, (x) capable of producing a medium-chain fatty acid, (xi) capable of expressing catalase activity, (xii) capable of having alpha-fucosidase activity, (xiii) capable of inducing Wnt activation, (xiv) capable of producing a B vitamin, (xv) capable of modulating host metabolism of endocannabinoid, (xvi) capable of producing a polyamine and/or modulating a host metabolism of a polyamine, (xvii) capable of reducing fecal levels of a sphingolipid, (xviii) capable of modulating host production of kynurenine, (xix) capable of reducing fecal calprotectin level, (xx) not capable of activating a toll-like receptor pathway (e.g., TLR4 or TLR5), (xxi) capable of activating a toll-like receptor pathway (e.g., TLR2), (xxii) not capable of producing ursodeoxycholic acid, (xxiii) capable of not being associated with clinical non-remission of an inflammatory bowel disease, (xxiv) capable of inhibiting apoptosis of intestinal epithelial cells, (xxv) capable of inducing an anti-inflammatory IL-10-skewed IL-10/IL-6 cytokine ratio in macrophages, (xxvi) capable of not inducing pro-inflammatory IL-6, TNFa, IL-1b, IL-23 or IL-12 production or gene expression in macrophages, (xxvii) capable of downmodulating one or more genes induced in IFN-γ treated colonic organoids (e.g., those associated with inflammatory chemokine signaling, NF-κB signaling, TNF family signaling, type I interferon signaling, type II interferon signaling, TLR signaling, lymphocyte trafficking, Th17 cell differentiation, Th1 differentiation, Th2 differentiation, apoptosis, inflammasomes, autophagy, oxidative stress, MHC class I and II antigen presentation, complement, mTor, nod-like receptor signaling, PI3K signaling, or combinations thereof), (xxix) capable of producing IL-18, (xxx) capable of inducing the activation of antigen presenting cells, (xxxi) capable of reducing the expression of one or more inhibitory receptors (e.g., TIGIT, TIM-3, or LAG-3) on T cells, (xxxii) capable of increasing expression of one or more genes/proteins associated with T cell activation and/or function (e.g., CD45RO, CD69, IL-24, TNF-α, perforin, or IFN-γ), (xxxiii) capable of enhancing the ability of CD8+ T cells to kill tumor cells, (xxxiv) capable of enhancing the efficacy of an immune checkpoint inhibitor therapy, (xxxv) capable of reducing colonic inflammation, (xxxvi) capable of promoting the recruitment of CD8+ T cells to tumors, and (xxxvii) any combination thereof.

In some aspects, the two or more features are selected from (i) capable of engrafting when administered to a subject; (ii) capable of having anti-inflammatory activity, (iii) not capable of inducing pro-inflammatory activity, (iv) capable of producing a secondary bile acid, (v) capable of producing a tryptophan metabolite, (vi) capable of restoring epithelial integrity as determined by a primary epithelial cell monolayer barrier integrity assay, (vii) capable of being associated with remission of an inflammatory bowel disease, (viii) capable of producing a short-chain fatty acid, (ix) capable of inhibiting a HDAC activity, (x) capable of producing a medium-chain fatty acid, or (xi) any combination thereof. In some aspects, the two or more features are selected from (i) capable of inhibiting HDAC activity, (ii) capable of producing short-chain fatty acids, (iii) capable of producing tryptophan metabolites, (iv) capable of producing IL-18, (v) capable of inducing the activation of antigen presenting cells, (vi) capable of reducing the expression of one or more inhibitory receptors (e.g., TIGIT, TIM-3, or LAG-3) on T cells, (vii) capable of increasing expression of one or more genes/proteins associated with T cell activation and/or function (e.g., CD45RO, CD69, IL-24, TNF-α, perforin, or IFN-γ), (viii) capable of enhancing the ability of CD8+ T cells to kill tumor cells, (ix) capable of enhancing the efficacy of an immune checkpoint inhibitor therapy, (x) capable of reducing colonic inflammation, (xi) capable of promoting the recruitment of CD8+ T cells to tumors, or (xii) any combination thereof.

In some aspects, the purified bacterial population of a composition disclosed herein comprises one or more bacteria having a 16S rDNA sequence that is at least 97%, at least 97.5%, at least 98%, at least 98.5%, at least 99%, at least 99.5%, or 100% identical to a 16S rDNA sequence set forth in SEQ ID NO: 215, SEQ ID NO: 112, SEQ ID NO: 113, SEQ ID NO: 114, SEQ ID NO: 115, SEQ ID NO: 116, SEQ ID NO: 188, SEQ ID NO: 212, SEQ ID NO: 160, SEQ ID NO: 186, SEQ ID NO: 104, SEQ ID NO: 208, SEQ ID NO: 189, SEQ ID NO: 187, SEQ ID NO: 207, SEQ ID NO: 190, SEQ ID NO: 191, SEQ ID NO: 211, SEQ ID NO: 209, SEQ ID NO: 110, SEQ ID NO: 159, SEQ ID NO: 175, SEQ ID NO: 158, SEQ ID NO: 210, or SEQ ID NO: 106.

In some aspects, the purified bacterial population comprises one or more bacteria having a 16S rDNA sequence that is at least 97%, at least 97.5%, at least 98%, at least 98.5%, at least 99%, at least 99.5%, or 100% identical to a 16S rDNA sequence set forth in SEQ ID NO: 185, SEQ ID NO: 183, SEQ ID NO: 161, SEQ ID NO: 206, SEQ ID NO: 102, SEQ ID NO: 214, SEQ ID NO: 184, SEQ ID NO: 204, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: SEQ ID NO: 166, SEQ ID NO: 167, SEQ ID NO: 168, SEQ ID NO: 169, SEQ ID NO: 109, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 192, SEQ ID NO: 176, SEQ ID NO: 177, SEQ ID NO: 178, SEQ ID NO: 107, SEQ ID NO: 137, SEQ ID NO: 198, SEQ ID NO: 199, SEQ ID NO: 200, SEQ ID NO: 201, SEQ ID NO: 202, SEQ ID NO: 133, SEQ ID NO: 193, SEQ ID NO: 194, SEQ ID NO: 195, SEQ ID NO: 196, SEQ ID NO: 197, SEQ ID NO: 126, SEQ ID NO: 127, SEQ ID NO: 103, SEQ ID NO: 108, SEQ ID NO: 124, SEQ ID NO: 165, SEQ ID NO: 136, SEQ ID NO: 125, SEQ ID NO: 111, SEQ ID NO: 164, SEQ ID NO: 205, SEQ ID NO: 128, SEQ ID NO: 129, SEQ ID NO: 130, SEQ ID NO: 131, SEQ ID NO: 132, SEQ ID NO: 162, SEQ ID NO: 117, SEQ ID NO: 118, SEQ ID NO: 105, SEQ ID NO: 119, SEQ ID NO: 120, SEQ ID NO: 121, SEQ ID NO: 122, SEQ ID NO: 123, SEQ ID NO: 170, SEQ ID NO: 171, SEQ ID NO: 172, SEQ ID NO: 173, SEQ ID NO: 174, SEQ ID NO: 163, SEQ ID NO: 182, SEQ ID NO: 135, SEQ ID NO: 134, SEQ ID NO: 179, SEQ ID NO: 180, SEQ ID NO: 181, SEQ ID NO: 203, or SEQ ID NO: 213.

Provided herein is a composition comprising a purified bacterial population, comprising two or more bacteria, wherein the two or more bacteria comprises a long-term engrafter and a transient engrafter.

In some aspects, the purified bacterial population further comprises one or more bacteria, which has one or more features selected from the group consisting of: (i) capable of engrafting when administered to a subject, (ii) capable of having anti-inflammatory activity, (iii) not capable of inducing pro-inflammatory activity, (iv) capable of producing a secondary bile acid, (v) capable of producing a tryptophan metabolite, (vi) capable of restoring epithelial integrity as determined by a primary epithelial cell monolayer barrier integrity assay, (vii) capable of being associated with remission of an inflammatory bowel disease, (viii) capable of producing a short-chain fatty acid, (ix) capable of inhibiting a HDAC activity, (x) capable of producing a medium-chain fatty acid, (xi) capable of expressing catalase activity, (xii) capable of having alpha-fucosidase activity, (xiii) capable of inducing Wnt activation, (xiv) capable of producing a B vitamin, (xv) capable of modulating host metabolism of endocannabinoid, (xvi) capable of producing a polyamine and/or modulating host metabolism of polyamines, (xvii) capable of reducing fecal levels of a sphingolipid, (xviii) capable of modulating host production of kynurenine, (xix) capable of reducing fecal calprotectin level, (xx) not capable of activating a toll-like receptor pathway (e.g., TLR4 or TLR5), (xxi) capable of activating a toll-like receptor pathway (e.g., TLR2), (xxii) not capable of producing ursodeoxycholic acid, (xxiii) capable of not being associated with clinical non-remission of an inflammatory bowel disease, (xxiv) capable of inhibiting apoptosis of intestinal epithelial cells, (xxv) capable of inducing an anti-inflammatory IL-10-skewed IL-10/IL-6 cytokine ratio in macrophages, (xxvi) capable of not inducing pro-inflammatory IL-6, TNFa, IL-1b, IL-23 or IL-12 production or gene expression in macrophages, (xxvii) capable of downmodulating one or more genes induced in IFN-γ treated colonic organoids (e.g., those associated with inflammatory chemokine signaling, NF-κB signaling, TNF family signaling, type I interferon signaling, type II interferon signaling, TLR signaling, lymphocyte trafficking, Th17 cell differentiation, Th1 differentiation, Th2 differentiation, apoptosis, inflammasomes, autophagy, oxidative stress, MHC class I and II antigen presentation, complement, mTor, nod-like receptor signaling, PI3K signaling, or combinations thereof), (xxix) capable of producing IL-18, (xxx) capable of inducing the activation of antigen presenting cells, (xxxi) capable of reducing the expression of one or more inhibitory receptors (e.g., TIGIT, TIM-3, or LAG-3) on T cells, (xxxii) capable of increasing expression of one or more genes/proteins associated with T cell activation and/or function (e.g., CD45RO, CD69, IL-24, TNF-α, perforin, or IFN-γ), (xxxiii) capable of enhancing the ability of CD8+ T cells to kill tumor cells, (xxxiv) capable of enhancing the efficacy of an immune checkpoint inhibitor therapy, (xxxv) capable of reducing colonic inflammation, (xxxvi) capable of promoting the recruitment of CD8+ T cells to tumors, and (xxxvii) any combination thereof.

In some aspects, the one or more features are selected from (i) capable of engrafting when administered to a subject; (ii) capable of having anti-inflammatory activity, (iii) not capable of inducing pro-inflammatory activity, (iv) capable of producing a secondary bile acid, (v) capable of producing a tryptophan metabolite, (vi) capable of restoring epithelial integrity as determined by a primary epithelial cell monolayer barrier integrity assay, (vii) capable of being associated with remission of an inflammatory bowel disease, (viii) capable of producing a short-chain fatty acid, (ix) capable of inhibiting a HDAC activity, (x) capable of producing a medium-chain fatty acid, or (xi) any combination thereof. In some aspects, the one or more features are selected from (i) capable of inhibiting HDAC activity, (ii) capable of producing short-chain fatty acids, (iii) capable of producing tryptophan metabolites, (iv) capable of producing IL-18, (v) capable of inducing the activation of antigen presenting cells, (vi) capable of reducing the expression of one or more inhibitory receptors (e.g., TIGIT, TIM-3, or LAG-3) on T cells, (vii) capable of increasing expression of one or more genes/proteins associated with T cell activation and/or function (e.g., CD45RO, CD69, IL-24, TNF-α, perforin, or IFN-γ), (viii) capable of enhancing the ability of CD8+ T cells to kill tumor cells, (ix) capable of enhancing the efficacy of an immune checkpoint inhibitor therapy, (x) capable of reducing colonic inflammation, (xi) capable of promoting the recruitment of CD8+ T cells to tumors, or (xii) any combination thereof.

In some aspects, a composition comprising a purified bacterial population disclosed herein comprises two, three, four, five, six, seven or more long-term engrafters. In certain aspects, the purified bacterial population comprises two, three, four, five, six, seven or more transient engrafters. In some aspects, the purified bacterial population comprises three or more transient engrafters and/or seven or more long-term engrafters.

In some aspects, the purified bacterial population comprises one or more bacteria that are capable of producing a tryptophan metabolite. In some aspects, the purified bacterial population comprises one or more bacteria that are capable of producing a secondary bile acid. In certain aspects, the purified bacterial population comprises one or more bacteria that are capable of having anti-inflammatory activity. In other aspects, the purified bacterial population comprises one or more bacteria that are not capable of inducing pro-inflammatory activity. In some aspects, the purified bacterial population comprises one or more bacteria that are capable of producing a short-chain fatty acid. In some aspects, the purified bacterial population comprises one or more bacteria that are capable of producing a medium-chain fatty acid. In some aspects, the purified bacterial population comprises one or more bacteria that are capable of inhibiting HDAC activity.

In some aspects, the tryptophan metabolite disclosed herein comprises indole, 3-methyl indole, indoleacrylate, or any combination thereof. In certain aspects, the tryptophan metabolite is indole. In certain aspects, the tryptophan metabolite is 3-methyl indole.

In some aspects, one or more bacteria capable of producing a secondary bile acid has 7α-dehydroxylase activity. In some aspects, the one or more bacteria capable of producing a secondary bile acid has bile salt hydrolase (BSH) activity. In certain aspects, the first purified bacterial population and/or the second purified bacterial population of a composition disclosed herein does not comprise a bacterium having 7β-hydroxysteroid dehydrogenase (7β-HSDH) activity. In some aspects, the secondary bile acid comprises deoxycholic acid (DCA), 3α 12-oxo-deoxycholic acid, 3β 12α-deoxycholic acid (3-isodeoxycholic acid), 7α 3-oxo-chenodeoxycholic acid, lithocholic acid (LCA), 3-oxo LCA, or any combination thereof.

In some aspects, one or more bacteria capable of having anti-inflammatory activity comprises (i) bacteria capable of producing a short-chain fatty acid, (ii) bacteria capable of inhibiting histone deacetylase (HDAC) activity, (iii) bacteria capable of inhibiting TNF-α-driven IL-8 secretion in epithelial cells in vitro, or (iv) any combination thereof. In some aspects, one or more bacteria not capable of inducing pro-inflammatory activity comprises (i) bacteria not capable of inducing IL-8 secretion in epithelial cells in vitro and/or (ii) bacteria not capable of activating Toll-like receptor 4 (TLR4) and/or Toll-like receptor 5 (TLR5) in vitro.

In some aspects, a short-chain fatty acid disclosed herein comprises formate, acetate, propionate, butyrate, isobutryate, valerate, isovalerate, or any combination thereof. In certain aspects, the short-chain fatty acid is propionate. In certain aspects, the short-chain fatty acid is butyrate. In some aspects, a medium-chain fatty acid comprises hexanoate, octanoate, decanoate, dodecanoate, or any combination thereof. In certain aspects, the medium-chain fatty acid is hexanoate or pentanoate.

In some aspects, a long-term engrafter that can be included in a composition disclosed herein has a 16S rDNA sequence that is at least 97%, at least 97.5%, at least 98%, at least 98.5%, at least 99%, at least 99.5%, or 100% identical to a 16S rDNA sequence of a long-term engrafter provided in Table 5. In certain aspects, the long-term engrafter has a 16S rDNA sequence that is at least 97%, at least 97.5%, at least 98%, at least 98.5%, at least 99%, at least 99.5%, or 100% identical to a 16S rDNA sequence set forth in SEQ ID NO: 161, SEQ ID NO: 211, SEQ ID NO: 185, SEQ ID NO: 208, SEQ ID NO: 203, SEQ ID NO: 111, SEQ ID NO: 117, SEQ ID NO: 206, SEQ ID NO: 159, SEQ ID NO: 182, SEQ ID NO: 183, SEQ ID NO: 135, SEQ ID NO: 165, SEQ ID NO: 209, SEQ ID NO: 179, SEQ ID NO: 180, SEQ ID NO: 181, or SEQ ID NO: 189.

In some aspects, a transient-engrafter disclosed herein has a 16S rDNA sequence that is at least 97%, at least 97.5%, at least 98%, at least 98.5%, at least 99%, at least 99.5%, or 100% identical to a 16S rDNA sequence of a transient engrafter provided in Table 5. In some aspects, the transient engrafter has a 16S rDNA sequence that is at least 97%, at least 97.5%, at least 98%, at least 98.5%, at least 99%, at least 99.5%, or 100% identical to a 16S rDNA sequence set forth in SEQ ID NO: 119, SEQ ID NO: 120, SEQ ID NO: 121, SEQ ID NO: 122, SEQ ID NO: 123, SEQ ID NO: 103, SEQ ID NO: 190, SEQ ID NO: 191, SEQ ID NO: 118, SEQ ID NO: 163, SEQ ID NO: 133, SEQ ID NO: 192, SEQ ID NO: 134, SEQ ID NO: 137, SEQ ID NO: 128, SEQ ID NO: 129, SEQ ID NO: 130, SEQ ID NO: 131, SEQ ID NO: 132, or SEQ ID NO: 175.

Provided herein is a composition comprising a purified bacterial population, which comprises one or more bacteria having a 16S rDNA sequence that is at least 97%, at least 97.5%, at least 98%, at least 98.5%, at least 99%, at least 99.5%, or 100% identical to a 16S rDNA sequence set forth in SEQ ID NO: 215, SEQ ID NO: 112, SEQ ID NO: 113, SEQ ID NO: 114, SEQ ID NO: 115, SEQ ID NO: 116, SEQ ID NO: 188, SEQ ID NO: 212, SEQ ID NO: 160, SEQ ID NO: 186, SEQ ID NO: 104, SEQ ID NO: 208, SEQ ID NO: 189, SEQ ID NO: 187, SEQ ID NO: 207, SEQ ID NO: 190, SEQ ID NO: 191, SEQ ID NO: 211, SEQ ID NO: 209, SEQ ID NO: 110, SEQ ID NO: 159, SEQ ID NO: 175, SEQ ID NO: 158, SEQ ID NO: 210, or SEQ ID NO: 106.

In some aspects, the purified bacterial population further comprises one or more bacteria having a 16S rDNA sequence that is at least 97%, at least 97.5%, at least 98%, at least 98.5%, at least 99%, at least 99.5%, or 100% identical to a 16S rDNA sequence set forth in SEQ ID NO: 185, SEQ ID NO: 183, SEQ ID NO: 161, SEQ ID NO: 206, SEQ ID NO: 102, SEQ ID NO: 214, SEQ ID NO: 184, SEQ ID NO: 204, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: SEQ ID NO: 166, SEQ ID NO: 167, SEQ ID NO: 168, SEQ ID NO: 169, SEQ ID NO: 109, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 192, SEQ ID NO: 176, SEQ ID NO: 177, SEQ ID NO: 178, SEQ ID NO: 107, SEQ ID NO: 137, SEQ ID NO: 198, SEQ ID NO: 199, SEQ ID NO: 200, SEQ ID NO: 201, SEQ ID NO: 202, SEQ ID NO: 133, SEQ ID NO: 193, SEQ ID NO: 194, SEQ ID NO: 195, SEQ ID NO: 196, SEQ ID NO: 197, SEQ ID NO: 126, SEQ ID NO: 127, SEQ ID NO: 103, SEQ ID NO: 108, SEQ ID NO: 124, SEQ ID NO: 165, SEQ ID NO: 136, SEQ ID NO: 125, SEQ ID NO: 111, SEQ ID NO: 164, SEQ ID NO: 205, SEQ ID NO: 128, SEQ ID NO: 129, SEQ ID NO: 130, SEQ ID NO: 131, SEQ ID NO: 132, SEQ ID NO: 162, SEQ ID NO: 117, SEQ ID NO: 118, SEQ ID NO: 105, SEQ ID NO: 119, SEQ ID NO: 120, SEQ ID NO: 121, SEQ ID NO: 122, SEQ ID NO: 123, SEQ ID NO: 170, SEQ ID NO: 171, SEQ ID NO: 172, SEQ ID NO: 173, SEQ ID NO: 174, SEQ ID NO: 163, SEQ ID NO: 182, SEQ ID NO: 135, SEQ ID NO: 134, SEQ ID NO: 179, SEQ ID NO: 180, SEQ ID NO: 181, SEQ ID NO: 203, or SEQ ID NO: 213.

Disclosed herein is a composition comprising a purified population of bacteria having 16S rDNA sequences that are at least 97%, at least 97.5%, at least 98%, at least 98.5%, at least 99%, at least 99.5%, or 100% identical to a 16S rDNA sequence selected from the group consisting of: (1) SEQ ID NO: 112, SEQ ID NO: 113, SEQ ID NO: 114, SEQ ID NO: 115, SEQ ID NO: 116, SEQ ID NO: 188, SEQ ID NO: 186, SEQ ID NO: 104, SEQ ID NO: 187; (2) SEQ ID NO: 186; (3) SEQ ID NO: 112, SEQ ID NO: 113, SEQ ID NO: 114, SEQ ID NO: 115, SEQ ID NO: 116, SEQ ID NO: 188, SEQ ID NO: 186, SEQ ID NO: 104, SEQ ID NO: 190, SEQ ID NO: 191, SEQ ID NO: 175; (4) SEQ ID NO: 112, SEQ ID NO: 113, SEQ ID NO: 114, SEQ ID NO: 115, SEQ ID NO: 116, SEQ ID NO: 188, SEQ ID NO: 186, SEQ ID NO: 203, SEQ ID NO: 104; (5) SEQ ID NO: 112, SEQ ID NO: 113, SEQ ID NO: 114, SEQ ID NO: 115, SEQ ID NO: 116, SEQ ID NO: 186, SEQ ID NO: 203, SEQ ID NO: 190, SEQ ID NO: 191, SEQ ID NO: 175; (6) SEQ ID NO: 112, SEQ ID NO: 113, SEQ ID NO: 114, SEQ ID NO: 115, SEQ ID NO: 116, SEQ ID NO: 104; (7) SEQ ID NO: 112, SEQ ID NO: 113, SEQ ID NO: 114, SEQ ID NO: 115, SEQ ID NO: 116, SEQ ID NO: 104, SEQ ID NO: 190, SEQ ID NO: 191, SEQ ID NO: 175; (8) SEQ ID NO: 112, SEQ ID NO: 113, SEQ ID NO: 114, SEQ ID NO: 115, SEQ ID NO: 116, SEQ ID NO: 203, SEQ ID NO: 104; (9) SEQ ID NO: 112, SEQ ID NO: 113, SEQ ID NO: 114, SEQ ID NO: 115, SEQ ID NO: 116, SEQ ID NO: 203, SEQ ID NO: 190, SEQ ID NO: 191, SEQ ID NO: 175; (10) SEQ ID NO: 159, SEQ ID NO: 190, SEQ ID NO: 191, SEQ ID NO: 211; (11) SEQ ID NO: 212, SEQ ID NO: 203, SEQ ID NO: 189, SEQ ID NO: 190, SEQ ID NO: 191, SEQ ID NO: 211, SEQ ID NO: 159, SEQ ID NO: 175, SEQ ID NO: 210; (12) SEQ ID NO: 212, SEQ ID NO: 203, SEQ ID NO: 189, SEQ ID NO: 190, SEQ ID NO: 191, SEQ ID NO: 211, SEQ ID NO: 159, SEQ ID NO: 175; (13) SEQ ID NO: 212, SEQ ID NO: 203, SEQ ID NO: 189, SEQ ID NO: 190, SEQ ID NO: 191, SEQ ID NO: 211, SEQ ID NO: 159; (14) SEQ ID NO: 212, SEQ ID NO: 203, SEQ ID NO: 190, SEQ ID NO: 191, SEQ ID NO: 211, SEQ ID NO: 159; (15) SEQ ID NO: 203, SEQ ID NO: 189, SEQ ID NO: 211, SEQ ID NO: 175; (16) SEQ ID NO: 203, SEQ ID NO: 190, SEQ ID NO: 191, SEQ ID NO: 211, SEQ ID NO: 175; (17) SEQ ID NO: 203, SEQ ID NO: 189, SEQ ID NO: 190, SEQ ID NO: 191, SEQ ID NO: 211, SEQ ID NO: 175; (18) SEQ ID NO: 203, SEQ ID NO: 208, SEQ ID NO: 190, SEQ ID NO: 191, SEQ ID NO: 211, SEQ ID NO: 159, SEQ ID NO: 175; (19) SEQ ID NO: 203, SEQ ID NO: 190, SEQ ID NO: 191, SEQ ID NO: 211, SEQ ID NO: 159, SEQ ID NO: 175; (20) SEQ ID NO: 203, SEQ ID NO: 208, SEQ ID NO: 190, SEQ ID NO: 191, SEQ ID NO: 211, SEQ ID NO: 159, SEQ ID NO: 175; (21) SEQ ID NO: 203, SEQ ID NO: 208, SEQ ID NO: 189, SEQ ID NO: 190, SEQ ID NO: 191, SEQ ID NO: 211, SEQ ID NO: 159, SEQ ID NO: 175; (22) SEQ ID NO: 203, SEQ ID NO: 208, SEQ ID NO: 190, SEQ ID NO: 191, SEQ ID NO: 21, SEQ ID NO: 209, SEQ ID NO: 159; (23) SEQ ID NO: 203, SEQ ID NO: 190, SEQ ID NO: 191, SEQ ID NO: 211, SEQ ID NO: 209, SEQ ID NO: 159; (24) SEQ ID NO: 215, SEQ ID NO: 160, SEQ ID NO: 158, SEQ ID NO: 106; and (25) any combination thereof.

In some aspects, the purified bacterial population further comprises 16S rDNA sequences that are at least 97%, at least 97.5%, at least 98%, at least 98.5%, at least 99%, at least 99.5%, or 100% identical to a 16S rDNA sequence selected from the group consisting of: (1) SEQ ID NO: 184, SEQ ID NO: 204, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 198, SEQ ID NO: 199, SEQ ID NO: 200, SEQ ID NO: 201, SEQ ID NO: 202, SEQ ID NO: 126, SEQ ID NO: 127, SEQ ID NO: 103, SEQ ID NO: 128, SEQ ID NO: 129, SEQ ID NO: 130, SEQ ID NO: 131, SEQ ID NO: 132, SEQ ID NO: 162, SEQ ID NO: 119, SEQ ID NO: 120, SEQ ID NO: 121, SEQ ID NO: 122, SEQ ID NO: 123; (2) SEQ ID NO: 204, SEQ ID NO: 103; (3) SEQ ID NO: 204, SEQ ID NO: 103, SEQ ID NO: 205; (4) SEQ ID NO: 185, SEQ ID NO: 204, SEQ ID NO: 176, SEQ ID NO: 177, SEQ ID NO: 178, SEQ ID NO: 117; (5) SEQ ID NO: 184, SEQ ID NO: 204, SEQ ID NO: 198, SEQ ID NO: 199, SEQ ID NO: 200, SEQ ID NO: 201, SEQ ID NO: 202, SEQ ID NO: 103, SEQ ID NO: 162, SEQ ID NO: 134; (6) SEQ ID NO: 184, SEQ ID NO: 204, SEQ ID NO: 198, SEQ ID NO: 199, SEQ ID NO: 200, SEQ ID NO: 201, SEQ ID NO: 202, SEQ ID NO: 103, SEQ ID NO: 165, SEQ ID NO: 162, SEQ ID NO: 182; (7) SEQ ID NO: 184, SEQ ID NO: 204, SEQ ID NO: 103, SEQ ID NO: 165, SEQ ID NO: 162, SEQ ID NO: 182, SEQ ID NO: 134; (8) SEQ ID NO: 184, SEQ ID NO: 204, SEQ ID NO: 166, SEQ ID NO: 167, SEQ ID NO: 168, SEQ ID NO: 169, SEQ ID NO: 176, SEQ ID NO: 177, SEQ ID NO: 178, SEQ ID NO: 137, SEQ ID NO: 103, SEQ ID NO: 128, SEQ ID NO: 129, SEQ ID NO: 130, SEQ ID NO: 131, SEQ ID NO: 132, SEQ ID NO: 162, SEQ ID NO: 118, SEQ ID NO: 119, SEQ ID NO: 120, SEQ ID NO: 121, SEQ ID NO: 122, SEQ ID NO: 123; (9) SEQ ID NO: 184, SEQ ID NO: 204, SEQ ID NO: SEQ ID NO: 166, SEQ ID NO: 167, SEQ ID NO: 168, SEQ ID NO: 169, SEQ ID NO: 176, SEQ ID NO: 177, SEQ ID NO: 178, SEQ ID NO: 137, SEQ ID NO: 103, SEQ ID NO: 162, SEQ ID NO: 118, SEQ ID NO: 134; (10) SEQ ID NO: 184, SEQ ID NO: 204, SEQ ID NO: SEQ ID NO: 166, SEQ ID NO: 167, SEQ ID NO: 168, SEQ ID NO: 169, SEQ ID NO: 176, SEQ ID NO: 177, SEQ ID NO: 178, SEQ ID NO: 137, SEQ ID NO: 103, SEQ ID NO: 162, SEQ ID NO: 118, SEQ ID NO: 182; (11) SEQ ID NO: 184, SEQ ID NO: 204, SEQ ID NO: SEQ ID NO: 166, SEQ ID NO: 167, SEQ ID NO: 168, SEQ ID NO: 169, SEQ ID NO: 176, SEQ ID NO: 177, SEQ ID NO: 178, SEQ ID NO: 137, SEQ ID NO: 103, SEQ ID NO: 162, SEQ ID NO: 118, SEQ ID NO: 182, SEQ ID NO: 134; (12) SEQ ID NO: 111, SEQ ID NO: 135, SEQ ID NO: 134; (13) SEQ ID NO: 166, SEQ ID NO: 167, SEQ ID NO: 168, SEQ ID NO: 169, SEQ ID NO: 176, SEQ ID NO: 177, SEQ ID NO: 178, SEQ ID NO: 137, SEQ ID NO: 111, SEQ ID NO: 135, SEQ ID NO: 134; (14) SEQ ID NO: 183, SEQ ID NO: 204, SEQ ID NO: 166, SEQ ID NO: 167, SEQ ID NO: 168, SEQ ID NO: 169, SEQ ID NO: 176, SEQ ID NO: 177, SEQ ID NO: 178, SEQ ID NO: 137, SEQ ID NO: 133, SEQ ID NO: 103, SEQ ID NO: 111, SEQ ID NO: 118, SEQ ID NO: 163, SEQ ID NO: 135, SEQ ID NO: 134; (15) SEQ ID NO: 166, SEQ ID NO: 167, SEQ ID NO: 168, SEQ ID NO: 169, SEQ ID NO: 176, SEQ ID NO: 177, SEQ ID NO: 178, SEQ ID NO: 137, SEQ ID NO: 193, SEQ ID NO: 194, SEQ ID NO: 195, SEQ ID NO: 196, SEQ ID NO: 197, SEQ ID NO: 111, SEQ ID NO: 118, SEQ ID NO: 170, SEQ ID NO: 171, SEQ ID NO: 172, SEQ ID NO: 173, SEQ ID NO: 174, SEQ ID NO: 135, SEQ ID NO: 134; (16) SEQ ID NO: 133, SEQ ID NO: 111, SEQ ID NO: 128, SEQ ID NO: 129, SEQ ID NO: 130, SEQ ID NO: 131, SEQ ID NO: 132, SEQ ID NO: 119, SEQ ID NO: 120, SEQ ID NO: 121, SEQ ID NO: 122, SEQ ID NO: 123, SEQ ID NO: 182, SEQ ID NO: 135, SEQ ID NO: 134; (17) SEQ ID NO: 111, SEQ ID NO: 182, SEQ ID NO: 135, SEQ ID NO: 134; (18) SEQ ID NO: 166, SEQ ID NO: 167, SEQ ID NO: 168, SEQ ID NO: 169, SEQ ID NO: 176, SEQ ID NO: 177, SEQ ID NO: 178, SEQ ID NO: 137, SEQ ID NO: 111, SEQ ID NO: 118, SEQ ID NO: 182, SEQ ID NO: 135, SEQ ID NO: 134; (19) SEQ ID NO: 184, SEQ ID NO: 166, SEQ ID NO: 167, SEQ ID NO: 168, SEQ ID NO: 169, SEQ ID NO: 176, SEQ ID NO: 177, SEQ ID NO: 178, SEQ ID NO: 137, SEQ ID NO: 111, SEQ ID NO: 118, SEQ ID NO: 135, SEQ ID NO: 134; (20) SEQ ID NO: 183, SEQ ID NO: 166, SEQ ID NO: 167, SEQ ID NO: 168, SEQ ID NO: 169, SEQ ID NO: 176, SEQ ID NO: 177, SEQ ID NO: 178, SEQ ID NO: 137, SEQ ID NO: 136, SEQ ID NO: 111, SEQ ID NO: 118, SEQ ID NO: 119, SEQ ID NO: 120, SEQ ID NO: 121, SEQ ID NO: 122, SEQ ID NO: 123, SEQ ID NO: 135, SEQ ID NO: 134; (21) SEQ ID NO: 185, SEQ ID NO: 183, SEQ ID NO: 161, SEQ ID NO: 206, SEQ ID NO 137, SEQ ID NO: 133, SEQ ID NO: 103, SEQ ID NO: 111, SEQ ID NO: 117, SEQ ID NO: 119, SEQ ID NO: 120, SEQ ID NO: 121, SEQ ID NO: 122, SEQ ID NO: 123, SEQ ID NO: 163; (22) SEQ ID NO: 183, SEQ ID NO: 161, SEQ ID NO: 206, SEQ ID NO: 137, SEQ ID NO: 103, SEQ ID NO: 111, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 163, SEQ ID NO: 134; (23) SEQ ID NO: 185, SEQ ID NO: 183, SEQ ID NO: 137, SEQ ID NO: 103, SEQ ID NO: 111, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 163, SEQ ID NO: 134; (24) SEQ ID NO: 206, SEQ ID NO: 137, SEQ ID NO: 103, SEQ ID NO: 111, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 182, SEQ ID NO: 13; (25) SEQ ID NO: 185, SEQ ID NO: 183, SEQ ID NO: 206, SEQ ID NO: 192, SEQ ID NO: 137, SEQ ID NO: 103, SEQ ID NO: 165, SEQ ID NO: 111, SEQ ID NO: 117, SEQ ID NO: 118, SEQ ID NO: 163; (26) SEQ ID NO: 185, SEQ ID NO: 183, SEQ ID NO: 206, SEQ ID NO: 137, SEQ ID NO: 103, SEQ ID NO: 111, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 163, SEQ ID NO: 182; (27) SEQ ID NO: 206, SEQ ID NO: 137, SEQ ID NO: 103, SEQ ID NO: 165, SEQ ID NO: 111, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 163, SEQ ID NO: 182; (28) SEQ ID NO: 185, SEQ ID NO: 183, SEQ ID NO: 206, SEQ ID NO: 137, SEQ ID NO: 103, SEQ ID NO: 165, SEQ ID NO: 111, SEQ ID NO: 117, SEQ ID NO: 119, SEQ ID NO: 120, SEQ ID NO: 121, SEQ ID NO: 122, SEQ ID NO: 123, SEQ ID NO: 163, SEQ ID NO: 182, SEQ ID NO: 135; (29) SEQ ID NO: 185, SEQ ID NO: 161, SEQ ID NO: 206, SEQ ID NO: 137, SEQ ID NO: 133, SEQ ID NO: 103, SEQ ID NO: 111, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 163, SEQ ID NO: 182, SEQ ID NO: 135; (30) SEQ ID NO: 185, SEQ ID NO: 183, SEQ ID NO: 206, SEQ ID NO: 192, SEQ ID NO: 137, SEQ ID NO: 133, SEQ ID NO: 103, SEQ ID NO: 165, SEQ ID NO: 111, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 163; (31) SEQ ID NO: 185, SEQ ID NO: 183, SEQ ID NO: 206, SEQ ID NO: 137, SEQ ID NO: 103, SEQ ID NO: 111, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 163, SEQ ID NO: 182, SEQ ID NO: 135; (32) SEQ ID NO: 185, SEQ ID NO: 183, SEQ ID NO: 161, SEQ ID NO: 206, SEQ ID NO: 192, SEQ ID NO: 137, SEQ ID NO: 133, SEQ ID NO: 103, SEQ ID NO: 165, SEQ ID NO: 111, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 163, SEQ ID NO: 182, SEQ ID NO: 135, SEQ ID NO: 134; (33) SEQ ID NO: 185, SEQ ID NO: 183, SEQ ID NO: 161, SEQ ID NO: 206, SEQ ID NO: 192, SEQ ID NO: 137, SEQ ID NO: 103, SEQ ID NO: 165, SEQ ID NO: 111, SEQ ID NO: 128, SEQ ID NO: 129, SEQ ID NO: 130, SEQ ID NO: 131, SEQ ID NO: 132, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 163, SEQ ID NO: 182, SEQ ID NO: 134, SEQ ID NO: 179, SEQ ID NO: 180, SEQ ID NO: 181; (34) SEQ ID NO: 185, SEQ ID NO: 161, SEQ ID NO: 206, SEQ ID NO: 137, SEQ ID NO: 103, SEQ ID NO: 111, SEQ ID NO: 128, SEQ ID NO: 129, SEQ ID NO: 130, SEQ ID NO: 131, SEQ ID NO: 132, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 163, SEQ ID NO: 182, SEQ ID NO: 179, SEQ ID NO: 180, SEQ ID NO: 181; (35) SEQ ID NO: 102, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 166, SEQ ID NO: 167, SEQ ID NO: 168, SEQ ID NO: 169, SEQ ID NO: 109, SEQ ID NO: 107, SEQ ID NO: 103, SEQ ID NO: 108, SEQ ID NO: 117, SEQ ID NO: 105, SEQ ID NO: 179, SEQ ID NO: 180, SEQ ID NO: 181; and (36) any combination thereof.

Provided herein is a composition comprising a purified bacterial population, which comprises one or more bacteria having a 16S rDNA sequence that is at least 97%, at least 97.5%, at least 98%, at least 98.5%, at least 99%, at least 99.5%, or 100% identical to a 16S rDNA sequence set forth in SEQ ID NOs: 151, 196, 190, 191, 192, 193, 194, 120, 121, 122, 123, 124, 125, 126, 127, 128, 129, 130, 131, 136, 200, 201, 202, 203, 204, 148, 149, 150, 107, 108, 109, 110, 111, 105, 182, 219, 153, 115, 213, 166, 167, 168, 169, 170, 171, 172, 173, 174, 175, 176, 177, 214, 215, 216, 103, 178, 161, 154, 155, 156, 157, 158, 119, 132, 133, 134, 135, 314, 315, 316, 317, 117, 205, 206, 207, 208, 209, 220, 221, 222, 197, 263, 102, 118, 159, 198, 112, 184, 104, 223, 189, 186, 224, 106, 199, 147, 211, 179, 180, 152, 195, 185, 116, 225, 226, 210, 212, 181, 114, 187, or combinations thereof.

Provided herein is a composition comprising a purified bacterial population, which comprises one or more bacteria having a 16S rDNA sequence that is at least 97%, at least 97.5%, at least 98%, at least 98.5%, at least 99%, at least 99.5%, or 100% identical to a 16S rDNA sequence set forth in SEQ ID NOs: 190, 191, 192, 193, 194, 200, 201, 202, 203, 204, 214, 215, 216, 178, 197, 263, 102, 104, 179, 180, 152, 210, 181, 196, 186, 106, 211, 212, 116, 187, or combinations thereof.

Provided herein is a composition comprising a purified bacterial population, which comprises one or more bacteria having a 16S rDNA sequence that is at least 97%, at least 97.5%, at least 98%, at least 98.5%, at least 99%, at least 99.5%, or 100% identical to a 16S rDNA sequence set forth in SEQ ID NOs: 178, 197, 263, 179, 180, 152, 116, 181, 187, or combinations thereof.

Also provided herein is a composition comprising a purified bacterial population, which comprises one or more bacteria having a 16S rDNA sequence that is at least 97%, at least 97.5%, at least 98%, at least 98.5%, at least 99%, at least 99.5%, or 100% identical to a 16S rDNA sequence set forth in SEQ ID NOs: 178, 197, 263, 179, 180, 152, 116, 181, 187, 196, 200, 201, 202, 203, 204, 148, 149, 150, 103, 132, 133, 134, 135, 314, 315, 316, 317, 102, 118, 186, 106, 211, 195, 226, 210, 212, or combinations thereof.

Present disclosure provides a composition comprising a purified bacterial population, which comprises one or more bacteria having a 16S rDNA sequence that is at least 97%, at least 97.5%, at least 98%, at least 98.5%, at least 99%, at least 99.5%, or 100% identical to a 16S rDNA sequence set forth in SEQ ID NOs: 178, 187, 196, 197, 263, 200, 201, 202, 203, 204, 226, 212, 152, 186, 210, 195, 211, 102, 179, 180, 116, 118, 106, 181, or combinations thereof.

In some aspects, a composition disclosed herein further comprises one or more enteric polymers.

Present disclosure also provides pharmaceutical formulation comprising any of the bacterial compositions disclosed herein, and a pharmaceutically acceptable excipient. In some aspects, the excipient is glycerol. In certain aspects, the composition is lyophilized. In further aspects, the composition is formulated for oral delivery.

Provided herein is a method of treating an inflammatory disease in a subject in need thereof, comprising administering to the subject an effective amount of a composition disclosed herein. In certain aspects, administering the effective amount of the composition ameliorates one or more signs or symptoms of the inflammatory disease or maintains a remission of the inflammatory disease. In some aspects, the inflammatory disease comprises an inflammatory bowel disease. In certain aspects, the inflammatory bowel disease comprises Crohn's disease, autoimmune-mediated gastrointestinal diseases, gastrointestinal inflammation, or colitis, such as ulcerative colitis, colitis ulcerosa, microscopic colitis, collagenous colitis, colitis polyposa, necrotizing enterocolitis, transmural colitis, or any combination thereof.

Also provided herein a use of a compositions disclosed herein (e.g., designed bacterial composition) in the manufacture of a medicament for treating an inflammatory disease in a subject in need thereof. Present disclosure also provides a composition disclosed herein for use in a method of treating an inflammatory disease, comprising administering the composition to the subject.

Provided herein is a method of modulating the level of a biological molecule in a subject in need thereof, comprising administering to the subject an effective amount of a composition disclosed herein. In certain aspects, the biological molecule comprises a fecal calprotectin, a secondary bile acid, a tryptophan metabolite, a short-chain fatty acid, a medium-chain fatty acid, a sphingolipid, a kynurenine, or any combination thereof.

In some aspects, the level of fecal calprotectin is reduced by at least about 10%, at least about 20%, at least about 30%, at least about 40%, at least about 50%, at least about 60%, at least about 70%, at least about 80%, or at least about 90% in the subject compared to a corresponding level in a reference.

In certain aspects, the level of a secondary bile acid is increased by at least about 10%, at least about 20%, at least about 30%, at least about 40%, at least about 50%, at least about 60%, at least about 70%, at least about 80%, or at least about 90% in the subject compared to a corresponding level in a reference. In some aspects, the secondary bile acid comprises deoxycholic acid (DCA), 3α 12-oxo-deoxycholic acid, 3β 12α-deoxycholic acid (3-isodeoxycholic acid), 7α 3-oxo-chenodeoxycholic acid, lithocholic acid (LCA), 3-oxo LCA, or any combination thereof.

In some aspects, the level of a tryptophan metabolite is increased by at least about 10%, at least about 20%, at least about 30%, at least about 40%, at least about 50%, at least about 60%, at least about 70%, at least about 80%, or at least about 90% in the subject compared to a corresponding level in a reference. In some aspects, the tryptophan metabolite is selected from the group consisting of indole, 3-methylindole, and combinations thereof.

In some aspects, the level of a short-chain fatty acid is increased by at least about 10%, at least about 20%, at least about 30%, at least about 40%, at least about 50%, at least about 60%, at least about 70%, at least about 80%, or at least about 90% in the subject compared to a corresponding level in a reference. In certain aspects, the short-chain fatty acid comprises formate, acetate, propionate, butyrate, isobutryate, valerate, isovalerate, or any combination thereof.

In some aspects, the reference is a predetermined level or a level in the subject prior to the administration. In some aspects, the modulation of the biological molecule is associated with remission of an inflammatory disease.

Also provided herein is a method of treating a cancer in a subject in need thereof, comprising administering to the subject an effective amount of a composition of the present disclosure. Present disclosure further provides the use of any of the compositions disclosed herein in the manufacture of a medicament for treating a cancer in a subject in need thereof. Also disclosed is a composition disclosed herein for use in a method of treating a cancer, comprising administering the composition to the subject.

Provided herein is a method for inhibiting a growth of a tumor or reducing the size of a tumor in a subject in need thereof, comprising administering to the subject an effective amount of a composition disclosed herein. Also provided is a use of a composition disclosed herein in the manufacture of a medicament for inhibiting a growth of a tumor or reducing the size of a tumor in a subject in need thereof. Also disclosed herein is a composition of the present disclosure for use in a method of treating a cancer, comprising administering the composition to the subject.

Provided herein is a method of enhancing an immune response in a subject in need thereof, comprising administering to the subject an effective amount of a composition disclosed herein. Also provided herein is a use of a composition of the present disclosure in the manufacture of a medicament for enhancing an immune response in a subject in need thereof. Also disclosed herein is a composition of the present disclosure for use in a method of enhancing an immune response in a subject in need thereof.

In some aspects, the subject has a cancer.

In some aspects, the methods, the use, or the composition for use further comprises administering an additional therapeutic agent to the subject. In certain aspects, the additional therapeutic agent comprises an immune checkpoint inhibitor. In some aspects, the immune checkpoint inhibitor comprises an anti-PD-1 antibody, an anti-PD-L1 antibody, or an anti-CTLA-4 antibody.

In some aspects, the cancer comprises a bladder cancer, breast cancer, uterine/cervical cancer, ovarian cancer, prostate cancer, testicular cancer, esophageal cancer, gastrointestinal cancer, pancreatic cancer, colorectal cancer, colon cancer, kidney cancer, head and neck cancer, lung cancer, stomach cancer, germ cell cancer, bone cancer, liver cancer, thyroid cancer, skin cancer, neoplasm of the central nervous system, lymphoma, leukemia, myeloma, sarcoma, virus-related cancer, or any combinations thereof.

In some aspects, administering a composition disclosed herein to a subject results in increased number of tumor infiltrating lymphocytes in a tumor of the subject. In some aspects, the number of tumor infiltrating lymphocytes in the tumor is increased by at least about 5%, at least about 10%, at least about 20%, at least about 30%, at least about 40%, at least about 50%, at least about 60%, at least about 70%, at least about 80%, or at least about 90% or more compared to a reference. In some aspects, the reference comprises the number of tumor infiltrating lymphocytes in a tumor of a subject that did not receive the composition.

### BRIEF DESCRIPTION OF THE DRAWINGS/FIGURES

FIG. 1 shows a comparison of the clinical remission (left graph) and endoscopic improvement (right graph) at 8 weeks post initial treatment in ulcerative colitis patients who received one of the following treatment regimens: (A) placebo pre-treatment/placebo once daily; (B) placebo pre-treatment/purified spore population derived from the feces of healthy human donors (healthy human spore product; HHSP) once weekly; (C) vancomycin pre-treatment/HHSP once weekly; or (D) vancomycin pre-treatment/HHSP once daily. Pretreatment period was 6 days and treatment period was 8 weeks. The percentages of patients from each of the groups who went into clinical remission (Total Modified Mayo (TMM) score of ≤2 plus endoscopic subscore of ≤1) or showed endoscopic improvement (decrease in endoscopic score of ≥1) are shown above the respective bars.

FIGS. 2A to 2C show a comparison of the number of “high confidence engrafting bacteria” species associated with HHSP detected in the fecal samples of ulcerative colitis patients from each of the 4 Arms (A, B, C, and D). In FIG. 2A, the total number of the relevant species of bacteria that engrafted were quantified in fecal samples at days 0, 3, 7, 10, 14, 56, and 84 after initiation of treatment with either placebo or an HHSP. In FIGS. 2B and 2C, the engrafting bacterial species were further divided into either long-term engrafting species (long-term engrafters) (FIG. 2B) or transient engrafting species (transient engrafters) (FIG. 2C). Engraftment was determined relative to the population of bacteria present at baseline (i.e., prior to the pre-treatment regimen). High confidence engrafting bacteria comprise species present in the drug product (i.e., HHSP) and not present in the pre-treatment baseline sample for an individual patient, but were observed in the patient at any time point post-treatment. This is a conservative measure of engraftment in that it does not include engraftment of a species that is present as a unique strain in the drug product and as a different strain of the same species in the patient microbiome at baseline.

FIG. 3 shows a comparison of the change in the spore-forming portion of the microbiome of ulcerative colitis patients from Arms A, B, C, and D, at various time points post initial dose of the HHSP. The change in the microbiome from the baseline composition is shown as a binary Jaccard distance between patients and their matched dose lot. Binary Jaccard measures the similarity of the spore-forming component of patient microbiomes to HHSP. A positive value indicates greater similarity to HHSP. The horizontal line indicates the composition of the spore-forming component of the patient microbiome at baseline (distance=0 by definition).

FIG. 4 shows a correlation between the concentrations of 7-α-dehydroxylated secondary bile acids and clinical outcome. At 8 weeks post initial treatment, ulcerative colitis patients from all treatment arms were categorized as being in remission or in non-remission. Then, the concentrations of the 7-α-dehydroxylated secondary bile acids were measured.

FIGS. 5A and 5B show the effects of secondary bile acids deoxycholic acid (DCA) and lithocholic acid (LCA) on the production of TNF-α (FIG. 5A) and IL-10 (FIG. 5B) in LPS-stimulated peripheral blood mononuclear cells (PBMCs) in vitro. In both FIGS. 5A and 5B, the bars shown correspond to a concentration of the bile acid used (12.5, 25, and 50 μM), with increase in concentration going from left to right.

FIGS. 6A, 6B, and 6C show a comparison of different tryptophan metabolite levels in the fecal samples of remitters (Remission) and non-remitters (Non-Remission) after HHSP administration (i.e., Arms B, C, and D) at 8 weeks post initial dosing (i.e., at the end of the treatment period). FIG. 6A shows a comparison of the indole level. FIG. 6B shows a comparison of the 3-methylindole level. FIG. 6C also shows a comparison of the 3-methylindole level, but the patient samples were divided based on the presence of Ruminococcus bromii and Eubacterium siraeum: (i) none “(0)”, (ii) one (i.e., either of the two species) “(1)”, or (iii) both “(2)”.

FIGS. 7A and 7B shows a comparison of the ability of different tryptophan metabolites (FIG. 7A) or bacterial supernatants (FIG. 7B) to induce AhR-mediated cyp1a1 expression relative to β-actin in epithelial colonic organoids. In FIG. 7A, the metabolites (3-indole acetic acid, 3-methylindole, indole, indoleacrylate, 3-indole butyric acid, and indolepropionic acid, IPA) were added at three different concentrations (50, 100, and 200 μM), with increasing concentrations from right to left. Untreated epithelial organoids (Untd) were used as a negative control. In FIG. 7B, supernatants were collected from cultures containing different bacteria (Clostridium sporogenes 1, Clostridium sporogenes 2, Peptostreptococcus stomatis, Clostridium glycolicum, Bacteroides sp. 4 1 36) and were provided to the epithelial organoids at two different concentrations (5% and 2% final concentration), with the left bar corresponding to the higher concentration. The SCFAs and tryptophan metabolites present in each supernatant (from FIGS. 17 and 18) are indicated. IPA: indolepropionic acid; IAcryl: Indole acrylate; 3Mind: 3-methylindole; I3Carb: indole-3-carbinol; C3: propionate; C4: butyrate; C5: valerate; C6: hexanoate; BCFA: branch chain fatty acids.

FIG. 8A provides a schematic diagram of the epithelial barrier integrity assay and FIG. 8B provides a comparison of the epithelial permeability after exposure to different concentrations of IFN-γ.

FIGS. 9A and 9B show a comparison of the ability of different bacterial metabolites (butyrate, propionate, and IPA) (FIG. 9A) and different bacterial species (FIG. 9B) to restore barrier integrity in the presence of IFN-γ, as measured by the epithelial barrier integrity assay shown in FIG. 8A. In FIG. 9A, each of the metabolites tested was added to the assay at four different concentrations (right to left: 0.625 mM, 1.25 mM, 5 mM, and 10 mM). Untreated samples (i.e., no metabolite, no IFN-γ) were used as a negative control. Samples treated with 5 ng/mL of IFN-γ alone (no metabolite) were used as a positive control. The dotted horizontal line represents the permeability of the negative control. Permeability values below the dotted line indicate barrier protection while values above represent additional barrier damage compared to that caused by INF-7 alone (no bacteria). In FIG. 9B, the culture supernatants of different bacterial species tested included Escherichia coli, Acidaminococcus sp. D21, Bacteroides fragilis, Collinsella intestinalis., Bifidobacterium bifzdum, Peptoniphilus harei (15% final supernatant concentration). Untreated samples (i.e., no bacteria, no IFN-γ) were used to measure the barrier permeability in the absence of IFN-γ driven barrier defect. Butyrate (5 mM) was added as a positive control as it is known to enhance epithelial barrier junction integrity via multiple mechanisms. Under these assay conditions, addition of 5 mM butyrate was known to decrease permeability by 50%.

FIG. 10 shows the treatment schedule for assessing the effect of spore-forming bacteria on ulcerative colitis in an adoptive T cell transfer animal model.

FIG. 11 shows a comparison of the total pathology score in the ulcerative colitis animal model after treatment with (i) antibiotics alone (ABX), (ii) an HHSP, or (iii) DE1 (a composition of 14 spore forming human commensal species obtained by axenic fermentation). Naïve animals and untreated disease animals (Disease) were used as negative and positive controls, respectively. All comparisons were made to the ABX arm. “**” indicates a p value of <0.01 compared to the antibiotics alone control. “***” indicates a p value of <0.001 compared to the antibiotics alone control.

FIGS. 12A, 12B, 12C, 12D, and 12E show a comparison of mRNA expression level measured by qPCR of different genes from the lamina propria of colons in the ulcerative colitis animal model after treatment with one of the following: (i) antibiotics alone (ABX), (ii) HHSP or (iii) DE1. Naïve animals, untreated disease animals (Disease) and ABX only animals were used as controls. FIGS. 12A and 12B show the expression level of the pro-inflammatory genes, Il1b and TNFa, respectively. FIGS. 12C, 12D, and 12E show the expression level of different epithelial tight junction protein molecules, Tjp1, Tjp2, and Ocln, respectively. In FIGS. 12A, 12B, 12C, 12D, and 12E, the mRNA expression level of the different genes is shown relative to GAPDH expression. Statistical comparisons are to ABX only animals.

FIG. 13 provides a table showing the ability of different bacterial strains to inhibit histone deacetylate (HDAC) activity. The bacterial strains tested were grown in PY medium supplemented with one of seven different nutrient sources at 0.5% final concentration (glucose, fucose, sucrose, pectin, fos/inulin, starch, or mucin). HDAC inhibition activity is shown as a fraction compared to media only controls (HDACi=1-(HDACsample/HDACmedium control). If a strain exhibits HDACi activity of at least 0.25 in any nutrient, or 0.18 in fucose, it is considered to have HDACi activity and it is marked with “1”. Strains that do not pass the cutoff are indicated by “0”. The different bacterial strains are categorized into 7 different clusters (0 to 6) based on the pattern of HDAC inhibition activity across nutrient sources (far right column).

FIGS. 14A and 14B show the ability of different bacterial metabolites (FIG. 14A) or a supernatant of a healthy human spore preparation (HHSP) (FIG. 14B) to inhibit IL-8 secretion by HT29 epithelial cells (IECs) after stimulation with TNF-α. In FIG. 14A, the SCFAs of butyrate (left set of bars), propionate (middle set of bars), and acetate (right set of bars) show a dose-dependent anti-inflammatory effect on IECs shown as percent IL-8 inhibition compared to TNF-α only control. FIG. 14B, shows a dose-dependent anti-inflammatory effect of supernatant of a HHSP culture shown as a decrease in the level of IL-8 protein produced by the IECs after TNF-α treatment. IECs that were either not stimulated with TNF-α or TNF-α alone were used as controls (negative and positive controls, respectively).

FIGS. 15A and 15B show the relationship between HDAC inhibition (x-axis) and anti-inflammatory effects in IECs (as measured by the relative decrease in IL-8 production after TNF-α stimulation) using supernatants from different bacterial species. Each circle represents a separate supernatant from a bacterial strain/nutrient combination as shown in FIG. 13. Positive y-axis values indicate anti-inflammatory activity. Negative y-axis values indicate higher IL-8 production than the TNF-α only control. FIG. 15A shows a general positive correlation between HDAC inhibition and anti-inflammatory activity (dashed line), although some supernatants had significantly lower anti-inflammatory activity than expected by HDAC. FIG. 15B separates data points with pro-inflammatory activity in a separate assay (increased IL-8 secretion in the absence of TNF-α stimulation). In these supernatants, HDAC inhibition did not translate into anti-inflammatory activity in IECs.

FIG. 16 shows the relationship between HDAC inhibition (x-axis) and Wnt activation (y-axis) in HEK-293 Wnt-STF (as measured by luciferase activity after bacterial supernatant stimulation) using supernatants from different bacterial species. Each circle represents a separate supernatant from a bacterial strain/nutrient combination as shown in FIG. 13.

FIG. 17 provides phenotypic screening results of multiple strains of a single Lachnospiraceae species. Each row corresponds to a unique strain, and each column corresponds to an in vitro screening phenotype. A dark shade indicates that the strain is positive for the particular phenotype; a light shade indicates that a strain is weakly positive for the phenotype; and white indicates the strain is negative. The different in vitro screening phenotypes include bile acid activities (bile salt hydrolase (BSH), hydroxysteroid dehydrogenase (HSDH), 7α-dehydroxylase) and pro-inflammatory effects (as measured by production of IL-8 by IECs when exposed to a culture supernatant from the individual strain).

FIG. 18 provides a table listing bacterial species and the short chain fatty acids (SCFAs), medium chain fatty acids (MCFAs), and branched chain fatty acids (BCFAs) produced by each of the species. “<LOD” indicates that the concentration of the fatty acid was less than the limit of detection. The limit of detection for each of the fatty acids is provided in the row labeled “Limit of Detection (LOD).” The SCFAs measured included: acetic acid, propanoic acid, and butanoic acid. The MCFAs measured included: pentanoic acid, hexanoic acid, heptanoic acid. The BCFAs measured included: 2-methyl-propanopic acid, 3-methyl-butanoic acid, and 4-methyl-pentaoic acid.

FIG. 19 provides a table listing bacterial species and tryptophan metabolites produced by the species. “<LOD” indicates that the concentration of the fatty acid was less than the limit of detection. The limit of detection for each of the fatty acids is provided in the row labeled “Limit of Detection (LOD).” The tryptophan metabolites measured included: indole, 3-methylindole, indol-3-propanoic acid, indole-3-butyric acid, 3-indoleacrylic acid, tryptamine, indole-3-acetic acid, 3-indole-glycoxylic acid, 2-picolinic acid, and 5-hydroxytryptamine.

FIGS. 20A to 20T provide a comparison of various functional attributes of eight DEs disclosed herein after they were cultured in vitro: (1) DE1 (DE286037.1); (2) DE3 (DE984662.1); (3) DE4 (DE002165.1); (4) DE5 (DE464167.1); (5) DE6 (DE522292.1); (6) DE7 (DE247030.1); (7) DE8 (DE349441.1); and (8) DE9 (DE821956.1). The following functional attributes are shown: (i) biomass (FIG. 20A); (ii) ability to inhibit HDAC activity (FIG. 20B); (iii) ability to inhibit IL-8 secretion by HT29 epithelial cells (IECs) after stimulation with TNF-α (FIG. 20C); (iv) ability to induce IL-8 production by IECs (FIG. 20D); (v) ability to restore barrier integrity in the presence of IFN-γ, as measured by the epithelial barrier integrity assay (FIG. 20E); (vi) ability to express catalase activity (FIG. 20F); (vii) ability to activate toll-like receptor 4 (TLR4) (FIG. 20G); (viii) ability to activate TLR5 (FIG. 20H); (ix) ability to produce butyrate (FIG. 20I); (x) ability to produce propionate (FIG. 20J); (xi) ability to produce valerate (FIG. 20K); (xii) ability to produce hexanoate (FIG. 20L); (xiii) ability to produce indole (FIG. 20M); (xiv) ability to downmodulate the transcription of CXCL1, CXCL2, CXCL3, and CXCL11 (pro-inflammatory cytokines expressed in ulcerative colitis (UC) patients) in epithelial colonic organoids (FIGS. 20N, 200, 20P, and 20Q, respectively); and (xv) ability to activate the Wnt signaling pathway, as determined by both CD44 and LRP6 gene expression, and HEK-293 Wnt-STF reporter assay (FIGS. 20R, 20S, and 20T, respectively).

FIGS. 21A to 21Q provide a comparison of various functional attributes of fourteen additional DEs disclosed herein after they were cultured in vitro: (1) DE1 (DE286037.1); (2) DE6 (DE522292.1); (1) DE10 (DE698478.1); (2) DE1 (DE559846.1); (3) DE12 (DE405816.1); (4) DE13 (DE056280.1); (5) DE14 (DE390874.1); (6) DE15 (DE299561.1); (7) DE16 (DE504874.1); (8) DE17 (DE608959.1); (9) DE18 (DE124702.1); (10) DE19 (DE211714.1); (11) DE20 (DE313669.1); (12) DE21 (DE762708.1); (13) DE22 (DE787951.1); and (14) DE23 (DE291114.1). For comparison purposes, DE1 and DE6 were included. The following functional attributes are shown: (i) biomass (FIG. 21A); (ii) ability to inhibit HDAC activity (FIG. 21); (iii) ability to inhibit IL-8 secretion by HT29 epithelial cells (IECs) after stimulation with TNF-α (FIG. 21C); (iv) ability to restore barrier integrity in the presence of IFN-γ, as measured by the epithelial barrier integrity assay (FIG. 21D); (v) ability to induce IL-8 production by IECs (FIG. 21E); (vi) ability to activate TLR4 (FIG. 21F); (v) ability to activate TLR5 (FIG. 21G); (vii) ability to produce butyrate (FIG. 21H); (viii) ability to produce propionate (FIG. 21I); (ix) ability to produce valerate and hexanoate (FIGS. 21J and 21K, respectively); (x) ability to produce indole and 3-methyl indole (FIGS. 21L and 21M, respectively); (x) bile salt hydrolase activity (as measured by the amount of primary bile acids produced) (FIG. 21N); and (xi) 7α-dehydroxylase, α-hydroxysteroid dehydrogenase, and 7β-hydroxysteroid dehydrogenase activity (as measured by the amount of different secondary bile acids produced) (FIGS. 21N, 21O, and 21P, respectively). In FIGS. 21B to 21E, DE9 (DE821956.1), which was designed not to be anti-inflammatory, was used as a negative control.

FIGS. 22A to 22R provide a comparison of various functional attributes of twelve different DEs disclosed herein after they were cultured in vitro: (1) DE24 (DE070875.1); (2) DE26 (DE343482.1); (3) DE25 (DE616787.1); (4) DE30 (DE068851.1); (5) DE28 (DE055548.1); (6) DE27 (DE033849.1); (7) DE29 (DE865106.1); (8) DE32 (DE779249.1); (9) DE33 (DE433598.1); (10) DE31 (DE502105.1); (11) DE34 (DE266386.1); and (12) DE35 (DE278442.1). As negative controls, DE9 and DE38 (DE533175.1) were used. As described herein, DE9 and DE38 are bacterial compositions that were designed to not have one or more of the functional properties disclosed herein (e.g., anti-inflammatory activity). The following functional attributes are shown: (i) biomass (FIG. 22A); (ii) ability to inhibit HDAC activity (FIG. 22B); (iii) anti-inflammatory activity (as measured by the ability to inhibit IL-8 secretion by HT29 epithelial cells (IECs) after stimulation with TNF-α (FIG. 22C); (iv) pro-inflammatory activity (as measured by the ability to induce IL-8 production by IECs) (FIG. 22D); (v) ability to restore barrier integrity in the presence of IFN-γ, as measured by the epithelial barrier integrity assay (FIG. 22E); (vi) ability to produce butyrate (FIG. 22F); (vii) ability to produce valerate (FIG. 22G); (viii) ability to produce hexanoate (FIG. 22H); (ix) ability to produce indole (FIG. 22I); (x) ability to produce 3-methyl indole (FIG. 22J); (xi) bile salt hydrolase activity (as measured by the amount of primary bile acids produced) (FIG. 22K); (xii) 7α-dehydroxylase activity (as measured by the amount of deoxycholic acid (DCA) and lithocholic acid (LCA) secondary bile acids produced) (FIG. 22L); (xiii) α-HSDH activity (as measured by the amount of oxo-secondary bile acids produced) (FIG. 22M); (xiv) ability to downmodulate the transcription of CXCL1 and ICAM1 (proteins associated with pro-inflammatory response) in epithelial colonic organoids (FIGS. 22N and 22P, respectively); (xv) ability to increase AhR-mediated Cyp1a1 expression in epithelial colonic organoids (FIG. 22O) (xvi) ability to activate TLR4 (FIG. 22Q); and (xvii) ability to activate TLR5 (FIG. 22R).

FIGS. 23A to 23Q provide comparison of various functional attributes of DEs disclosed herein after they were cultured in vitro: (1) DE37 (DE935045.1), (2) DE39 (DE935045.2), (3) DE9 (DE821956.1), and (4) DE916091.1. In FIGS. 23L-230, HHSP (“Pilot lot 20”) is included for comparison purposes. The following functional attributes are shown: (i) ability to inhibit HDAC activity (FIG. 23A); (ii) ability to produce acetate (FIG. 23B); (iii) ability to produce propionate (FIG. 23C); (iv) ability to produce butyrate (FIG. 23D); (v) ability to produce valerate (FIG. 23E); (vi) ability to produce hexanoate; (vii) ability to produce indole (FIG. 23G); (viii) ability to produce 3-methyl indole (skatole) (FIG. 23H); (ix) ability to activate TLR4 (FIG. 23I); (x) ability to activate TLR5 (FIG. 23J); (xi) ability to restore barrier integrity in the presence of IFN-γ, as measured by the epithelial barrier integrity assay (FIG. 23K); (xii) ability to downmodulate the production of MCP-1 in the presence of IFN-γ in epithelial colonic organoids (FIG. 23L); (xiii) ability to downmodulate the production of MCP-1 and IP10 in the presence of TNF-α in epithelial colonic organoids (FIGS. 23M and 23N, respectively); (xiv) ability to inhibit IL-8 production by HT29 epithelial cells (IECs) after stimulation with TNF-α (FIG. 23O and FIG. 23P); and (xv) ability to stimulate IL-8 production by HT29 epithelial cells (IECs) in the absence of TNF-α (FIG. 23Q).

FIGS. 24A to 24H provide comparison of additional properties (e.g., functional features) of DEs disclosed herein to FMT (fecal microbiota transplantation) and HHSP (spore-prep composition). In FIGS. 24A to 24D, both DE1 (DE286037.1) and DE2 (DE924221.1) are compared to FMT and HHSP. In FIGS. 24E to 24H, DE1 is compared to HHSP. The different properties shown include: (i) biomass (FIG. 24A); (ii) inhibition of HDAC activity (FIG. 24B); (iii) pro-inflammatory activity (FIG. 24C); (iv) anti-inflammatory activity (FIG. 24D); (v) valerate production (FIG. 24E); (vi) hexanoate production (FIG. 24F); (vii) indole production (FIG. 24G); and (viii) 3-methyl indole (skatole) production (FIG. 24H).

FIGS. 25A and 25B shows on x-axis the differential gene expression observed in colonic biopsies in subjects with IBD compared to subjects without IBD in the HMP2 database; on the y-axis shows differential gene expression in colonic organoids when exposed to media alone compared to media plus TNFα; each point corresponds to a gene measured in vitro in colonic organoids and in colonic biopsies of human subjects. Each point is based on the change in gene expression when colonic organoids are exposed to supernatant from cultured HSSP, a spore preparation from healthy donors (24A, left) or from DE1 (DE286037.1) (24B, right). Only genes that were differentially expressed in organoids after treatment with TNFα (p<0.05) are shown. Ligher shaded points represent genes that were differentially expressed both in organoids after TNFα treatment and HMP2, and were not significantly changed by treatment with bacterial supernatants. Darker shaded points represent genes that were differentially expressed both in organoids after TNFα treatment and HMP2, and responded to bacterial supernatant treatment (i.e. their expression was elevated in organoids treated with TNF and lowered with supernatant treatment, or if their expression was decreased in organoids treated with TNF but increased with supernatant treatment).

FIGS. 26A to 26C provide a comparison of DE1, FMT, and HHSP in their ability to downmodulate the transcription of TNF-α-mediated CXCL1 (FIG. 26A), CXCL3 (FIG. 26B), and ICAM1(FIG. 26C) expression in epithelial colonic organoids. For FMT, two of the samples were from a healthy donor (FMT #1 and FMT #3) and one sample was from a patient with ulcerative colitis (FMT #2). “Media (+)” (media with TNF-α) and “Media (−)” (media alone, no TNF-α) were used as positive and negative controls, respectively.

FIGS. 27A and 27B provide a comparison of the different DEs disclosed herein to FMT and DXE (HHSP) in their ability to produce indole and butyrate, respectively.

FIGS. 28A to 28C show the efficacy of the combination of DE1 and anti-PD-1 antibody in treating MC38 tumor in an animal model. FIG. 28A shows the treatment schedule. All of the animals were treated with the DE1 composition. Some of the animals additionally received the anti-PD-1 antibody, while the control animals received an isotype control antibody. FIG. 28B shows a comparison of tumor volume in the animals from the different treatment groups from days 6 to 17 post tumor inoculation. FIG. 28C provides a comparison of the percentage of CD8 T cells (left graph) and CD8 T cell:Treg ratio (right graph) in the tumors of the animals from the different treatment groups.

FIGS. 29A to 29C show the efficacy of the combination of DE2 and anti-PD-1 antibody in treating MC38 tumor in an animal model. Overall treatment schedule is the same as in FIG. 28A. Instead of DE1, the animals were treated with the DE2 composition. Some of the animals additionally received the anti-PD-1 antibody, while the control animals received an isotype control antibody. FIG. 29A shows a comparison of tumor volume in the animals from the different treatment groups from days 6 to 17 post tumor inoculation. FIGS. 29B and 29C provide a comparison of the percentage of CD8 T cells and CD8 T cell:Treg ratio, respectively, in the tumors of the animals from the different treatment groups.

FIGS. 30A to 30E show the efficacy of the combination of DE1 and anti-PD-1 antibody in treating BP tumor in an animal model. FIG. 30A shows the treatment schedule. All of the animals were treated with the DE1 composition. Some of the animals additionally received the anti-PD-L1 antibody, while the control animals received an isotype control antibody. FIG. 30B shows a comparison of tumor volume in the animals from the different treatment groups over a course of 15 days from tumor inoculation. FIGS. 30C, 30D, and 30E show a comparison of the percentage of CD8 T cells, CD8 T cell:Treg ratio, and percentage of CD4 T cells, respectively, in the tumors of the animals from the different treatment groups.

FIG. 31 provides a table identifying the bacterial species included in the designed compositions DE1-DE9. SEQ ID NOs for the 16S sequences of the bacterial species are also provided. “0” indicates that the bacterial species is not included; “1” indicates that the bacterial species is included in the given composition.

FIG. 32 provides a table identifying the bacterial species included in the designed compositions DE10-DE23. SEQ ID NOs for the 16S sequences of the bacterial species are also provided. “0” indicates that the bacterial species is not included; “1” indicates that the bacterial species is included in the given composition.

FIG. 33 provides a table identifying the bacterial species included in the designed compositions DE24-DE42. SEQ ID NOs for the 16S sequences of the bacterial species are also provided. “0” indicates that the bacterial species is not included; “1” indicates that the bacterial species is included in the given composition.

FIG. 34 provides a table identifying the bacterial species included in the designed compositions DE43-DE56. SEQ ID NOs for the 16S sequences of the bacterial species are also provided. “0” indicates that the bacterial species is not included; “1” indicates that the bacterial species is included in the given composition.

FIGS. 35A to 35F show the effect of different bacterial compositions on the expression of inflammation pathways as measured in IFN-γ treated epithelial colonic organoids using a 770 gene Human Autoimmune panel (Nanostring). The bacterial compositions tested include: (1) DE821956.1 (DE9); (2) DE935045.1 (DE37); (3) DE935045.2 (DE39); and (4) HHSP (see Example 1). Media with IFN-γ alone (“IFN”) and media alone (i.e., no IFN and bacterial composition) (“media”) were used as positive and negative controls, respectively. Pathway scores, representing a high-level view of gene expression changes for each pathway, were obtained using NSolver software Advanced analysis. Scores were Z-normalized. Higher score represents higher overall expression of the pathways. FIG. 35A shows the effect on chemokine signaling. Non-limiting examples of Individual chemokine genes that were assessed are shown in FIG. 41A. FIG. 35B shows the effect on NF-κB signaling. FIG. 35C shows the effect on TNF family signaling. FIG. 35D shows the effect on type I interferon signaling. FIG. 35E shows the effect on type II interferon signaling. FIG. 35F shows the effect on TLR signaling.

FIGS. 36A to 36D show the effect of different bacterial compositions on the expression of genes associated with lymphocyte trafficking (FIG. 36A) and genes associated with differentiation of Th17, Th1, and Th2 T cells (FIGS. 36B, 36C, and 36D, respectively) as measured in IFN-γ treated epithelial colonic organoids. Pathway scores representing a high-level view of gene expression changes for each pathway were obtained using NSolver software Advanced analysis. Scores were Z-normalized. Higher score represents higher overall expression of the pathways. The bacterial compositions tested include: (1) DE821956.1 (DE9); (2) DE935045.1 (DE37); (3) DE935045.2 (DE39); and (4) HHSP. Media with IFN-γ alone (“IFN”) and media alone (i.e., no IFN and bacterial composition) (“media”) were used as positive and negative controls, respectively.

FIGS. 37A to 37D show the effect of different bacterial compositions on the expression of genes associated with apoptosis (FIG. 37A), inflammasomes (FIG. 37B), autophagy (FIG. 37C), and oxidative stress (FIG. 37D) as measured in IFN-γ treated epithelial colonic organoids. The bacterial compositions tested include: (1) DE821956.1 (DE9); (2) DE935045.1 (DE37); (3) DE935045.2 (DE39); and (4) HHSP. Media with IFN-7 alone (“IFN”) and media alone (i.e., no IFN and bacterial composition) (“media”) were used as positive and negative controls, respectively. Pathway scores representing a high-level view of gene expression changes for each pathway were obtained using NSolver software Advanced analysis. Scores were Z-normalized. Higher score represents higher overall expression of the pathways.

FIGS. 38A and 38B show the effect of different bacterial compositions on the expression of genes associated with MHC class I and II antigen presentation, respectively, as measured in IFN-γ treated epithelial colonic organoids. The bacterial compositions tested include: (1) DE821956.1 (DE9); (2) DE935045.1 (DE37); (3) DE935045.2 (DE39); and (4) HHSP. Media with IFN-γ alone (“IFN”) and media alone (i.e., no IFN and bacterial composition) (“media”) were used as positive and negative controls, respectively. Pathway scores representing a high-level view of gene expression changes for each pathway were obtained using NSolver software Advanced analysis. Scores were Z-normalized. Higher score represents higher overall expression of the pathways.

FIGS. 39A, 39B, and 39C show the effect of different bacterial compositions on the expression of genes associated with complement system (FIG. 39A), mTOR (FIG. 39B), and nod-like receptors (NLR) (FIG. 39C) as measured in IFN-γ treated epithelial colonic organoids. The bacterial compositions tested include: (1) DE821956.1 (DE9); (2) DE935045.1 (DE37); (3) DE935045.2 (DE39); and (4) HHSP. Media with IFN-γ alone (“IFN”) and media alone (i.e., no IFN and bacterial composition) (“media”) were used as positive and negative controls, respectively. Pathway scores representing a high-level view of gene expression changes for each pathway were obtained using NSolver software Advanced analysis. Scores were Z-normalized. Higher score represents higher overall expression of the pathways.

FIGS. 40A and 40B show correlation between gene-level changes in various pairwise comparisons of bacterial compositions effects as measured in IFN-γ treated epithelial colonic organoids. Log 2 changes in gene expression in the HHSP+IFN-γ vs. IFN-γ alone comparison was plotted against one of the following: (i) DE935045.2 (DE39)+IFN-γ vs. IFN-γ alone treatment (FIG. 40A); or (ii) DE821956.1 (DE9) (i.e., negative control)+IFN-γ vs. IFN-γ alone treatment (FIG. 40B). Each dot represents an individual gene in the Human Autoimmune Panel (Nanostring). The linear fit equation and R² value for each of the correlations are depicted for the respective linear trendlines.

FIGS. 41A and 41B show the ability of different bacterial compositions to modulate the transcription of various disease relevant individual genes as measured in IFN-γ treated epithelial colonic organoids. The bacterial compositions tested include: (1) DE935045.1 (DE37); (2) DE935045.2 (DE39); (3) DE821956.1 (DE9); and (4) HHSP. Media with IFN-7 alone (“IFN”) and media alone (i.e., no IFN and bacterial composition) (“media”) were used as positive and negative controls, respectively. FIG. 41A shows the effect on the transcription of different genes involved in cytokine signaling. FIG. 41B shows the effect on the transcription of different genes involved in apoptosis, antigen presentation (MHC class I and II presentation pathways), and PI3K signaling. The effect on transcription of the different genes is shown as the average (“AVRG”) normalized counts and standard deviation as measured using NSolver software Advanced analysis.

FIG. 42 shows the similarity between reversal of inflammatory pathway level gene expression changes observed in colonic organoids treated with various bacterial compositions in the presence of IFN-γ and those observed in patient colonic biopsies in the phase 1B clinical trial described in Example 1. The bacterial compositions tested include: (1) DE935045.2 (DE39); (2) DE821956.1 (DE9); and (3) HHSP. In this experiment, organoid gene expression was assessed by RNASeq and compared to gene expression changes in colonic biopsies of HHSP Phase 1b clinical trial (see, e.g., Example 1), Remitters vs Non Remitters at the end of the induction period (visit 12). Pathway enrichment analysis was performed on differential gene expression data using the R package for fast pre-ranked gene set enrichment analysis (fgsea v 1.10.1). Data for select list of disease-relevant KEGG pathways is shown. For each pathway, normalized enrichment score (NES) and P-values are shown for the indicated pairwise comparison's.

FIGS. 43A to 43H show the effects of different bacterial compositions on the expression of genes involved in lymphocyte activation (FIG. 43A), metabolism (FIG. 43B), cell cycle and apoptosis (FIG. 43C), cytokine signaling (FIG. 43D), chemokine signaling (FIG. 43E), interferon signaling (FIG. 43F), TLR signaling (FIG. 43G), and antigen presentation (FIG. 43H) as measured in human macrophages treated with 1% bacterial supernatants. The bacterial compositions tested include: (1) DE821956.1 (DE9), (2) DE935045.2 (DE39), (3) HHSP #1, (4) HHSP #2, and (5) HHSP #3. Bacterial broth (“broth”) and media alone (i.e., no bacterial composition) (“media”) were used as controls.

FIGS. 44A to 44C provide a comparison of the viability of macrophages treated with one of the following: (1) media alone; (2) bacterial broth (i.e., bacterial culture medium in which all bacterial communities were grown); (3) DE935045.2 (DE39); (4) HHSP #1 (PNP167020), (5) HHSP #2 (PNP167021), or (6) HHSP #3 (PNP167022). The bacterial compositions were added to the macrophages as 1% culture supernatant (FIG. 44A), 1% culture supernatant plus multiplicity of infection (MOI) 20 bacterial cells (FIG. 44B), or MOI20 bacterial cells (FIG. 44C). Viability of the macrophages are shown as the amount of ATP produced by the macrophages (normalized to macrophages treated with media alone).

FIGS. 45A to 45C provide a comparison of the IL-10/IL-6 production ratio in macrophages treated with one of the following: (1) media alone; (2) bacterial broth (i.e., bacterial culture medium in which all bacterial communities were grown); (3) DE935045.2 (DE39); (4) HHSP #1 (PNP167020), (5) HHSP #2 (PNP167021), or (6) HHSP #3 (PNP167022). The bacterial compositions were added to the macrophages as 1% culture supernatant (FIG. 45A), 1% culture supernatant plus multiplicity of infection (MOI) 20 bacterial cells (FIG. 45B), or MOI20 bacterial cells (FIG. 45C).

FIGS. 46A to 46E provide a comparison of IL-6 production in macrophages treated with either DE935045.2 (DE39) composition or complex healthy bacterial spore-preparations (HHSP) (PNP167020, PNP167021, PNP167022). Macrophages treated with media alone (“media”) or bacterial broth (i.e., bacterial culture medium in which all bacterial communities were grown) were used as control. FIGS. 46A and 46B show the effect on IL6 gene transcription in macrophages treated with 1% bacterial culture supernatant (FIG. 46A) or 1% bacterial supernatant and a multiplicity of infection (MOI) 20 bacterial cells (FIG. 46B). FIGS. 46C, 46D, and 46E show the effect on IL-6 protein production from macrophages treated with 1% supernatant (FIG. 46C), 1% supernatant plus MOI20 bacterial cells (FIG. 46D), or MOI20 bacterial cells alone (FIG. 46E).

FIGS. 47A to 47E provide a comparison of TNF-α production in macrophages treated with either DE935045.2 (DE39) composition or complex healthy bacterial spore-preparations (HHSP) (PNP167020, PNP167021, PNP167022). Macrophages treated with media alone or bacterial broth were used as control. FIGS. 47A and 47B show the effect on TNF gene transcription in macrophages treated with 1% bacterial culture supernatant (FIG. 47A) or 1% bacterial supernatant and a multiplicity of infection (MOI) 20 bacterial cells (FIG. 47B). FIGS. 47C, 47D, and 47E show the effect on TNF-α protein production from macrophages treated with 1% supernatant (FIG. 47C), 1% supernatant plus MOI20 bacterial cells (FIG. 47D), or MOI20 bacterial cells alone (FIG. 47E).

FIGS. 48A to 48E provide a comparison of IL-1 production in macrophages treated with either DE935045.2 (DE39) composition or complex healthy bacterial spore-preparations (HHSP) (PNP167020, PNP167021, PNP167022). Macrophages treated with media alone or bacterial broth were used as control. FIGS. 48A and 48B show the effect on IL1B gene transcription in macrophages treated with 1% bacterial culture supernatant (FIG. 48A) or 1% bacterial supernatant and a multiplicity of infection (MOI) 20 bacterial cells (FIG. 48B). FIGS. 48C, 48D, and 48E show the effect on IL-1β protein production from macrophages treated with 1% supernatant (FIG. 48C), 1% supernatant plus MOI20 bacterial cells (FIG. 48D), or MOI20 bacterial cells alone (FIG. 48E).

FIGS. 49A to 49E provide a comparison of IL-23 production in macrophages treated with either DE935045.2 (DE39) composition or complex healthy bacterial spore-preparations (HHSP) (PNP167020, PNP167021, PNP167022 Macrophages treated with media alone or bacterial broth were used as control. FIGS. 49A and 49B show the effect on IL23 gene transcription in macrophages treated with 1% bacterial culture supernatant (FIG. 49A) or 1% bacterial supernatant and a multiplicity of infection (MOI) 20 bacterial cells (FIG. 49B). FIGS. 49C, 49D, and 49E show the effect on IL-23 protein production from macrophages treated with 1% supernatant (FIG. 49C), 1% supernatant plus MOI20 bacterial cells (FIG. 49D), or MOI20 bacterial cells alone (FIG. 49E).

FIGS. 50A to 50C provide a comparison of IL12 gene transcript expression in macrophages treated with 1% bacterial supernatant (FIG. 50A), 1% bacterial supernatant and MOI20 bacterial cells (FIG. 50B), or MOI20 bacterial cells alone (FIG. 50C). The bacterial supernant and cells were derived from DE935045.2 (DE39) composition or complex healthy bacterial spore-preparations (HHSP) (PNP167020, PNP167021, PNP167022). Macrophages treated with bacterial broth or media alone were used as control.

FIGS. 51A to 51Q show the therapeutic effects of different bacterial compositions in an IL-10 knockout (KO) animal model. FIG. 51A provides a schematic of the experimental design. As shown, the animals were colonized with one of the following: (1) DE935045.2 (DE39); (2) DE916091.1; or (3) FMT from UC patients. FIGS. 51B and 51C provide comparison of body weight and fecal lipocalin levels over a course of 7 weeks in the animals from the different groups. FIGS. 51D, 51E, and 51F provide histological score based on the amount of inflammatory damage observed in the ileum, cecum, and proximal colon, respectively, of the animals from the different groups. FIG. 51G provides a comparison of the total frequency of CD4+ T cells within the colon of the animals from the different treatment groups. FIGS. 51H, 51I, and 51J provide comparison of the frequency of different Treg populations in the animals from the different groups. FIGS. 51K and 51L provide comparison of the frequency of Th17 and Th1 effector cells, respectively, in the animals from the different groups. FIG. 51M shows the frequency of total CD8+ T cells, and FIG. 51N shows the frequency of activated CD8+ T cells (based on IFN-γ production) in the animals from the different groups. FIGS. 51O, 51P, and 51Q provide the ratio of peripheral colonic Tregs to Th1 cells, Th17 cells, and total CD8+ T cells, respectively.

FIGS. 52A to 52H show the therapeutic effects of different bacterial compositions in a DSS-induced colitis mouse model. FIG. 52A provides a schematic of the experimental design. As shown, the animals were colonized with one of the following: (1) DE935045.2 (DE39); (2) DE935045.1 (DE37); or (3) DE916091.1 (a pro-inflammatory composition).

FIGS. 52B, 52C, and 52D provide comparison of the frequency of total CD4+ T cells, activated CD4+ T cells (based on IFN-γ production), and Th17 cells, respectively, within the colon of the animals from the different groups. FIGS. 52E and 52F provide comparison of the frequency of total Tregs and peripheral colonic Tregs, respectively, in the animals from the different groups. FIGS. 52G and 52H provide the ratio of the peripheral colonic Tregs to Th1 cells and Th17 cells, respectively.

FIGS. 53A to 53C show the effect of bacterial compositions on the anti-tumor efficacy of the combination of anti-PD-1 antibody and anti-CTLA-4 antibody treatment in a mouse MC38 tumor model. FIG. 53A provides a schematic of the administration schedule. FIG. 53B provides a comparison of the tumor volume at various time points post antibody administration. FIG. 53C provides a comparison of the frequency of IFNγ+CD8+ Tcells in the tumor draining lymph nodes of the animals from the different treatment groups at the terminal time-point (i.e., at day 19 post initial antibody administration).

FIG. 54 provides a table showing the effect of two different designed compositions (i.e., DE935045.2 (DE39) and DE821956.1 (DE9)) on the expression of various gene pathways involved in T cell function as measured using a Nanostring gene expression panel. Bacterial media was used as control. Each test article (i.e., DE935045.2, DE821956.1 and the bacterial media) was tested in duplicate. Values represent pathway scores as determined by NSolved Advanced software and provide a high-level overview of the changes in gene expression for the measured genes in each pathway. An increase in value represents greater expression of genes associated with the particular T cell function.

FIGS. 55A to 55F show the expression of several genes associated with activation and/or effector activity in T cells stimulated with α-CD3 and α-CD28 beads in combination with one of the following: (1) bacterial media, (2) DE916091.1, (3) DE821956.1 (DE9), (4) DE935045.2 (DE39), (5) HHSP #1, (6) HHSP #2, and (7) HHSP #3. The expression of the following genes are shown: CD45RA (FIG. 55A), CD45RO (FIG. 55B), CD69 (FIG. 55C), IL-24 (FIG. 55D), TNF-α (FIG. 55E), and perforin (FIG. 55F). The expression of the CD45RA and CD45RO genes were measured using a Nanostring gene expression panel. The expression of the CD69, IL-24, TNF-α, and perforin genes were measured using a custom design Nanostring multiplex panel.

FIGS. 56A to 56E show the expression of different inhibitory receptors in T cells stimulated with α-CD3 and α-CD28 beads in combination with one of the following: (1) bacterial media, (2) DE916091.1, (3) DE821956.1 (DE9), and (4) DE935045.2 (DE39). Naïve T cells (i.e., not stimulated with either the α-CD3 and α-CD28 beads or the bacterial compositions) were used as control. The expression of the following genes are shown: PD-1 (FIG. 56A), CTLA-4 (FIG. 56B), TIGIT (FIG. 56C), TIM-3 (FIG. 56D), and LAG-3 (FIG. 56E).

FIGS. 57A to 57C show the ability of different bacterial compositions to induce IFN-γ production in T cells. The T cells were stimulated with α-CD3 and α-CD28 beads in combination with one of the following: (1) bacterial media, (2) DE916091.1, (3) DE821956.1 (DE9), (4) DE935045.2 (DE39), (5) HHSP #1, (6) HHSP #2, and (7) HHSP #3. Naïve T cells (i.e., not stimulated with either the α-CD3 and α-CD28 beads or the bacterial compositions) were used as control. FIG. 57A shows the effect of the different stimulation on the transcription of IFNg as measured using Nanostring. FIG. 57B shows the effect of the different stimulation on IFN-γ protein production as measured using flow cytometry. FIG. 57C shows the effect of the different stimulation on IFN-γ protein production as measured in the culture supernatant using Luminex.

FIG. 58 shows the effect of different bacterial compositions on the ability of CD8 T cells to kill tumor cells as measured in an in vitro CD8 cytotoxicity assay. HT29 cells were either cultured alone or with CD8 T cells that were activated as described in Example 15, in the presence of the following: (1) bacterial media; (2) DE916091.1; (3) DE821956.1 (DE9); and (4) DE935045.2 (DE39). Viability of the target HT29 cells was determined by flow cytometry.

FIGS. 59A to 59E provide comparison of the amount of secondary bile acids produced by the following bacterial compositions: (1) DE935045.2 (DE39); (2) HHSP #1; (3) HHSP #2; (4) HHSP #3; (5) DE821956.1 (DE9); and (6) DE916091.1. Each of the designed bacterial compositions were run in triplicate. Media alone (“Media”) was used as a negative control. The bile acids measured include: deoxycholic acid (DCA) (FIG. 59A), 12-oxo-cholic acid (12-oxo-3a) (FIG. 59B), 3-oxo-chenodeoxycholic acid (3-oxo-7a) (FIG. 59C), 3β 12α-deoxycholic acid (3b 12a) (FIG. 59D), and ursodeoxycholic acid (UDCA) (FIG. 59E).

### DETAILED DESCRIPTION OF DISCLOSURE

Applicant has discovered that bacterial compositions comprising certain species of commensal bacteria exhibit certain functional features (e.g., those disclosed herein) and that such compositions can be used to treat and/or prevent a range of diseases and disorders, e.g., those associated with dysbiosis of the intestinal microbiome. Accordingly, Applicant has identified species of commensal bacteria that can be combined to design bacterial compositions disclosed herein. Detailed disclosure of the bacterial species and the functional features of interest are provided in the present disclosure.

### I. Bacterial (Microbiome) Compositions

Bacteria discovered to be associated with certain functional features (e.g., those described herein) can be used to design therapeutic compositions (e.g., bacterial compositions) for treating and/or preventing a range of diseases and disorders, such as those associated with dysbiosis of the intestinal microbiome. Such compositions can include material directly derived from feces of healthy humans. The compositions comprising material directly derived from human feces can, in some cases, contain spore-forming bacteria (SFB) derived from human feces as the sole type of bacteria present in the composition. In other aspects, such compositions can comprise spores as the sole type of bacteria present in the composition (healthy human spore product; HHSP). Collectively, SFB and HHSP are referred to herein as “spore compositions.” Examples of HHSPs described in the present application include HHSP #1 (also referred to herein as PNP167020), HHSP #2 (also referred to herein as PNP167021), and HHSP #3 (also referred to herein as PNP167022). Each of these HHSP compositions were derived from a different healthy human donor. In some aspects, the HHSP is that described in, e.g., Examples 1-4.

In some cases, one or more bacteria associated with improvement in a disease or disorder (e.g., inflammatory disease) can be combined to produce the designed compositions (DEs) disclosed herein. In certain aspects, one or more bacteria associated with certain functional features of interest (e.g., those described herein) can be combined in the bacterial compositions disclosed herein. By combining different bacterial species disclosed herein, the designed compositions disclosed herein can target different biological pathways. Not to be bound by any particular theory, such ability allows the designed compositions disclosed herein to be useful for the treatment of a wide range of diseases and disorders, e.g., those associated with a dysbiosis of the intestinal microbiome (e.g., those described herein). Species in a designed composition can be spore-formers (in some cases, in spore form), non-spore formers, or a combination thereof. Species in a designed composition can include material directly derived from feces of healthy humans or such compositions can include material fermented from bacterial cultures, including a biologically pure culture. Collectively, spore compositions and designed compositions are referred to herein as “microbiome compositions.” Applicants have therefore discovered that efficacious microbiome compositions can be manufactured and/or designed based on a combination of identified features.

Accordingly, provided herein are bacteria and combinations of bacteria useful for treating and/or preventing one or more signs or symptoms of a disease or disorder associated with dysbiosis of the gastrointestinal microbiome, e.g., ulcerative colitis. In general, such compositions include one or more of the bacteria described herein as exhibiting one or more of the functional features of interest disclosed herein (e.g., associated with remission in UC or having one or more features associated with remission in UC).

In some aspects, the amount, level, identity, presence, and/or ratio of bacteria in the microbiome (e.g., gastrointestinal microbiome) of a subject is manipulated to treat, prevent, delay, or ameliorate one or more signs or symptoms of a disease or disorder associated with dysbiosis of the gastrointestinal microbiome (e.g., an IBD, such as ulcerative colitis, or a cancer).

The term “microbial engraftment” or “engraftment” refers to the establishment of OTUs (bacterial species or strains) comprising a therapeutic microbial composition, e.g., a bacterial composition, in a target niche that are absent or undetectable in a treated subject prior to treatment. The microbes comprising the engrafted ecology are present in the therapeutic microbial composition and establish as constituents of the subject's microbial ecology. Engrafted OTUs can establish for a transient period of time, or demonstrate long-term stability in the microbial ecology that populates the subject post treatment with a therapeutic microbial composition. Without committing to any theory, the drug product (i.e., bacterial compositions disclosed herein) may catalyze a shift from a dysbiotic ecology to one representative of a healthy state, either by engraftment of drug product species, promoting ecological conditions favorable for the growth of non-product commensal microbes present in the patient (augmentation), or both.

As used herein, engraftment is indicated by one or more of the following outputs: (i) strain level engraftment, (ii) species-level population engraftment, (iii) species-level subject engraftment, and (iv) putative engraftment. “Strain level engraftment” is determined using any relevant method known in the art. In some aspects, strain level engraftment is determined using an assay in which single nucleotide variant (SNV) frequencies unique to the drug product composition are used to determine whether strains of species detected in treated subjects are significantly more similar to strains in the composition compared to strains of species detected in subjects prior to treatment. Strain level engraftment is measured on a per-subject and per-species basis. Non-limiting examples of other methods of determining strain level engraftment include the use of probes, e.g., nanostring probes that can be targeted to unique regions of the strain genome, relative to other known genomic sequences of the same species, or compared to metagenomics datasets from healthy subjects; or specific PCR probes for the particular species or strain of interest. “Species-level population engraftment” refers to significantly increased prevalence (p<=0.05) of a species in treated subjects relative to non-treated subjects at any post-treatment time point as measured with a Fisher's exact test, with the requirement that the species was not detected in treated subjects prior to treatment but was detected in the composition. Species-level population engraftment is a population-level measure and requires a significant (p<=0.05) difference across the population treated with a particular regimen compared to placebo. “Species-level subject engraftment” refers to the detection of a species present in the HHSP in a subject post-treatment when said species was not detected pre-treatment in that subject. “Putative engraftment” refers to significantly increased prevalence (p<=0.05) of a species in treated subjects relative to non-treated subjects at any post-treatment time point as measured with a Fisher's exact test. The putative engraftment further requires that the species was detected in the drug product composition and may or may not be present in the treated subject prior to treatment. “Putative engraftment” is a population level statistic. Putative engraftment can be further evaluated using strain level metrics for engraftment.

In some aspects, the term engraftment can be further divided into long-term engraftment and transient engraftment. “Long-term engraftment” refers to the ability of bacterial species or strains disclosed herein to durably reside in the gastrointestinal tracts of subjects after treatment. Such species or strains are described herein as “long-term engrafter” (LTE). In some aspects, long-term engrafters continue to be present in the subject (e.g., in the gastrointestinal tract) for about 4 weeks, about 8 weeks, about 12 weeks or longer after the start of dosing of a bacterial composition disclosed herein. “Transient engraftment” refers to the ability of bacterial species or strains (e.g., those disclosed herein) to reside in the gastrointestinal tracts of subjects after treatment, but are only detected in the fecal samples of subjects for a limited period of time. In some aspects, if bacteria or combinations of bacteria are detected in the fecal sample of a subject, it is generally believed that those bacteria or combinations of bacteria remain present within the gastrointestinal tract. Such species or strains are described herein as “transient engrafter” (TE). In some embodiments, transient-engrafters are detected at one or more time points and not detected at another time point. In some aspects, transient-engrafters are no longer detected in the subject (e.g., no longer detected in the fecal sample of the subject) about 1 week, about 2 weeks, or about 4 weeks after the start of dosing (i.e., administering a bacterial composition disclosed herein. Non-limiting examples of LTEs and TEs are provided in Table 5.

It is a key feature of a microbiome composition (e.g., designed compositions) as provided herein that one or more species or OTUs of bacteria in the microbiome composition engraft in a subject treated with the composition, e.g., a subject that responds to the treatment by an improvement in at least one sign or symptom of the disease being treated. In some aspects, a microbiome composition disclosed herein comprises one or more species or OTUs of bacteria that are long-term engrafters. In other aspects, a microbiome composition comprises one or more species or OTUs of bacteria that are transient engrafters. In certain aspects, a microbiome composition comprises both long-term engrafters and transient engrafters. In certain aspects, a bacterial composition disclosed herein comprises two, three, four, five, six, seven, eight, nine, ten or more long-term engrafters. In some aspects, a bacterial composition comprises two, three, four, five, six, seven, eight, nine, ten or more transient engrafters. In further aspects, a bacterial composition disclosed herein comprises three or more transient engrafters and/or seven or more long-term engrafters.

As used herein, “augmentation” refers to the establishment or significant increase of a population of microbes, or selected species or OTUs, that are (i) absent or undetectable (as determined by the use of known and/or specified genomic or microbiological techniques) in an administered therapeutic microbiome composition, (ii) absent, undetectable, or present at low frequencies in the host niche (as example: gastrointestinal tract (GI tract), skin, anterior-nares, or vagina) before treatment with the microbiome composition compared to after treatment with the microbiome composition, and (iii) are found in the host (subject) after the administration of the microbiome composition or are significantly increased after treatment, for instance about 2-fold, about 5-fold, about 1×10², about 1×10³, about 1×10⁴, about 1×10⁵, about 1×10⁶, about 1×10⁷ fold, or greater than 1×10⁸ fold, in cases where they were present at low frequencies. Microbes comprising an augmented population can be derived from exogenous sources such as food and the environment or grow out from micro-niches within the host where they reside at low frequency. In some aspects of the invention, after treatment with a microbiome composition as provided herein, one or more species or OTUs of bacteria are augmented in the treated subject, e.g., a subject that responds to the treatment by an improvement in at least one sign or symptom of the disease being treated.

Without committing to any theory, administration of a therapeutic microbiome composition may induce a shift in the target niche, e.g., the GI tract, that promotes favorable conditions for the growth of certain commensal microbes causing them to increase in abundance, i.e., they are augmented. In the absence of treatment with a therapeutic microbiome composition, although the host may be exposed to or harbor these commensal microbes, sustained growth and the positive health effects associated with those microbes are not observed or are less frequently observed in a population treated with the microbiome composition.

In some aspects, a bacterial composition comprises a population of bacteria that has been purified from a biological material (e.g., fecal materials, such as feces or materials isolated from the various segments of the small and large intestines) obtained from a mammalian donor subject (e.g., a healthy human). In some aspects, the biological material (e.g., fecal material) is obtained from multiple donors (e.g., 2, 3, 4, 5, 6, 7, 8, 9, 10, 15, 20, 25, 30, 35, 40, 45, 50, 75, 100, 200, 300, 400, 500, 750, 1000, or from greater than 1000 donors), and the materials are pooled prior to purification or after purification of the desired bacteria. In other aspects, the biological material (sample) can be obtained from a single donor subject at multiple times and two or more samples pooled, e.g., 2, 3, 4, 5, 6, 7, 8, 9, 10, 15, 20, 25, 30, 32, 35, 40, 45, 48, 50, 100 samples from a single donor. Methods of making such preparations include treatment of the feces with chloroform, acetone, ethanol, and the like, e.g., see PCT/US2014/014745 and U.S. Pat. No. 9,011,834, which are incorporated herein by reference in their entirety.

In aspects, a microbiome composition derived from feces is depleted in residual habitat products. “Residual habitat products” refers to material derived from the habitat of a microbiota within or on a human or animal excluding the microbiota. An individual's microbiota is in, for example, feces in the gastrointestinal tract, on the skin itself, in saliva, mucus of the respiratory tract, or secretions of the genitourinary tract, all of which contain biological and other matter associated with the microbial community. “Substantially free of residual habitat products” means that the bacterial composition contains a reduced amount of the biological matter associated with the microbial environment on or in the human or animal subject and is about 100% free, about 99% free, about 98% free, about 97% free, about 96% free, or about 95% free of any contaminating biological matter associated with the microbial community or the contaminating matter is below a level of detection. Residual habitat products can include abiotic materials (including undigested food) or it can include unwanted microorganisms. Substantially free of residual habitat products can also mean that the bacterial composition contains no detectable cells from a human or animal and that only microbial cells are detectable. In some aspects, substantially free of residual habitat products can mean that the bacterial composition contains no detectable viral (including bacterial viruses (i.e., phage)), fungal, mycoplasmal contaminants. In other aspects, it means that fewer than about 1×10⁻²%, about 1×10⁻³%, about 1×10⁻⁴%, about 1×10⁻⁵%, about 1×10⁻⁶%, about 1×10⁻⁷%, about 1×10⁻⁸% of the viable cells in the bacterial composition are human or animal, as compared to microbial cells. There are multiple ways to accomplish reduced presence of residual habitat products, none of which are limiting. Thus, contamination can be reduced by isolating desired constituents through multiple steps of streaking to single colonies on solid media until replicate (such as, but not limited to, two) streaks from serial single colonies have shown only a single colony morphology. Alternatively, reduction of contamination can be accomplished by multiple rounds of serial dilutions to single desired cells (e.g., a dilution of about 10⁻⁸ or about 10⁻⁹), such as through multiple 10-fold serial dilutions. This can further be confirmed by showing that multiple isolated colonies have similar cell shapes and Gram staining behavior. Other methods for confirming adequate reduction of residual habitat products include genetic analysis (e.g., PCR, DNA sequencing), serology and antigen analysis, enzymatic and metabolic analysis, and methods using instrumentation such as flow cytometry with reagents that distinguish desired constituents from contaminants.

HHSP Compositions

Generally, in an HHSP composition disclosed herein (as well as other microbiome compositions of the present disclosure), the bacterial material is substantially composed of viable bacterial spores as the live component. In some aspects, in a microbiome composition disclosed herein, the bacterial mixture is substantially composed of viable bacteria in the vegetative-state as the live component. In some aspects, in a microbiome composition disclosed herein, the bacterial mixture is composed of viable bacterial spores and viable bacteria in the vegetative state as the live component.

As used herein, the term “spore” or “endospore” refers to an entity, particularly a bacterial entity, which is in a dormant, non-vegetative and non-reproductive stage. Spores are generally resistant to environmental stress such as radiation, desiccation, enzymatic treatment, temperature variation, nutrient deprivation, oxygen, and chemical disinfectants. In some aspects, a spore or spore population is resistant to 50% ethanol.

A “spore population” refers to a plurality of spores present in a composition. Synonymous terms used herein include spore composition, spore preparation, ethanol treated spore fraction and spore ecology. A spore population can be purified from a fecal donation, e.g., via ethanol or heat treatment, or a density gradient separation or any combination of methods described herein to increase the purity, potency and/or concentration of spores in a sample. Alternatively, a spore population can be derived through culture methods starting from isolated spore former species or spore former OTUs or from a mixture of such species, either in vegetative or spore form.

In some aspects, the spore preparation comprises spore forming species wherein residual non-spore forming species have been inactivated by chemical or physical treatments including ethanol, detergent, heat, sonication, and the like; or wherein the non-spore forming species have been removed from the spore preparation by various separations steps including density gradients, centrifugation, filtration and/or chromatography; or wherein inactivation and separation methods are combined to make the spore preparation. In yet another aspect, the spore preparation comprises spore forming species that are enriched over viable non-spore formers or vegetative forms of spore formers. In this aspect, spores are enriched by about 2-fold, about 5-fold, about 10-fold, about 50-fold, about 100-fold, about 1000-fold, about 10,000-fold or greater than about 10,000-fold compared to all vegetative forms of bacteria. In yet another aspect, the spores in the spore preparation undergo partial germination during processing and formulation such that the final composition comprises spores and vegetative bacteria derived from spore forming species.

The term “germinant” refers to a material or composition or physical-chemical process capable of inducing vegetative growth of a bacterium that is in a dormant spore form, or group of bacteria in the spore form, either directly or indirectly in a host organism and/or in vitro.

The term “sporulation induction agent” refers to a material or physical-chemical process that is capable of inducing sporulation in a bacterium, either directly or indirectly, in a host organism and/or in vitro.

The term “increase production of bacterial spores” includes an activity or a sporulation induction agent. “Production” in this context includes conversion of vegetative bacterial cells into spores and augmentation of the rate of such conversion, as well as decreasing the germination of bacteria in spore form, decreasing the rate of spore decay in vivo, or ex vivo, or to increasing the total output of spores (e.g., via an increase in volumetric output of fecal material).

In some aspects, the preparation of an HHSP (i.e., a spore composition) includes suspending a sample in ethanol, e.g., at least about 30%, at least about 40%, at least about 50%, at least about 55%, at least about 60%, at least about 65%, at least about 70%, at least about 75%, at least about 80%, at least about 85%, at least about 90%, at least about 95%, or at least about 100%. In some cases, the preparation of an HHSP (i.e., a spore composition) includes suspending a sample in about 30 to about 100% ethanol, about 40 to about 80% ethanol, about 50 to about 80% ethanol, about 30% ethanol, about 40% ethanol, about 50% ethanol, about 55% ethanol, about 60% ethanol, about 65% ethanol, about 70% ethanol, about 75% ethanol, about 80% ethanol, about 85% ethanol, about 90% ethanol, about 95% ethanol, or about 100%.

As used herein, the terms “purify”, “purified” and “purifying” refer to the state of a population (e.g., a plurality of known or unknown amount and/or concentration) of desired bacteria or bacterial spores, that have undergone one or more processes of purification, e.g., a selection or an enrichment of the desired bacterium and/or bacterial spores, or alternatively a removal or reduction of residual habitat products as described herein. In some aspects, a purified population has no detectable undesired activity or, alternatively, the level or amount of the undesired activity is at or below an acceptable level or amount. In other aspects, a purified population has an amount and/or concentration of desired bacteria or bacterial spores, e.g., in general or of selected species, at or above an acceptable amount and/or concentration. In other aspects, the ratio of desired-to-undesired activity (e.g., spores compared to vegetative bacteria), has changed by about 2-fold, about 5-fold, about 10-fold, about 30-fold, about 100-fold, about 300-fold, about 1×10⁴, about 1×10⁵, about 1×10⁶, about 1×10⁷, about 1×10⁸, or greater than about 1×10⁸. In other aspects, a purified population of bacterial spores is enriched as compared to the starting material (e.g., a fecal material) from which the population is obtained. This enrichment can be by about 10%, about 20%, about 30%, about 40%, about 50%, about 60%, about 70%, about 80%, about 90%, about 95%, about 96%, about 97%, about 98%, about 99%, about 99.9%, about 99.99%, about 99.999%, about 99.9999%, about 99.9999%, or greater than about 99.9999990% as compared to the starting material.

In some aspects, a purified population of bacteria has reduced or undetectable levels of one or more pathogens (e.g., pathogenic bacteria, viruses, or fungi), or one or more pathogenic activities, such as toxicity, an ability to cause infection of the mammalian recipient subject, an undesired immunomodulatory activity, an autoimmune response, a metabolic response, or an inflammatory response or a neurological response. In some aspects, the pathogen or pathogenic activity of the bacteria is reduced by at least about 5%, at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99% compared to the reference pathogen or bacteria. In some aspects, a purified population of bacteria has reduced sensory components as compared to fecal matter, such as reduced odor, taste, appearance, and umami.

In some aspects, a bacterial composition disclosed herein is substantially free of residual habitat products and/or substantially free of a detectable level of a pathogenic material (e.g., contains no detectable viral (including bacterial viruses (i.e., phage)), fungal, mycoplasmal, or toxoplasmal contaminants, or eukaryotic parasites, such as a helminth; or has an acceptable level of the foregoing. In some aspects, a bacterial composition is substantially free of acellular material (e.g., DNA, viral coat material, or non-viable bacterial material).

Designed Compositions (DEs)

Applicant has discovered that certain families, genera, species, and OTUs of bacteria (e.g., in an HHSP (see, e.g., Examples 1-4) or DEs) are associated with an improvement (e.g., clinical remission) of a disease or disorder associated with dysbiosis of the gastrointestinal microbiome (e.g., ulcerative colitis). Furthermore, some of those families, genera, species, and OTUs were associated with engraftment. In addition, some families, genera, species, and OTUs were not present and/or not detected in a subject suffering from a disease or disorder associated with dysbiosis of the gastrointestinal tract (e.g., in an ulcerative colitis patient) and were augmented in a subject whose disease state was improved after treatment with an HHSP. Such bacteria that are associated with improvement in a subject are useful in compositions for treating a disease or disorder associated with dysbiosis (e.g., an inflammatory disease such as an IBD, e.g., ulcerative colitis, or cancer). Furthermore, applicant has discovered that certain species are negatively associated with an improvement in disease or disorder associated with dysbiosis. In general, such species are not included in a composition useful for treating such diseases. Applicants have further identified families, genera, species, and OTUs of bacteria that exhibit certain functional features that can be useful in treating a wide range of diseases and disorders, including those associated with dysbiosis of the gastrointestinal tract (e.g., inflammatory diseases or cancers).

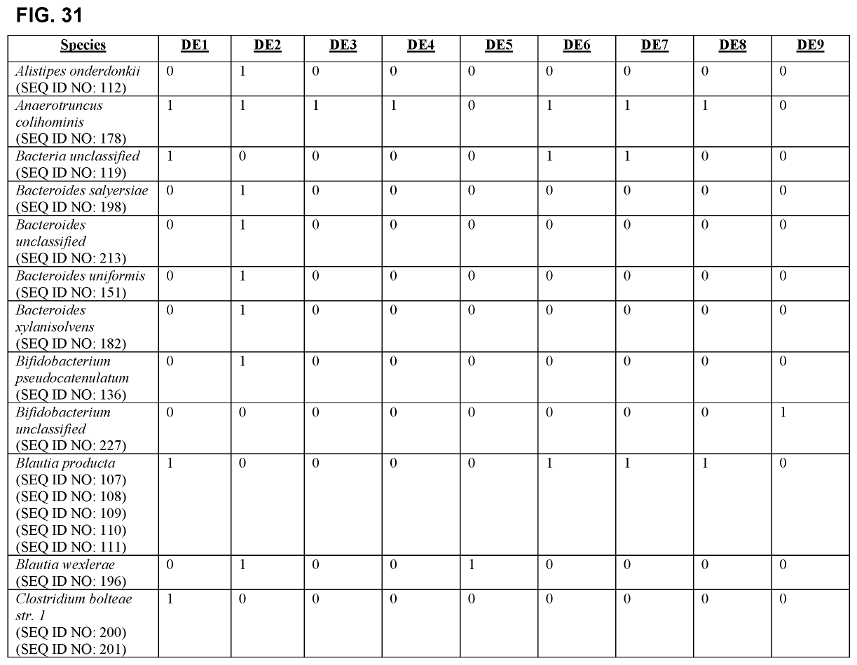

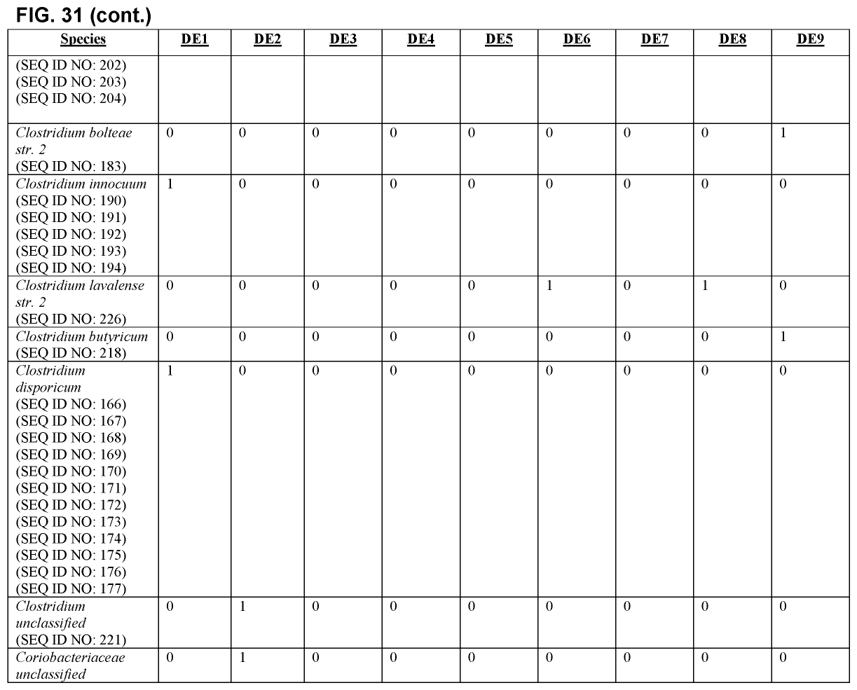

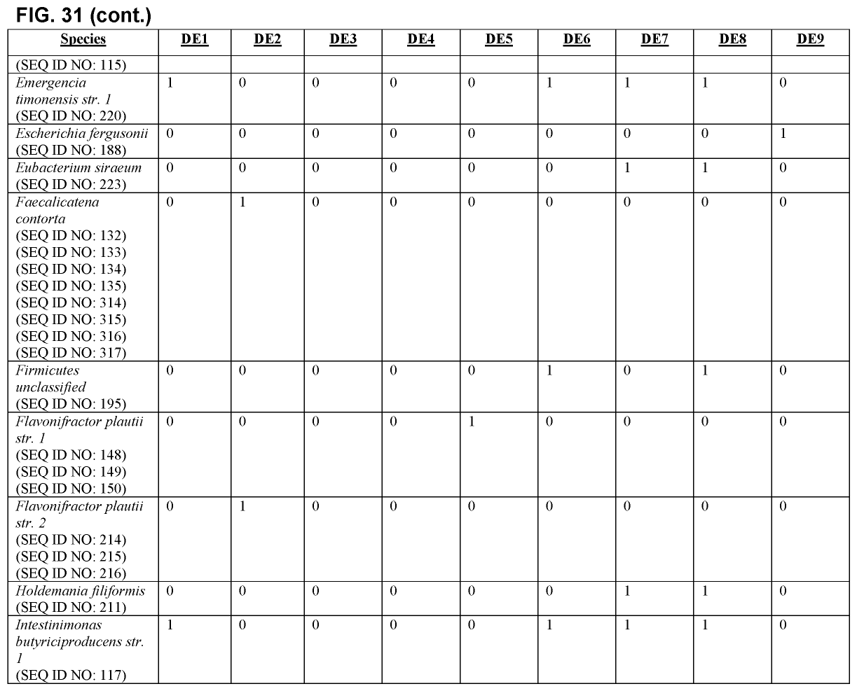

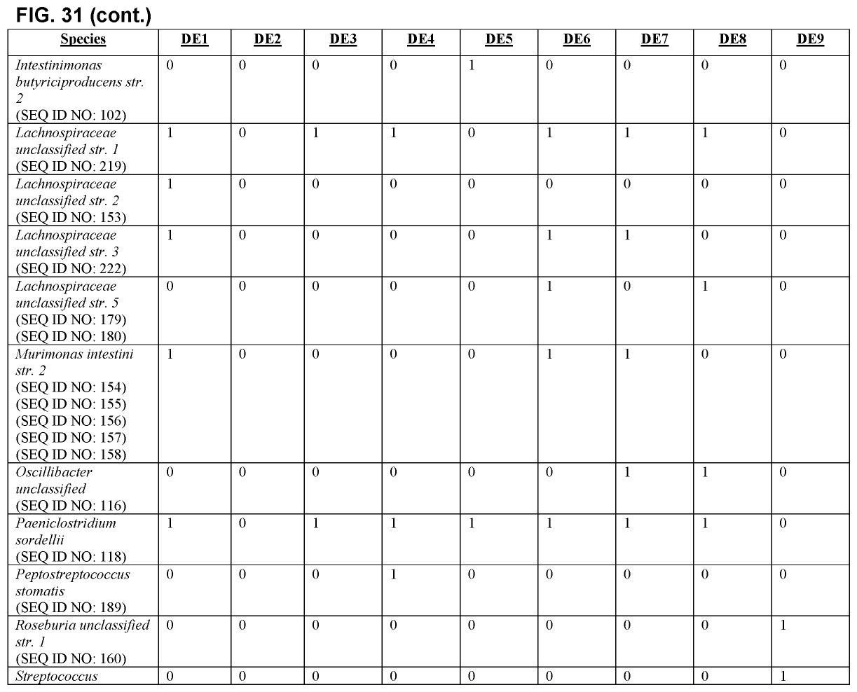

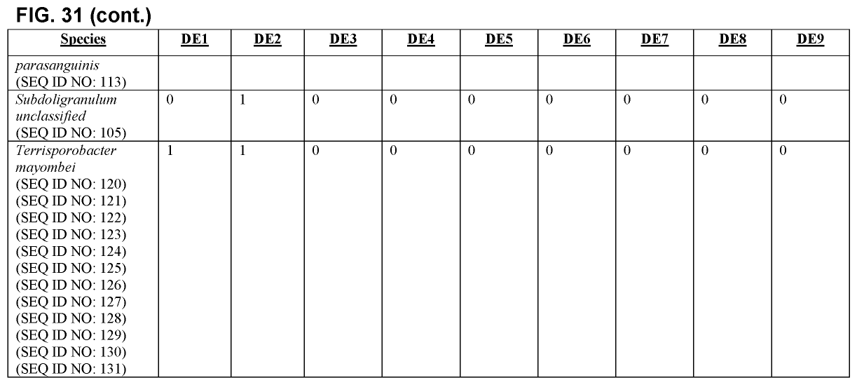

Accordingly, disclosed herein are microbiome compositions that have been designed to exhibit certain features. Non-limiting examples of such features include: (i) capable of engrafting when administered to a subject, (ii) capable of having anti-inflammatory activity, (iii) not capable of inducing pro-inflammatory activity, (iv) capable of producing a secondary bile acid (7α-deydroxylase and bile salt hydrolase activity), (v) not capable of producing ursodeoxycholic acid (7β-hydroxysteroid dehydrogenase activity); (vi) capable of producing a tryptophan metabolite (e.g., indole, 3-methyl indole, indolepropionic acid), (vii) capable of restoring epithelial integrity as determined by a primary epithelial cell monolayer barrier integrity assay, (viii) capable of being associated with remission of an inflammatory bowel disease, (ix) capable of not being associated with clinical non-remission of an inflammatory bowel disease, (x) capable of producing a short-chain fatty acid (e.g., butyrate, propionate), (xi) capable of inhibiting a HDAC activity, (xii) capable of producing a medium-chain fatty acid (e.g., valerate, hexanoate), (xiii) capable of expressing catalase activity, (xiv) capable of having alpha-fucosidase activity, (xv) capable of inducing Wnt activation, (xvi) capable of producing a B vitamin, (xvii) capable of modulating host metabolism of endocannabinoid, (xviii) capable of producing a polyamine and/or modulating host metabolism of a polyamine, (xix) capable of reducing fecal levels of a sphingolipid, (xx) capable of modulating host production of kynurenine, (xxi) capable of reducing fecal calprotectin level, (xxii) not capable of activating a toll-like receptor pathway (e.g., TLR4 or TLR5), (xxiii) capable of activating a toll-like receptor pathway (e.g., TLR2), (xxiv) capable of inhibiting apoptosis of intestinal epithelial cells, (xxv) capable of inducing an anti-inflammatory IL-10-skewed IL-10/IL-6 cytokine ratio in macrophages, (xxvi) capable of not inducing pro-inflammatory IL-6, TNFa, IL-1b, IL-23 or IL-12 production or gene expression in macrophages, (xxvii) capable of downmodulating one or more genes induced in IFN-γ treated colonic organoids (e.g., those associated with inflammatory chemokine signaling, NF-κB signaling, TNF family signaling, type I interferon signaling, type II interferon signaling, TLR signaling, lymphocyte trafficking, Th17 cell differentiation, Th1 differentiation, Th2 differentiation, apoptosis, inflammasomes, autophagy, oxidative stress, MHC class I and II antigen presentation, complement, mTor, nod-like receptor signaling, PI3K signaling, or combinations thereof), (xxix) capable of producing IL-18, (xxx) capable of inducing the activation of antigen presenting cells, (xxxi) capable of reducing the expression of one or more inhibitory receptors (e.g., TIGIT, TIM-3, or LAG-3) on T cells, (xxxii) capable of increasing expression of one or more genes/proteins associated with T cell activation and/or function (e.g., CD45RO, CD69, IL-24, TNF-α, perforin, or IFN-γ), (xxxiii) capable of enhancing the ability of CD8+ T cells to kill tumor cells, (xxxiv) capable of enhancing the efficacy of an immune checkpoint inhibitor therapy, (xxxv) capable of reducing colonic inflammation, (xxxvi) capable of promoting the recruitment of CD8+ T cells to tumors, or (xxviii) any combination thereof. Such microbiome compositions are described herein as “designed compositions” or DEs. Non-limiting examples of designed compositions are described, e.g., in FIGS. 31, 32, 33, and 34. In some aspects, a designed composition disclosed herein comprises one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, or all of the above features. In certain aspects, a designed composition of the present disclosure can comprise features that target multiple biological pathways, such that the same composition can be used to treat a wide range of diseases and disorders.

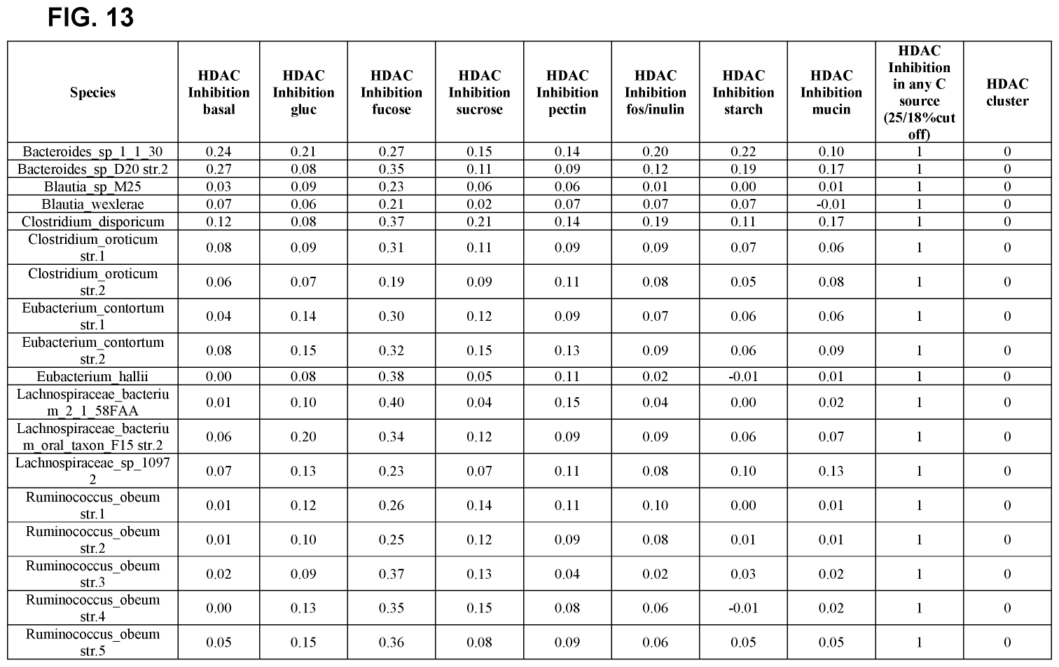

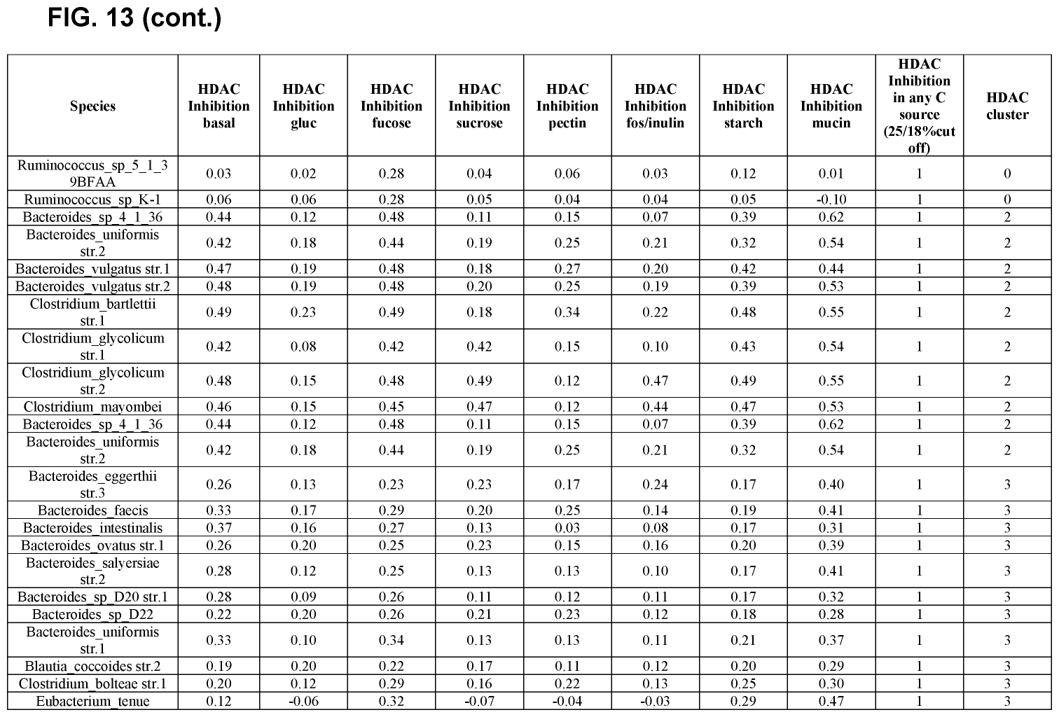

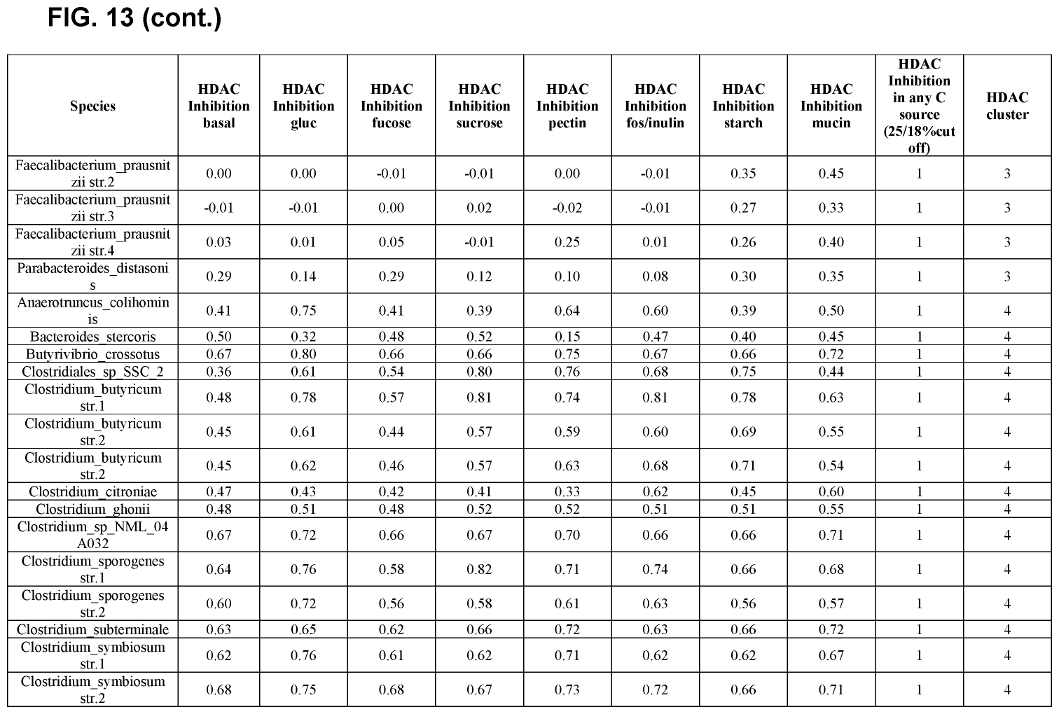

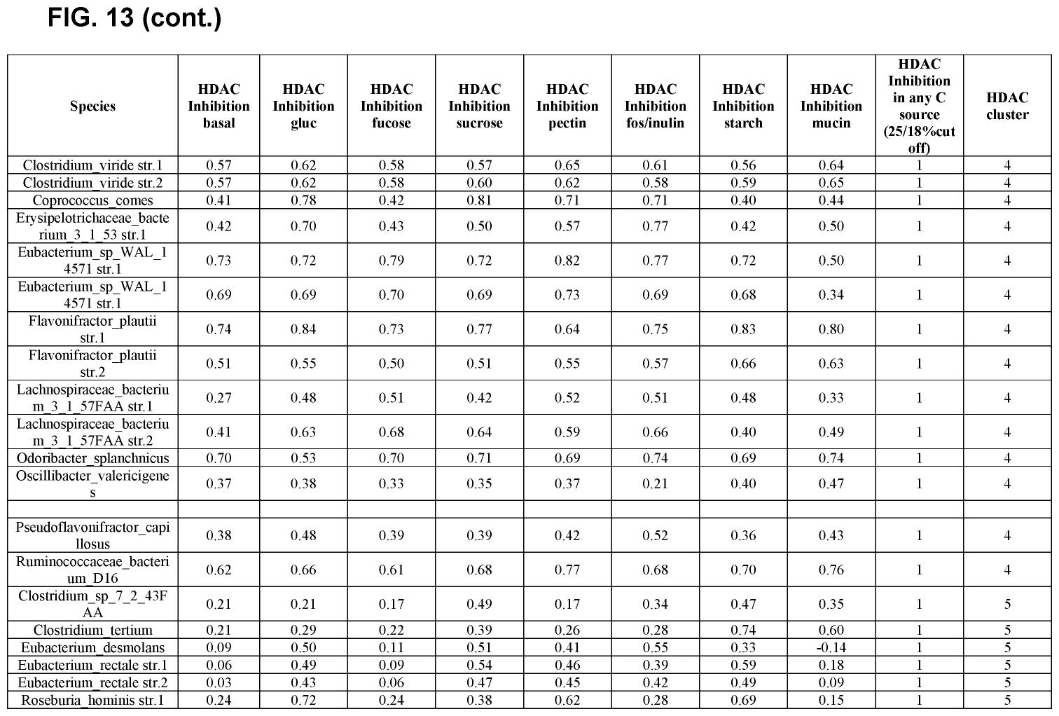

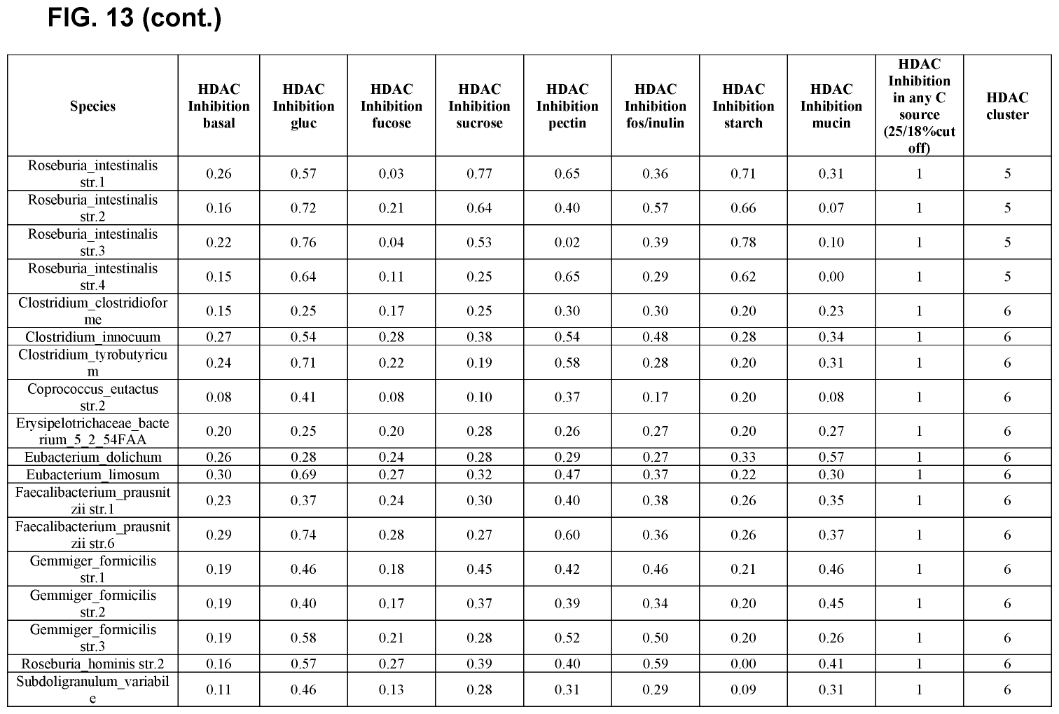

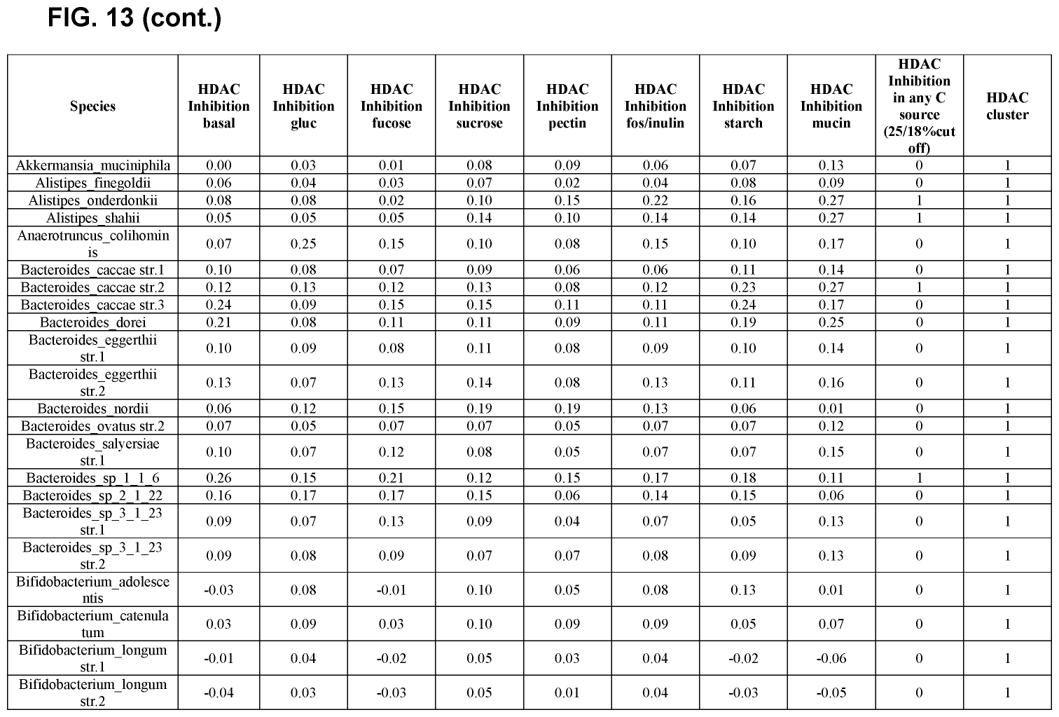

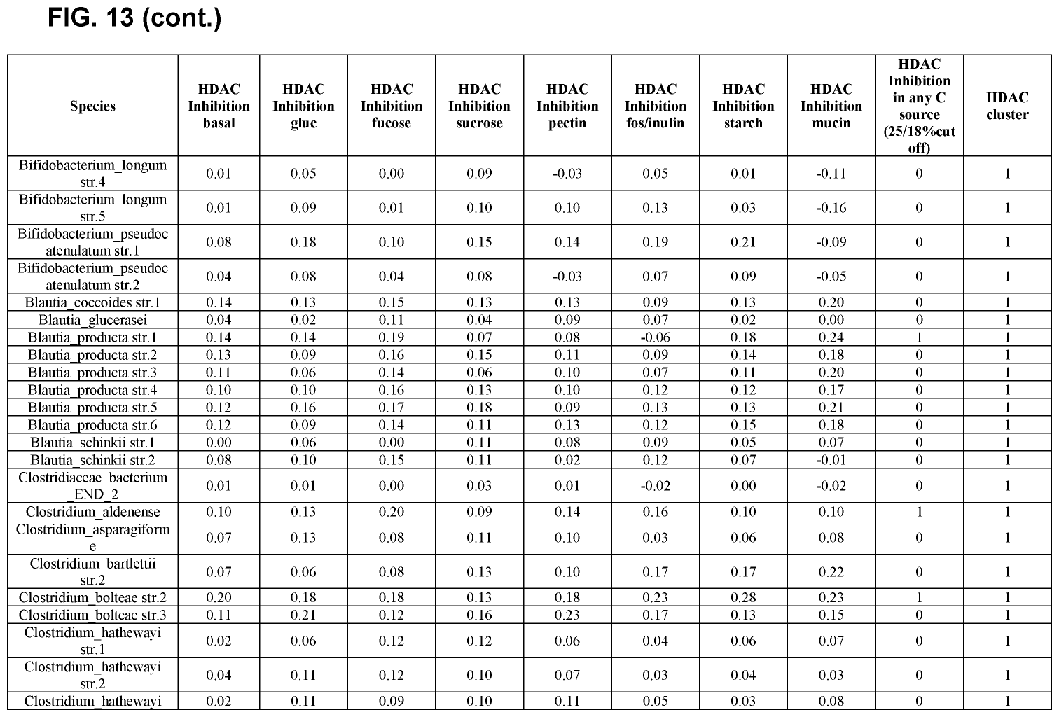

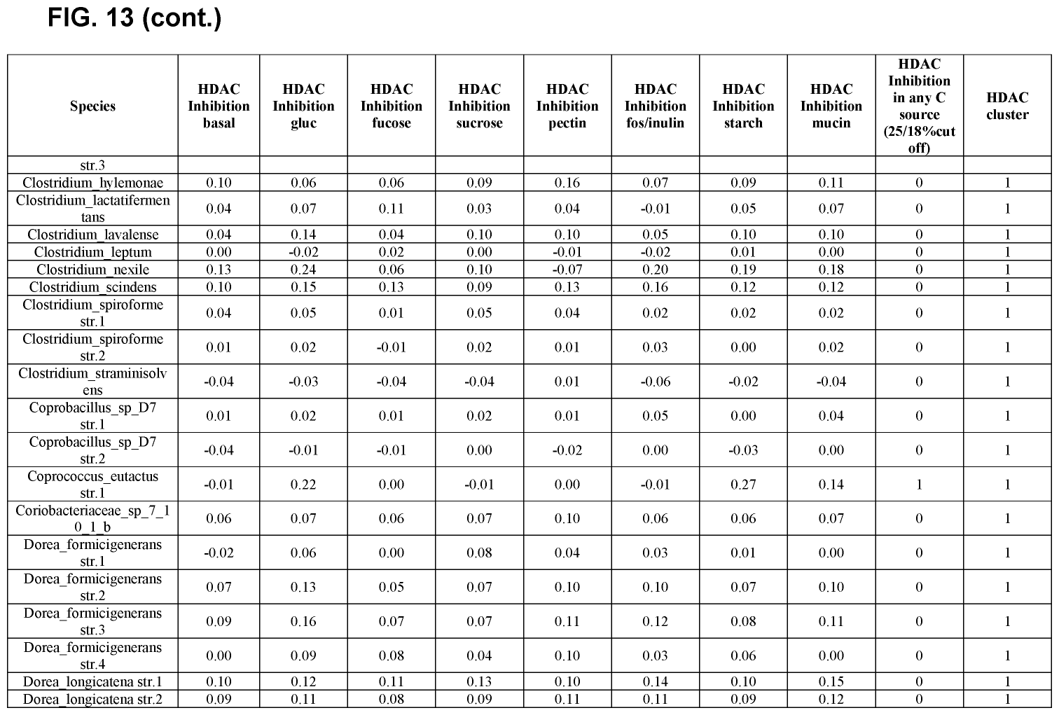

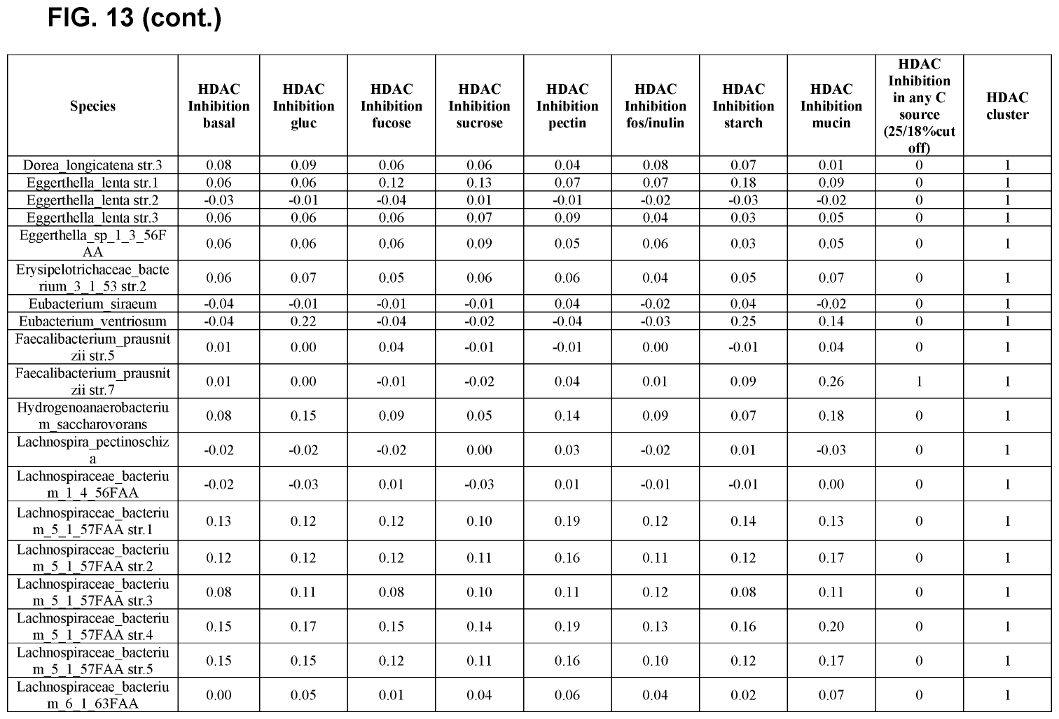

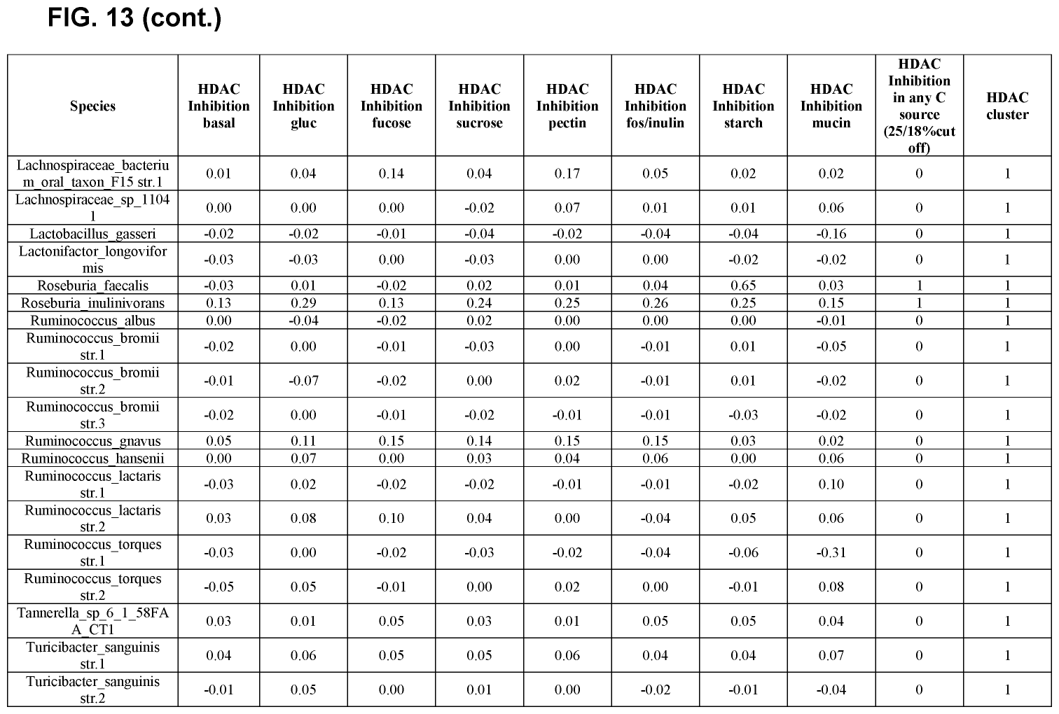

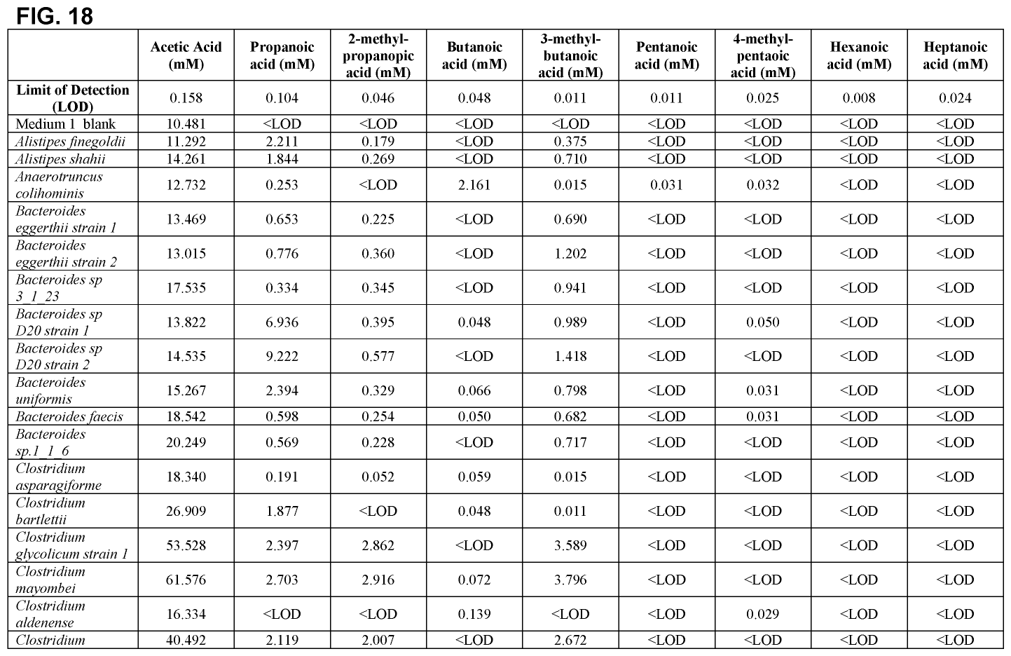

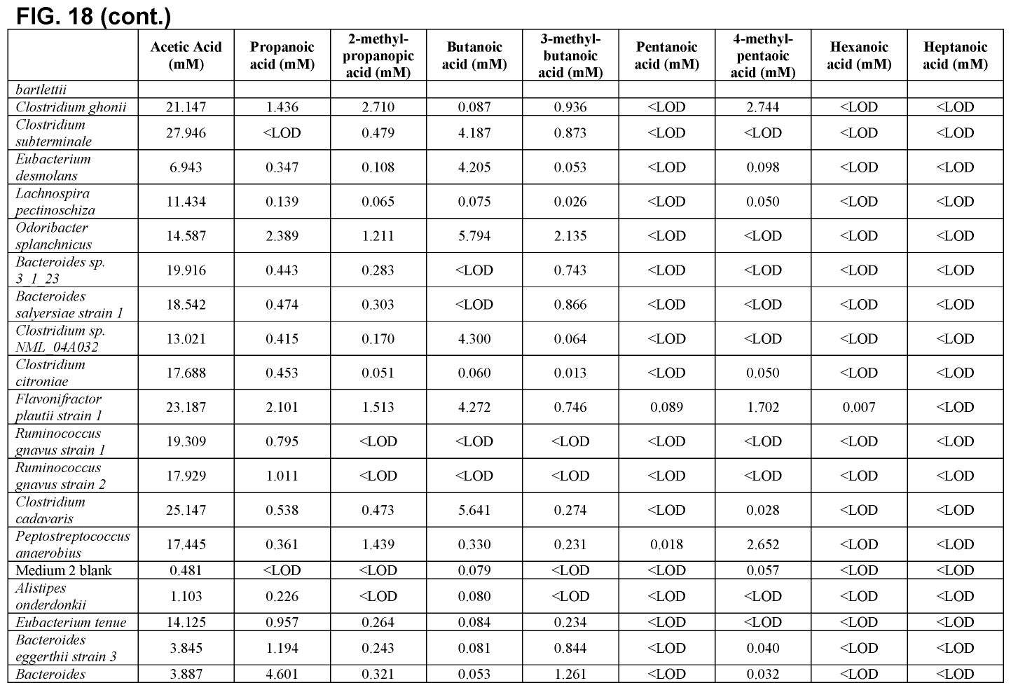

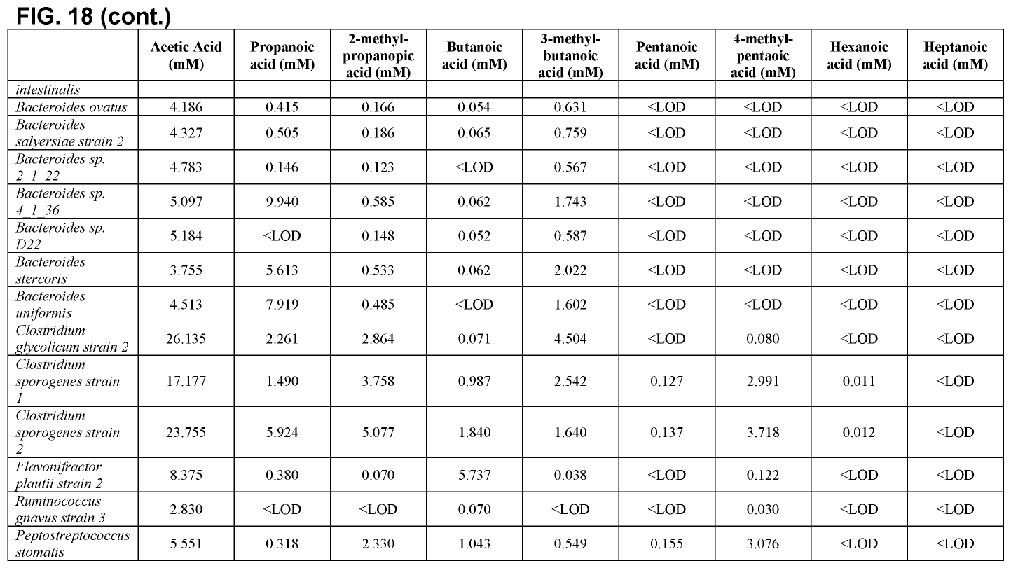

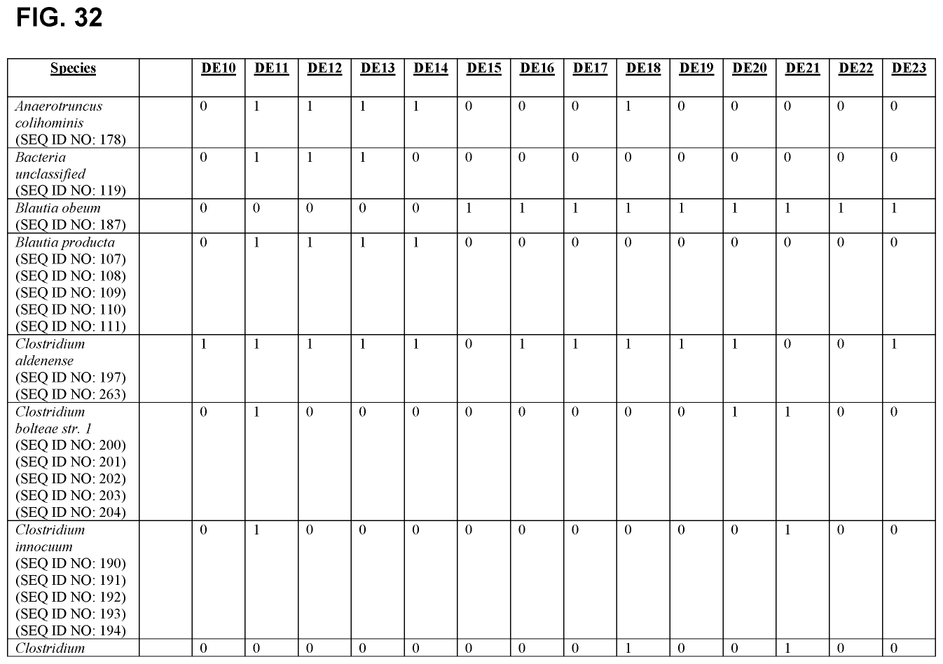

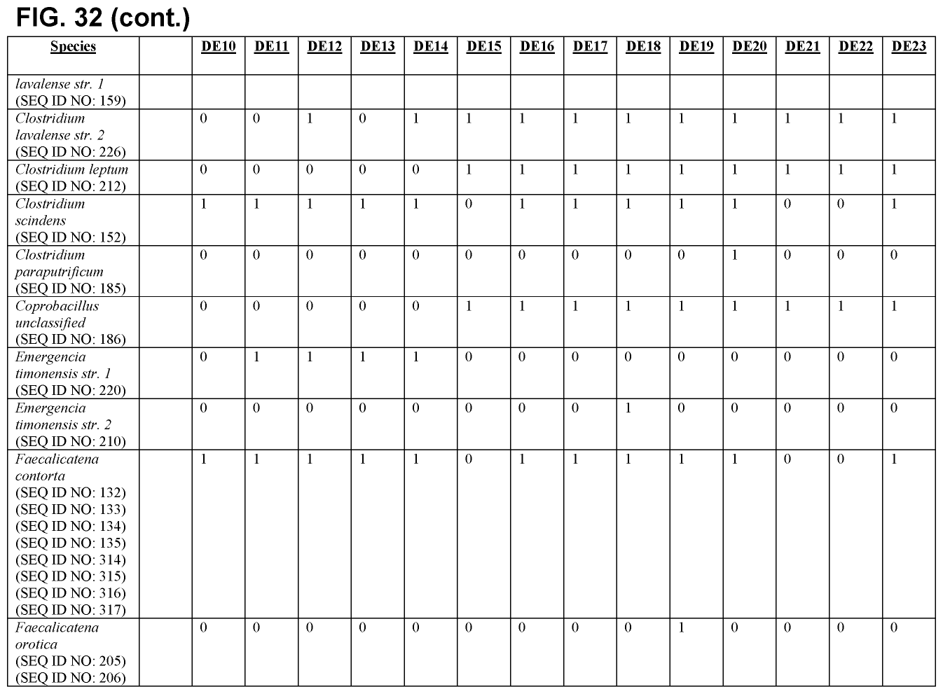

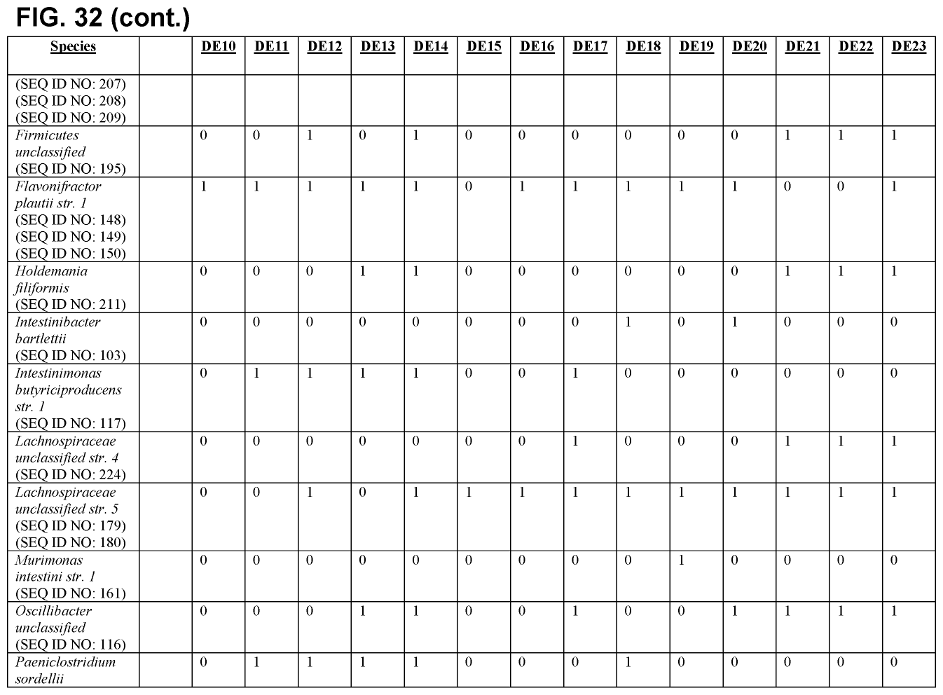

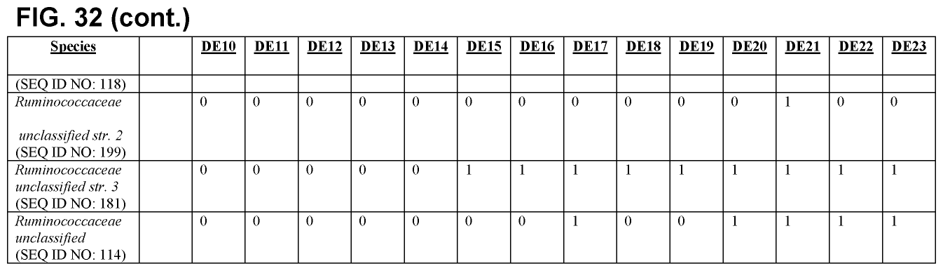

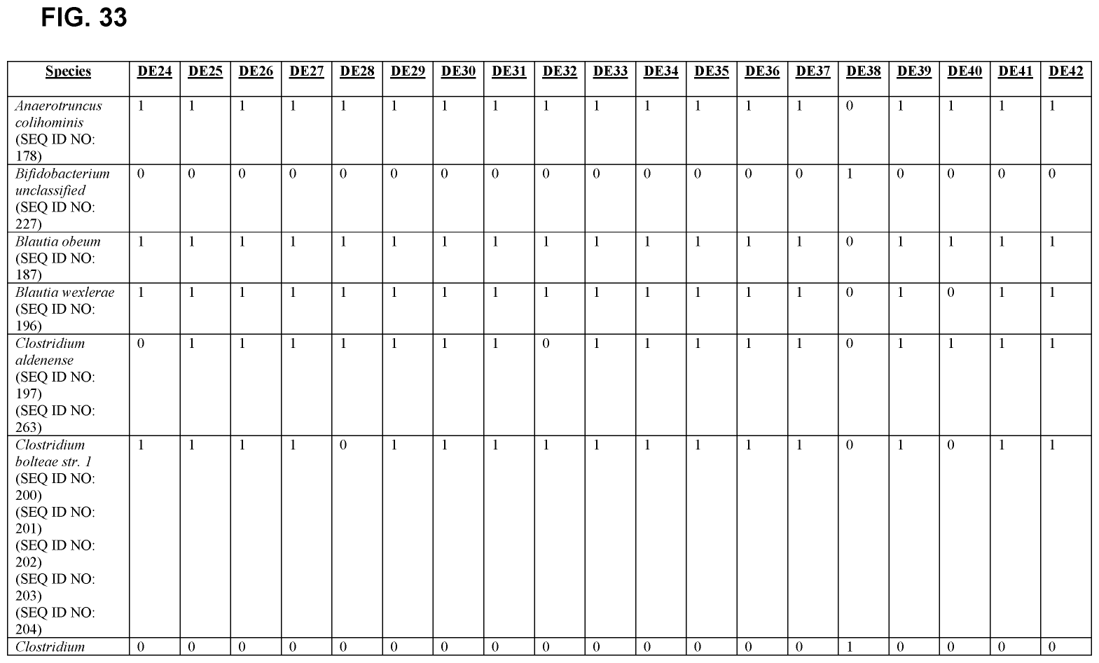

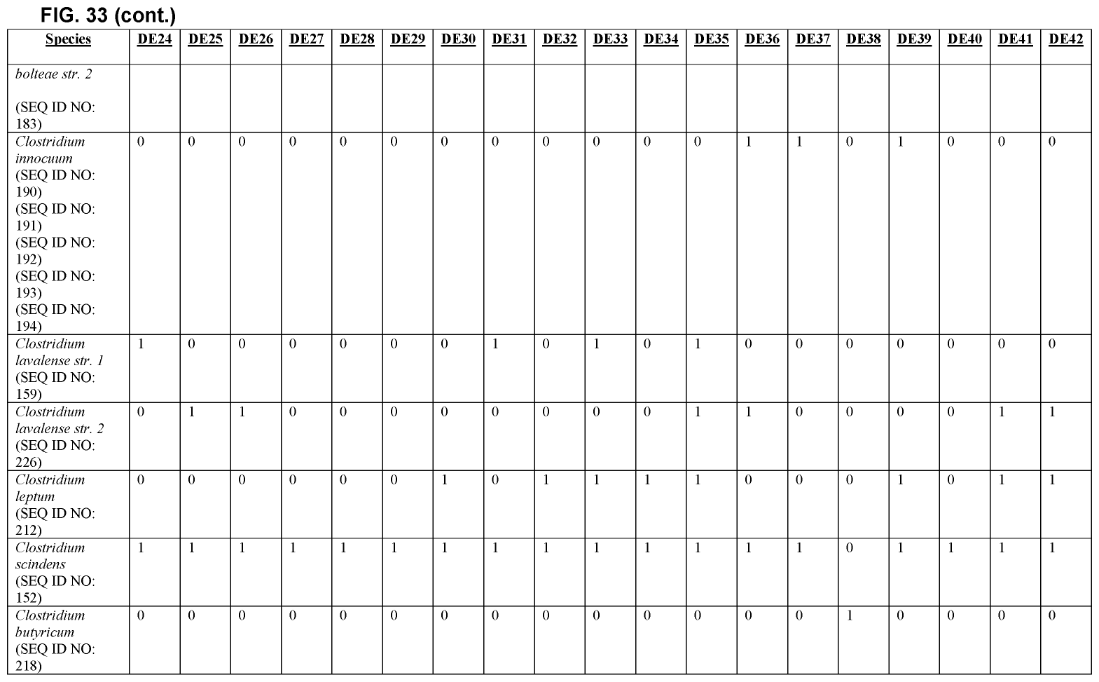

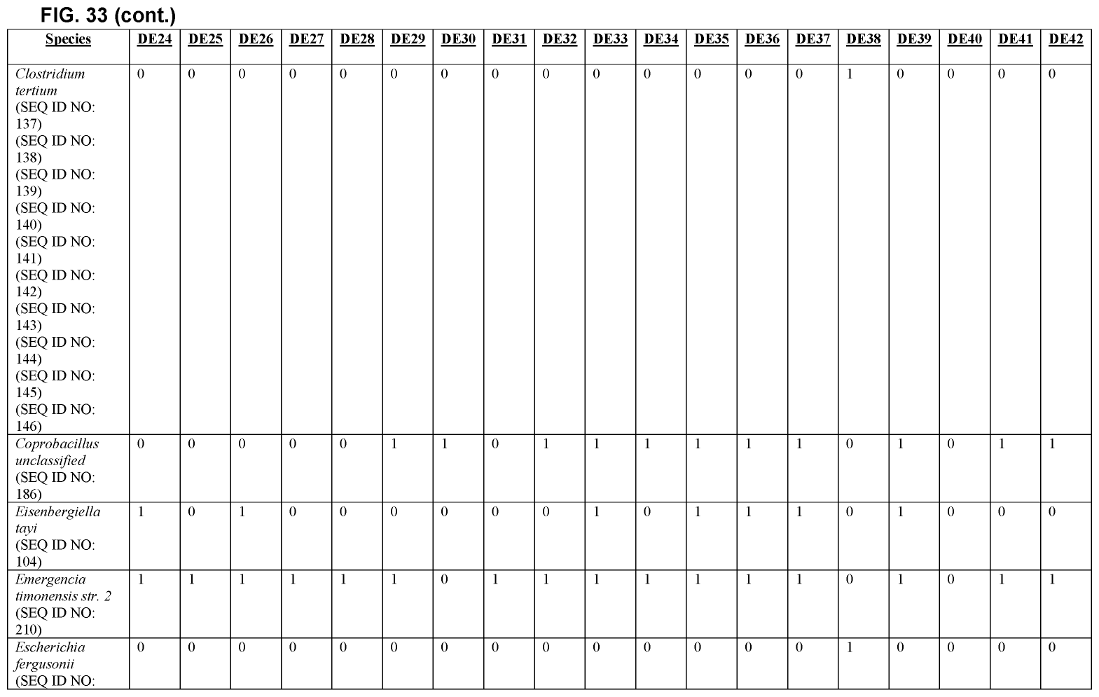

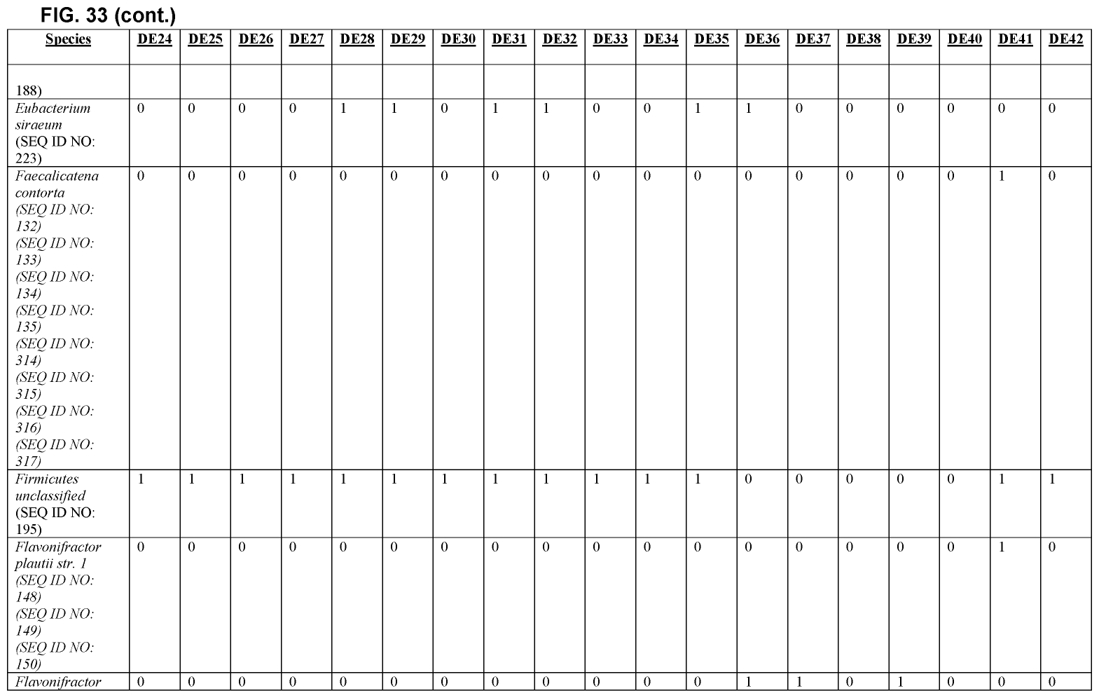

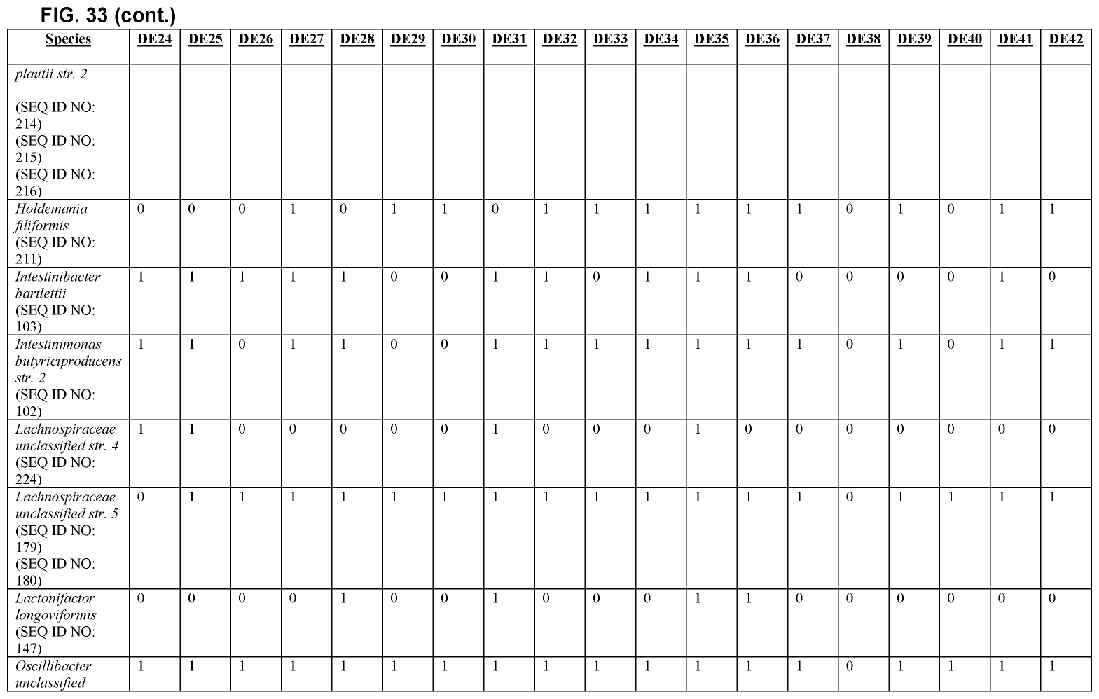

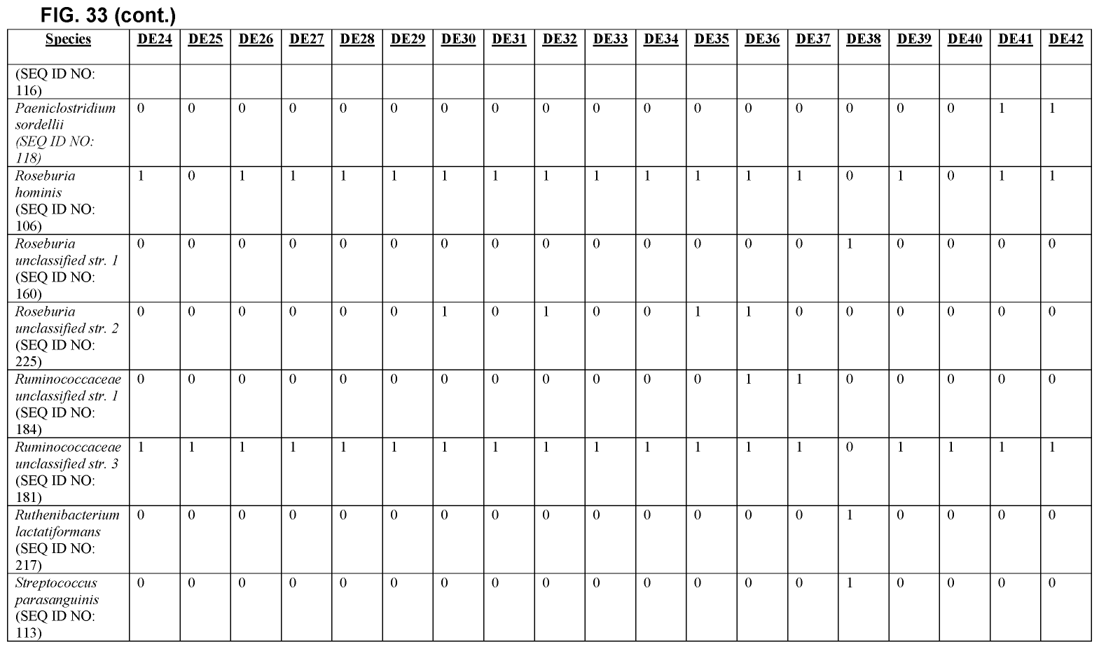

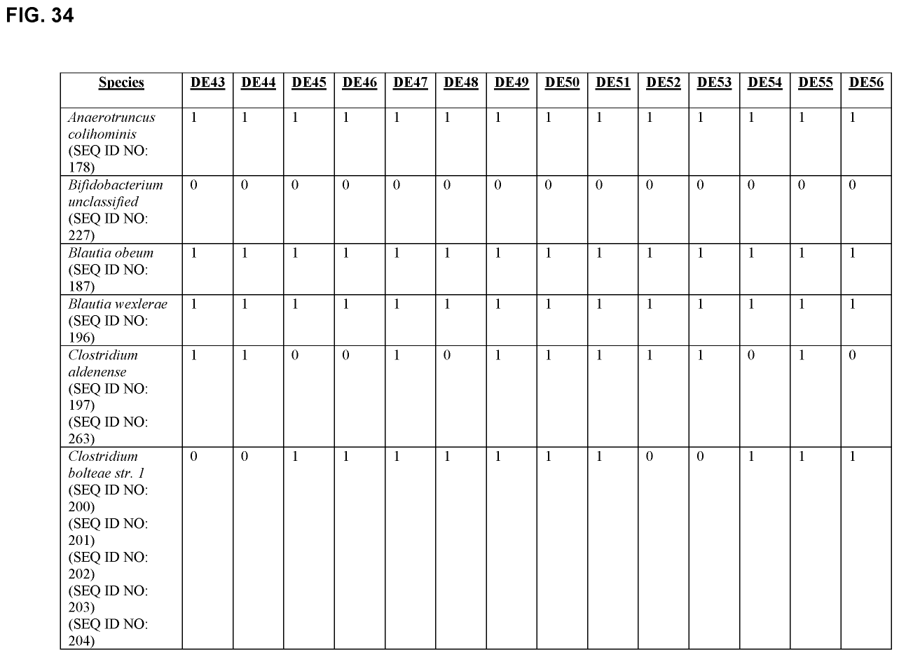

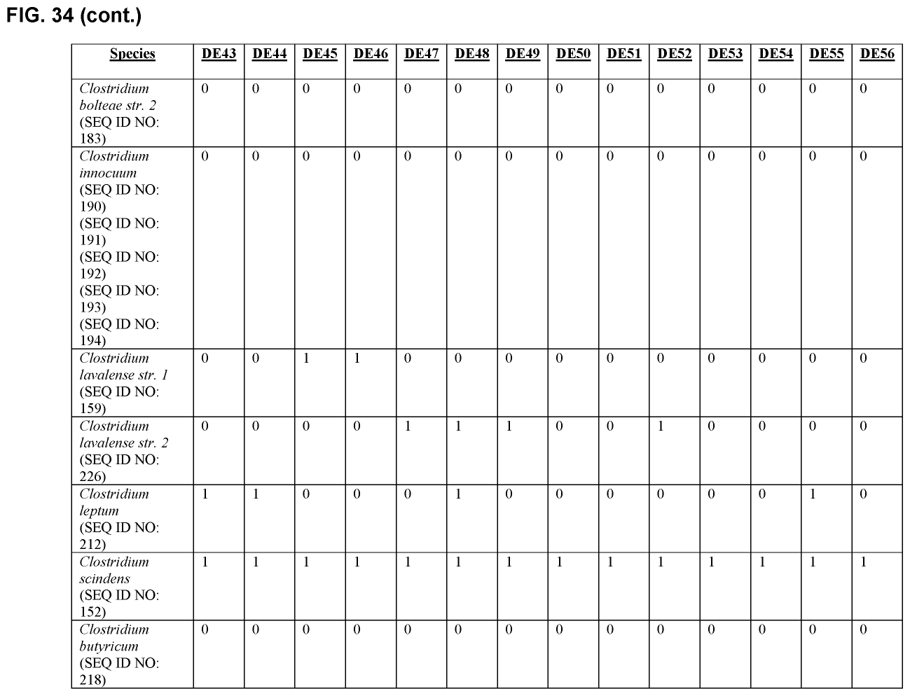

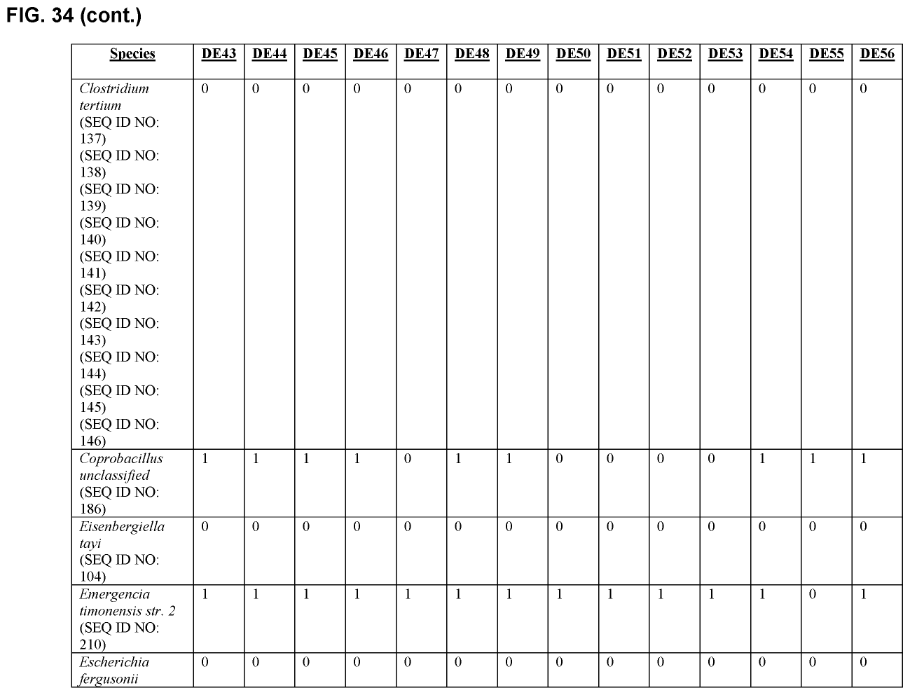

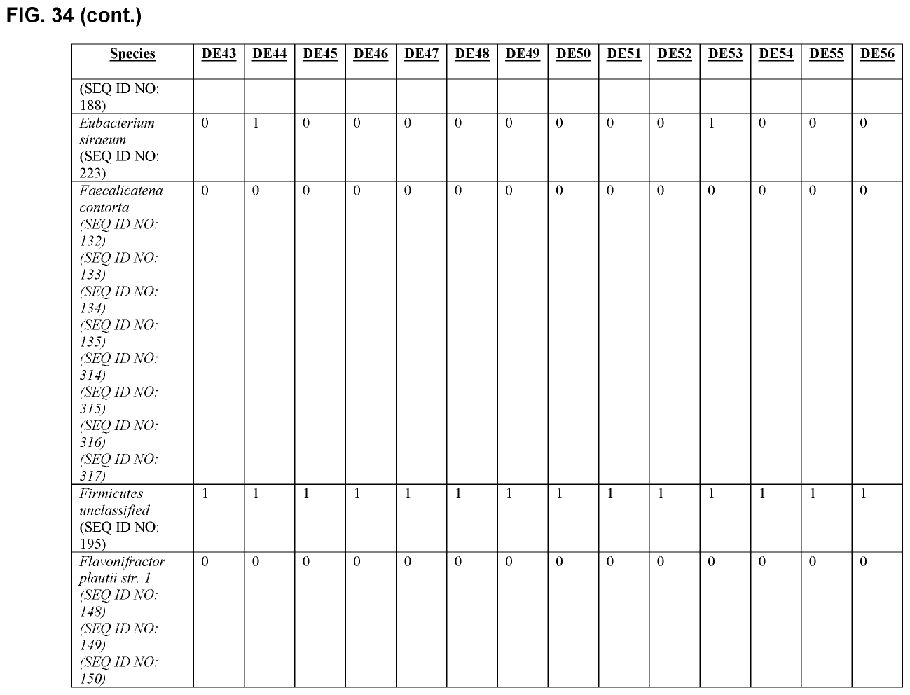

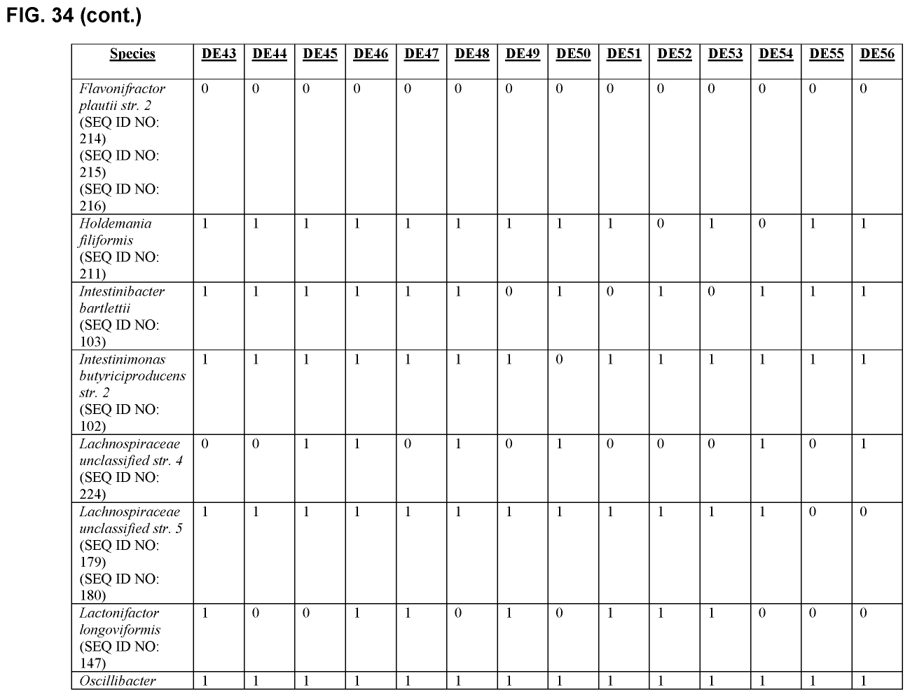

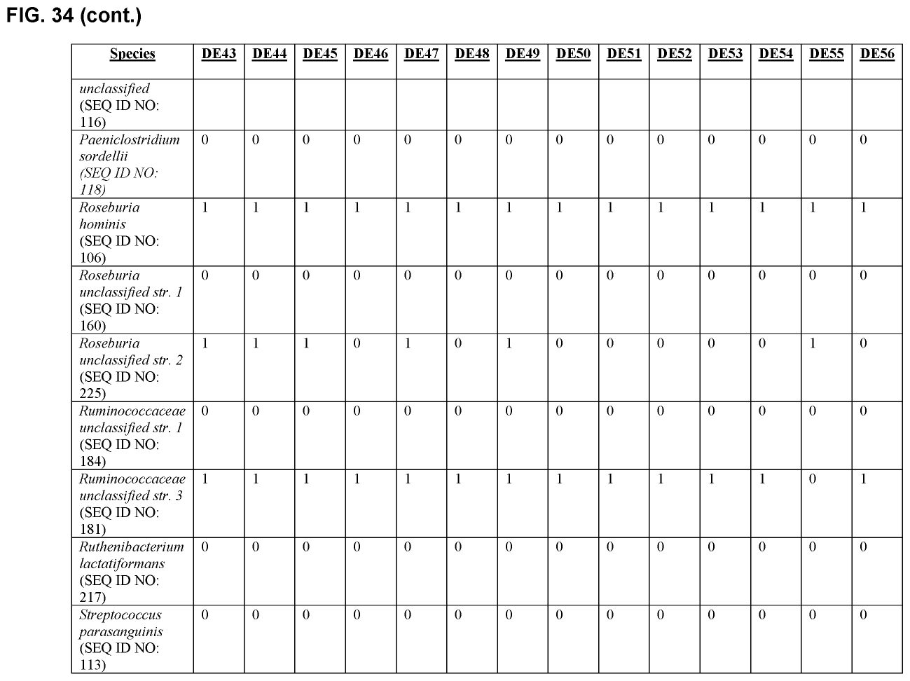

In some aspects, a bacterial composition disclosed herein comprises one or more features selected from (i) capable of engrafting when administered to a subject; (ii) capable of having anti-inflammatory activity, (iii) not capable of inducing pro-inflammatory activity, (iv) capable of producing a secondary bile acid, (v) capable of producing a tryptophan metabolite, (vi) capable of restoring epithelial integrity as determined by a primary epithelial cell monolayer barrier integrity assay, (vii) capable of being associated with remission of an inflammatory bowel disease, (viii) capable of producing a short-chain fatty acid, (ix) capable of inhibiting a HDAC activity, (x) capable of producing a medium-chain fatty acid, (xi) capable of inducing Wnt activation, or (xi) any combination thereof. In some aspects, the bacteria in a microbiome composition comprise one or more families, genera, species, or OTUs that are increased in the GI microbiome of a patient suffering from a disease or disorder associated with dysbiosis of the gastrointestinal tract (e.g., an ulcerative colitis or cancer patient) or population of patients prior to treatment with a complex microbiome composition, e.g., an HHSP or DE composition, and increased in a subject or a population of subjects after treatment with an HHSP or DE composition. In some aspects, a bacterial composition disclosed herein comprises selected families, genera, species, or OTUs of bacteria. In general, the bacteria are commensal bacteria initially derived from, for example, a GI tract, typically the GI tract of a human, isolated and grown into pure cultures that can be used in a DE. These bacteria are selected for desired properties as described herein and used in designed composition. In some aspects, a bacterial composition (e.g., designed compositions disclosed herein) comprises more than two types of bacteria. Accordingly, in some aspects, a bacterial composition of the present disclosure comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or at least 21, 22, 23, 24, 25, 26, 27, 28, 29 30, 31, 32, 33, 34, 35, 36, 37, 38, 39, or at least 40, at least 50, or greater than 50 types of bacteria, as defined by species or operational taxonomic unit (OTU), or otherwise as provided herein. The bacteria in a composition may be present in approximately equal amounts of viable bacteria or each family, genus, species of OTU. In other aspects of the invention, the bacteria are present in varying amounts in the composition. Non-limiting examples of bacterial species that can be used in designing the microbiome compositions disclosed herein are provided in Table 4, Table 5, FIG. 13, FIG. 17, FIG. 18, FIG. 31, FIG. 32, FIG. 33, FIG. 34.

In some aspects, the bacteria in a microbiome composition disclosed herein are from a family, genus, species, or OTU depleted in a subject suffering from a disease or disorder, such as those associated with a dysbiosis (e.g., ulcerative colitis or cancer patients) and/or typically present only at low levels or are absent in patients diagnosed with a disease or disorder, such as those associated with dysbiosis (e.g., ulcerative colitis or cancer). In some aspects, a bacterial composition includes one or more additional bacteria that are present with high frequency in a population of healthy humans or subjects with a disease or disorder associated with dysbiosis (e.g., ulcerative colitis or cancer patients) but who are not exhibiting symptoms associated with active disease (i.e., in clinical remission).

In some aspects, a bacterial composition disclosed herein comprises one or more bacteria from the family Ruminococcaceae, Lachnospiraceae, Sutterellaceae, Clostridiaceae, Erysipelotrichaceae, Bacteroidaceae, Akkermansiaceae, Peptostreptococcaceae, Eubacteriaceae, or Desulfovibrionaceae. In some aspects, a bacterial composition can comprise at least one, two, three, four, five, six, seven, or all of the families listed.

In some aspects, a bacterial composition comprises bacteria having at least about 97%, e.g., at least about 99%, identity to a 16S rDNA sequence (e.g., a full length or variable region of a 16S DNA sequence) to one or more of the following bacterial species: Gemmiger formicilis, Roseburia hominis, Clostridium bolteae, Parasutterella excrementihominis, Holdemania filiformis, Holdemania massiliensis, Bacteroides ovatus, Akkermansia muciniphila, Clostridium leptum, Bilophila wadsworthia, Dielma fastidiosa, Clostridium symbiosum, Eubacterium siraeum, Clostridium innocuum, Agathobaculum desmolans, Agathobaculum butyriciproducens, or Bacteroides vulgatus. In some aspects, one or more of the bacteria in a composition has at least about 97% identity, e.g., about 99% identity, to a 16S rDNA of the foregoing species. In some aspects, a bacterial composition can comprise at least one, two, three, four, five, six, seven, eight, nine, ten, eleven, twelve, thirteen, fourteen, fifteen, or all of the species listed.

In some aspects, a bacterial composition comprises bacteria having at least about 97% identity, e.g., about 99% identity, to a 16S rDNA sequence (e.g., a full length or variable region or a 16S DNA sequence) to one or more of the following bacterial species: Gemmiger formicilis, Roseburia hominis, Clostridium bolteae, Parasutterella excrementihominis, Holdemania filiformis, Holdemania massiliensis, Bacteroides ovatus, Akkermansia muciniphila, Clostridium leptum, Bilophila wadsworthia, Dielma fastidiosa, Clostridium symbiosum, Eubacterium siraeum, Clostridium] innocuum, Erysipelotrichaceae SC11, Roseburia sp CAG 45 SC195, Lachnospiraceae SC188, Lachnospiraceae SC52, Clostridium SC125, Flintibacter SC49, Agathobaculum desmolans, Agathobaculum butyriciproducens, or Bacteroides vulgatus. In some aspects, one or more of the bacteria in a composition has at least 97% identity, e.g., 99% identity, to a 16S rDNA of the foregoing species. In some aspects, a bacterial composition can comprise at least one, two, three, four, five, six, seven, eight, nine, ten, eleven, twelve, thirteen, fourteen, fifteen, or all of the species listed.

In some aspects, a bacterial composition comprises one or more bacteria selected from the group consisting of Gemmiger formicilis, Roseburia hominis, Clostridium bolteae, Holdemania filiformis, Holdemania massiliensis, Clostridium leptum, Dielma fastidiosa, Clostridium symbiosum, Eubacterium siraeum, and combinations thereof. In some aspects, one or more of the bacteria in a composition has at least about 97% identity, e.g., about 99% identity, to a 16S rDNA of the foregoing species. In some aspects, a bacterial composition can comprise at least one, two, three, four, five, six, seven, eight, or all of the bacterial species listed.

In some aspects, a bacterial composition comprises one or more of the following bacterial species: Anaerotruncus colihominis, Blautia producta, Clostridium bolteae, Clostridium disporicum, Clostridium ghonii, Clostridium glycolicum, Clostridium innocuum, Clostridium lactatifermentans, Clostridium viride, Eubacterium sp. WAL 14571, Lachnospiraceae bacterium 3 1 57FA, Lachnospiraceae bacterium oral taxon F15, Lactonifactor longoviformis, or Ruminococcus lactaris. In some aspects, one or more of the bacteria in a composition has at least 97% identity, e.g., 99% identity, to a 16S rDNA of the foregoing species.

In some aspects, a bacterial composition (e.g., designed composition) disclosed herein comprises one or more of the bacterial species disclosed in Table 4, Table 5, FIG. 13, FIG. 17, FIG. 18, FIG. 31, FIG. 32, FIG. 33, and/or FIG. 34.

In some aspects, a bacterial composition of the present disclosure comprises one or more bacteria comprising a 16S rDNA sequence that is at least about 85%, at least about 90%, at least about 95%, at least about 96%, at least about 97%, at least about 97.5%, at least about 98%, at least about 98.5%, at least about 99%, at least about 99.5%, or about 100% identical to a 16S rDNA sequence set forth in SEQ ID NOs: 1-14, 16-30, 32-36, 39, 41, 44, 45, 47-51, 59-62, 64-68, 72-76, and 102-398.

In some aspects, a bacterial composition disclosed herein comprises at least one, at least two, at least three, at least four, at least five, at least six, at least seven, at least eight, at least nine, at least 10, at least 11, at least 12, at least 13, or all 14 bacterial species of DE1. In some aspects, a bacterial composition comprises at least one, at least two, at least three, at least four, at least five, at least six, at least seven, at least eight, at least nine, at least 10, at least 11, at least 12, at least 13, or all 14 bacterial species of DE2. In some aspects, a bacterial composition comprises at least one, at least two, or all three bacterial species of DE3. In some aspects, a bacterial composition comprises at least one, at least two, at least three, or all four bacterial species of DE4. In some aspects, a bacterial composition comprises at least one, at least two, at least three, or all four bacterial species of DE5. In some aspects, a bacterial composition comprises at least one, at least two, at least three, at least four, at least five, at least six, at least seven, at least eight, at least nine, at least 10, at least 11, at least 12, at least 13, or all 14 bacterial species of DE6. In some aspects, a bacterial composition comprises at least one, at least two, at least three, at least four, at least five, at least six, at least seven, at least eight, at least nine, at least 10, at least 11, at least 12, at least 13, or all 14 bacterial species of DE7. In some aspects, a bacterial composition comprises at least one, at least two, at least three, at least four, at least five, at least six, at least seven, at least eight, at least nine, at least 10, at least 11, at least 12, at least 13, or all 14 bacterial species of DE8. In some aspects, a bacterial composition comprises at least one, at least two, at least three, or all four bacterial species of DE10. In some aspects, a bacterial composition of the present disclosure comprises at least one, at least two, at least three, at least four, at least five, at least six, at least seven, at least eight, at least nine, at least 10, at least 11, or all 12 bacterial species of DE11. In some aspects, a bacterial composition of the present disclosure comprises at least one, at least two, at least three, at least four, at least five, at least six, at least seven, at least eight, at least nine, at least 10, at least 11, at least 12, or all 13 bacterial species of DE12. In some aspects, a bacterial composition comprises at least one, at least two, at least three, at least four, at least five, at least six, at least seven, at least eight, at least nine, at least 10, at least 11, or all 12 bacterial species of DE13. In some aspects, a bacterial composition comprises at least one, at least two, at least three, at least four, at least five, at least six, at least seven, at least eight, at least nine, at least 10, at least 11, at least 12, at least 13, or all 14 bacterial species of DE14. In some aspects, a bacterial composition comprises at least one, at least two, at least three, at least four, at least five, or all six bacterial species of DE15. In some aspects, a bacterial composition comprises at least one, at least two, at least three, at least four, at least five, at least six, at least seven, at least eight, at least nine, or all 10 bacterial species of DE16. In some aspects, a bacterial composition comprises at least one, at least two, at least three, at least four, at least five, at least six, at least seven, at least eight, at least nine, at least 10, at least 11, at least 12, at least 13, or all 14 bacterial species of DE17. In some aspects, a bacterial composition comprises at least one, at least two, at least three, at least four, at least five, at least six, at least seven, at least eight, at least nine, at least 10, at least 11, at least 12, at least 13, at least 14, or all 15 bacterial species of DE18. In some aspects, a bacterial composition comprises at least one, at least two, at least three, at least four, at least five, at least six, at least seven, at least eight, at least nine, at least 10, at least 11, or all 12 bacterial species of DE19. In some aspects, a bacterial composition of the present disclosure comprises at least one, at least two, at least three, at least four, at least five, at least six, at least seven, at least eight, at least nine, at least 10, at least 11, at least 12, at least 13, at least 14, or all 15 bacterial species of DE20. In some aspects, a bacterial composition comprises at least one, at least two, at least three, at least four, at least five, at least six, at least seven, at least eight, at least nine, at least 10, at least 11, at least 12, at least 13, at least 14, or all 15 bacterial species of DE21. In some aspects, a bacterial composition comprises at least one, at least two, at least three, at least four, at least five, at least six, at least seven, at least eight, at least nine, at least 10, or all 11 bacterial species of DE22. In some aspects, a bacterial composition comprises at least one, at least two, at least three, at least four, at least five, at least six, at least seven, at least eight, at least nine, at least 10, at least 11, at least 12, at least 13, at least 14, or all 15 bacterial species of DE23. In some aspects, a bacterial composition comprises at least one, at least two, at least three, at least four, at least five, at least six, at least seven, at least eight, at least nine, at least 10, at least 11, at least 12, at least 13, at least 14, or all 15 bacterial species of DE24. In some aspects, a bacterial composition comprises at least one, at least two, at least three, at least four, at least five, at least six, at least seven, at least eight, at least nine, at least 10, at least 11, at least 12, at least 13, at least 14, or all 15 bacterial species of DE25. In some aspects, a bacterial composition disclosed herein comprises at least one, at least two, at least three, at least four, at least five, at least six, at least seven, at least eight, at least nine, at least 10, at least 11, at least 12, at least 13, at least 14, or all 15 bacterial species of DE26. In some aspects, a bacterial composition comprises at least one, at least two, at least three, at least four, at least five, at least six, at least seven, at least eight, at least nine, at least 10, at least 11, at least 12, at least 13, at least 14, or all 15 bacterial species of DE27. In some aspects, a bacterial composition comprises at least one, at least two, at least three, at least four, at least five, at least six, at least seven, at least eight, at least nine, at least 10, at least 11, at least 12, at least 13, at least 14, or all 15 bacterial species of DE28. In some aspects, a bacterial composition comprises at least one, at least two, at least three, at least four, at least five, at least six, at least seven, at least eight, at least nine, at least 10, at least 11, at least 12, at least 13, at least 14, or all 15 bacterial species of DE29. In some aspects, a bacterial composition comprises at least one, at least two, at least three, at least four, at least five, at least six, at least seven, at least eight, at least nine, at least 10, at least 11, at least 12, at least 13, at least 14, or all 15 bacterial species of DE30. In some aspects, a bacterial composition comprises at least one, at least two, at least three, at least four, at least five, at least six, at least seven, at least eight, at least nine, at least 10, at least 11, at least 12, at least 13, at least 14, at least 15, at least 16, at least 17, or all 18 bacterial species of DE31. In some aspects, a bacterial composition comprises at least one, at least two, at least three, at least four, at least five, at least six, at least seven, at least eight, at least nine, at least 10, at least 11, at least 12, at least 13, at least 14, at least 15, at least 16, at least 17, or all 18 bacterial species of DE32. In some aspects, a bacterial composition comprises at least one, at least two, at least three, at least four, at least five, at least six, at least seven, at least eight, at least nine, at least 10, at least 11, at least 12, at least 13, at least 14, at least 15, at least 16, at least 17, or all 18 bacterial species of DE33. In some aspects, a bacterial composition comprises at least one, at least two, at least three, at least four, at least five, at least six, at least seven, at least eight, at least nine, at least 10, at least 11, at least 12, at least 13, at least 14, at least 15, at least 16, or all 17 bacterial species of DE34. In some aspects, a bacterial composition comprises at least one, at least two, at least three, at least four, at least five, at least six, at least seven, at least eight, at least nine, at least 10, at least 11, at least 12, at least 13, at least 14, at least 15, at least 16, at least 17, at least 18, at least 19, at least 20, at least 21, at least 22, at least 23, or all 24 bacterial species of DE35. In some aspects, a bacterial composition comprises at least one, at least two, at least three, at least four, at least five, at least six, at least seven, at least eight, at least nine, at least 10, at least 11, at least 12, at least 13, at least 14, at least 15, at least 16, at least 17, at least 18, at least 19, at least 20, at least 21, at least 22, or all 23 bacterial species of DE36. In some aspects, a bacterial composition of the present disclosure comprises at least one, at least two, at least three, at least four, at least five, at least six, at least seven, at least eight, at least nine, at least 10, at least 11, at least 12, at least 13, at least 14, at least 15, at least 16, at least 17, or all 18 bacterial species of DE37. In some aspects, a bacterial composition of the present disclosure comprises at least one, at least two, at least three, at least four, at least five, at least six, at least seven, at least eight, at least nine, at least 10, at least 11, at least 12, at least 13, at least 14, at least 15, at least 16, at least 17, or all 18 bacterial species of DE39. In some aspects, a bacterial composition disclosed herein comprises at least one, at least two, at least three, at least four, at least five, at least six, or all seven bacterial species of DE40. In some aspects, a bacterial composition comprises at least one, at least two, at least three, at least four, at least five, at least six, at least seven, at least eight, at least nine, at least 10, at least 11, at least 12, at least 13, at least 14, at least 15, at least 16, at least 17, at least 18, at least 19, at least 20, or all 21 bacterial species of DE41. In some aspects, a bacterial composition comprises at least one, at least two, at least three, at least four, at least five, at least six, at least seven, at least eight, at least nine, at least 10, at least 11, at least 12, at least 13, at least 14, at least 15, at least 16, at least 17, or all 18 bacterial species of DE42. In some aspects, a bacterial composition comprises at least one, at least two, at least three, at least four, at least five, at least six, at least seven, at least eight, at least nine, at least 10, at least 11, at least 12, at least 13, at least 14, at least 15, at least 16, at least 17, or all 18 bacterial species of DE43. In some aspects, a bacterial composition comprises at least one, at least two, at least three, at least four, at least five, at least six, at least seven, at least eight, at least nine, at least 10, at least 11, at least 12, at least 13, at least 14, at least 15, at least 16, at least 17, or all 18 bacterial species of DE44. In some aspects, a bacterial composition comprises at least one, at least two, at least three, at least four, at least five, at least six, at least seven, at least eight, at least nine, at least 10, at least 11, at least 12, at least 13, at least 14, at least 15, at least 16, at least 17, or all 18 bacterial species of DE45. In some aspects, a bacterial composition comprises at least one, at least two, at least three, at least four, at least five, at least six, at least seven, at least eight, at least nine, at least 10, at least 11, at least 12, at least 13, at least 14, at least 15, at least 16, at least 17, or all 18 bacterial species of DE46. In some aspects, a bacterial composition comprises at least one, at least two, at least three, at least four, at least five, at least six, at least seven, at least eight, at least nine, at least 10, at least 11, at least 12, at least 13, at least 14, at least 15, at least 16, at least 17, or all 18 bacterial species of DE47. In some aspects, a bacterial composition comprises at least one, at least two, at least three, at least four, at least five, at least six, at least seven, at least eight, at least nine, at least 10, at least 11, at least 12, at least 13, at least 14, at least 15, at least 16, at least 17, or all 18 bacterial species of DE48. In some aspects, a bacterial composition comprises at least one, at least two, at least three, at least four, at least five, at least six, at least seven, at least eight, at least nine, at least 10, at least 11, at least 12, at least 13, at least 14, at least 15, at least 16, at least 17, or all 18 bacterial species of DE49. In some aspects, a bacterial composition comprises at least one, at least two, at least three, at least four, at least five, at least six, at least seven, at least eight, at least nine, at least 10, at least 11, at least 12, at least 13, at least 14, or all 15 bacterial species of DE50. In some aspects, a bacterial composition comprises at least one, at least two, at least three, at least four, at least five, at least six, at least seven, at least eight, at least nine, at least 10, at least 11, at least 12, at least 13, at least 14, or all 15 bacterial species of DE51. In some aspects, a bacterial composition comprises at least one, at least two, at least three, at least four, at least five, at least six, at least seven, at least eight, at least nine, at least 10, at least 11, at least 12, at least 13, at least 14, or all 15 bacterial species of DE52. In some aspects, a bacterial composition comprises at least one, at least two, at least three, at least four, at least five, at least six, at least seven, at least eight, at least nine, at least 10, at least 11, at least 12, at least 13, at least 14, or all 15 bacterial species of DE53. In some aspects, a bacterial composition comprises at least one, at least two, at least three, at least four, at least five, at least six, at least seven, at least eight, at least nine, at least 10, at least 11, at least 12, at least 13, at least 14, or all 15 bacterial species of DE54. In some aspects, a bacterial composition comprises at least one, at least two, at least three, at least four, at least five, at least six, at least seven, at least eight, at least nine, at least 10, at least 11, at least 12, at least 13, at least 14, or all 15 bacterial species of DE55. In some aspects, a bacterial composition comprises at least one, at least two, at least three, at least four, at least five, at least six, at least seven, at least eight, at least nine, at least 10, at least 11, at least 12, at least 13, at least 14, or all 15 bacterial species of DE56. Bacterial species present in each of DE1-DE8, DE10-DE37, and DE39-DE56 are provided in FIGS. 31, 32, 33, and 34.

In some aspects, a bacterial composition disclosed herein comprises at least one, at least two, at least three, at least four, at least five, at least six, at least seven, at least eight, at least nine, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50 of the bacterial species (or strains) provided in FIG. 31.

In some aspects, a bacterial composition disclosed herein comprises at least one, at least two, at least three, at least four, at least five, at least six, at least seven, at least eight, at least nine, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50 of the bacterial species (or strains) provided in FIG. 32.

In some aspects, a bacterial composition disclosed herein comprises at least one, at least two, at least three, at least four, at least five, at least six, at least seven, at least eight, at least nine, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50 of the bacterial species (or strains) provided in FIG. 33

In some aspects, a bacterial composition disclosed herein comprises at least one, at least two, at least three, at least four, at least five, at least six, at least seven, at least eight, at least nine, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50 of the bacterial species (or strains) provided in FIG. 34

In some aspects, a bacterial composition disclosed herein comprises at least one, at least two, at least three, at least four, at least five, at least six, at least seven, at least eight, at least nine, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50 of the bacterial species (or strains) provided in FIG. 13.

In some aspects, a bacterial composition disclosed herein comprises at least one, at least two, at least three, at least four, at least five, at least six, at least seven, at least eight, at least nine, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, or all 48 of the bacterial species (or strains) provided in FIG. 18.

In some aspects, a bacterial composition disclosed herein comprises a 16S rDNA sequence set forth in SEQ ID NOs: 151, 196, 190, 191, 192, 193, 194, 120, 121, 122, 123, 124, 125, 126, 127, 128, 129, 130, 131, 136, 200, 201, 202, 203, 204, 148, 149, 150, 107, 108, 109, 110, 111, 105, 182, 219, 153, 115, 213, 166, 167, 168, 169, 170, 171, 172, 173, 174, 175, 176, 177, 214, 215, 216, 103, 178, 161, 154, 155, 156, 157, 158, 119, 132, 133, 134, 135, 314, 315, 316, 317, 117, 205, 206, 207, 208, 209, 220, 221, 222, 197, 263, 102, 118, 159, 198, 112, 184, 104, 223, 189, 186, 224, 106, 199, 147, 211, 179, 180, 152, 195, 185, 116, 225, 226, 210, 212, 181, 114, 187, or combinations thereof. In certain aspects, a bacterial composition disclosed herein comprises a 16S rDNA sequence that is at least 97% identical to the 16S rDNA sequence set forth in SEQ ID NOs: 151, 196, 190, 191, 192, 193, 194, 120, 121, 122, 123, 124, 125, 126, 127, 128, 129, 130, 131, 136, 200, 201, 202, 203, 204, 148, 149, 150, 107, 108, 109, 110, 111, 105, 182,219, 153, 115, 213, 166, 167, 168, 169, 170, 171, 172, 173, 174, 175, 176, 177, 214, 215, 216, 103, 178, 161, 154, 155, 156, 157, 158, 119, 132, 133, 134, 135, 314, 315, 316, 317, 117, 205, 206, 207, 208, 209, 220, 221, 222, 197, 263, 102, 118, 159, 198, 112, 184, 104, 223, 189, 186, 224, 106, 199, 147, 211, 179, 180, 152, 195, 185, 116, 225, 226, 210, 212, 181, 114, 187, or combinations thereof.

In some aspects, a bacterial composition disclosed herein comprises a 16S rDNA sequence set forth in SEQ ID NOs: 190, 191, 192, 193, 194, 200, 201, 202, 203, 204, 214, 215, 216, 178, 197, 263, 102, 104, 179, 180, 152, 210, 181, 196, 186, 106, 211, 212, 116, 187, or combinations thereof. In certain aspects, a bacterial composition disclosed herein comprises a 16S rDNA sequence that is at least 97% identical to the 16S rDNA sequence set forth in SEQ ID NOs: 190, 191, 192, 193, 194, 200, 201, 202, 203, 204, 214, 215, 216, 178, 197, 263, 102, 104, 179, 180, 152, 210, 181, 196, 186, 106, 211, 212, 116, 187, or combinations thereof.

In some aspects, a bacterial composition disclosed herein comprises a 16S rDNA sequence set forth in SEQ ID NOs: 178, 197, 263, 179, 180, 152, 116, 181, 187, or combinations thereof. In certain aspects, a bacterial composition disclosed herein comprises a 16S rDNA sequence that is at least 97% identical to the 16S rDNA sequence set forth in SEQ ID NOs: 178, 197, 263, 179, 180, 152, 116, 181, 187, or combinations thereof.

In some aspects, a bacterial composition disclosed herein comprises a 16S rDNA sequence set forth in SEQ ID NOs: 178, 197, 263, 179, 180, 152, 116, 181, 187, 196, 200, 201, 202, 203, 204, 148, 149, 150, 103, 132, 133, 134, 135, 314, 315, 316, 317, 102, 118, 186, 106, 211, 195, 226, 210, 212, or combinations thereof. In certain aspects, a bacterial composition disclosed herein comprises a 16S rDNA sequence that is at least 97% identical to the 16S rDNA sequence set forth in SEQ ID NOs: 178, 197, 263, 179, 180, 152, 116, 181, 187, 196, 200, 201, 202, 203, 204, 148, 149, 150, 103, 132, 133, 134, 135, 314, 315, 316, 317, 102, 118, 186, 106, 211, 195, 226, 210, 212, or combinations thereof.

In some aspects, a bacterial composition disclosed herein comprises a 16S rDNA sequence set forth in SEQ ID NOs: 178, 187, 196, 197, 263, 200, 201, 202, 203, 204, 226, 212, 152, 186, 210, 195, 211, 102, 179, 180, 116, 118, 106, 181, or combinations thereof. In certain aspects, a bacterial composition disclosed herein comprises a 16S rDNA sequence that is at least 97% identical to the 16S rDNA sequence set forth in SEQ ID NOs: 178, 187, 196, 197, 263, 200, 201, 202, 203, 204, 226, 212, 152, 186, 210, 195, 211, 102, 179, 180, 116, 118, 106, 181, or combinations thereof.

In some aspects, a bacterial composition disclosed herein comprises a 16S rDNA sequence set forth in SEQ ID NOs: 178, 187, 196, 197, 263, 212, 152, 186, 210, 195, 211, 103, 102, 179, 180, 147, 116, 106, 225, 181, or combinations thereof. In certain aspects, a bacterial composition disclosed herein comprises a 16S rDNA sequence that is at least 97% identical to the 16S rDNA sequence set forth in SEQ ID NOs: 178, 187, 196, 197, 263, 212, 152, 186, 210, 195, 211, 103, 102, 179, 180, 147, 116, 106, 225, 181, or combinations thereof.

In some aspects, a bacterial composition disclosed herein comprises a 16S rDNA sequence set forth in SEQ ID NOs: 178, 187, 196, 197, 263, 212, 152, 186, 210, 223, 195, 211, 103, 102, 179, 180, 116, 106, 225, 181, or combinations thereof. In certain aspects, a bacterial composition disclosed herein comprises a 16S rDNA sequence that is at least 97% identical to the 16S rDNA sequence set forth in SEQ ID NOs: 178, 187, 196, 197, 263, 212, 152, 186, 210, 223, 195, 211, 103, 102, 179, 180, 116, 106, 225, 181, or combinations thereof.

In some aspects, a bacterial composition disclosed herein comprises a 16S rDNA sequence set forth in SEQ ID NOs: 178, 187, 196, 200, 201, 202, 203, 204, 159, 152, 186, 210, 223, 195, 211, 103, 102, 224, 179, 180, 116, 106, 225, 181, or combinations thereof. In certain aspects, a bacterial composition disclosed herein comprises a 16S rDNA sequence that is at least 97% identical to the 16S rDNA sequence set forth in SEQ ID NOs: 178, 187, 196, 200, 201, 202, 203, 204, 159, 152, 186, 210, 223, 195, 211, 103, 102, 224, 179, 180, 116, 106, 225, 181, or combinations thereof.

In some aspects, a bacterial composition disclosed herein comprises a 16S rDNA sequence set forth in SEQ ID NOs: 178, 187, 196, 200, 201, 202, 203, 204, 159, 152, 186, 210, 195, 211, 103, 102, 224, 179, 180, 147, 116, 106, 181, or combinations thereof. In certain aspects, a bacterial composition disclosed herein comprises a 16S rDNA sequence that is at least 97% identical to the 16S rDNA sequence set forth in SEQ ID NOs: 178, 187, 196, 200, 201, 202, 203, 204, 159, 152, 186, 210, 195, 211, 103, 102, 224, 179, 180, 147, 116, 106, 181, or combinations thereof.

In some aspects, a bacterial composition disclosed herein comprises a 16S rDNA sequence set forth in SEQ ID NOs: 178, 187, 196, 197, 263, 200, 201, 202, 203, 204, 226, 152, 210, 195, 211, 103, 102, 179, 180, 147, 116, 106, 225, 181, or combinations thereof. In certain aspects, a bacterial composition disclosed herein comprises a 16S rDNA sequence that is at least 97% identical to the 16S rDNA sequence set forth in SEQ ID NOs: 178, 187, 196, 197, 263, 200, 201, 202, 203, 204, 226, 152, 210, 195, 211, 103, 102, 179, 180, 147, 116, 106, 225, 181, or combinations thereof.

In some aspects, a bacterial composition disclosed herein comprises a 16S rDNA sequence set forth in SEQ ID NOs: 178, 187, 196, 200, 201, 202, 203, 204, 226, 212, 152, 186, 210, 195, 211, 103, 102, 224, 179, 180, 116, 106, 181, or combinations thereof. In certain aspects, a bacterial composition disclosed herein comprises a 16S rDNA sequence that is at least 97% identical to the 16S rDNA sequence set forth in SEQ ID NOs: 178, 187, 196, 200, 201, 202, 203, 204, 226, 212, 152, 186, 210, 195, 211, 103, 102, 224, 179, 180, 116, 106, 181, or combinations thereof.

In some aspects, a bacterial composition disclosed herein comprises a 16S rDNA sequence set forth in SEQ ID NOs: 178, 187, 196, 197, 263, 200, 201, 202, 203, 204, 226, 152, 186, 210, 195, 211, 102, 179, 180, 147, 116, 106, 225, 181, or combinations thereof. In certain aspects, a bacterial composition disclosed herein comprises a 16S rDNA sequence that is at least 97% identical to the 16S rDNA sequence set forth in SEQ ID NOs: 178, 187, 196, 197, 263, 200, 201, 202, 203, 204, 226, 152, 186, 210, 195, 211, 102, 179, 180, 147, 116, 106, 225, 181, or combinations thereof.

In some aspects, a bacterial composition disclosed herein comprises a 16S rDNA sequence set forth in SEQ ID NOs: 178, 187, 196, 197, 263, 200, 201, 202, 203, 204, 152, 210, 195, 211, 103, 224, 179, 180, 116, 106, 181, or combinations thereof. In certain aspects, a bacterial composition disclosed herein comprises a 16S rDNA sequence that is at least 97% identical to the 16S rDNA sequence set forth in SEQ ID NOs: 178, 187, 196, 197, 263, 200, 201, 202, 203, 204, 152, 210, 195, 211, 103, 224, 179, 180, 116, 106, 181, or combinations thereof.

In some aspects, a bacterial composition disclosed herein comprises a 16S rDNA sequence set forth in SEQ ID NOs: 178, 187, 196, 197, 263, 200, 201, 202, 203, 204, 152, 210, 195, 211, 102, 179, 180, 147, 116, 106, 181, or combinations thereof. In certain aspects, a bacterial composition disclosed herein comprises a 16S rDNA sequence that is at least 97% identical to the 16S rDNA sequence set forth in SEQ ID NOs: 178, 187, 196, 197, 263, 200, 201, 202, 203, 204, 152, 210, 195, 211, 102, 179, 180, 147, 116, 106, 181, or combinations thereof.

In some aspects, a bacterial composition disclosed herein comprises a 16S rDNA sequence set forth in SEQ ID NOs: 178, 187, 196, 197, 263, 226, 152, 210, 195, 103, 102, 179, 180, 147, 116, 106, 181, or combinations thereof. In certain aspects, a bacterial composition disclosed herein comprises a 16S rDNA sequence that is at least 97% identical to the 16S rDNA sequence set forth in SEQ ID NOs: 178, 187, 196, 197, 263, 226, 152, 210, 195, 103, 102, 179, 180, 147, 116, 106, 181, or combinations thereof.

In some aspects, a bacterial composition disclosed herein comprises a 16S rDNA sequence set forth in SEQ ID NOs: 178, 187, 196, 197, 263, 152, 210, 223, 195, 211, 102, 179, 180, 147, 116, 106, 181, or combinations thereof. In certain aspects, a bacterial composition disclosed herein comprises a 16S rDNA sequence that is at least 97% identical to the 16S rDNA sequence set forth in SEQ ID NOs: 178, 187, 196, 197, 263, 152, 210, 223, 195, 211, 102, 179, 180, 147, 116, 106, 181, or combinations thereof.

In some aspects, a bacterial composition disclosed herein comprises a 16S rDNA sequence set forth in SEQ ID NOs: 178, 187, 196, 200, 201, 202, 203, 204, 152, 186, 210, 195, 103, 102, 224, 179, 180, 116, 106, 181, or combinations thereof. In certain aspects, a bacterial composition disclosed herein comprises a 16S rDNA sequence that is at least 97% identical to the 16S rDNA sequence set forth in SEQ ID NOs: 178, 187, 196, 200, 201, 202, 203, 204, 152, 186, 210, 195, 103, 102, 224, 179, 180, 116, 106, 181, or combinations thereof.]

In some aspects, a bacterial composition disclosed herein comprises a 16S rDNA sequence set forth in SEQ ID NOs: 178, 187, 196, 197, 263, 200, 201, 202, 203, 204, 212, 152, 186, 195, 211, 103, 102, 116, 106, 225, or combinations thereof. In certain aspects, a bacterial composition disclosed herein comprises a 16S rDNA sequence that is at least 97% identical to the 16S rDNA sequence set forth in SEQ ID NOs: 178, 187, 196, 197, 263, 200, 201, 202, 203, 204, 212, 152, 186, 195, 211, 103, 102, 116, 106, 225, or combinations thereof.

In some aspects, a bacterial composition disclosed herein comprises a 16S rDNA sequence set forth in SEQ ID NOs: 178, 187, 196, 200, 201, 202, 203, 204, 152, 186, 210, 195, 211, 103, 102, 224, 116, 106, 181, or combinations thereof. In certain aspects, a bacterial composition disclosed herein comprises a 16S rDNA sequence that is at least 97% identical to the 16S rDNA sequence set forth in SEQ ID NOs: 178, 187, 196, 200, 201, 202, 203, 204, 152, 186, 210, 195, 211, 103, 102, 224, 116, 106, 181, or combinations thereof.

In some aspects, the bacterial composition described in each of the aspects of the above paragraphs is capable of engrafting (long-term and/or transient) when administered to a subject. In some aspects, the bacterial composition described in each of the aspects of the above paragraphs can have anti-inflammatory activity. In some aspects, the bacterial composition described in each of the aspects of the above paragraphs is not capable of inducing pro-inflammatory activity. In further aspects, the bacterial composition described in each of the aspects of the above paragraphs is capable of producing a secondary bile acid. In certain aspects, the bacterial composition described in each of the aspects of the above paragraphs is capable of producing a tryptophan metabolite. In some aspects, the bacterial composition described in each of the aspects of the above paragraphs is capable of restoring epithelial integrity as determined by a primary epithelial cell monolayer barrier integrity assay. In some aspects, the bacterial composition described in each of the aspects of the above paragraphs is capable of being associated with remission of an inflammatory bowel disease. In further aspects, the bacterial composition described in each of the aspects of the above paragraphs is capable of producing a short-chain fatty acid. In some aspects, the bacterial composition described in each of the aspects of the above paragraphs is capable of producing a medium-chain fatty acid. In still further aspects, the bacterial composition described in each of the aspects of the above paragraphs is capable of inhibiting HDAC activity. In some aspects, the bacterial composition described in each of the aspects of the above paragraphs is capable of inducing Wnt activity. In some aspects, the bacterial composition described in each of the aspects of the above paragraphs is capable of expressing catalase activity. In some aspects, the bacterial composition described in each of the aspects of the above paragraphs is capable of having alpha-fucosidase activity. In some aspects, the bacterial composition described in each of the aspects of the above paragraphs is capable of providing B vitamins. In some aspects, the bacterial composition described in each of the aspects of the above paragraphs is capable of modulating host metabolism of endocannabinoids. In some aspects, the bacterial composition described in each of the aspects of the above paragraphs is capable of producing polyamines and/or modulating host metabolism of polyamines. In some aspects, the bacterial composition described in each of the aspects of the above paragraphs is capable of reducing fecal levels of sphingolipids. In some aspects, the bacterial composition described in each of the aspects of the above paragraphs is capable of modulating host production of kynurenine. In some aspects, the bacterial composition described in each of the aspects of the above paragraphs is capable of reducing fecal calprotectin level. In some aspects, the bacterial composition described in each of the aspects of the above paragraphs is not capable of activating a toll-like receptor pathway (e.g., TLR4 or TLR5). In some aspects, the bacterial composition described in each of the aspects of the above paragraphs is capable of activating a toll-like receptor pathway (e.g., TLR2). In some aspects, the bacterial composition described in each of the aspects of the above paragraphs is capable of all the functional characteristics recited in this paragraph.

In a first aspect, a bacterial composition described herein consists of, or consists essentially of, the eight common bacterial species of DE27, DE28, DE29, DE30, DE31, DE32, DE33, DE34, DE35, DE36, DE37, DE39, DE41, DE42, DE43, DE44, DE45, DE46, DE47, DE48, DE49, DE50, DE51, DE52, DE53, and DE54.

In a second aspect, a bacterial composition described herein consists of, or consists essentially of, the 10 common bacterial species of DE27, DE29, DE30, DE32, DE33, DE34, DE35, DE36, DE37, DE39, DE41, DE42, DE45, DE46, DE47, DE48, DE49, DE50, and DE51.

In a third aspect, a bacterial composition described herein consists of, or consists essentially of, the eleven common bacterial species of DE27, DE29, DE32, DE33, DE34, D35, DE36, DE37, DE39, DE41, DE42, DE45, DE46, DE47, DE48, DE49, DE50, and DE51.

In a fourth aspect, a bacterial composition described herein consists of, or consists essentially of, the twelve common bacterial species of DE29, DE32, DE33, DE34, DE35, DE36, DE37, DE39, DE41, DE42, DE45, DE46, DE48, and DE49.

In a fifth aspect, a bacterial composition described herein consists of, or consists essentially of, the thirteen common bacterial species of DE32, DE33, DE34, DE35, DE36, DE37, DE39, DE41, DE42, DE45, DE46, DE48, and DE49.

In a sixth aspect, a bacterial composition described herein consists of, or consists essentially of, the fifteen common bacterial species of DE33, DE35, DE36, DE37, and DE39.

In a seventh aspect, a bacterial composition described herein consists of, or consists essentially of, the seventeen common bacterial species of DE36, DE37, and DE39. As used herein, the term “consists essentially of” allows for the inclusion of additional components (e.g., bacterial species, such as those disclosed herein) that do not affect the overall property of a bacterial composition described herein. For instance, in some aspects, the term consists essentially of allows for the addition of one or more additional bacterial species to a bacterial composition described in each of the above seven aspects, where the one or more additional bacterial species share the same functional features (e.g., those described herein) as a bacterial species already present in the composition.

In some aspects, the bacterial composition described in each of the seven aspects provided above is capable of engrafting (long-term and/or transient) when administered to a subject. In some aspects, the bacterial composition described in each of the seven aspects provided above can have anti-inflammatory activity. In some aspects, the bacterial composition described in each of the seven aspects provided above is not capable of inducing pro-inflammatory activity. In further aspects, the bacterial composition described in each of the seven aspects provided above is capable of producing a secondary bile acid. In certain aspects, the bacterial composition described in each of the seven aspects provided above is capable of producing a tryptophan metabolite. In some aspects, the bacterial composition described in each of the seven aspects provided above is capable of restoring epithelial integrity as determined by a primary epithelial cell monolayer barrier integrity assay. In some aspects, the bacterial composition described in each of the seven aspects provided above is capable of being associated with remission of an inflammatory bowel disease. In further aspects, the bacterial composition described in each of the seven aspects provided above is capable of producing a short-chain fatty acid. In some aspects, the bacterial composition described in each of the seven aspects provided above is capable of producing a medium-chain fatty acid. In still further aspects, the bacterial composition described in each of the seven aspects provided above is capable of inhibiting HDAC activity. In some aspects, the bacterial composition described in each of the seven aspects provided above is capable of inducing Wnt activity. In some aspects, the bacterial composition described in each of the seven aspects provided above is capable of expressing catalase activity. In some aspects, the bacterial composition described in each of the seven aspects provided above is capable of having alpha-fucosidase activity. In some aspects, the bacterial composition described in each of the seven aspects provided above is capable of providing B vitamins. In some aspects, the bacterial composition described in each of the seven aspects provided above is capable of modulating host metabolism of endocannabinoids. In some aspects, the bacterial composition described in each of the seven aspects provided above is capable of producing polyamines and/or modulating host metabolism of polyamines. In some aspects, the bacterial composition described in each of the seven aspects provided above is capable of reducing fecal levels of sphingolipids. In some aspects, the bacterial composition described in each of the seven aspects provided above is capable of modulating host production of kynurenine. In some aspects, the bacterial composition described in each of the seven aspects provided above is capable of reducing fecal calprotectin level. In some aspects, the bacterial composition described in each of the seven aspects provided above is not capable of activating a toll-like receptor pathway (e.g., TLR4 or TLR5). In some aspects, the bacterial composition described in each of the seven aspects provided above is capable of activating a toll-like receptor pathway (e.g., TLR2). In some aspects, the bacterial composition described in each of the seven aspects provided above is capable of inhibiting apoptosis of intestinal epithelial cells. In some aspects, the bacterial composition described in each of the seven aspects provided above is capable of inducing an anti-inflammatory IL-10-skewed IL-10/IL-6 cytokine ratio in macrophages. In some aspects, the bacterial composition described in each of the seven aspects provided above is capable of not inducing pro-inflammatory IL-6, TNFa, IL-1b, IL-23 or IL-12 production or gene expression in macrophages. In some aspects, the bacterial composition described in each of the seven aspects provided above is capable of all the functional characteristics recited in this paragraph.

The term “16S sequencing” or “16S rDNA” or “16S” refers to sequence derived by characterizing the nucleotides that comprise the 16S ribosomal RNA gene(s). The bacterial 16S rDNA is approximately 1500 nucleotides in length and is used in reconstructing the evolutionary relationships and sequence similarity of one bacterial isolate to another using phylogenetic approaches. 16S sequences are used for phylogenetic reconstruction as they are in general highly conserved, but contain specific hypervariable regions that harbor sufficient nucleotide diversity to differentiate genera and species of most bacteria.

The term “V1-V9 regions” of the 16S rRNA refers to the first through ninth hypervariable regions of the 16S rRNA gene that are used for genetic typing of bacterial samples. These regions in bacteria are defined by nucleotides 69-99, 137-242, 433-497, 576-682, 822-879, 986-1043, 1117-1173, 1243-1294 and 1435-1465 respectively using numbering based on the E. coli system of nomenclature. Brosius et al., Complete nucleotide sequence of a 16S ribosomal RNA gene from Escherichia coli, PNAS 75(10):4801-4805 (1978). In some aspects, at least one of the V1, V2, V3, V4, V5, V6, V7, V8, and V9 regions are used to characterize an OTU. In some aspects, the V1, V2, and V3 regions are used to characterize an OTU. In another aspect, the V3, V4, and V5 regions are used to characterize an OTU. In another aspect, the V4 region is used to characterize an OTU. A person of ordinary skill in the art can identify the specific hypervariable regions of a candidate 16S rRNA by comparing the candidate sequence in question to a reference sequence and identifying the hypervariable regions based on similarity to the reference hypervariable regions, or alternatively, one can employ Whole Genome Shotgun (WGS) sequence characterization of microbes or a microbial community.

In some aspects, a bacterial composition disclosed herein (e.g., designed compositions) comprises both a spore-forming bacteria and a non-spore forming bacteria. In some aspects, a bacterial composition comprises only spore-forming bacteria. In some cases, the bacteria of the composition are in spore form.

Applicant has also discovered that certain bacterial species are associated with exacerbation or non-improvement of at least one sign or symptom of a disease or disorder associated with dysbiosis of the gastrointestinal microbiome (e.g., ulcerative colitis). The presence of such species in a bacterial composition can be undesirable. Accordingly, in some aspects, a bacterial composition (e.g., designed compositions) does not include one or more of the following bacterial species: Eubacterium contortum, Clostridium hathewayi, Erysipelatoclostridum ramosum, Bifidobacterium dentium, Dialister invisus, Prevotella copri, Veillonella atypica, Veillonella dispar, Veillonella parvula, or Veillonella ratti. In certain aspects, a bacterial composition does not include one or more bacteria that has at least about 97%, e.g., about 99% identity, to a 16S rDNA of the foregoing species. In some aspects, a bacterial composition does not include at least one, two, three, four, five, six, seven, eight, nine, ten, eleven, or all of the species listed.

In some aspects, a bacterial composition of the present disclosure does not comprise one or more bacteria comprising a 16S rDNA sequence that is at least about 85%, at least about 90%, at least about 95%, at least about 96%, at least about 97%, at least about 97.5%, at least 98%, at least about 98.5%, at least about 99%, at least about 99.5%, or about 100% identical to a 16S rDNA sequence set forth in SEQ ID NO: 15, 31, 37, 38, 40, 42, 43, 46, 52-58, 63, 69-71, and 83-101.

As described supra, Applicant has discovered that bacteria that are beneficial for the treatment of a disease or disorder associated with dysbiosis (e.g., ulcerative colitis) are associated with certain biological functions. Accordingly, in some aspects, types of bacteria present in a bacterial composition disclosed herein (e.g., designed compositions) are associated with certain biological functions, which are useful in treating, preventing, delaying, or ameliorating one or more signs or symptoms associated with a disease or disorder disclosed herein (e.g., ulcerative colitis). Non-limiting examples of relevant functional features are further described below. See also International Application No. PCT/US2019/034069, which is incorporated herein by reference in its entirety.

Functional Features

In some aspects of the invention, a microbiome composition disclosed herein (e.g., designed compositions) is a composition that includes bacteria that can carry out certain functions identified by applicant as being useful for treating and/or preventing a disease or disorder associated with dysbiosis. Non-limiting examples of such disease or disorder are provided elsewhere in the present disclosure (e.g., an IBD, such as UC; and cancer).

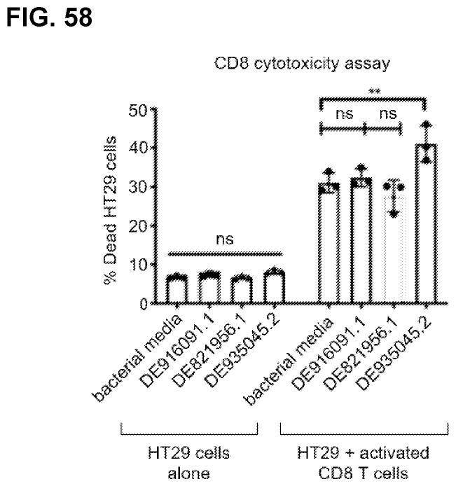

In certain aspects, bacterial species that are useful for the present disclosure comprises one or more of the following features: (1) capable of engrafting (long-term and/or transient) when administered to a subject; (2) capable of having anti-inflammatory (e.g., inhibiting TNF-α-driven IL-8 secretion in epithelial cells in vitro, ability to downmodulate expression of inflammatory genes (e.g., CXCL1, CXCL2, CXCL3, CXCL11, ICAM1), ability to downmodulate one or more inflammatory genes induced by IFN-γ as measured in the colonic organoids (e.g., such as those described in Example 13)); (3) not capable of inducing pro-inflammatory activity (e.g., does not induce IL-8 production by IECs); (4) capable of producing secondary bile acids (e.g., 7α-dehydroxylase and bile salt hydrolase activity) (e.g., production of DCA, 12-oxo-3a, 3-oxo-7a, and/or 3β 12α-deoxycholic acid; see, e.g., FIGS. 58A-58D); (5) not capable of producing ursodeoxycholic acid (e.g., 7p-hydroxysteroid dehydrogenase activity); (6) capable of producing tryptophan metabolites (e.g., indole, 3-methyl indole, indolepropionic acid); (7) capable of producing medium-chain (e.g., valerate and hexanoate) and/or short-chain fatty acids (e.g., butyrate and propionate); (8) capable of inhibiting HDAC activity; (9) capable of restoring epithelial integrity, as determined by a primary epithelial cell monolayer barrier integrity assay; (10) capable of being associated with clinical remission of an inflammatory bowel disease; (11) capable of not being associated with clinical non-remission of an inflammatory bowel disease (12) capable of expressing catalase activity; (13) capable of having alpha-fucosidase activity; (14) capable of inducing Wnt activation; (15) capable of producing B vitamins (e.g., thiamin (B1) and pyridoxamine (B6)); (16) capable of modulating host metabolism of endocannabinoids; (17) capable of producing polyamines and/or modulating host metabolism of polyamines; 18) capable of reducing fecal levels of sphingolipids; (19) capable of modulating host production of kynurenine; (20) capable of reducing fecal calprotectin level; (21) not capable of activating a toll-like receptor pathway (e.g., TLR4 or TLR5); (22) capable of activating a toll-like receptor pathway (e.g., TLR2); (23) capable of producing a short-chain fatty acid (e.g., butyrate, propionate); (24) capable of inhibiting IFN-γ-induced expression of caspases and other genes involved in apoptosis in IECs; (25) capable of inducing expression and/or secretion of anti-inflammatory cytokines (e.g. IL-10) by macrophages; (26) not capable of inducing expression or secretion of pro-inflammatory cytokines (e.g. IL-6, IL-1b, TNFa, IL-23, or IL-12) by macrophages; (27) capable of downmodulating one or more genes induced in IFN-γ treated colonic organoids (e.g., those associated with inflammatory chemokine signaling, NF-κB signaling, TNF family signaling, type I interferon signaling, type II interferon signaling, TLR signaling, lymphocyte trafficking, Th17 cell differentiation, Th1 differentiation, Th2 differentiation, apoptosis, inflammasomes, autophagy, oxidative stress, MHC class I and II antigen presentation, complement, mTor, nod-like receptor signaling, PI3K signaling, or combinations thereof), (28) capable of producing IL-18, (29) capable of inducing the activation of antigen presenting cells, (30) capable of reducing the expression of one or more inhibitory receptors (e.g., TIGIT, TIM-3, or LAG-3) on T cells, (31) capable of increasing expression of one or more genes/proteins associated with T cell activation and/or function (e.g., CD45RO, CD69, IL-24, TNF-α, perforin, or IFN-γ), (32) capable of enhancing the ability of CD8+ T cells to kill tumor cells, (33) capable of enhancing the efficacy of an immune checkpoint inhibitor therapy, (34) capable of reducing colonic inflammation, (35) capable of promoting the recruitment of CD8+ T cells to tumors, or (36) any combination thereof. In certain aspects, species that are useful for the present disclosure comprises one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, or all of the above features.

Additional disclosure relating to exemplary functional features are provided below and also elsewhere in the present disclosure (see, e.g., Section III).

Engraftment

As described supra, a key feature of the bacterial compositions disclosed herein is the ability of one or more bacterial species (or OTUs of bacteria) included in the compositions to engraft in a subject when administered to the subject. Accordingly, Applicant has identified bacteria and combinations of bacteria that are capable of engrafting when administered to a subject. Not to be bound by any one theory, engraftment of bacteria and combinations of bacteria disclosed herein can repopulate the gastrointestinal microbiome of a subject. In some aspects, once engrafted, bacteria and combinations of bacteria disclosed herein prevent (e.g., by outcompeting for growth nutrients) the growth of non-commensal microbes (e.g., pathogenic bacteria, such as Clostridium difficile) that may result in inflammatory responses in the host. In further aspects, once engrafted, bacteria and combinations of bacteria disclosed herein can promote or augment the growth of other commensal bacteria within the subject. In further aspects, the engrafting bacteria and combinations of bacteria can produce various factors (e.g., tryptophan metabolites, fatty acids, secondary bile acids) or exert other functions (e.g., those disclosed herein) to help treat and/or prevent one or more symptoms associated with a disease or disorder disclosed herein.

Whether bacteria or combinations of bacteria are capable of engrafting can be determined by various methods known in the art. Subject samples can first be collected (e.g., by whole stool samples, rectal swaps, tissue biopsies, or mucosal samples) before and/or after administration of bacteria or combinations of bacteria. Subsequently, these samples can be characterized to identify the bacteria or combinations of bacteria. Administered bacterial strains can be identified in samples based on genotypic, phenotypic, and other molecular properties of the strains, for example: a) the sequence of certain genes (e.g., 16S rRNA sequence) b) the presence and/or sequence identity of one or more regions of DNA (i.e., linear segments) that are rarely present in other strains, rarely present in other microbiome samples, rarely present in the target patient population, or absent from the microbiome of the particular subject(s) before administration of the bacteria, c) DNA variants including SNVs, insertions and deletions (i.e., indels), structural variation, gene copy number variation, or other DNA variants that are rarely present in other strains, rarely present in other microbiome samples, rarely present in the target patient population, or absent from the microbiome of the particular subject(s) before administration of the bacteria, d) other identifying phenotypic, genomic, proteomic, metabolomic or other properties of the administered strains. Molecular technologies used to identify administered bacteria or combinations of bacteria include but are not limited various DNA sequencing technologies including PCR and qPCR, amplicon sequencing, whole genome sequencing, shotgun metagenomic sequencing; other molecular technologies can be used included but not limited to microarray, multiplexed molecular barcode (e.g., available from NanoString Technologies), and mass spectrometry. Bioinformatic methods used to analyze these data may include sequence alignment and mapping, genome or metagenome assembly, or other methods. Microbiological and culturing methods can also be used to identify and characterize strains. These mentioned methods of identification and characterization of administered bacteria or combinations of bacteria can be used alone or in combination.

In some aspects, one or more of the bacterial species included in the bacterial compositions disclosed herein are capable of engrafting when administered to a subject. In certain aspects, each of the bacterial species included in a bacterial composition is capable of engrafting. In some aspects, the bacteria and combinations of bacteria that are capable of engrafting are long-term engrafters. In certain aspects, the bacteria and combinations of bacteria that are capable of engrafting are transient engrafters. In some aspects, the bacterial compositions disclosed herein (e.g., designed compositions) comprise one or more long-term engrafters and one or more transient engrafters. In certain aspects, a bacterial composition disclosed herein comprises two, three, four, five, six, seven, eight, nine, ten or more long-term engrafters. In some aspects, a bacterial composition comprises two, three, four, five, six, seven, eight, nine, ten or more transient engrafters. In further aspects, a bacterial composition disclosed herein comprises three or more transient engrafters and/or seven or more long-term engrafters. Non-limiting examples of long-term engrafters and/or transient engrafters that can be used with the present disclosure are provided in Table 5.

Bile Acids

Applicant has discovered that certain secondary bile acids are associated with the treatment and/or prevention of a disease or disorder, such as those associated with a dysbiosis (e.g., remission of UC). The term “bile acids” refers to a family of molecules, composed of a steroid structure with four rings, a five or eight carbon side-chain terminating in a carboxylic acid joined at the 17-position of the steroid scaffold, and the presence and orientation of different numbers of hydroxy groups. Depending on the tissue, the structure of the bile acids can vary. For instance, upon their synthesis in the liver, the bile acids are conjugated to either taurine or glycine residues (“conjugated primary bile acids” also known as bile salts) and subsequently excreted and stored in the gall bladder. During digestion, the conjugated primary bile acids are then secreted into the intestinal lumen. In some aspects, the primary conjugated bile acids are glycocholic acid (gCA), taurocholic acid (tCA), glycochenodeoxycholic acid (gCDCA), or taurochenodeoxycholic acid (tCDCA).

Within the intestinal lumen, the resident intestinal bacteria express enzymes (e.g., bile salt hydrolase (BSH)), which deconjugate the conjugated primary bile acids to produce “primary bile acids.” In some aspects, the primary bile acids comprise cholic acid (CA) or chenodeoxycholic acid (CDCA). Primary bile acids are then further processed (via enzymes, such as hydroxysteroid dehydrogenase (HSDH) or 7α-dehydroxylase) to become “secondary bile acids.” Accordingly, in some aspects, the phrase “capable of producing a secondary bile acid” comprises the ability to deconjugate primary bile acids to produce the secondary bile acids. In some aspects, the secondary bile acids comprise deoxycholic acid (DCA), (3 or 12)-oxo-deoxycholic acid, (3 or 12)-iso-deoxycholic acid, (3, 7 or 12)-oxo-cholic acid, (3, 7 or 12)-iso-cholic acid, lithocholic acid (LCA), oxo-LCA, iso-LCA, (3 or 7)-oxo-chenodeoxy cholic acid, or (3 or 7)-iso-chenodeoxy cholic acid.

The secondary bile acids produced in the intestinal lumen can circulate back to the liver, where they are reconjugated to become “conjugated secondary bile acids.” In some aspects, the secondary conjugated bile acids of the present disclosure comprise (3 or 12)-glyco-iso-deoxycholic acid, (3 or 12)-tauro-iso-deoxycholic acid, glyco-deoxycholic acid, tauro-deoxycholic acid, (3, 7 or 12)-glyco-iso-cholic acid, (3, 7 or 12)-tauro-iso-cholic acid, sulfo-lithocholic acid, glyco-sulfo-lithocholic acid, tauro-sulfo-lithocholic acid, (3 or 7)-glyco-iso-chenodeoxycholic acid, (3 or 7)-tauro-iso-chenodeoxycholic acid, (3 or 7)-glyco-oxo-chenodeoxycholic acid, or (3 or 7)-tauro-oxo-chenodeoxycholic acid.

In some aspects, one or more of the bacterial species that can be used in constructing the designed compositions disclosed herein comprise an enzyme involved in secondary bile acid production. In certain aspects, the enzyme comprises BSH or HSDH. In some aspects, a bacterial species useful for the present disclosure comprises both BSH and HSDH. Accordingly, in some aspects, bacteria and combinations of bacteria disclosed herein can increase the level of a bile acid (e.g., a secondary bile acid, e.g., deoxycholic acid (DCA), 3-α-12-oxo-deoxycholic acid, 3-β-12-α-deoxycholic acid (3-isodeoxycholic acid), 7-α-3-oxo-chenodeoxycholic acid, lithocholic acid (LCA), 3-oxoLCA, oxo-LCA, iso-LCA, and combinations thereof) in a subject.

In some aspects, the level of a secondary bile acid is increased by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, compared to a corresponding level in a reference sample. In some aspects, the reference sample is a biological sample (e.g., fecal sample) obtained from a subject prior to the administration of a bacterial composition disclosed herein. In other aspects, the reference sample is a biological sample (e.g., fecal sample) obtained from a subject with an active symptom of a disease or disorder, such as those associated with dysbiosis (e.g., ulcerative colitis flare-up).

In some aspects, the increase in the level of a secondary bile acid can reduce the level of a pro-inflammatory mediators (e.g., TNF-α or IL-8) produced by activated cells (e.g., LPS-stimulated monocytes, LPS-stimulated PBMCs, or TNF-α-stimulated intestinal epithelial cells). In some aspects, the increase in the level of a secondary bile acid can increase the level of anti-inflammatory mediators (e.g., IL-10) produced by activated cells. In some aspects, the increase in the level of a secondary bile acid is correlated with an improvement of at least one aspect of the disease state (e.g., clinical remission or endoscopic/histologic response or reduced levels of fecal calprotectin).

In certain aspects, the amount of pro-inflammatory mediators produced by activated cells is decreased by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, compared to a reference sample (e.g., activated cells not treated with increased concentration of a secondary bile acid). In some aspects, the level of anti-inflammatory mediators produced is increased by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to a reference sample (e.g., activated cells not treated with increased concentration of a secondary bile acid).

In some aspects, reducing the level of certain secondary bile acids can be important in the effective treatment of a disease or disorder disclosed herein. A non-limiting example of such a secondary bile acid is ursodeoxycholic acid. Accordingly, in certain aspects, bacteria and combinations of bacteria that are useful for the present disclosure are capable of reducing the level of a secondary bile acid in a subject. In some aspects, the level of a secondary bile acid is reduced by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, compared to a corresponding level in a reference sample. In some aspects, the reference sample is a biological sample (e.g., fecal sample) obtained from a subject prior to the administration of a bacterial composition disclosed herein. In other aspects, the reference sample is a biological sample (e.g., fecal sample) obtained from a subject with an active symptom of a disease or disorder, such as those associated with dysbiosis (e.g., ulcerative colitis flare-up).

Anti-Inflammatory Activity

Applicant has identified bacteria and combinations of bacteria that are capable of exhibiting anti-inflammatory activity when administered to a subject. As used herein, the term “anti-inflammatory activity” refers to the ability to prevent and/or reduce inflammation The term “inflammation” or “pro-inflammatory” refers to the complex biological response of an individual's immune system to harmful stimuli, such as pathogens, damaged cells, or irritants, and includes secretion of pro-inflammatory mediators, such as pro-inflammatory cytokines, i.e., cytokines which are produced predominantly by activated immune cells, such as macrophages and dendritic cells, and are involved in the amplification of inflammatory reactions.

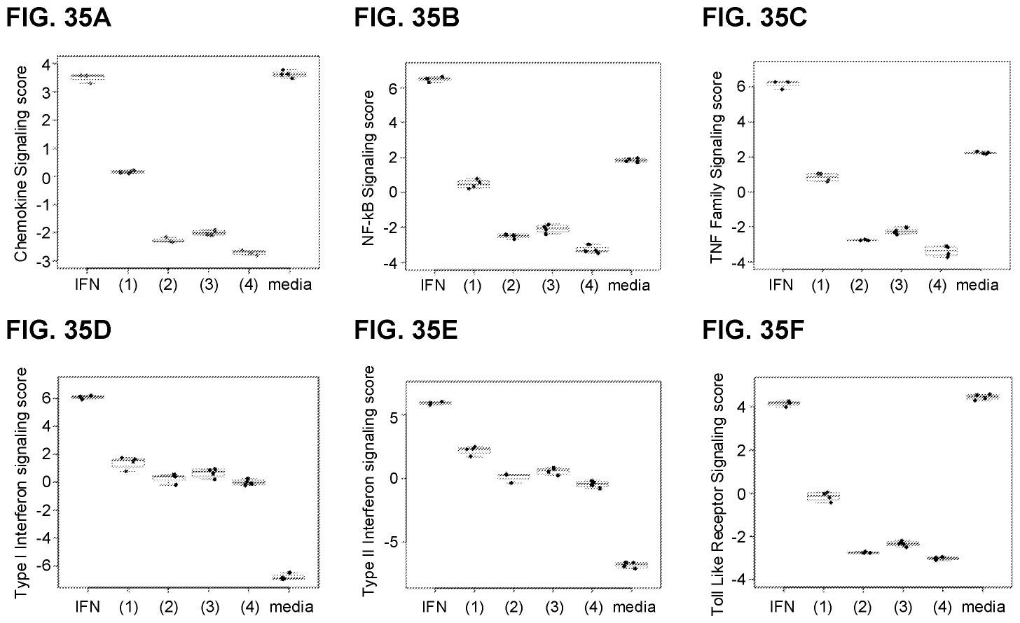

Without being limited to any one particular theory, the anti-inflammatory activity observed with the bacteria and combinations of bacteria disclosed herein can be related to the other functional aspects of the bacteria or combinations of bacteria. For example, in some aspects, the anti-inflammatory activity is related to the ability of the bacteria or combinations of bacteria to produce a secondary bile acid, a tryptophan metabolite, a short-chain fatty acid, inhibit HDAC inhibition, and/or inhibit TNF-α-driven IL-8 secretion in epithelial cells in vitro. In some aspects, the anti-inflammatory activity are related to the ability of the bacteria or combinations of bacteria to downmodulate one or genes that are induced by inflammatory cytokines (e.g., such as those observed in IFN-γ treated colonic organoids; see, e.g., FIGS. 35A-35E, FIGS. 36A-36D, 37A-37D, 38A-38D, 39A-39C, 40A-40B, 41A-41B, 42, and Example 13). Accordingly, in some aspects, the bacteria and combinations of bacteria that have anti-inflammatory activity have one or more of the following features: (i) capable of producing a short-chain fatty acid, (ii) capable of inhibiting histone deacetylase (HDAC) activity, (iii) capable of inhibiting TNF-α-driven IL-8 secretion in epithelial cells in vitro, (iv) capable of inhibiting NF-κB and NF-κB target genes, (v) capable of downmodulating one or more genes induced in IFN-γ treated colonic organoids (e.g., those associated with inflammatory chemokine signaling, NF-κB signaling, TNF family signaling, type I interferon signaling, type II interferon signaling, TLR signaling, lymphocyte trafficking, Th17 cell differentiation, Th1 differentiation, Th2 differentiation, apoptosis, inflammasomes, autophagy, oxidative stress, MHC class I and II antigen presentation, complement, mTor, nod-like receptor signaling, PI3K signaling, or combinations thereof); (vi) capable of inducing anti-inflammatory IL-10 production in macrophages in vitro; (vii) capable of not inducing pro-inflammatory IL-6, TNFa, IL-1b, IL-23 or IL-12 production or gene expression in macrophages; or (viii) any combination thereof. Whether bacteria or combinations of bacteria have anti-inflammatory activity can be measured using assays known in the art, including but not limited to methods to measure metabolites like short-chain fatty acids (e.g., MS, LC-MS, GS-MS, LC-MS/MS), methods of measuring gene expression at the RNA and/or protein level (e.g., multiplexed bead-based (e.g., available from Luminex) cytokine panels, microarray, multiplexed molecular barcode (e.g., available from NanoString Technologies), flow cytometry, and RNA-sequencing).

In some aspects, the anti-inflammatory activity of the bacteria and combinations of bacteria disclosed herein can reduce the amount of pro-inflammatory mediators produced and/or present in a subject (e.g., suffering from a disease or disorder disclosed herein). In certain aspects, the amount of pro-inflammatory mediators produced and/or present in the subject is decreased by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, compared to a reference sample. In some aspects, the reference sample is a biological sample obtained from a subject prior to the administration of a bacterial composition disclosed herein. In other aspects, the reference sample is a biological sample obtained from a subject with an active symptom of a disease or disorder, such as those associated with dysbiosis (e.g., ulcerative colitis flare-up).

In some aspects, the anti-inflammatory activity of the bacteria and combinations of bacteria disclosed herein can increase the amount of anti-inflammatory mediators in a subject. Non-limiting examples of anti-inflammatory mediators include, but are not limited to, IL-1 receptor antagonists (IL-lRA), IL-4, IL-6, IL-10, IL-11, IL-13, TGF-β, and combinations thereof. In certain aspects, the bacteria and combinations of bacteria that are capable of exhibiting anti-inflammatory activity can increase the amount of anti-inflammatory mediators in a subject by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, compared to a reference sample. In some aspects, the reference sample is a biological sample obtained from a subject prior to the administration of a bacterial composition disclosed herein. In other aspects, the reference sample is a biological sample obtained from a subject with an active symptom of a disease or disorder, such as those associated with dysbiosis (e.g., ulcerative colitis flare-up).

Tryptophan Metabolism and Aryl Hydrocarbon Receptor

As used herein, the term “tryptophan” refers to the essential amino acid tryptophan, which is an α-amino acid and has a chemical formula of C₁₁H₁₂N₂O₂. Besides its use in protein synthesis, tryptophan is important in a number of pathways leading to the production of, for example, serotonin (5-hydroxytryptamine), melatonin, kynurenines, and tryptamine. Tryptophan and its metabolites can affect, for example, immunosuppression, immune function, cancer, inflammatory disease, epithelial barrier function, and infection.

Certain tryptophan pathway products have been shown to function as aryl hydrocarbon receptor (Ahr) agonists. The metabolites include, for example, indole, indole-3 aldehyde, indole-3 acetate, indole-3 propionic acid, indole, 3-methylindole, indole-3 acetaldehyde, indole-3 acetonitrile, 6-formylindolo[3,2-b]carbazole (FICZ), and tryptamine. Ahr plays a role in controlling the differentiation and activity of specific T cell subpopulations. It reportedly can influence adaptive immune responses through its effects on both T cells and antigen presenting cells (APCs). Ahr is thought to be involved in development and maintenance of CD4+ T regulatory cells (Tregs) as well as FoxP3-IL-10+ CD4+ Tr1, and induction of Th17 cells. Ahr also alters cytokine expression by Type 3 innate lymphoid cells (ILC3s). These cellular effects include increased production of IL-22. AhR induction by Trp metabolites has been reported to enhance epithelial barrier integrity and ameliorate colitis in in vivo models.

In some aspects, bacteria or combination of bacteria disclosed herein can increase the level of a tryptophan metabolite in a subject. In some aspects, tryptophan metabolite comprises indole, 3-methyl indole, indoleacrylate, or any combination thereof. In certain aspects, bacteria or combination of bacteria disclosed herein can increase the level of indole and/or 3-methylindole in the subject.

In some aspects, the level of a tryptophan metabolite is increased by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, compared to a corresponding level in a reference sample. In some aspects, the reference sample is a biological sample (e.g., fecal sample) obtained from a subject prior to the administration of a bacterial composition disclosed herein. In other aspects, the reference sample is a biological sample (e.g., fecal sample) obtained from a subject with an active symptom of a disease or disorder, such as those associated with dysbiosis (e.g., ulcerative colitis flare-up).

In some aspects, bacteria or combination of bacteria disclosed herein can increase the level of AhR-mediated Cyp1a1 expression in a subject. In some aspects, the level of AhR-mediated Cyp1a1 expression is increased by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, compared to a corresponding level in a reference sample. In some aspects, the reference sample is a biological sample (e.g., fecal sample) obtained from a subject prior to the administration of a bacterial composition disclosed herein. In other aspects, the reference sample is a biological sample (e.g., fecal sample) obtained from a subject with an active symptom of a disease or disorder, such as those associated with dysbiosis (e.g., ulcerative colitis flare-up).

Without being limited to a specific mechanism, in some aspects, bacteria disclosed herein increase the level of AhR-mediated Cyp1a1 expression through an increase in tryptophan metabolite production. In some aspects, increase in a tryptophan metabolite (e.g., indole or 3-methylindole) level is correlated with improvement of a disease or disorder disclosed herein (e.g., clinical remission). Accordingly, in some aspects, increase in the level of AhR-mediated Cyp1a1 expression is correlated with one or more features associated with an improvement in a subject's condition, e.g., a subject diagnosed with a disease or disorder, such as those associated with dysbiosis (e.g., an IBD, such as ulcerative colitis).

In some aspects, reducing the level of a tryptophan metabolite in a subject might be useful in treating a disease or disorder. Accordingly, in certain aspects, bacteria and combinations of bacteria disclosed herein are capable of reducing the level of a tryptophan metabolite in a subject. In some aspects, the level of a tryptophan metabolite is reduced by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, compared to a corresponding level in a reference sample. In some aspects, the reference sample is a biological sample (e.g., fecal sample) obtained from a subject prior to the administration of a bacterial composition disclosed herein. In other aspects, the reference sample is a biological sample (e.g., fecal sample) obtained from a subject with an active symptom of a disease or disorder, such as those associated with dysbiosis.

Fatty Acids

Applicant has identified bacteria and combinations of bacteria that are capable of producing certain fatty acids in a subject. In some aspects, fatty acids comprise short-chain fatty acids. In other aspects, fatty acids comprise medium-chain fatty acids. As used herein, the term “short-chain fatty acids” refer to fatty acids with less than six carbon atoms. Non-limiting examples of short-chain fatty acids include formate, acetate, propionate, butyrate, isobutryate, valerate, isovalerate, and combinations thereof. In certain aspects, short-chain fatty acid comprises acetate, propionate, butyrate, or combinations thereof. As used herein, the term “medium-chain fatty acids” refer to fatty acids with aliphatic tails of 6 to 12 carbon atoms, which can form medium-chain triglycerides. Non-limiting examples of medium-chain fatty acids include hexanoate, oxtanoate, decanoate, dodecanoate, and combinations thereof. In some aspects, medium-chain fatty acid comprises hexanoate.

In some aspects, bacteria or combination of bacteria disclosed herein increases the level of a short-chain fatty acid in a subject. In certain aspects, short-chain fatty acid comprises formate, acetate, propionate, butyrate, isobutryate, valerate, isovalerate, or any combination thereof. In some aspects, the short-chain fatty acid comprises propionate, butyrate, acetate, or combinations thereof. In some aspects, the level of a short-chain fatty acid in the subject is increased by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, compared to a corresponding level in a reference sample. In some aspects, the reference sample is a biological sample (e.g., fecal sample) obtained from a subject prior to the administration of a bacterial composition disclosed herein. In other aspects, the reference sample is a biological sample (e.g., fecal sample) obtained from a subject with an active symptom of a disease or disorder, such as those associated with dysbiosis (e.g., ulcerative colitis flare-up).

In some aspects, bacteria or combination of bacteria disclosed herein increases the level of a medium-chain fatty acid in a subject. In certain aspects, the medium-chain fatty acid comprises hexanoate. In some aspects, the level of a medium-chain fatty acid in the subject is increased by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, compared to a corresponding level in a reference sample. In some aspects, the reference sample is a biological sample (e.g., fecal sample) obtained from a subject prior to the administration of a bacterial composition disclosed herein. In other aspects, the reference sample is a biological sample (e.g., fecal sample) obtained from a subject with an active symptom of a disease or disorder, such as those associated with dysbiosis (e.g., ulcerative colitis flare-up).

Inhibition of Histone Deacetylase (HDAC) Activity

Histone deacetylases (HDACs) are a family of enzymes that can remove acetyl residues from specific sites in the N-terminal end of histones, which are part of the DNA chromatin structure in eukaryotic cells. The steady state of histone acetylation is a result of the balance of acetylation by histone acetyltransferase (HAT) enzymes and deacetylation by HDACs. When HDACs are inhibited but HATs activity continues, histones become hyperacetylated, thus disrupting high order chromatin structure and stimulating transcription by RNA polymerase III. The effect of HDAC inhibition in gene expression is not generalized, as only 2% of mammalian genes are affected by HDAC inhibition.

Some short chain fatty acids (SCFAs) produced by the intestinal human microbiome are HDAC inhibitors. Butyrate in particular has been identified as an HDAC inhibitor in vitro and in vivo, leading to the accumulation of hyperacetylated histones H3 and H4 (Candido et al., 1978 Cell 14:105-113; Boffa et al. 1978 J Biol Chem 253:3364-3366; Vidali et al. 1978 Proc Natl Acad Sci USA 75:2239-2243; Davie. 2003 J Nutrition 133:2485S-2493S). Other SCFAs, such as propionate, isobutyrate, isovalerate, valerate, lactate, and acetate, can also inhibit histone deacetylation, although reportedly less effectively than butyrate (Sealy and Chalkley. 1978 Cell 14:115-121; Latham et al. Nucl Acids Res 40:4794-4803, Waldecker et al. 2008 J Nutr Biochem 19:587-593). Certain therapeutic effects of butyrate are reportedly mediated, at least in part, by inhibition of HDACs.

In some aspects, bacteria and combinations of bacteria disclosed herein are capable of inhibiting (or reducing) HDAC activity. In some aspects, bacteria and combinations of bacteria disclosed herein can inhibit (or reduce) HDAC activity in a subject by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, compared to a reference sample. In some aspects, the reference sample is a biological sample obtained from a subject prior to the administration of a bacterial composition disclosed herein. In other aspects, the reference sample is a biological sample obtained from a subject with an active symptom of a disease or disorder, such as those associated with dysbiosis (e.g., ulcerative colitis flare-up).

In some aspects, the bacteria disclosed herein that are capable of inhibiting HDAC activity can be further grouped into one of seven phenotypic clusters (represented as 0-6 in FIG. 13; termed herein “HDAC clusters”) based on their ability to inhibit HDAC activity when grown in different nutrient sources. Non-limiting examples of nutrient sources that can be used include, but are not limited to, peptone/yeast extract medium (PY) alone or supplemented with 0.5% of one of seven C sources (glucose, fucose, sucrose, starch, pectin, FOS/inulin, or mucin). As used herein, “HDAC cluster 0” corresponds to strains that are capable of inhibiting HDAC when grown on fucose (a sugar found as a component of mucin glycoproteins) but not on other substrates. These strains can utilize fucose as a substrate for propionate production, but not amino acids present in the basal media or other simple and complex carbohydrates added in other conditions. “HDAC cluster 1” corresponds to strains that are not capable of inhibiting HDAC when grown in any of the nutrient sources disclosed herein. “HDAC cluster 2” corresponds to strains that are capable of inhibiting HDAC and have reduced inhibition when grown in the presence of sucrose, inulin, glucose, or pectin. “HDAC cluster 3” corresponds to strains that are capable of inhibiting HDAC and have reduced inhibition when grown in the presence of sucrose, inulin, glucose, or pectin. Strains belonging to HDAC cluster 3 are capable of having increased inhibition of HDAC when grown in the presence of mucin. “HDAC cluster 4” corresponds to strains that are capable of inhibiting HDAC in all conditions disclosed herein. Moreover, the addition of sugars, polysaccharides, or mucin does not increase or decrease the HDAC inhibition activity of these strains. “HDAC cluster 5” corresponds to strains that are capable of inhibiting HDAC when grown only in the presence of sucrose, FOS/inulin, glucose, pectin, or starch. “HDAC cluster 6” corresponds to strains that are capable of increasing HDAC inhibition when grown in the presence of sucrose, FOS/inulin, glucose, pectin, or mucin.

Other Functional Features

As described supra, in addition to the specific functions detailed above, in some aspects, bacteria or combinations of bacteria disclosed herein can further comprise one or more of the following functional features: (i) capable of inducing Wnt activation, (ii) capable of producing B vitamins (e.g., thiamin (B1) and pyridoxamine (B6)), (iii) capable of modulating host metabolism of endocannabinoids, (iv) capable of producing polyamines and/or modulating host metabolism of polyamines, (v) capable of reducing fecal levels of sphingolipids, (vi) capable of modulating host production of kynurenine, (vii) capable of reducing fecal calprotectin level, or (viii) any combination thereof. In further aspects, bacteria or combinations of bacteria disclosed herein are not capable of activating a toll-like receptor pathway (e.g., TLR4 or TLR5). In certain aspects, bacteria or combinations of bacteria disclosed herein are capable of activating a toll-like receptor pathway (e.g., TLR2). In some aspects, bacteria or combinations of bacteria described herein are capable of inhibiting apoptosis of intestinal epithelial cells.

As described elsewhere in the present disclosure, the designed bacterial compositions described herein can also be used to treat a cancer, and therefore, exhibit one or more properties that are useful in the treatment of a cancer. Non-limiting examples of such features include: inhibition of HDAC activity, production of short-chain fatty acids, production of tryptophan metabolites, production of IL-18, activation of CD8+ T cells by metabolites (e.g., short-chain fatty acids) or macromolecules, activation of antigen presenting cells such as dendritic cells by bacterial antigens, macromolecules and metabolites, reducing expression of one or more inhibitory receptors (e.g., TIGIT, TIM-3, or LAG-3) on CD8+ T cells, increasing expression of one or more genes/proteins associated with T cell activation and/or function (e.g., CD45RO, CD69, IL-24, TNF-α, perforin, or IFN-γ), enhancing the ability of CD8+ T cells to kill tumor cells, enhancing the efficacy of an immune checkpoint inhibitor, or reduced colonic inflammation (e.g., through upregulation of Tregs), or enabling recruitment of CD8+ T cells to tumors located distally.

The levels of any of the biological molecules (e.g., those described above) in a subject suffering from a disease or disorder disclosed herein (can be measured as described in the present disclosure (see, e.g., Examples) or by any other methods known in the art.

In some aspects, a bacterial composition of the present disclosure (e.g., designed compositions) comprises one or more bacteria that are capable of forming spores (i.e., spore-forming bacteria). Accordingly, in some aspects, a bacterial composition comprises a purified population of bacteria, wherein the bacteria are in the form of spores. In some aspects, all the bacteria are in the form of spores. In other aspects, some of the bacteria are in the form of spores, while other bacteria are not in the form of spores (i.e., vegetative-state). In some aspects, the bacterial composition comprises a purified population of spore-forming bacteria, wherein the bacteria are all in the vegetative-state.

In some aspects, a bacterial composition comprises a population of bacteria that are sensitive to one or more antibiotics that can be used in a human. In some aspects, bacteria of the composition are resistant to one or more antibiotics that are used to prophylactically treat patients with a disease or disorder, such as those associated with dysbiosis of the gastrointestinal tract (e.g., an active IBD (e.g., flare of Crohn's disease)). Such antibiotics include, but are not limited to, β-lactams, vancomycin, aminoglycosides, fluoroquinolones, and daptomycin.

In some aspects, the strain of an OTU useful for the present disclosure (e.g., an OTU disclosed herein) can be obtained from a public biological resource center such as the ATCC (atcc.org), the DSMZ (dsmz.de), or the Riken BioResource Center (en.brc.riken.jp). Methods for determining sequence identity are known in the art.

In some aspects, the composition is a designed composition. DE1 is an example of such a designed composition. Non-limiting examples of additional designed compositions are provided in FIGS. 31, 32, 33, and 34. As used herein, the term “DE1” refers to a synthetic composition consisting of 14 spore-forming bacterial species. See FIG. 31. DE1 (as well as the other exemplary DEs disclosed herein) was designed to capture key functional and phylogenetic attributes that applicant identified as associated with clinical remission (e.g., of a disease or disorder disclosed herein) and/or shown to have properties reflecting anti-inflammatory activity and/or enhancement of epithelial barrier integrity. Accordingly, DE1 integrates clinical insights of functional and phylogenetic correlates of clinical remission together with in vitro screening data on strain functional phenotypes. Specifically, DE1 was designed to provide a bacterial composition with the following functional attributes: a) tryptophan metabolic capacity, specifically the ability to produce indole and 3-methylindole, b) HDAC inhibition capacity across diverse nutrient conditions (e.g. the ability to produce SCFAs), c) the ability to produce medium-chain fatty acids, specifically valerate and hexanoate, d) production of deoxycholic acid (DCA) and lithocholic acid (LCA) from cholate and chenodeoxycholate, e) the ability to suppress induction of IL-8 in intestinal epithelial cells, f) the ability to induce regulatory T cells, and g) the ability to activate Wnt signaling pathway. While ensuring these functional properties are present in DE1, phylogenetic diversity and coverage of phylogenetic clades associated with remission of UC in FMT studies were represented. Non-limiting examples of other DEs that share one or more of the functional properties of DE1 include DE2, DE37 (also referred to herein as DE935045.1), and DE39 (also referred to herein as DE935045.2).

### II. Formulations

Further provided herein are formulations for administration to humans and other subjects in need thereof (e.g., subject suffering from a disease or disorder disclosed herein). Generally, a bacterial composition as described herein is combined with additional active and/or inactive materials to produce a formulation. In some aspects, a bacterial composition is formulated in a unit dosage form, each dosage form containing, e.g. from about 10² to about 10⁹ spores, for example, about 10⁴ to about 10⁸ spores. In other aspects, a bacterial composition is formulated in a multi-dose format. The formulation disclosed herein can be effective over a wide dosage range and is generally administered in a pharmaceutically effective amount.

The term “effective dose” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A therapeutically effective amount or dosage of a drug includes a “prophylactically effective amount” or a “prophylactically effective dosage”, which is any amount of the drug that, when administered alone or in combination with another therapeutic agent to a subject at risk of developing a disease or of suffering a recurrence of disease, inhibits the development or recurrence of the disease. The ability of a therapeutic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

As used herein, the term “dosage” can refer to the total number of colony forming units (CFUs) of each individual species or strain; or can refer to the total number of microorganisms in the dose. It is understood in the art that determining the number of organisms in a dosage is not exact and can depend on the method used to determine the number of organisms present. If the composition includes spores, for example, the number of spores in a composition can be determined using any suitable methods known in the art, e.g., a dipicolinic acid assay (Fichtel et al., FEMS Microbiol Ecol 61: 522-532 (2007)), or a single colony forming unit (SCFU) assay. Effective doses can be extrapolated from dose-response curves derived from in vitro or animal model test systems.

As used herein, the term “unit dosage forms” or “dosage unit forms” refers to physically discrete units suitable as unitary dosages for human subjects and other mammals, each unit containing a predetermined quantity of active component calculated to produce the desired therapeutic effect, in association with a suitable pharmaceutical excipient. For instance, in some aspects, a unit dosage form can be in the form of a solid (e.g., capsules, tablets, caplets, pills, troches, lozenges, powders, and granules). In some aspects, a unit dosage form can be in the form of a liquid (e.g., liquid suspension). In some cases, more than one-unit dosage form (e.g., two separate capsules or one capsule and a liquid suspension) constitutes a dose. For example, a single dose can be one-unit dosage form, two dosage unit forms, three dosage unit forms, four unit dosage forms, five unit dosage forms, or more. In some cases, the number of unit dosage forms constituting a single dose is 1, 2, 3, 4, 5, 6, 7, 8, 9, 10, 11, 12, 13, 14, 15, 20, 25, or 30 unit dosage forms. A single dose can be, e.g., about 10³ to about 10⁹ CFUs, for example, about 10⁴ to about 10⁸ CFUs. In some aspects, a dose is 1, 2, 3, or 4 capsules containing a total of between about 10² and about 10⁸ CFUs in the dose. In the case of a single dose having multiple dosage forms, the dosage forms are generally delivered within a prescribed period, e.g., within 1 hour, 2 hours, 5 hours, 10 hours, 15 hours, or 24 hours.

In some aspects, a formulated bacterial composition comprises at least one carbohydrate. A “carbohydrate” refers to a sugar or polymer of sugars. The terms “saccharide,” “polysaccharide,” “carbohydrate,” and “oligosaccharide” can be used interchangeably. Most carbohydrates are aldehydes or ketones with many hydroxyl groups, usually one on each carbon atom of the molecule. Carbohydrates generally have the molecular formula CₙH₂ₙOₙ. A carbohydrate can be a monosaccharide, a disaccharide, trisaccharide, oligosaccharide, or polysaccharide. The most basic carbohydrate is a monosaccharide, such as glucose, sucrose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates can contain modified saccharide units such as 2′-deoxyribose wherein a hydroxyl group is removed, 2′-fluororibose wherein a hydroxyl group is replaced with a fluorine, or N-acetylglucosamine, a nitrogen-containing form of glucose (e.g., 2′-fluororibose, deoxyribose, and hexose). Carbohydrates can exist in many different forms, for example, conformers, cyclic forms, acyclic forms, stereoisomers, tautomers, anomers, and isomers.

In some aspects, a formulated bacterial composition comprises at least one lipid. As used herein a “lipid” includes fats, oils, triglycerides, cholesterol, phospholipids, fatty acids in any form including free fatty acids. Fats, oils and fatty acids can be saturated, unsaturated (cis or trans) or partially unsaturated (cis or trans). In some aspects the lipid comprises at least one fatty acid selected from lauric acid (12:0), myristic acid (14:0), palmitic acid (16:0), palmitoleic acid (16:1), margaric acid (17:0), heptadecenoic acid (17:1), stearic acid (18:0), oleic acid (18:1), linoleic acid (18:2), linolenic acid (18:3), octadecatetraenoic acid (18:4), arachidic acid (20:0), eicosenoic acid (20:1), eicosadienoic acid (20:2), eicosatetraenoic acid (20:4), eicosapentaenoic acid (20:5) (EPA), docosanoic acid (22:0), docosenoic acid (22:1), docosapentaenoic acid (22:5), docosahexaenoic acid (22:6) (DHA), and tetracosanoic acid (24:0). In some aspects, the formulated bacterial composition comprises at least one modified lipid, for example a lipid that has been modified by cooking.

In some aspects, a formulated bacterial composition comprises at least one supplemental mineral or mineral source. Examples of minerals include, without limitation: chloride, sodium, calcium, iron, chromium, copper, iodine, zinc, magnesium, manganese, molybdenum, phosphorus, potassium, and selenium. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof.

In some aspects, a formulated bacterial composition comprises at least one supplemental vitamin. The at least one vitamin can be fat-soluble or water-soluble vitamins. Suitable vitamins include but are not limited to vitamin C, vitamin A, vitamin E, vitamin B12, vitamin K, riboflavin, niacin, vitamin D, vitamin B6, folic acid, pyridoxine, thiamine, pantothenic acid, and biotin. Suitable forms of any of the foregoing are salts of the vitamin, derivatives of the vitamin, compounds having the same or similar activity of the vitamin, and metabolites of the vitamin.

In some aspects, a formulated bacterial composition comprises an excipient. Non-limiting examples of suitable excipients include a buffering agent, a diluent, a preservative, a stabilizer, a binder, a compaction agent, a lubricant, a dispersion enhancer, a disintegration agent, a flavoring agent, a sweetener, and a coloring agent.

In some aspects, the excipient is a buffering agent. Non-limiting examples of suitable buffering agents include sodium citrate, magnesium carbonate, magnesium bicarbonate, calcium carbonate, and calcium bicarbonate.

In some aspects, the excipient serves as a diluent. In such aspects, the excipient can be a solid, semi-solid, or liquid material that acts as a vehicle, carrier, or medium for the active component (e.g., bacteria of the composition disclosed herein). Thus, a formulation can be in the form of, e.g., a tablet, pill, powder, lozenge, sachet, cachet, elixir, suspension, emulsion, solution, syrup, aerosol (as a solid or in a liquid medium), ointment containing, for example, up to 10% by weight of the active component, soft capsule, hard capsule, gel-cap, tablet, suppository, solution, or packaged powder. In some cases, maximizing delivery of viable bacteria is enhanced by including gastro-resistant polymers, adhesion enhancers, or controlled release enhancers in a formulation.

In some aspects, the excipient comprises a preservative. Non-limiting examples of suitable preservatives include antioxidants, such as alpha-tocopherol and ascorbate, and antimicrobials, such as parabens, chlorobutanol, and phenol.

In some aspects, a formulated bacterial composition comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, and combinations thereof.

In some aspects, a formulated bacterial composition comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

In some aspects, a formulated bacterial composition comprises a dispersion enhancer as an excipient. Non-limiting examples of suitable dispersants include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In some aspects, a formulated bacterial composition comprises a disintegrant as an excipient. In some aspects, the disintegrant is a non-effervescent disintegrant. Non-limiting examples of suitable non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, and tragacanth. In some aspects, the disintegrant is an effervescent disintegrant. Non-limiting examples of suitable effervescent disintegrants include sodium bicarbonate in combination with citric acid, and sodium bicarbonate in combination with tartaric acid.

In some aspects, the excipient comprises a flavoring agent. Flavoring agents can be chosen from synthetic flavor oils and flavoring aromatics; natural oils; extracts from plants, leaves, flowers, and fruits; and combinations thereof. In some aspects, the flavoring agent is selected from cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot.

In some aspects, the excipient comprises a sweetener. Non-limiting examples of suitable sweeteners include glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, sylitol, and the like. Also contemplated are hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

In some aspects, a formulated bacterial composition comprises a coloring agent. Non-limiting examples of suitable color agents include food, drug and cosmetic colors (FD&C), drug and cosmetic colors (D&C), and external drug and cosmetic colors (Ext. D&C). The coloring agents can be used as dyes or their corresponding lakes.

Additional suitable excipients include, for example, saline, phosphate buffered saline (PBS), cocoa butter, polyethylene glycol, polyalcohols (e.g., glycerol, sorbitol, or mannitol) and prebiotic oligosaccharides such as inulin, Crystalean® starch, or dextrin. Excipients can also be selected to account, at least in part, for the ability of the OTUs in a particular composition to withstand gastric pH (if being delivered orally or directly to the GI tract) and/or bile acids, or other conditions encountered by the formulation upon delivery to a subject (e.g., an ulcerative colitis patient).

The weight fraction of the excipient or combination of excipients in the formulation is usually about 99% or less, such as about 95% or less, about 90% or less, about 85% or less, about 80% or less, about 75% or less, about 70% or less, about 65% or less, about 60% or less, about 55% or less, 50% or less, about 45% or less, about 40% or less, about 35% or less, about 30% or less, about 25% or less, about 20% or less, about 15% or less, about 10% or less, about 5% or less, about 2% or less, or about 1% or less of the total weight of the composition.

In preparing a formulation of the present disclosure, the bacterial composition can be milled to provide the appropriate particle size prior to combining with the other ingredients, e.g., those described herein. In some aspects, a bacterial composition is formulated so as to provide quick, sustained, or delayed release of the active component after administration to a subject, for example, for release in the colon, by employing methods and forms known in the art.

The bacterial compositions disclosed herein can be formulated into a variety of forms and administered by a number of different means. A bacterial composition (e.g., that which has been formulated as described herein) can be administered orally, rectally, or parenterally, in formulations containing conventionally acceptable carriers, adjuvants, and vehicles as desired. The term “parenteral” as used herein includes subcutaneous, intravenous, intramuscular, or intrasternal injection and infusion techniques. In an exemplary aspect, the bacterial composition (e.g., that which has been formulated as described herein) is administered orally.

Solid dosage forms for oral administration include capsules, tablets, caplets, pills, troches, lozenges, powders, and granules. A capsule typically comprises a core material comprising a bacterial composition (e.g., that which has been formulated as described herein) and a shell wall that encapsulates the core material. In some aspects the core material comprises at least one of a solid, a liquid, and an emulsion. In some aspects the shell wall material comprises at least one of a soft gelatin, a hard gelatin, and a polymer. Suitable polymers include, but are not limited to: cellulosic polymers such as hydroxypropyl cellulose, hydroxyethyl cellulose, hydroxypropyl methyl cellulose (HPMC), methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, such as those formed from acrylic acid, methacrylic acid, methyl acrylate, ammonio methylacrylate, ethyl acrylate, methyl methacrylate and/or ethyl methacrylate (e.g., those copolymers sold under the trade name “Eudragit”); vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac (purified lac). In some aspects at least one polymer functions as taste-masking agents.

Tablets, pills, and the like can be compressed, multiply compressed, multiply layered, and/or coated. The coating can be single or multiple. In some aspects, the coating material comprises at least one of a saccharide, a polysaccharide, and glycoproteins extracted from at least one of a plant, a fungus, and a microbe. Non-limiting examples include corn starch, wheat starch, potato starch, tapioca starch, cellulose, hemicellulose, dextrans, maltodextrin, cyclodextrins, inulins, pectin, mannans, gum arabic, locust bean gum, mesquite gum, guar gum, gum karaya, gum ghatti, tragacanth gum, funori, carrageenans, agar, alginates, chitosans, or gellan gum. In some aspects the coating material comprises a protein. In some aspects the coating material comprises at least one of a fat and an oil. In some aspects the at least one of a fat and an oil is high temperature melting. In some aspects the at least one of a fat and an oil is hydrogenated or partially hydrogenated. In some aspects the at least one of a fat and an oil is derived from a plant. In some aspects the at least one of a fat and an oil comprises at least one of glycerides, free fatty acids, and fatty acid esters. In some aspects the coating material comprises at least one edible wax. The edible wax can be derived from animals, insects, or plants. Non-limiting examples include beeswax, lanolin, bayberry wax, carnauba wax, and rice bran wax.

In some aspects, a tablet or pill comprises an inner component surrounding the composition (e.g., that which has been formulated as described herein) and an outer component, the latter serving as an envelope over the former. The two components can be separated by an enteric coating layer that can resist disintegration in the stomach and permits the inner component to pass intact into the duodenum or to be delayed in release.

Alternatively, powders or granules embodying a bacterial composition disclosed herein (e.g., that which has been formulated as described herein) can be incorporated into a food product. In some aspects, the food product is a drink for oral administration. Non-limiting examples of a suitable drink include fruit juice, a fruit drink, an artificially flavored drink, an artificially sweetened drink, a carbonated beverage, a sports drink, a liquid diary product, a shake, an alcoholic beverage, a caffeinated beverage, infant formula and so forth. Other suitable means for oral administration include aqueous and nonaqueous solutions, emulsions, suspensions and solutions and/or suspensions reconstituted from non-effervescent granules, containing at least one of suitable solvents, preservatives, emulsifying agents, suspending agents, diluents, sweeteners, coloring agents, and flavoring agents.

In some aspects, the food product is a solid foodstuff. Suitable examples of a solid foodstuff include without limitation a food bar, a snack bar, a cookie, a brownie, a muffin, a cracker, an ice cream bar, a frozen yogurt bar, and the like.

In some aspects, a bacterial composition disclosed herein (e.g., that which has been formulated as described herein) is incorporated into a therapeutic food. In some aspects, the therapeutic food is a ready-to-use food that optionally contains some or all essential macronutrients and micronutrients. In some aspects, a bacterial composition disclosed herein (e.g., that which has been formulated as described herein) is incorporated into a supplementary food that is designed to be blended into an existing meal. In some aspects, the supplemental food contains some or all essential macronutrients and micronutrients. In some aspects, a bacterial composition disclosed herein (e.g., that which has been formulated as described herein) is blended with or added to an existing food to fortify the food's protein nutrition. Examples include food staples (grain, salt, sugar, cooking oil, margarine), beverages (coffee, tea, soda, beer, liquor, sports drinks), snacks, sweets and other foods.

In some aspects, the formulations are filled into gelatin capsules for oral administration. An example of an appropriate capsule is a 250 mg gelatin capsule containing from 10 (up to 100 mg) of lyophilized powder (10⁸ to 10¹¹ bacteria), 160 mg microcrystalline cellulose, 77.5 mg gelatin, and 2.5 mg magnesium stearate. In other aspects, from about 10⁵ to about 10¹² bacteria can be used, about 10⁵ to about 10⁷, about 10⁶ to about 10⁷, or about 10⁸ to about 10¹⁰, with attendant adjustments of the excipients if necessary. In further aspects, an enteric-coated capsule or tablet or with a buffering or protective composition can be used. The use of enteric polymers (such as those used to coat a capsule or tablet described herein) can be useful when formulating a bacterial composition disclosed herein for oral administration. In certain aspects, the enteric polymers allow for more efficient delivery of the bacterial compositions disclosed herein to a subject's gastrointestinal tract. In some aspects, the enteric-coated capsule or tablet release their contents (i.e., bacteria or combinations of bacteria disclosed herein) when the pH becomes alkaline after the enteric-coated capsule or tablet passes through the stomach. When a pH sensitive composition (e.g., enteric polymers) is used for formulating the bacterial composition, the pH sensitive composition is preferably a polymer whose pH threshold of the decomposition of the composition is 6.8 to 7.5. In some aspects, the pH threshold range can be lower or higher, e.g., about 5.5 or about 6.0. Such a numeric value range is a range where the pH shifts toward the alkaline side at a distal portion of the stomach, and hence is a suitable range for use in the delivery to the colon.

Moreover, an approach to improving delivery of a bacterial composition disclosed herein (e.g., which can be formulated as described herein) to the colon specifically can include a composition which ensures the delivery to the gastrointestinal tract by delaying the release of the contents by approximately 3 to 5 hours, which corresponds to the small intestinal transit time. In an example of formulating a pharmaceutical preparation comprising the composition for delaying the release, a hydrogel is used as a shell. The hydrogel is hydrated and swells upon contact with gastrointestinal fluid, so that the contents are effectively released. Furthermore, the delayed release dosage units include drug-containing compositions having a material which coats or selectively coats a drug. Examples of such a selective coating material include in vivo degradable polymers, gradually hydrolyzable polymers, gradually water-soluble polymers, and/or enzyme degradable polymers. A preferred coating material for efficiently delaying the release is not particularly limited, and examples thereof include cellulose-based polymers such as hydroxypropyl cellulose, acrylic acid polymers and copolymers such as methacrylic acid polymers and copolymers, and vinyl polymers and copolymers such as polyvinylpyrrolidone.

Additional compositions that target delivery to the colon include bioadhesive compositions which specifically adhere to the colonic mucosal membrane (for example, a polymer described in the specification of U.S. Pat. No. 6,368,586), and compositions into which a protease inhibitor is incorporated for protecting particularly a bacterial composition disclosed herein (e.g., which can be formulated as described herein) in the gastrointestinal tracts from decomposition due to an activity of a protease.

An additional colon-delivery mechanism is via pressure change, such that the contents are released from the colon by generation of gas in bacterial fermentation at a distal portion of the stomach. Such pressure-change is not particularly limited, and a more specific example thereof is a capsule which has contents dispersed in a suppository base and which is coated with a hydrophobic polymer (for example, ethyl cellulose).

A further composition for delivery to the colon includes, for example, a bacterial composition disclosed herein (e.g., which can be formulated as described herein) comprising a component that is sensitive to an enzyme (for example, a carbohydrate hydrolase or a carbohydrate reductase) present in the colon. Such a composition is not particularly limited, and more specific examples thereof include compositions that use food components such as non-starch polysaccharides, amylose, xanthan gum, and azopolymers.

In some aspects, a bacterial composition disclosed herein is formulated with a germinant to enhance engraftment or efficacy. In some aspects, a bacterial composition is formulated or administered with a prebiotic substance to enhance engraftment or efficacy.

In some aspects, the number of bacteria of each type can be present in the same level or amount or in different levels or amounts. For example, in a bacterial composition (e.g., which can be formulated as described herein) with two types of bacteria, the bacteria can be present in from about a 1:10,000 ratio to about a 1:1 ratio, from about a 1:10,000 ratio to about a 1:1,000 ratio, from about a 1:1,000 ratio to about a 1:100 ratio, from about a 1:100 ratio to about a 1:50 ratio, from about a 1:50 ratio to about a 1:20 ratio, from about a 1:20 ratio to about a 1:10 ratio, from about a 1:10 ratio to about a 1:1 ratio. For bacterial compositions (e.g., which can be formulated as described herein) comprising at least three types of bacteria, the ratio of type of bacteria can be chosen pairwise from ratios for bacterial compositions (e.g., which can be formulated as described herein) with two types of bacteria. For example, in a bacterial composition (e.g., which can be formulated as described herein) comprising bacteria A, B, and C, at least one of the ratio between bacteria A and B, the ratio between bacteria B and C, and the ratio between bacteria A and C can be chosen, independently, from the pairwise combinations above. Non-limiting examples of additional bacterial formulations that can be used with the present disclosure are provided in WO 2020/118054, which is incorporated herein by reference in its entirety.

### III. Methods of Treating a Subject

The compositions and formulations disclosed herein can be used for the treatment and/or prevention of a disease or disorder, such as those associated with dysbiosis of a gastrointestinal tract (e.g., an IBD, for example, ulcerative colitis or Crohn's Disease), e.g., by ameliorating one or more sign or symptom of the disease (e.g., induce clinical remission), and/or to reduce the recurrence of active disease (e.g., maintain clinical remission). In some aspects, a disease or disorder that can be treated with the present disclosure is an IBD (e.g., ulcerative colitis). In some aspects, a disease or disorder that can be treated with the present disclosure is a cancer. In some aspects, a disease or disorder that can be treated with the present disclosure comprises both IBD and a cancer.

The terms “treat,” “treating,” and “treatment,” as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition or biochemical indicia associated with a disease or enhancing overall survival. Treating can include reducing at least one sign or symptom associated with a disease or disorder disclosed herein, e.g., ulcerative colitis or cancer. Treatment can be of a subject having a disease or a subject who does not have a disease (e.g., for prophylaxis). It is understood that “preventing” can mean reducing the risk of disease, increasing the length of remission, or reducing the rate of relapse.

In some aspects, treatment with a formulation or a bacterial composition described herein is associated with at least one of the following: (i) an increase in the diversity of the gastrointestinal (GI) microbiome in a subject, (ii) a reduction in GI inflammation in a subject, (iii) improvement in mucosal and/or epithelial barrier integrity in a subject compared to a reference control (e.g., untreated patients or the subject prior to treatment), (iv) promotion of mucosal healing and (v) other improvements of at least one sign or symptom of a disease or disorder disclosed herein. Such improvements can also include, for example, improvements detected via biomarkers, such as a decrease or increase in the level of certain biological molecules (e.g., fecal calprotectin, secondary bile acids, tryptophan metabolites, short-chain and medium-chain fatty acids, sphingolipids, and kynurenine) following treatment.

In some aspects, when treating a subject suffering from an inflammatory disease (e.g., ulcerative colitis), an improvement in the disease, such as mucosal healing, can be assessed by a reduction in endoscopic Mayo score. Mayo scores are known in the art, e.g., see globalrph.com/mayo clinic score.htm. A reduction in total Mayo score from a pre-treatment score (i.e., baseline) and/or improvements in rectal bleeding and/or endoscopic subscores are indicative of a therapeutic effect.

In some aspects, the improvement rate (e.g., clinical remission rate) after treatment with a formulation or a bacterial composition disclosed herein is at least about 20%, at least about 25%, at least about 30%, at least about 40%, at least about 50%, at least about 60%, at least about 70%, at least about 75%, at least about 80%, at least about 85%, at least about 90%, at least about 95%, or about 100%. In some aspects, the improvement rate (e.g., clinical remission rate) is improved compared to placebo, e.g., at least 25% versus 10%, respectively. In some aspects, clinical remission is a Mayo score of 2 points, no individual subscore >1.

In some aspects, the clinical response to treatment with a formulation or a bacterial composition of the present disclosure is improved versus placebo, e.g., at least 25% compared to 10%, respectively. When treating a subject suffering from an inflammatory disease, e.g., ulcerative colitis, mucosal healing is defined as a 0 or 1 on the endoscopy subscore of the Mayo score. A clinical response is, in some aspects, a decrease from baseline in the Mayo score by ≥30% and/or ≥3 points, accompanied by a decrease in the rectal bleeding subscore of 1 or a rectal bleeding subscore of 0 or 1. In some aspects, clinical response is defined as a decrease of ≥3 points in Total Modified Mayo Score (TMMS) from baseline, along with at least one of a decrease of >1 point in rectal bleeding subscore or absolute rectal bleeding subscore of 0 or 1. Complete remission is defined as a TMMS <2 and an endoscopic subscore of 0 with no erythema, no blood, and no evidence of inflammation. Endoscopic improvement is defined as a decrease in the endoscopic subscore of >1.

Formulations disclosed herein (e.g., comprising a designed bacterial composition) can be used to treat any disease or disorder associated with a dysbiosis of the gastrointestinal tract. Non-limiting examples of such diseases or disorders are provided throughout the present disclosure.

Formulations or a bacterial composition as described herein are useful for administration to a subject, e.g., a mammal, such as a human in need of treatment, e.g., to prevent or treat a disease or disorder disclosed herein or a sign or symptom of a disease or disorder disclosed herein or to prevent recurrence of a disease or disorder disclosed herein. In some aspects, the mammalian subject is a human subject. In some aspects, the human subject (e.g., patient) has one or more signs or symptoms of a disease or disorder, such as those associated with a dysbiosis. Non-limiting examples of such signs or symptoms can include, but are not limited to, diarrhea (e.g., containing blood or pus); abdominal pain and cramping; rectal pain; rectal bleeding; urgency to defecate; inability to defecate despite urgency; weight loss; fatigue; fever; failure to grow (in children); severe bleeding; perforated colon; severe dehydration; liver disease; osteoporosis; inflammation of the skin, joints, or eyes; mouth sores; increased risk of colon cancer; toxic megacolon; or increased risk of blood clots in veins and arteries. A therapeutically effective treatment using a formulation or a bacterial composition provided herein can ameliorate one or more of such signs and symptoms of a disease or disorder disclosed herein. In some aspects, the patient is in remission and the microbial composition is administered to increase the duration of remission through maintenance therapy.

Efficacy of a treatment can be determined by evaluating signs and or symptoms and according to whether induction of improvement and/or maintenance of a remission or improved condition is achieved, e.g., for at least about 1 week, at least about two weeks, at least about three weeks, at least about four weeks, at least about 8 weeks, or at least about 12 weeks. For example, in cases of a disease or disorder disclosed herein (e.g., colitis), mucosal healing (as judged endoscopically, histologically, or via imaging techniques) can be used to evaluate the efficacy of a treatment. In certain aspects, such an approach can be particularly useful for predicting long term clinical outcome in a subject diagnosed with the disease or disorder. Remission or signs or symptoms can be determined using clinical indices, such as, for Crohn's disease, the Crohn's Disease Activity Index (CDAI), the PCDAI, or the amelioration or one or more elements of the PCDAI or CDAI, e.g., number of liquid or soft stools, abdominal pain, general well-being, presence of complications (such as arthralgia or arthritis, uveitis; inflammation of the iris; presence of erythema nodosum, pyoderma gangrenosum, or aphthous ulcers; anal fissures, fistulae, or abscesses; other fistulae, or fever), taking opiates or diphenoxylate/atropine for diarrhea, presence of an abdominal mass, hematocrit of <0.47 (males) or <0.42 (females); or percentage deviation from standard weight. In some aspects a subject treated according to a method described herein attains and/or remains at a CDAI below 150. In some aspects, a positive response to a method is a reduction of a subject's CDAI by at least 70 points.

For ulcerative colitis, indications of therapeutic efficacy include, for example, normalization of stool frequency, lack of urgency, or absence of blood in stools. Clinical improvement (e.g., clinical remission) is considered achieved if at least one sign or symptom is reduced after completion of the treatment. Mucosal healing is one example of a measure of clinical improvement. Other signs/symptoms can include normalization of C-reactive protein and/or other acute phase indicators, decrease in levels of fecal calprotectin and/or lactoferrin, and subjective indicia such as those related to quality of life. Other examples of indicia can include improvement from moderate to mild using the Montreal Classification, the Mayo Score (with or without endoscopy subscore), or the Pediatric Ulcerative Colitis Index. In general, methods and compositions described herein are useful for treating a subject diagnosed with a colitis.

Other indicators of efficacy of a therapeutic composition and/or method for treating a disease or disorder, such as those associated with dysbiosis include engraftment of at least one bacterial species or OTU identified in a microbiome composition, for example, at about 2 weeks, about 3 weeks, about 4 weeks, about 5 weeks, about 6 weeks, about 7 weeks, about 8 weeks, about 9 weeks, about 10 weeks, about 11 weeks, about 12 weeks, or longer after initial dosing with the microbiome composition; clinical remission at 0 weeks, about 1 week, about 4 weeks, about 5 weeks, about 6 weeks, about 7 weeks, about 8 weeks, about 9 weeks, about 10 weeks, about 11 weeks, about 12 weeks, or longer after initial dosing with the microbiome composition (e.g., for colitis, a Mayo score <=2 with no subscore >1); or endoscopic remission at 0 weeks, about 1 week, about 4 weeks, about 5 weeks, about 6 weeks, about 7 weeks, about 8 weeks, about 9 weeks, about 10 weeks, about 11 weeks, about 12 weeks, or longer after initial dosing with the microbiome composition (e.g., for colitis, Mayo endoscopy score of 0).

In some aspects, treatment with a formulation or a bacterial composition disclosed herein can improve a dysbiosis, including, but not limited to, an improvement in the representation of one or more OTUs identified as reduced in a population of subjects suffering from a disease or disorder associated with dysbiosis (e.g., UC patients or cancer patients with active disease). In some aspects, treatment with a formulation or a bacterial composition of the present disclosure can reduce the representation of one or more microbial species that are associated with a disease or disorder disclosed herein.

In some aspects, treatment with a formulation or a bacterial composition disclosed herein can increase the representation of microbial species that are associated with an improvement (e.g., clinical remission) of a disease or disorder disclosed herein.

In some aspects, a formulation or a bacterial composition can increase the prevalence of one or more of the following bacterial species in a subject suffering from a disease or disorder disclosed herein (e.g., in the GI microbiome): Gemmiger formicilis, Roseburia hominis, Clostridium bolteae, Parasutterella excrementihominis, Holdemania filiformis, Holdemania massiliensis, Bacteroides ovatus, Akkemansia muciniphila, Clostridium leptum, Bilophila wadsworthia, Dielma fastidiosa, Clostridium symbiosum, Eubacterium siraeum, Agathobaculum desmolans, Agathobaculum butyriciproducens, or Bacteroides vulgatus, or Flintibacter SC49. In some aspects, a formulation or a bacterial composition disclosed herein can increase the prevalence of one or more bacteria selected from the group consisting of Gemmiger formicilis, Roseburia hominis, Clostridium bolteae, Holdemania fihformis, Holdemania massiliensis, Clostridium leptum, Dielma fastidiosa, Clostridium symbiosum, Eubacterium siraeum, and combinations thereof. In certain aspects, a formulation comprising a designed composition disclosed herein can increase the prevalence of one or more bacteria selected from those disclosed in Table 4, Table 5, FIG. 13, FIG. 17, FIG. 18, FIG. 31, FIG. 32, FIG. 33, and/or FIG. 34. In some aspects, a formulation or a bacterial composition can increase the prevalence of one or more bacteria comprising a 16S rDNA sequence that is at least about 85%, at least about 90%, at least about 95%, at least about 96%, at least about 97%, at least about 97.5%, at least about 98%, at least about 98.5%, at least about 99%, at least about 99.5%, or about 100% identical to a 16S rDNA sequence set forth in SEQ ID NOs: 1-14, 16-30, 32-36, 39, 41, 44, 45, 47-51, 59-62, 64-68, 72-76, 102-398, or any of the foregoing species.

In some aspects, a formulation or a bacterial composition disclosed herein can increase, in a treated patient, representation of one or more bacterial phyla, genera, or species such as clade 155, e.g., Bacteroides faecis, which are reduced in subjects suffering from a disease or disorder disclosed herein.

In some aspects, treatment with a formulation or a bacterial composition disclosed herein can improve a GI function that is reduced or otherwise aberrant in subjects that have a disease or disorder disclosed herein (e.g., UC or cancer). In some aspects, a formulation disclosed herein or a bacterial composition can increase or decrease the level of certain biological molecules (e.g., fecal calprotectin, secondary bile acids, tryptophan metabolites, short-chain and medium-chain fatty acids, sphingolipids, and kynurenine) in a treated subject. In some aspects, the increase or decrease of such biological molecules is correlated with an improvement of the disease (e.g., clinical remission).

Formulations and bacterial compositions disclosed herein can be useful in a variety of clinical situations. For example, the formulation or a bacterial composition can be administered as a complementary treatment to standard treatment regimens for a disease or disorder, such as those disclosed herein. In some aspects, formulations and bacterial compositions of the present disclosure can be administered as an alternative to standard treatment regimens. In some aspects, the formulation and bacterial composition disclosed herein has a comparable, if not better, clinical efficacy (e.g., clinical remission rate) compared to standard treatment regimens (e.g., antibiotics or anti-inflammatory drugs, e.g., LIALDA®, PENTASA®, UCERIS®, REMICADE®, ENTYVIO®, SIMPONI®). In some aspects, formulations and bacterial compositions of the present disclosure can be administered simultaneously with standard treatment regimens to enhance their activity. In some aspects, formulations and bacterial compositions of the present disclosure can be administered simultaneously with standard treatment regimens without exacerbating their adverse event profile.

In some aspects, a subject to be treated with a formulation or bacterial formulation has mild to moderate disease or disorder, such as those disclosed herein (e.g., ulcerative colitis, e.g., a Mayo score of ≥4 and ≤10). In some aspects, the patient is failing standard of care. In some aspects, the formulation or a bacterial composition is used to maintain clinical remission or clinical benefit in a patient with moderate to severe disease being treated with an immunomodulator or immunosuppressant, including anti-TNF, anti-IL23, anti-integrin or other antibody treatments.

In some aspects, a subject receives a pretreatment protocol prior to administration of the formulation or a bacterial composition, wherein the pretreatment protocol prepares the gastrointestinal tract to receive the bacterial composition. In certain aspects, the pretreatment protocol comprises an oral antibiotic treatment, wherein the antibiotic treatment alters the bacteria in the patient. In specific aspects, the antibiotic is not absorbed through the gut or minimally bioavailable for systemic distribution. In other aspects, the pretreatment protocol comprises a colonic cleansing (e.g., enema), wherein the colonic cleansing substantially empties the contents of the patient's colon. As used herein, “substantially emptying the contents of the colon” refers to removal of at least about 75%, at least about 80%, at least about 90%, at least about 95%, or about 100% of the contents of the ordinary volume of colon contents. Antibiotic treatment can precede the colon-cleansing protocol.

In some aspects, a pretreatment protocol is administered to a subject at least 1 day, 2 days, 3 days, 5 days, 6 days, 7 days, 10 days, or 15 days prior to administration of a formulation or a bacterial composition described herein. In some aspects, the subject receives multiple doses of a formulation or a bacterial composition. In some aspects, the subject has at least one sign or symptom of a disease or disorder, such as those disclosed herein prior to administration of the formulation or a bacterial composition. In other aspects, the subject does not exhibit a sign or symptom of a disease or disorder, such as those disclosed herein prior to administration of the formulation or a bacterial composition, e.g., formulation or a bacterial composition is administered prophylactically to reduce the risk of a sign or symptom of a disease or disorder, such as those disclosed herein.

In some aspects, a formulation or a bacterial composition described herein is administered enterically, in other words, by a route of access to the gastrointestinal tract. This includes oral administration, rectal administration (including enema, suppository, or colonoscopy), by an oral or nasal tube (nasogastric, nasojejunal, oral gastric, or oral jejunal), or any other method known in the art.

In some aspects, a formulation or a bacterial composition is administered to at least one region of the gastrointestinal tract, including the mouth, esophagus, stomach, small intestine, large intestine, and rectum. In other aspects, a formulation or a bacterial composition is administered to all regions of the gastrointestinal tract. In certain aspects, a formulation is administered orally in the form of medicaments such as powders, capsules, tablets, gels or liquids. The formulation or a bacterial composition can also be administered in gel or liquid form by the oral route or through a nasogastric tube, or by the rectal route in a gel or liquid form, by enema or instillation through a colonoscope or by a suppository.

In some aspects, the bacteria and bacterial compositions are provided in a dosage form. In some aspects, the dosage form is designed for administration of at least one OTU or combination thereof disclosed herein, wherein the total amount of bacterial composition administered is selected from about 0.1 ng to about 10 g, about 10 ng to about 1 g, about 100 ng to about 0.1 g, about 0.1 mg to about 500 mg, about 1 mg to about 1000 mg, from about 1000 to about 5000 mg, or more.

In some aspects, the treatment period is at least about 1 day, at least about 2 days, at least about 3 days, at least about 4 days, at least about 5 days, at least about 6 days, at least about 1 week, at least about 2 weeks, at least about 3 weeks, at least about 4 weeks, at least about 1 month, at least about 2 months, at least about 3 months, at least about 4 months, at least about 5 months, at least about 6 months, or at least about 1 year. In some aspects, the treatment period is from about 1 day to 1 week, from about 1 week to 4 weeks, from about 1 month, to 3 months, from about 3 months to 6 months, from about 6 months to 1 year, or for over a year.

In some aspects, from about 10⁵ and about 10¹² microorganisms total is administered to the patient in a given dosage form. In certain aspects, an effective amount can be provided in from about 1 to about 500 ml or from about 1 to about 500 grams of the bacterial composition having from about 10⁷ to about 10¹¹ bacteria per ml or per gram, or a capsule, tablet, or suppository having from about 1 mg to about 1000 mg lyophilized powder having from about 10⁷ to about 10¹¹ bacteria. In some aspects, those receiving acute treatment receive higher doses than those who are receiving chronic administration (such as hospital workers or those admitted into long-term care facilities).

In some aspects, a formulation or a bacterial composition described herein is administered once, on a single occasion or on multiple occasions, such as once a day for several days or more than once a day on the day of administration (including twice daily, three times daily, or up to five times daily). In some aspects, a formulation or a bacterial composition is administered intermittently according to a set schedule, e.g., once a day, once weekly, or once monthly, or when the patient relapses from clinical improvement (e.g., clinical remission) of a disease or disorder, such as those disclosed herein, or exhibits a sign or symptoms of a disease or disorder, such as those disclosed herein. In other aspects, a formulation or a bacterial composition is administered on a long-term basis to individuals who are at risk for active disease or disorder, such as those disclosed herein or are diagnosed as being at risk for developing a disease or disorder (e.g., have a family history of UC or a history of isotretinoin use by the individual).

In some aspects, a bacterial composition of the present disclosure is administered with other agents (e.g., anti-microbial agents or prebiotics) as a combination therapy mode. In certain aspects, the administration is sequential, over a period of hours or days. In other aspects, the administration is simultaneous.

In some aspects, a bacterial composition is included in combination therapy with one or more anti-microbial agents, which include anti-bacterial agents, anti-fungal agents, anti-viral agents and anti-parasitic agents.

Anti-bacterial agents include cephalosporin antibiotics (cephalexin, cefuroxime, cefadroxil, cefazolin, cephalothin, cefaclor, cefamandole, cefoxitin, cefprozil, and ceftobiprole); fluoroquinolone antibiotics (cipro, Levaquin, floxin, tequin, avelox, and norflox); tetracycline antibiotics (tetracycline, minocycline, oxytetracycline, and doxycycline); penicillin antibiotics (amoxicillin, ampicillin, penicillin V, dicloxacillin, carbenicillin, vancomycin, and methicillin); and carbapenem antibiotics (ertapenem, doripenem, imipenem/cilastatin, and meropenem).

Anti-viral agents include Abacavir, Acyclovir, Adefovir, Amprenavir, Atazanavir, Cidofovir, Darunavir, Delavirdine, Didanosine, Docosanol, Efavirenz, Elvitegravir, Emtricitabine, Enfuvirtide, Etravirine, Famciclovir, Foscarnet, Fomivirsen, Ganciclovir, Indinavir, Idoxuridine, Lamivudine, Lopinavir Maraviroc, MK-2048, Nelfinavir, Nevirapine, Penciclovir, Raltegravir, Rilpivirine, Ritonavir, Saquinavir, Stavudine, Tenofovir Trifluridine, Valaciclovir, Valganciclovir, Vidarabine, Ibacitabine, Amantadine, Oseltamivir, Rimantidine, Tipranavir, Zalcitabine, Zanamivir and Zidovudine.

Examples of antifungal compounds include, but are not limited to polyene antifungals such as natamycin, rimocidin, filipin, nystatin, amphotericin B, candicin, and hamycin; imidazole antifungals such as miconazole, ketoconazole, clotrimazole, econazole, omoconazole, bifonazole, butoconazole, fenticonazole, isoconazole, oxiconazole, sertaconazole, sulconazole, and tioconazole; triazole antifungals such as fluconazole, itraconazole, isavuconazole, ravuconazole, posaconazole, voriconazole, terconazole, and albaconazole; thiazole antifungals such as abafungin; allylamine antifungals such as terbinafine, naftifine, and butenafine; and echinocandin antifungals such as anidulafungin, caspofungin, and micafungin. Other compounds that have antifungal properties include, but are not limited to polygodial, benzoic acid, ciclopirox, tolnaftate, undecylenic acid, flucytosine or 5-fluorocytosine, griseofulvin, and haloprogin.

In some aspects, a bacterial composition is included in combination therapy with one or more corticosteroids, mesalazine, mesalamine, sulfasalazine, sulfasalazine derivatives, immunosuppressive drugs, cyclosporin A, mercaptopurine, azathiopurine, prednisone, methotrexate, antihistamines, glucocorticoids, epinephrine, theophylline, cromolyn sodium, anti-leukotrienes, anti-cholinergic drugs for rhinitis, anti-cholinergic decongestants, mast-cell stabilizers, monoclonal anti-IgE antibodies, vaccines, and combinations thereof.

A prebiotic is a selectively fermented ingredient that allows specific changes, both in the composition and/or activity in the gastrointestinal microbiota that confers benefits upon a treated subject's well-being and health. Prebiotics can include complex carbohydrates, amino acids, peptides, or other essential nutritional components for the survival of the bacterial composition. Prebiotics include, but are not limited to, amino acids, biotin, fructooligosaccharide, galactooligosaccharides, inulin, lactulose, mannan oligosaccharides, oligofructose-enriched inulin, oligofructose, oligodextrose, tagatose, trans-galactooligosaccharide, and xylooligosaccharides.

To evaluate a subject, signs or symptoms of an adverse event or disease recurrence are evaluated post-treatment ranging from, e.g., about 1 day to about 6 months after administration of a formulation or a bacterial composition. One method of evaluation involves obtaining fecal material from the subject and assessment of microbes present in the gastrointestinal tract, e.g., using 16S rDNA or metagenomic shotgun sequencing analysis or other analyses known in the art. Population of the gastrointestinal tract by bacterial species present the formulation or a bacterial composition as well as augmentation by commensal microbes not present in the formulation or the bacterial composition can be used to indicate an improvement in the GI dysbiosis associated with e.g., UC, and therefore a decreased risk of an adverse event or a decrease in the severity of an adverse event.

In addition to treating the different inflammatory diseases disclosed herein (e.g., colitis), Applicant has surprisingly discovered that the designed compositions disclosed herein can be also used to treat diseases or disorders that are generally not associated with pro-inflammatory responses. A non-limiting example of such a disease or disorder is cancer. In some aspects, the bacterial compositions disclosed herein (e.g., designed compositions) can be used to treat certain cancers, e.g., when administered in combination with other anti-cancer agents. Without being limited to any one particular theory, the compositions disclosed herein are designed to have functional features that target multiple biological pathways. In some aspects, the functional features are important for the treatment of inflammatory diseases. In other aspects, the functional features are important for the treatment of cancers. In certain aspects, the functional features are important for the treatment of both inflammatory diseases and cancers. Non-limiting examples of functional features that can be important for the treatment of both inflammatory diseases and cancers include, but are not limited to, inhibition of HDAC activity, production of short-chain fatty acids, production of tryptophan metabolites, production of IL-18, activation of CD8+ T cells by metabolites (e.g., short-chain fatty acids) or macromolecules, activation of antigen presenting cells such as dendritic cells by bacterial antigens, macromolecules and metabolites, reducing expression of one or more inhibitory receptors (e.g., TIGIT, TIM-3, or LAG-3) on CD8+ T cells, increasing expression of one or more genes/proteins associated with T cell activation and/or function (e.g., CD45RO, CD69, IL-24, TNF-α, perforin, or IFN-γ), enhancing the ability of CD8+ T cells to kill tumor cells, enhancing the efficacy of an immune checkpoint inhibitor, or reduced colonic inflammation (e.g., through upregulation of Tregs), or enabling recruitment of CD8+ T cells to tumors located distally.

In some aspects, a designed composition disclosed herein is administered in combination with an additional therapeutic agent used for the treatment of cancers. Such additional therapeutic agents can include, for example, chemotherapy drugs, small molecule drugs or antibodies that stimulate the immune response to a given cancer. In some instances, therapeutic compositions can include an immune checkpoint inhibitor, e.g., an anti-PD-1 antibody, an anti-PD-L1 antibody, an anti-CTLA-4 antibody, or any combination thereof. Non-limiting examples of other antibodies that can be used in combination with the designed compositions of the present disclosure include an anti-OX40 (also known as CD134, TNFRSF4, ACT35 and/or TXGP1L) antibody, an anti-CD137 antibody, an anti-LAG-3 antibody, or an anti-GITR antibody.

In some aspects, a designed composition disclosed herein, when administered in combination with an anti-cancer agent (e.g., immune checkpoint inhibitor, e.g., anti-PD-1 antibody or an anti-PD-L1 antibody), can reduce tumor volume in a subject. In certain aspects, tumor volume is decreased by at least about 10%, at least about 20%, at least about 30%, at least about 40%, at least about 50%, at least about 60%, at least about 70%, at least about 80%, or at least about 90% in the subject, compared to a reference (e.g., tumor volume in the subject prior to the administration or a corresponding subject that did not receive the compositions disclosed herein).

In some aspects, a designed composition disclosed herein, when administered in combination with an anti-cancer agent (e.g., immune checkpoint inhibitor, e.g., anti-PD-1 antibody or an anti-PD-L1 antibody), can increase the percentage of CD8 T cells and/or CD4 T cells (tumor infiltrating lymphocytes) in the tumor of a subject. In some aspects, the percentage of CD8 T cells and/or CD4 T cells in the tumor is increased by at least about 10%, at least about 20%, at least about 30%, at least about 40%, at least about 50%, at least about 60%, at least about 70%, at least about 80%, or at least about 90% in the subject, compared to a reference (e.g., tumor volume in the subject prior to the administration or a corresponding subject that did not receive the compositions disclosed herein). As a result of the increase in the percentage of CD8 T cells, in some aspects, the ratio of CD8 T cells to regulatory T cells in the tumor is increased, e.g., by at least about 10%, at least about 20%, at least about 30%, at least about 40%, at least about 50%, at least about 60%, at least about 70%, at least about 80%, or at least about 90% in the subject, compared to a reference.

Non-limiting examples of cancers that can be treated with the present disclosure include squamous cell carcinoma, small-cell lung cancer, non-small cell lung cancer, squamous non-small cell lung cancer (NSCLC), nonsquamous NSCLC, glioma, gastrointestinal cancer, renal cancer (e.g., clear cell carcinoma), ovarian cancer, liver cancer, colorectal cancer, endometrial cancer, kidney cancer (e.g., renal cell carcinoma (RCC)), prostate cancer (e.g., hormone refractory prostate adenocarcinoma), thyroid cancer, neuroblastoma, pancreatic cancer, glioblastoma (glioblastoma multiforme), cervical cancer, stomach cancer, bladder cancer, hepatoma, breast cancer, colon carcinoma, and head and neck cancer (or carcinoma), gastric cancer, germ cell tumor, pediatric sarcoma, sinonasal natural killer, melanoma (e.g., metastatic malignant melanoma, such as cutaneous or intraocular malignant melanoma), bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, neoplasm of the central nervous system (CNS), primary CNS lymphoma, tumor angiogenesis, spinal axis tumor, brain cancer, brain stem glioma, pituitary adenoma, Kaposi's sarcoma, epidermoid cancer, squamous cell cancer, T-cell lymphoma, environmentally-induced cancers including those induced by asbestos, virus-related cancers or cancers of viral origin (e.g., human papilloma virus (HPV-related or -originating tumors)), and hematologic malignancies derived from either of the two major blood cell lineages, i.e., the myeloid cell line (which produces granulocytes, erythrocytes, thrombocytes, macrophages and mast cells) or lymphoid cell line (which produces B, T, NK and plasma cells), such as all types of leukemias, lymphomas, and myelomas, e.g., acute, chronic, lymphocytic and/or myelogenous leukemias, such as acute leukemia (ALL), acute myelogenous leukemia (AML), chronic lymphocytic leukemia (CLL), and chronic myelogenous leukemia (CML), undifferentiated AML (MO), myeloblastic leukemia (Ml), myeloblastic leukemia (M2; with cell maturation), promyelocytic leukemia (M3 or M3 variant [M3V]), myelomonocytic leukemia (M4 or M4 variant with eosinophilia [M4E]), monocytic leukemia (M5), erythroleukemia (M6), megakaryoblastic leukemia (M7), isolated granulocytic sarcoma, and chloroma; lymphomas, such as Hodgkin's lymphoma (HL), non-Hodgkin's lymphoma (NHL), B cell hematologic malignancy, e.g., B-cell lymphomas, T-cell lymphomas, lymphoplasmacytoid lymphoma, monocytoic B-cell lymphoma, mucosa-associated lymphoid tissue (MALT) lymphoma, anaplastic (e.g., Ki 1+) large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; and lymphoma/leukaemia (T-Lbly/T-ALL), peripheral T-cell lymphoma, lymphoblastic lymphoma, post-transplantation lymphoproliferative disorder, true histiocytic lymphoma, primary central nervous system lymphoma, primary effusion lymphoma, B cell lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC) (also called mycosis fungoides or Sezary syndrome), and lymphoplasmacytoid lymphoma (LPL) with Waldenstrom's macroglobulinemia; myelomas, such as IgG myeloma, light chain myeloma, nonsecretory myeloma, smoldering myeloma (also called indolent myeloma), solitary plasmocytoma, and multiple myelomas, chronic lymphocytic leukemia (CLL), hairy cell lymphoma; hematopoietic tumors of myeloid lineage, tumors of mesenchymal origin, including fibrosarcoma and rhabdomyoscarcoma; seminoma, teratocarcinoma, tumors of the central and peripheral nervous, including astrocytoma, schwannomas; tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) of the T-cell type; a/d T-NHL hepatosplenic lymphoma; peripheral/post-thymic T cell lymphoma (pleomorphic and immunoblastic subtypes); angiocentric (nasal) T-cell lymphoma; cancer of the head or neck, renal cancer, rectal cancer, cancer of the thyroid gland; acute myeloid lymphoma, as well as any combinations of said cancers. The methods described herein can also be used for treatment of metastatic cancers, unresectable, refractory cancers (e.g., cancers refractory to previous immunotherapy, e.g., with a blocking CTLA-4 or PD-1 antibody), and/or recurrent cancers.

### IV. Methods of Identifying Suitable FMT Donors

Applicant has discovered that certain microbiome profiles, e.g., families, genera, and/or species, are associated with improved clinical efficacy in a disease or disorder, such as those disclosed herein (e.g., ulcerative colitis patients). Accordingly, in certain aspects, the present disclosure provides a method of selecting donors whose feces are useful for preparing bacterial compositions and formulations disclosed herein. In some aspects, the method comprises: a) obtaining a microbiome sample from a subject (i.e., potential donor), and b) determining the prevalence of a family, genera, and/or species of bacteria in the microbiome sample.

In some aspects, the subject is a suitable donor if the microbiome sample comprises one or more bacteria from the family Ruminococcaceae, Lachnospiraceae, Sutterellaceae, Clostridiaceae, Erysipelotrichaceae, Bacteroidaceae, Akkermansiaceae, Peptostreptococcaceae, Eubacteriaceae, or Desulfovibrionaceae. In some aspects, the subject is a suitable donor if the microbiome sample comprises one or more of the following bacterial species: Gemmiger formicilis, Roseburia hominis, Clostridium bolteae, Parasutterella excrementihominis, Holdemania filformis, Holdemania massiliensis, Bacteroides ovatus, Akkemansia muciniphila, Clostridium leptum, Bilophila wadsworthia, Dielma fastidiosa, Clostridium symbiosum, Eubacterium siraeum, Agathobaculum desmolans, Agathobaculum butyriciproducens, or Bacteroides vulgatus. In some aspects, the subject is a suitable donor if the microbiome sample comprises one or more of the following bacterial species: Anaerotruncus colihominis, Blautia producta, Clostridium bolteae, Clostridium disporicum, Clostridium ghonii, Clostridium glycolicum, Clostridium innocuum, Clostridium lactatifermentans, Clostridium viride, Eubacterium sp. WAL 14571, Lachnospiraceae bacterium 3 1 57FA, Lachnospiraceae bacterium oral taxon F15, Lactonifactor longoviformis, or Ruminococcus lactaris. In certain aspects, the subject is a suitable donor if the microbiome sample comprises one or more bacteria disclosed in Table 4, Table 5, FIG. 13, FIG. 17, FIG. 18, FIG. 31, FIG. 32, FIG. 33, and/or FIG. 34. In some aspects, the subject is a suitable donor if the microbiome sample comprises one or more bacteria comprising a 16S rDNA sequence that is at least about 85%, at least about 90%, at least about 95%, at least about 96%, at least about 97%, at least about 97.5%, at least about 98%, at least about 98.5%, at least about 99%, at least about 99.5%, or about 100% identical to a 16S rDNA sequence set forth in SEQ ID NOs: 1-14, 16-30, 32-36, 39, 41, 44, 45, 47-51, 59-62, 64-68, 72-76, 102-398, or any of the foregoing species.

In some aspects, a donor is selected that produce relatively higher concentrations of spores in fecal material than other donors. In further aspects, a donor is selected that provide fecal material from which spores having increased efficacy are purified; this increased efficacy is measured using in vitro or in animal studies as described herein or by any other method known in the art. In some aspects, a donor can be subjected to one or more pre-donation treatments to reduce undesired material in the fecal material, and/or increase desired spore populations.

It is advantageous to screen the health of a donor subject prior to and optionally, one or more times after, the collection of the fecal material. Such screening identifies donors carrying pathogenic materials such as viruses (HIV, hepatitis, polio) and pathogenic bacteria. Post-collection, donors are screened about one week, two weeks, three weeks, one month, two months, three months, six months, one year or more than one year, and the frequency of such screening can be daily, weekly, bi-weekly, monthly, bi-monthly, semi-yearly or yearly. In some aspects, donors that are screened and do not test positive, either before or after donation or both, are considered “validated” or suitable donors.

V. Methods of Identifying a Candidate for Treatment with a Designed Composition

Applicant has discovered that certain microbiome profiles, e.g., families, genera, and/or species, are associated with an exacerbation or non-improvement (e.g., no clinical remission) of a disease or disorder, such as those disclosed herein (e.g., ulcerative colitis). Accordingly, in certain aspects, the present disclosure provides a method of identifying a subject with a reduced likelihood of responding to a bacterial composition or formulation disclosed herein. Alternatively, provided herein is a method for identifying a subject who is likely to respond (e.g., clinical remission) to a bacterial composition or formulation disclosed herein. In some aspects, the method comprises: a) obtaining a microbiome sample from a subject (e.g., ulcerative colitis patient who received a bacterial composition disclosed herein), and b) determining the prevalence of a family, genera, and/or species of bacteria in the microbiome sample.

In some aspects, the subject is not likely to respond to a treatment disclosed herein if the microbiome sample comprises one or more of the following bacterial species: Eubacterium contortum, Clostridium hathewayi, Erysipelatoclostridum ramosum, Bifidobacterium dentium, Dialister invisus, Prevotella copri, Veillonella atypica, Veillonella dispar, Veillonella parvula, or Veillonella ratti. In some aspects, the subject is not likely to respond if the microbiome sample comprises one or more bacteria comprising a 16S rDNA sequence that is at least about 85%, at least about 90%, at least about 95%, at least about 96%, at least about 97%, at least about 97.5%, at least about 98%, at least about 98.5%, at least about 99%, at least about 99.5%, or about 100% identical to a 16S rDNA sequence set forth in SEQ ID NO: 15, 31, 37, 38, 40, 42, 43, 46, 52-58, 63, 69-71, and 83-101 or any of the foregoing species.

In some aspects, the subject is likely to respond to a treatment disclosed herein if the microbiome sample does not comprise one or more of the following bacterial species: Eubacterium contortum, Clostridium hathewayi, Erysipelatoclostridum ramosum, Bifidobacterium dentium, Dialister invisus, Prevotella copri, Veillonella atypica, Veillonella dispar, Veillonella parvula, or Veillonella ratti. In some aspects, the subject is likely to respond to treatment if the microbiome sample does not comprise one or more bacteria comprising a 16S rDNA sequence that is at least about 85%, at least about 90%, at least about 95%, at least about 96%, at least about 97%, at least about 97.5%, at least 98%, at least about 98.5%, at least 99%, at least about 99.5%, or about 100% identical to a 16S rDNA sequence set forth in SEQ ID NO: 15, 31, 37, 38, 40, 42, 43, 46, 52-58, 63, 69-71, and 83-101 or any of the foregoing species.

In some aspects, the subject, e.g., an individual diagnosed with a disease or disorder, such as those disclosed herein, is a candidate for treatment with a composition disclosed herein if a GI microbiome sample from the subject comprises one or more of the following bacterial species: Gemmiger formicilis, Roseburia hominis, Clostridium bolteae, Parasutterella excrementihominis, Holdemania fihformis, Holdemania massiliensis, Bacteroides ovatus, Akkemansia muciniphila, Clostridium leptum, Bilophila wadsworthia, Dielma fastidiosa, Clostridium symbiosum, Eubacterium siraeum, Agathobaculum desmolans, Agathobaculum butyriciproducens, or Bacteroides vulgatus. In some aspects, the subject is a candidate for treatment with a composition disclosed herein if a GI microbiome sample comprises Anaerotruncus colihominis, Blautia producta, Clostridium bolteae, Clostridium disporicum, Clostridium ghonii, Clostridium glycolicum, Clostridium innocuum, Clostridium lactatifermentans, Clostridium viride, Eubacterium sp. WAL 14571, Lachnospiraceae bacterium 3 1 57FA, Lachnospiraceae bacterium oral taxon F15, Lactonifactor longoviformis, or Ruminococcus lactaris. In some aspects, the subject is a suitable donor if the microbiome sample from the subject comprises one or more bacteria disclosed in Table 4, Table 5, FIG. 13, FIG. 17, FIG. 18, FIG. 31, FIG. 32, FIG. 33, and/or FIG. 34. In some aspects, the subject is a candidate for treatment with a composition disclosed herein if the microbiome sample comprises one or more bacteria comprising a 16S rDNA sequence that is at least about 85%, at least about 90%, at least about 95%, at least about 96%, at least about 97%, at least about 97.5%, at least about 98%, at least about 98.5%, at least about 99%, at least about 99.5%, or about 100% identical to a 16S rDNA sequence set forth in SEQ ID NOs: 1-14, 16-30, 32-36, 39, 41, 44, 45, 47-51, 59-62, 64-68, 72-76, 102-398 or any of the foregoing species. A candidate for treatment is a subject likely to respond to treatment with a composition provided herein by improvement in one or more signs or symptoms of a disease or disorder, such as those associated with a dysbiosis.

### VI. Additional Information

Certain terms used in the present application are defined as follows. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related.

Units, prefixes, and symbols are denoted in their Systeme International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, 5 percent, 3 percent, 2 percent, or 1 percent; up or down (higher or lower).

The term “clade” refers to the OTUs or members of a phylogenetic tree that are downstream of a statistically valid node in a phylogenetic tree. The clade comprises a set of terminal leaves in the phylogenetic tree that is a distinct monophyletic evolutionary unit and that share some extent of sequence similarity.

The term “microbiota” refers to the ecological community of microorganisms that occur (sustainably or transiently) in and on an animal subject, typically a mammal such as a human, including eukaryotes, archaea, bacteria, and viruses (including bacterial viruses i.e., phage).

The term “microbiome” refers to the microbes that live in and on the human body, both sustainably and transiently, including eukaryotes, archaea, bacteria, and viruses (including bacterial viruses (i.e., phage)). As used herein, “genetic content” includes genomic DNA, RNA such as ribosomal RNA, the epigenome, plasmids, and all other types of genetic information.

The term “ecological niche” or “niche” refers to the ecological space in which an organism or group of organisms occupies. Niche describes how an organism or population or organisms responds to the distribution of resources, physical parameters (e.g., host tissue space) and competitors (e.g., by growing when resources are abundant, and when predators, parasites and pathogens are scarce) and how it in turn alters those same factors (e.g., limiting access to resources by other organisms, acting as a food source for predators and a consumer of prey).

The term “dysbiosis” refers to a state of the microbiota of the GI tract or other body area in a subject, including mucosal or skin surfaces in which the normal diversity and/or function of the ecological network is disrupted. This unhealthy state can be due to a decrease in diversity, the overgrowth of one or more pathogens or pathobionts, symbiotic organisms able to cause disease only when certain genetic and/or environmental conditions are present in a subject, or the shift to an ecological microbial network that no longer provides an essential function to the host subject, and therefore no longer promotes health.

As used herein, the term “operational taxonomic units” or “OTU” (or plural, “OTUs”) refers to a terminal leaf in a phylogenetic tree and is defined by a nucleic acid sequence, e.g., the entire genome, or a specific genetic sequence, and all sequences that share sequence identity to this nucleic acid sequence at the level of species. In some aspects the specific genetic sequence can be the 16S rDNA sequence or a portion of the 16S rDNA sequence. In other aspects, the entire genomes of two entities are sequenced and compared. In another aspect, select regions such as multilocus sequence tags (MLST), specific genes, or sets of genes can be genetically compared. In 16S aspects, OTUs that share ≥97% average nucleotide identity across the entire 16S or a variable region of the 16S rDNA, e.g., a V4 region, are considered the same OTU (see, e.g., Claesson M J, Wang Q, O'Sullivan O, Greene-Diniz R, Cole J R, Ros R P, and O'Toole P W. 2010. Comparison of two next-generation sequencing technologies for resolving highly complex microbiome composition using tandem variable 16S rRNA gene regions. Nucleic Acids Res 38: e200. Konstantinidis K T, Ramette A, and Tiedje J M. 2006. The bacterial species definition in the genomic era. Philos Trans R Soc Lond B Biol Sci 361: 1929-1940). In aspects involving the complete genome, MLSTs, specific genes, or sets of genes OTUs that share ≥95% average nucleotide identity are considered the same OTU (see, e.g., Achtman M, and Wagner M. 2008. Microbial diversity and the genetic nature of microbial species. Nat. Rev. Microbiol. 6: 431-440. Konstantinidis K T, Ramette A, and Tiedje J M. 2006. The bacterial species definition in the genomic era. Philos Trans R Soc LondB Biol Sci 361: 1929-1940.). OTUs are frequently defined by comparing sequences between organisms. Generally, sequences with less than 95% sequence identity are not considered to form part of the same OTU. In some cases, an OTU is characterized by a combination of nucleotide markers, genes, and/or single nucleotide variants (SNVs). In some cases, the referenced genes are highly conserved genes (e.g., “house-keeping” genes). The features defining an OTU can be a combination of the foregoing. Such characterization employs, e.g., WGS data or a whole genome sequence.

As used herein, the term “phylogenetic tree” refers to a graphical representation of the evolutionary relationships of one genetic sequence to another that is generated using a defined set of phylogenetic reconstruction algorithms (e.g., parsimony, maximum likelihood, or Bayesian). Nodes in the tree represent distinct ancestral sequences and the confidence of any node is provided by a bootstrap or Bayesian posterior probability, which measures branch uncertainty.

The specification is most thoroughly understood in light of the teachings of the references cited within the specification. The aspects within the specification provide an illustration of aspects and should not be construed to limit the scope. The skilled artisan readily recognizes that many other aspects are encompassed. All publications and patents cited in this disclosure are incorporated by reference in their entirety. To the extent the material incorporated by reference contradicts or is inconsistent with this specification, the specification will supersede any such material. The citation of any references herein is not an admission that such references are prior art.

As used herein, the term “subject” refers to any animal subject including humans, laboratory animals (e.g., primates, rats, mice), livestock (e.g., cows, sheep, goats, pigs, turkeys, and chickens), and household pets (e.g., dogs, cats, and rodents). The subject can be suffering from a dysbiosis, including, but not limited to, an infection due to a gastrointestinal pathogen or can be at risk of developing or transmitting to others an infection due to a gastrointestinal pathogen. In some aspects, the subject is suffering from an ulcerative colitis.

Ulcerative colitis (UC) is a disease of the large intestine (colon) characterized by chronic diarrhea with cramping abdominal pain, rectal bleeding, and loose discharges of blood, pus and mucus. The manifestations of ulcerative colitis vary widely. A pattern of exacerbations and improvements typifies the clinical course of most UC patients (70%), although continuous symptoms without improvement are present in some patients with UC. Local and systemic complications of UC include arthritis, eye inflammation such as uveitis, skin ulcers and liver disease. In addition, ulcerative colitis and especially long-standing, extensive disease is associated with an increased risk of colon carcinoma. Bacterial compositions provided herein can be used to ameliorate one or more characteristics of ulcerative colitis or other IBD.

Several pathologic features characterize UC in distinction to other inflammatory bowel diseases. Ulcerative colitis is a diffuse disease that usually extends from the most distal part of the rectum for a variable distance proximally. The term left-sided colitis describes an inflammation that involves the distal portion of the colon, extending as far as the splenic flexure. Sparing of the rectum or involvement of the right side (proximal portion) of the colon alone is unusual in ulcerative colitis. The inflammatory process of ulcerative colitis is limited to the colon and does not involve, for example, the small intestine, stomach or esophagus. In addition, ulcerative colitis is distinguished by a superficial inflammation of the mucosa that generally spares the deeper layers of the bowel wall. Crypt abscesses, in which degenerated intestinal crypts are filled with neutrophils, also are typical of ulcerative colitis (Rubin and Farber, supra, 1994).

Ulcerative colitis can be further categorized as “mild,” “moderate,” “severe,” or “fulminant” (very severe). In some aspects, the ulcerative colitis to be treated is mild to moderate, e.g., a Mayo score of ≥4 and ≤10. In some aspects a patient to be treated with a microbiome composition has been diagnosed with moderately to severely active UC. In some aspects, the patient diagnosed with UC has had an inadequate response to, loss of response, or is intolerant to conventional or biologic therapy. In some aspects, a subject treated with a microbiome composition exhibits one of more of the following improvements: clinical response based on a Mayo score, e.g., modified Mayo score (MMS), endoscopic remission based on the MMS Endoscopic Subscore (ES), symptomatic remission based on MMS Stool Frequency (SF) and Rectal Bleeding (RB) subscores, symptomatic response based on MMS SF and RB subscores, mucosal healing based on a histologic disease activity index (Geboes score or Robards Histology Index), endoscopic response based on the MMS ES, UC symptoms based on NRS scores, Health Related Quality of Life based on IBDQ score, and change from baseline to Week 7, 8, or 12 in fecal calprotectin levels.

In addition to ulcerative colitis, the bacterial compositions disclosed herein can also be useful for the treatment of other diseases or disorders, including those associated with a dysbiosis of the gastrointestinal tract. Without being bound by any one theory, bacterial compositions disclosed herein can treat such diseases or disorders by engrafting and repopulating the gastrointestinal tract of a subject, and thereby shift the subject's microbiome from one of dysbiosis to one that more resembles a healthy state. In some aspects, bacterial compositions disclosed herein can prevent the growth of a pathogen associated with a disease or disorder disclosed herein (e.g., by outcompeting for growth nutrients). In some aspects, a bacterial composition disclosed herein can be designed to produce various factors that can, e.g., reduce and/or inhibit a pro-inflammatory immune response (e.g., by producing factors, such as tryptophan metabolites, fatty acids, secondary bile acid, or by inhibiting HDAC activation).

Non-limiting examples of such diseases or disorders include immune-mediated gastrointestinal disorders, including, but not limited to, Crohn's disease, lymphocytic colitis; microscopic colitis; collagenous colitis: autoimmune enteropathy, including autoimmune enteritis and autoimmune enterocolitis; allergic gastrointestinal disease; and eosinophilic gastrointestinal disease, including eosinophilic gastroenteritis and eosinophilic enteropathy. Non-limiting examples of other immune-mediated disorders that may be treated with a composition described herein include: arthritis (acute and chronic, rheumatoid arthritis including juvenile-onset rheumatoid arthritis and stages such as rheumatoid synovitis, gout or gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, osteoarthritis, arthritis chronica progrediente, arthritis deformans, polyarthritis chronica primaria, reactive arthritis, menopausal arthritis, estrogen-depletion arthritis, and ankylosing spondylitis/rheumatoid spondylitis), autoimmune lymphoproliferative disease, inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, hives, dermatitis herpetiformis, nummular dermatitis, seborrheic dermatitis, non-specific dermatitis, primary irritant contact dermatitis, and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica (NMO), inflammatory bowel disease (IBD) (for example, Crohn's disease, autoimmune-mediated gastrointestinal diseases, gastrointestinal inflammation, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, graft-versus-host disease, angioedema such as hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN (RPGN), proliferative nephritis, autoimmune polyglandular endocrine failure, balanitis including balanitis circumscripta plasmacellularis, balanoposthitis, erythema annulare centrifugum, erythema dyschromicum perstans, eythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, food allergies, drug allergies, insect allergies, rare allergic disorders such as mastocytosis, allergic reaction, eczema including allergic or atopic eczema, asteatotic eczema, dyshidrotic eczema, and vesicular palmoplanar eczema, asthma such as asthma bronchiale, bronchial asthma, and auto-immune asthma, conditions involving infiltration of T cells and chronic inflammatory responses, immune reactions against foreign antigens such as fetal A-B-0 blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, SLE, such as cutaneous SLE or subacute cutaneous SLE, neonatal lupus syndrome (NLE), and lupus erythematosus disseminatus, juvenile onset (Type I) diabetes mellitus, including pediatric IDDM, adult onset diabetes mellitus (Type II diabetes), autoimmune diabetes, idiopathic diabetes insipidus, diabetic retinopathy, diabetic nephropathy, diabetic colitis, diabetic large-artery disorder, immune responses associated with acute and delayed hypersensitivity mediated by cytokines and T-lymphocytes, tuberculosis, sarcoidosis, granulomatosis including lymphomatoid granulomatosis, agranulocytosis, vasculitides (including large-vessel vasculitis such as polymyalgia rheumatica and giant-cell (Takayasu's) arteritis, medium-vessel vasculitis such as Kawasaki's disease and polyarteritis nodosa/periarteritis nodosa, immunovasculitis, CNS vasculitis, cutaneous vasculitis, hypersensitivity vasculitis, necrotizing vasculitis such as fibrinoid necrotizing vasculitis and systemic necrotizing vasculitis, ANCA-negative vasculitis, and ANCA-associated vasculitis such as Churg-Strauss syndrome (CSS), Wegener's granulomatosis, and microscopic polyangiitis), temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia(s), cytopenias such as pancytopenia, leukopenia, diseases involving leukocyte diapedesis, CNS inflammatory disorders, Alzheimer's disease, Parkinson's disease, multiple organ injury syndrome such as those secondary to septicemia, trauma or hemorrhage, antigen-antibody complex-mediated diseases, anti-glomerular basement membrane disease, anti-phospholipid antibody syndrome, motoneuritis, allergic neuritis, Behcet's disease/syndrome, Castleman's syndrome, Goodpasture's syndrome, Reynaud's syndrome, Sjogren's syndrome, Stevens-Johnson syndrome, pemphigoid or pemphigus such as pemphigoid bullous, cicatricial (mucous membrane) pemphigoid, skin pemphigoid, pemphigus vulgaris, paraneoplastic pemphigus, pemphigus foliaceus, pemphigus mucus-membrane pemphigoid, and pemphigus erythematosus, epidermolysis bullosa acquisita, ocular inflammation, including allergic ocular inflammation such as allergic conjunctivis, linear IgA bullous disease, autoimmune-induced conjunctival inflammation, autoimmune polyendocrinopathies, Reiter's disease or syndrome, thermal injury due to an autoimmune condition, preeclampsia, an immune complex disorder such as immune complex nephritis, antibody-mediated nephritis, neuroinflammatory disorders, polyneuropathies, chronic neuropathy such as IgM polyneuropathies or IgM-mediated neuropathy, thrombocytopenia (as developed by myocardial infarction patients, for example), including thrombotic thrombocytopenic purpura (TTP), post-transfusion purpura (PTP), heparin-induced thrombocytopenia, and autoimmune or immune-mediated thrombocytopenia including, for example, idiopathic thrombocytopenic purpura (ITP) including chronic or acute ITP, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, Grave's eye disease (ophthalmopathy or thyroid-associated ophthalmopathy), polyglandular syndromes such as autoimmune polyglandular syndromes, for example, type I (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant-cell hepatitis, chronic active hepatitis or autoimmune chronic active hepatitis, pneumonitis such as lymphoid interstitial pneumonitis (LIP), bronchiolitis obliterans (non-transplant) vs. NSIP, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia such as mixed cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, keratitis such as Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal or segmental or focal segmental glomerulosclerosis (FSGS), endocrine ophthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia areata, alopecia totalis, CREST syndrome (calcinosis, Raynaud's phenomenon, esophageal dysmotility, sclerodactyly, and telangiectasia), male and female autoimmune infertility, e.g., due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, fibrosing mediastinitis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, SCID, acquired immune deficiency syndrome (AIDS), echovirus infection, sepsis (systemic inflammatory response syndrome (SIRS)), endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant-cell polymyalgia, chronic hypersensitivity pneumonitis, conjunctivitis, such as vernal catarrh, keratoconjunctivitis sicca, and epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders (cerebral vascular insufficiency) such as arteriosclerotic encephalopathy and arteriosclerotic retinopathy, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica (sympathetic ophthalmitis), neonatal ophthalmitis, optic neuritis, orchitis granulomatosa, pancreatitis, polyradiculitis acuta, pyoderma gangrenosum, Quervain's thyreoiditis, acquired spenic atrophy, non-malignant thymoma, lymphofollicular thymitis, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, autoimmune polyendocrinopathies, oophoritis, primary myxedema, autoimmune atrophic gastritis, rheumatic diseases, mixed connective tissue disease, nephrotic syndrome, insulitis, polyendocrine failure, autoimmune polyglandular syndromes, including polyglandular syndrome type I, adult-onset idiopathic hypoparathyroidism (AOIH), cardiomyopathy such as dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, allergic sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, spondyloarthropathies, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism such as chronic arthrorheumatism, lymphadenitis, reduction in blood pressure response, vascular dysfunction, tissue injury, cardiovascular ischemia, hyperalgesia, renal ischemia, cerebral ischemia, and disease accompanying vascularization, allergic hypersensitivity disorders, glomerulonephritides, reperfusion injury, ischemic re-perfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, and endometriosis

The “colonization” of a host organism includes the non-transitory residence of a bacterium or other microscopic organism. In the case of treatment, the host is generally refered to herein as a “subject”, typically a human or other mammal. As used herein, “reducing colonization” of a host subject's gastrointestinal tract (or any other microbiotal niche) by a pathogenic bacterium includes a reduction in the residence time of the pathogen in the gastrointestinal tract as well as a reduction in the number (or concentration) of the pathogen in the gastrointestinal tract or adhered to the luminal surface of the gastrointestinal tract. Measuring reductions of adherent pathogens can be demonstrated, e.g., by a biopsy sample, or reductions can be measured indirectly, e.g., by measuring the pathogenic burden in the stool of a mammalian host.

A “combination” of two or more bacteria includes the physical co-existence of the two bacteria, either in the same material or product or in physically connected products, as well as the temporal co-administration or co-localization of the two bacteria.

A “cytotoxic” activity or bacterium includes the ability to kill a bacterial cell, such as a pathogenic bacterial cell. A “cytostatic” activity or bacterium includes the ability to inhibit, partially or fully, growth, metabolism, and/or proliferation of a bacterial cell, such as a pathogenic bacterial cell.

To be free of “non-comestible products” means that a bacterial composition or other material provided herein does not have a substantial amount of a non-comestible product, e.g., a product or material that is inedible, harmful or otherwise undesired in a product suitable for administration, e.g., oral administration, to a human subject. Non-comestible products are often found in preparations of bacteria from the prior art.

A “biologically pure culture” is a culture a culture of bacteria in a medium in which only selected viable species are present and no other viable species of microorganisms are detected.

For nucleic acids, the term “substantial homology” indicates that two nucleic acids, or designated sequences thereof, when optimally aligned and compared, are identical, with appropriate nucleotide insertions or deletions, in at least about 80% of the nucleotides, at least about 90% to 95%, or at least about 98% to 99.5% of the nucleotides. Alternatively, substantial homology exists when the segments will hybridize under selective hybridization conditions, to the complement of the strand.

For polypeptides, the term “substantial homology” indicates that two polypeptides, or designated sequences thereof, when optimally aligned and compared, are identical, with appropriate amino acid insertions or deletions, in at least about 80% of the amino acids, at least about 90% to 95%, or at least about 98% to 99.5% of the amino acids.

The percent identity between two sequences is a function of the number of identical positions shared by the sequences (i.e., % homology=# of identical positions/total # of positions ×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available at worldwideweb.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4: 11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at worldwideweb.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences described herein can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the nucleic acid molecules described herein. BLAST protein searches can be performed with the XBLAST program, score=50, wordlength=3 to obtain amino acid sequences homologous to the protein molecules described herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See worldwideweb.ncbi.nlm.nih.gov. Other methods of determining identity that are known in the art can be used.

The term “patient” includes human and other mammalian subjects that receive either prophylactic or therapeutic treatment.

As used herein, the term “subject” includes any human or non-human animal. For example, the methods and compositions described herein can be used to treat a subject having cancer. The term “non-human animal” includes all vertebrates, e.g., mammals and non-mammals, such as non-human primates, sheep, dog, cow, chickens, amphibians, reptiles, etc.

As used herein, the terms “ug” and “uM“are used interchangeably with” g” and “μM,” respectively.

Various aspects described herein are described in further detail throughout the specification.

Unless otherwise indicated, all numbers expressing quantities of ingredients, reaction conditions, and so forth used in the specification, including claims, are to be understood as being modified in all instances by the term “about.” Accordingly, unless otherwise indicated to the contrary, the numerical parameters are approximations and can vary depending upon the desired properties sought to be obtained. At the very least, and not as an attempt to limit the application of the doctrine of equivalents to the scope of the claims, each numerical parameter should be construed in light of the number of significant digits and ordinary rounding approaches.

The following examples are offered by way of illustration and not by way of limitation. The contents of all references cited throughout this application are expressly incorporated herein by reference.

### EXAMPLES

### Example 1: Effect of Administration of a Spore Preparation (an HHSP) on Clinical Efficacy in Ulcerative Colitis Patients

A Phase 1b multicenter, randomized, double-blind, placebo-controlled multiple dose study (ClinicalTrials.gov Identifier: NCT02618187) was conducted to evaluate the safety and tolerability of a composition comprising purified spore population derived from the feces of healthy human donors (HHSP) for the treatment of mild to moderate ulcerative colitis in patients who had failed standard-of-care. Specific inclusion/exclusion criteria are available at worldwideweb.clinicaltrials.gov/ct2/show/NCT02618187?term=SERES-101&rank=1.

Briefly, 58 mild to moderate UC subjects (Mayo score 4-10) were randomly assigned to one of four 8-week induction treatment arms preceded by a 6-day pretreatment phase as follows: Arm A) placebo/placebo (n=11); Arm B) placebo/weekly HHSP (n=15); Arm C) vancomycin (vanco)/HHSP weekly (qwk) (n=17); or Arm D) vanco/HHSP daily (qD) (n=15). Clinical efficacy (i.e., improvement of ulcerative colitis) was determined based on one or more of the following criteria: (i) clinical remission (Total Modified Mayo (TMM) score of ≤2 plus endoscopic subscore of ≤1); and (ii) endoscopic improvement (decrease in endoscopic score of ≥1).

The patient characteristics at baseline is provided in Table 1, below.

TABLE 1
Baseline Characteristics

| | | Arm B | Arm C | Arm D |
|---|---|---|---|---|
| | Arm A | (Placebo/ | (Vanco/ | (Vanco/ |
| | (Placebo/ | HHSP | HHSP | HHSP |
| | Placebo) | Weekly) | Weekly) | Daily) |
| | (n = 11) | (n = 15) | (n = 17) | (n = 15) |
| Average Mayo | 7.3 | 6.8 | 6.4 | 6.99 |
| Score (Baseline) | | | | |
| Mild (n) | 3 (27%) | 6 (40%) | 10 (59%) | 6 (40%) |
| Moderate (n) | 8 (73%) | 9 (60%) | 7 (41%) | 9 (60%) |

Endoscopy Score at Baseline

| | | | | |
|---|---|---|---|---|
| Score = 1 | 1 (9%) | 3 (20%) | 5 (29%) | 3 (20%) |
| Score = 2 | 5 (45%) | 7 (47%) | 7 (41%) | 9 (60%) |
| Score = 3 | 5 (45%) | 5 (33%) | 5 (29%) | 3 (20%) |

### Clinical Remission and Endoscopic Healing

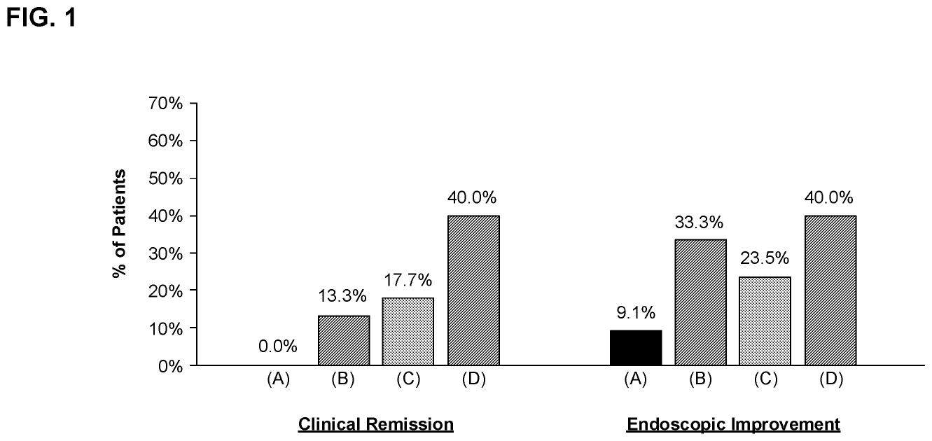

All treatment arms compared to Placebo (Arm A) resulted in increased clinical remission in patients, demonstrating that an HHSP can be used to treat ulcerative colitis. The greatest impact on remission was observed in Arm D (FIG. 1, left graph, vanco/HHSP daily) with approximately 40% of the patients going into remission. In Arm B (i.e., placebo/HHSP weekly) and Arm C (i.e., vanco/HHSP weekly), approximately 13.3% and 17.7% of the patients went into remission, respectively. Similarly, all treatment arms resulted in endoscopic improvement above the rate observed in Placebo; a higher percentage of patients that received the daily administration of HHSP (Arm D, 40%) exhibited endoscopic improvement compared to patients that received placebo alone (Arm A, 9.1%) or weekly administration of an HHSP (Arms B and C, 33.3% and 23.5%, respectively). (FIG. 1, right graph).

These data demonstrate that a spore composition derived from the feces of a healthy human can be used to ameliorate ulcerative colitis and that the parameters of clinical remission and endoscopic improvement can be used to evaluate the efficacy of a microbiome composition for treating ulcerative colitis. These data also demonstrate that a ‘complete’ microbiome as provided by FMT, is not necessary to effectively ameliorate UC.

### Long-Term Clinical Remission

To determine the long-term clinical efficacy of HHSP administration on ulcerative colitis, patients who were in remission at the end of the 8-week induction treatment period, treated patients in remission were followed for an additional 26 weeks and the number of remitters with a flare-up of disease was determined. The continuity of remission after inducing remission in a subject is termed “maintenance.”

As shown in Table 2 below, none of the remitters had a UC flare-up during the 26-week period. This was true regardless of whether the patients had received HHSP weekly (Arms B and C) or daily (Arm D).

These data demonstrate that a microbiome composition, e.g., an HHSP, can evoke a durable effect on remission.

TABLE 2
Number of Remitting Subjects with UC Flare-Up

| | | Arm B | Arm C | Arm D |
|---|---|---|---|---|
| | Arm A | (Placebo/ | (Vanco/ | (Vanco/ |
| | (Placebo/ | HHSP | HHSP | HHSP |
| | Placebo) | Weekly) | Weekly) | Daily) |
| | (n = 0) | (n = 2) | (n = 3) | (n = 6) |
| Number of | N/A | 0 | 0 | 0 |
| Remitters with | | | | |
| Flare-Up | | | | |

### Adverse Events

As part of the clinical trial protocol, adverse events were recorded and assessed at the end of the 8-week induction period. In general, patients treated with an HHSP had fewer gastrointestinal-related adverse events compared to the placebo control. The most significant difference was observed in patients that received HHSP daily (Arm D), which is consistent with a dosage-dependent effect of an HHSP.

The low level of adverse events associated with treatment with a microbiome composition demonstrated that a microbiome composition comprising a purified spore population derived from the feces of healthy human donors can safely be used to treat ulcerative colitis, including mild to moderate UC. The greatest difference in the adverse events between placebo and treated subjects was in the category of GI disorders (45.5% in placebo arm vs. 13.3% in daily treatment arm). This difference was most prominent in patients who received daily administration of the purified spore population (45.5% in placebo vs. 13.3% in Arm D).

### Example 2: Engraftment and/or Augmentation in Ulcerative Colitis Patients Treated with a Spore Preparation (HHSP)

As described in Example 1, treatment of an HHSP was able to provide a durable treatment effect in UC patients. One potential advantage of a microbiome composition for treating disease is that the microbiome composition may provide a durable effect because at least some beneficial species of the microbiome composition can engraft in the treated subject, thereby providing an ongoing source of beneficial functions and may facilitate the proliferation of advantageous bacteria not in the composition (augmentation). Not only is the lack of a durable effect an issue with pharmaceuticals that must be dosed regularly to achieve therapeutic levels, it has been noted that many probiotics must be taken with high frequency to maintain a therapeutic effect (Walter J., et al., Curr Opin Biotechnol 49: 129-139, 2018). The ability to engraft is therefore a desirable feature for bacteria in a microbiome composition, enabling, among other features, less frequent dosing than may be required with a pharmaceutical or non-engrafting probiotic. A second novel feature of certain microbiome compositions is enhancement of beneficial bacterial species not detectable or present in low levels in a patient prior to treatment with a microbiome composition.

Applicants have identified specific OTUs or species that engraft or augment and are also associated with remission. Such OTUs or species are useful in designed compositions for treating and IBD, e.g., ulcerative colitis.

To determine whether a microbiome composition can engraft and/or augment, complementary genomic methods were used to characterize the microbiota of ulcerative colitis patients at pretreatment (baseline) and up to 12 weeks post initial treatment with an HHSP (i.e., up to four weeks after the last treatment with an HHSP). The fecal microbiomes of UC subjects and HHSP doses were characterized using Whole Genome Shotgun Sequencing (WGS). WGS is a high-resolution method widely used and reported in the literature (e.g., Lloyd-Price et al., Nature 550:61-66, 2017) that enables species-level taxonomic identifications (Truong et al., Nature Meth 12:203-209, 2015). The relative abundance of species present in the fecal samples and the HHSP was obtained using the open-source software MetaPhlAn2 (ver 2.6.0) coupled with a proprietary internal database update. For analyses of engraftment, the set of species identified by MetaPhlAn2 in UC patients and HHSP was filtered against a proprietary, curated database of spore-forming species.

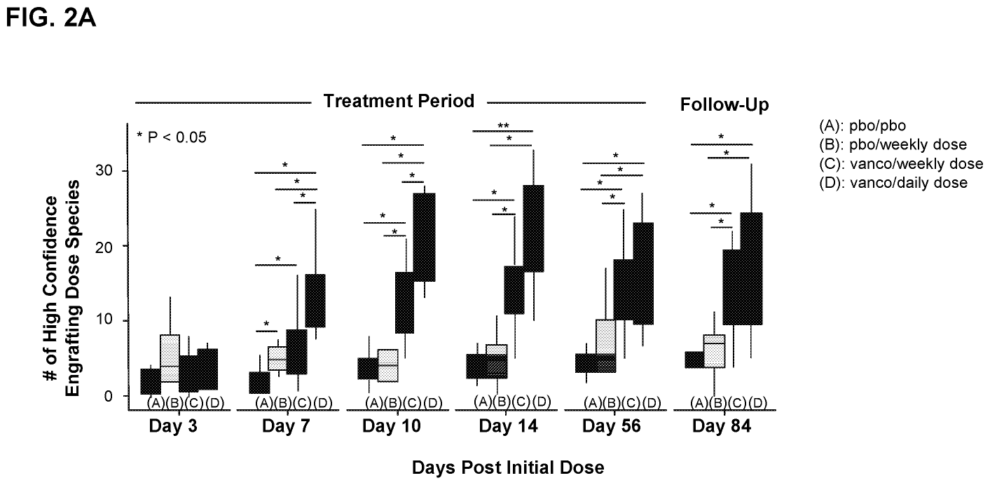

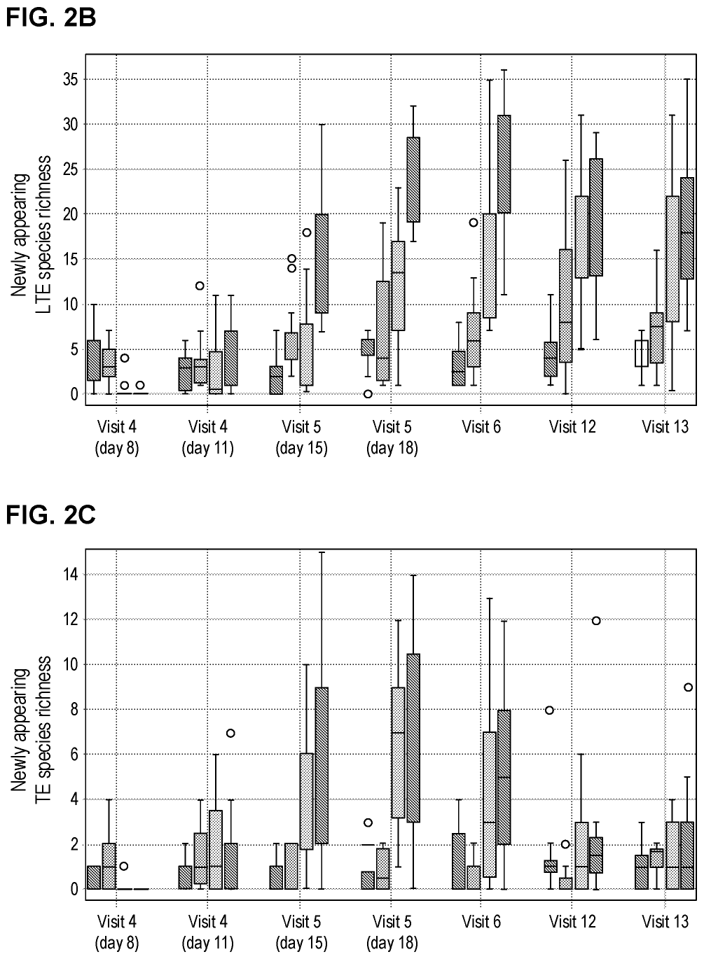

As shown in FIG. 2A, an analysis of the number of engrafting species identified in an HHSP showed that engraftment of HHSP species occurred as early as 1 week after the initial dose of an HHSP in all treatment arms (i.e., Arms B, C, and D) compared to the placebo control (Arm A). Determinations of engraftment were made based on assessing the presence or absence of spore forming bacterial species in the HHSP in a subject's stool after the initiation of treatment. Engraftment was greater in patients that were pretreated with vancomycin (e.g., Arm B v. Arm C). The highest engraftment was observed in patients that were pre-treated with vancomycin and then, received HHSP daily. Engraftment was also durable for at least 4 weeks after the final HHSP administration (see 56 days and 84 days in FIG. 2A). Interestingly, as shown in FIGS. 2B and 2C, the engrafting species could be further divided into those that were long-term engrafters (FIG. 2B) and those that were transient engrafters (FIG. 2C). The classification of a species into long-term versus transient engrafters was determined based on the identification of two distinct clusters of co-occuring engrafting species across patient samples. Transient engrafters (TE) peaked in engraftment 1-2 weeks after the start of dosing with HHSP, and show similar engraftment profiles in Arm C and Arm D. Long-term engrafters (LTE) showed a dose-dependent response at early timepoints and remained durably in patients at least 4 weeks beyond administration of the last dose (Visit 13). Table 5 provides a list of different bacterial species that were identified to be either a long-term engrafter or a transient engrafter. Importantly, many species that were present in HHSP did not engraft at detectable levels, showing that engraftment is not a universal property of species in HHSP.

This engraftment data reflects the requirements to disrupt a stable yet dysbiotic microbiome in UC patients. Across many ecological systems, communities are stable except when they experience a strong disruption. Here, vancomycin pretreatment is required to disrupt the existing UC microbiome and open a niche for engraftment of HHSP bacteria. After disruption of an ecological system, a succession of communities often appear before a final stable climax community is reached. Intermediate communities, referred to as seral communities (or seres), are often necessary to change the environment enabling establishment of subsequent communities. After the disruption of the UC microbiome with vancomycin, TE species form a seral community that is followed by establishment of LTE species, which form the stable climax community. Thus, durable therapeutic intervention can require administration of both TE and LTE species (after disrupting the existing community with vancomycin); TE and LTE species can play distinct roles that are both required to alter the environment of the gastrointestinal tract in UC.

Supporting the distinct role of TE and LTE species, comparative genomic analysis of these two groups of species showed that they were functionally distinct. For example, pathways for oxygen and reactive oxygen species metabolism were enriched in TE species, including catalase, superoxide dismutase, osmoprotectant transport systems, and superoxide reductase. As reactive oxygen species are produced by the host during inflammation, this can be an important feature for early engraftment of TE species in an inflamed gut. Removal of reactive oxygen species by TE species can enable subsequent engraftment of LTE species.

### Example 3: Effect of Treatment on Microbiome of Ulcerative Colitis Patients

To determine whether the increased engraftment had any effect on the microbiome of the ulcerative colitis patients, the spore former composition of the treated patients' microbiomes was compared to baseline (i.e., pre-HHSP administration) at various time points after the initial HHSP administration. Specifically, the binary-Jaccard distances between the spore-forming fraction of subject microbiomes and pooled HHSP dose species content were calculated for all arms at all time points sampled. The Binary Jaccard distance ranges between −1 and 1, with 0 indicating samples sharing the exact same set of species, and 1 indicating samples that have no species in common. The abundance of species is not considered in calculations of the metric. A higher value for the similarity metric indicates greater similarity between subject microbiomes and HHSP.

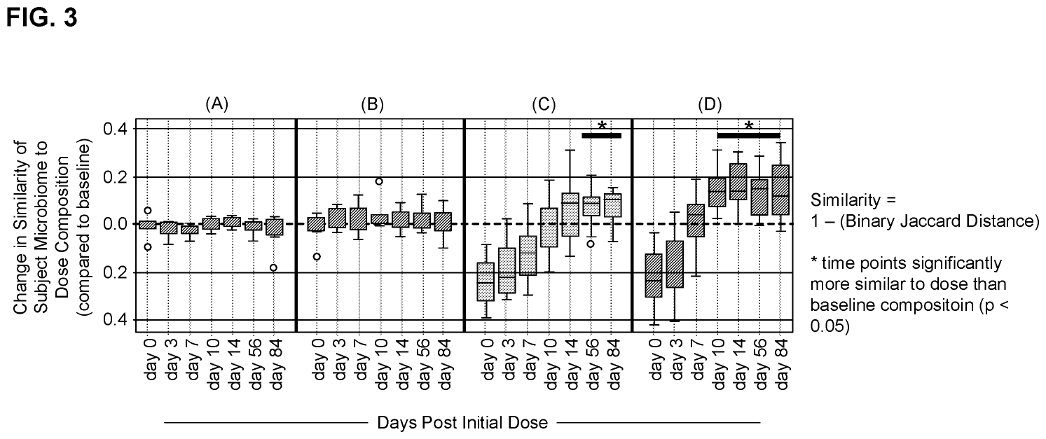

As shown in FIG. 3, at the end of the 8-week induction therapy treatment, the spore former portion of the microbiome of patients from Arms C (vancomycin pre-treatment/HHSP weekly) and D (vancomycin pre-treatment/HHSP daily) were more similar to that of the HHSP composition than to the baseline. As observed with clinical efficacy (see Example 1), the effect was more profound in patients that were pre-treated with vancomycin and daily dose of an HHSP (Arm D), compared to the other treatment arms.

### Example 4: Association of Microbiome Change with Clinical Outcome

Treatment with an HHSP composition changed both the spore former and non-spore former portion of the microbiome in remitters and non-remitters. Further analyses were conducted to determine whether specific species of bacteria were associated with clinical remission observed in the clinical trial subjects. Taxonomic profiles of subject fecal microbiomes and HHSP obtained with a MetaPhlAn database (as described supra) were used to identify species associated with clinical outcome in Arm D, using bootstrapped lasso logistic regression.

Applicants found that as early as 7 days after the initial HHSP dosing, there was a clear distinction in the prevalence of certain bacterial species present in patients in remission (remitters) and patients that were not in remission (non-remitters). This distinction persisted for at least 4 weeks after the end of the treatment period, consistent with the observation of durability of treatment effect (maintenance) associated with HHSP treatment.

In total, 31 different bacterial species were identified as predictive of clinical outcome. The identified species included species that were present in at least some HHSP compositions, as well as those that were augmented by treatment (i.e., either were not present in the HHSP composition or were present at concentrations below the limit of detection). Twenty of the species were associated with remission and 11 were associated with non-remission. Table 3 provides the SEQ ID NOs for a 16S rDNA sequence of the 31 identified bacterial species, along with the name of a reference species having a 16S rDNA sequence with at least 99% sequence identity.

TABLE 3
Bacterial Species Associated with Clinical Outcome

| | Associated | | Reference Strain | SEQ ID NO |
|---|---|---|---|---|
| | Clinical | Engrafter or | with ≥99% 16S rDNA | for 16S rDNA |
| Species | Outcome | Augmenter | full length match | sequence |
| Parasutterella | Remitter | Augment (non- | Parasutterella | 68 |
| excrementihominis | | spore former) | excrementihominis | |
| | | | strain YIT 11859 | |
| Coprobacillus | Remitter | Engrafter | None | 47 |
| unclassified | | | | |
| Holdemania | Remitter | Engrafter | Holdemania filiformis | 66, 67 |
| unclassified | | | strain J1-31B-1, | |
| | | | Holdemania | |
| | | | massiliensis strain AP2 | |
| Bacteroides ovatus | Remitter | Augment (non- | Bacteroides ovatus | 19-22 |
| | | spore former) | strain JCM 5824 | |
| Akkermansia | Remitter | Augment (non- | Akkermansia | 16-18 |
| muciniphila | | spore former) | muciniphila strain | |
| | | | ATCC BAA-8 35 | |
| Clostridium leptum | Remitter | Engrafter | Clostridiumleptum | 44, 45 |
| | | | strain DSM 753 | |
| Roseburia unclassified | Remitter | Engrafter | None | 76 |
| Lachnospiraceae | Remitter | Engrafter | None | 49 |
| unclassified | | | | |
| Bilophila unclassified | Remitter | Augment (non- | Bilophila | 32-36 |
| | | spore former) | wadsworthia 3 1 6 | |
| Lachnospiraceae | Remitter | Engrafter | None | 50 |
| unclassified | | | | |
| Dielma fastidiosa | Remitter | Engrafter | Dielma fastidiosa | 39 |
| | | | strain JC13 | |
| Roseburia hominis | Remitter | Engrafter | Roseburia hominis | 72-75 |
| | | | strain A2-183 | |
| Clostridium | Remitter | Engrafter | Clostridium | 51 |
| symbiosum | | | symbiosum strain | |
| | | | ATCC 14940 | |
| Eubacterium siraeum | Remitter | Engrafter | Eubacterium | 59-62 |
| | | | siraeum strain ATCC | |
| | | | 29066 | |
| Butyricicoccus | Remitter | Engrafter | None | 48 |
| unclassified | | | | |
| Bacteroides vulgatus | Remitter | Augment (non- | Bacteroides vulgatus | 23-30 |
| | | spore former) | strain JCM 5826 | |
| Clostridium bolteae | Remitter | Engrafter | Clostridium | 41 |
| | | | bolteae strain JCM | |
| | | | 12243 | |
| Ruminococcaceae | Remitter | Engrafter | None | 64 |
| unclassified | | | | |
| Subdoligranulum | Remitter | Engrafter | None | 65 |
| unclassified | | | | |
| Clostridium innocuum | Remitter | Engrafter | Clostridium innocuum | |
| | | | B-3 ATCC 14501 | |
| Lachnospiraceae | Non- | Engrafter | None | 15 |
| unclassified | Remitter | | | |
| Lachnospiraceae | Non- | Engrafter | None | 83 |
| unclassified | Remitter | | | |
| Prevotella copri | Non- | Augment (non- | Prevotella copri | 69-71 |
| | Remitter | spore former) | strain JCM 13464 | |
| Faecalicatena contorta | Non- | Engrafter | Eubacterium | 55-58 |
| | Remitter | | contortum strain | |
| | | | DSM 3982 | |
| Dialister invisus | Non- | Augment (non- | Dialister invisus | 52-54 |
| | Remitter | spore former) | strain E7.25 | |
| Clostridiales | Non- | Engrafter | None | 37, 38 |
| unclassified | Remitter | | | |
| Ruminococcus gnavus | Non- | Engrafter | Ruminococcus | 77-82 |
| | Remitter | | gnavus strain A TCC | |
| | | | 29149 | |
| Erysipelatoclostridium | Non- | Engrafter | Erysipelatoclostridium | 46 |
| ramosum | Remitter | | ramosum strain | |
| | | | JCM 1298 | |
| Veillonella unclassified | Non- | Augment (non- | Veillonella atypica | 84-101 |
| | Remitter | spore former) | strain KON; | |
| | | | Veillonella dispar | |
| | | | strain ATCC | |
| | | | 17748; Veillonella | |
| | | | parvula strain A TCC | |
| | | | 10790; Veillonella | |
| | | | ratti strain JCM | |
| | | | 6512; Veillonella | |
| | | | criceti strain JCM | |
| | | | 6511 | |
| Hungatella effluvii | Non- | Engrafter | Clostridium | 42, 43 |
| | Remitter | | hathewayi strain | |
| | | | 1313 | |
| Bifidobacterium | Non- | Augment (non- | Bifidobacterium | 31 |
| dentium | Remitter | spore former) | dentium strain B764 | |

Bacterial species in Table 3 that are associated with remission are useful in DEs. Accordingly, in some aspects of the invention, a microbiome composition comprises at least one of the remitter-associated species identified in Table 3 or a species that has a 16S rDNA that has at least 9700 identity to a remitter-associated species. In some cases, the microbiome composition is an HHSP. In other cases, the microbiome composition is a DE. In general, if the composition is a DE, is does not include a bacterium associated with non-remission.

In some aspects, an HHSP or material used in the manufacture of a spore composition is tested for one or more species associated with non-remission. Presence of such species may be used as a criterion for excluding the HHSP or material in a microbiome composition. In some aspects, an HHSP or material used in the manufacture of a spore composition is tested for the presence of bacterial species associated with remission and the presence of one or more of such species is a criterion for using the HHSP or material in microbiome composition.

### Example 5: Metabolomic Analyses

It is known in the art that multiple bacterial species may be able to carry out similar functions. Applicants posited that by identifying key functions of bacteria associated with remission, compositions can be designed that include bacteria having such functions using bacteria identified as associated with remission in Table 3 and/or bacterial species not identified in Table 3 but otherwise demonstrated to have one or more identified functions. Accordingly, Applicants further characterized the metabolic signatures of bacteria associated with clinical remission and non-remission in patients from all the treatment Arms. Their correlations with the identified bacterial species was determined as described below.

All methods utilized a Waters ACQUITY Ultra Performance liquid chromatography (UPLC®) and a Thermo Scientific Q-Exactive™ high resolution/accurate mass spectrometer interfaced with a heated electrospray ionization (HESI-II) source and Orbitrap mass analyzer operated at 35,000 mass resolution. Four different combinations of ionic and chromatographically optimized conditions were used to capture a variety of hydrophilic and hydrophobic compounds.

The MS analysis alternated between MS and data-dependent MSn scans using dynamic exclusion. The scan range varied slighted between methods but covered 70-1000 m/z.

Metabolites were identified by comparison to library entries of purified standards based on the retention time/index (RI), mass to charge ratio (m/z), and chromatographic data (including MS/MS spectral data). While there can be similarities between these molecules based on one of these factors, the use of all three data points can be utilized to distinguish and differentiate biochemicals. Peaks were quantified using area-under-the-curve.

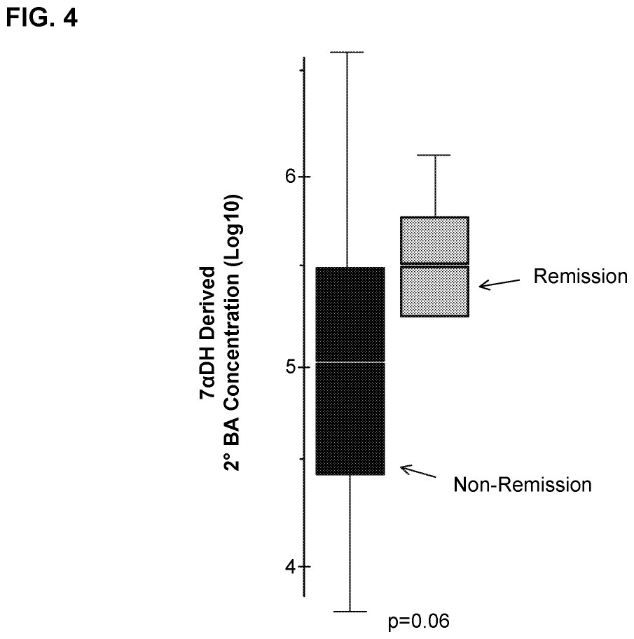

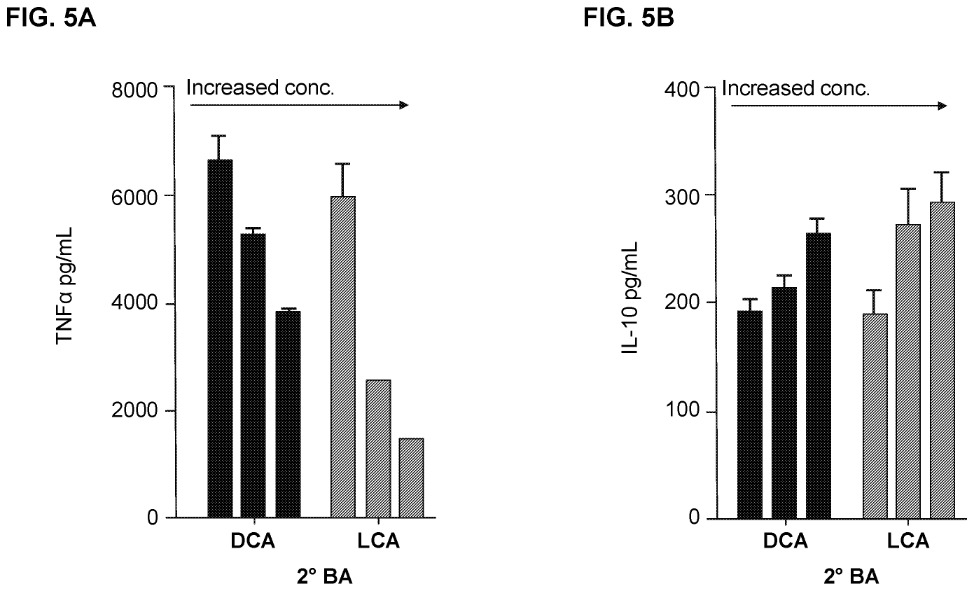

The results of these analyses demonstrated a strong correlation between species associated with clinical outcome and certain metabolites. For instance, as shown in FIG. 4, ulcerative colitis patients (regardless of treatment arm) who went into remission had significantly higher levels of 7-α-dehydroxylated secondary bile acids in their fecal sample, compared to those patients who did not go into clinical remission. Two such secondary bile acids (deoxycholic acid and lithocholic acid) were able to not only decrease TNF-α production but also increase IL-10 production by the LPS-stimulated PBMCs. See FIGS. 5A and 5B, respectively. Other non-limiting examples of metabolites associated with clinical outcome included the following: (i) tryptophan-derived metabolites (e.g., indole and 3-methylindole), (ii) medium-chain fatty acids, (iii) endocannabinoids, (iv) sphingolipids, and (v) kynurenine. Surprisingly, certain SCFAs were negatively associated with remission. The strong correlation appeared to suggest that these species may mediate the activity of key metabolites that are associated with clinical outcome. The metabolomics signature of clinical remission included many diverse functional pathways, with many implicated in inflammatory bowel disease, e.g., ulcerative colitis.

Correlation of Metabolites with Clinical Outcome

To confirm the above identified correlation between species and certain metabolites, the level of selected identified metabolites (i.e., selected tryptophan metabolites (indole and 3-methylindole)) were compared between remitters and non-responders from all treatment arms of the clinical trial (Arms B, C, and D) at the end of the 8-week treatment period.

Standard analysis of paired taxonomic and metabolomic profiles generally involves pairwise correlation (e.g., Spearman or Pearson correlation) between species and metabolite abundance to identify those species whose abundance is correlated with the abundance of metabolites. This type of correlational analysis typically results in large groups of species being correlated with large groups of metabolites, as has been seen in both cohort and interventional studies. This means that this type of standard correlational analysis does not adequately identify those species truly mechanistically involved in a selected metabolic function.

To address the inadequacy of standard correlational analyses, Applicants used a novel approach to identify specific species-metabolite relationships in paired taxonomic and metabolomic profiles. Computational analyses were performed analyzing the relationship between (i) the presence and level of different metabolites and (ii) the presence of individual bacterial species and combinations of bacterial species. In addition, analyses were performed assessing the relative abundance of a bacterial species and a metabolite.

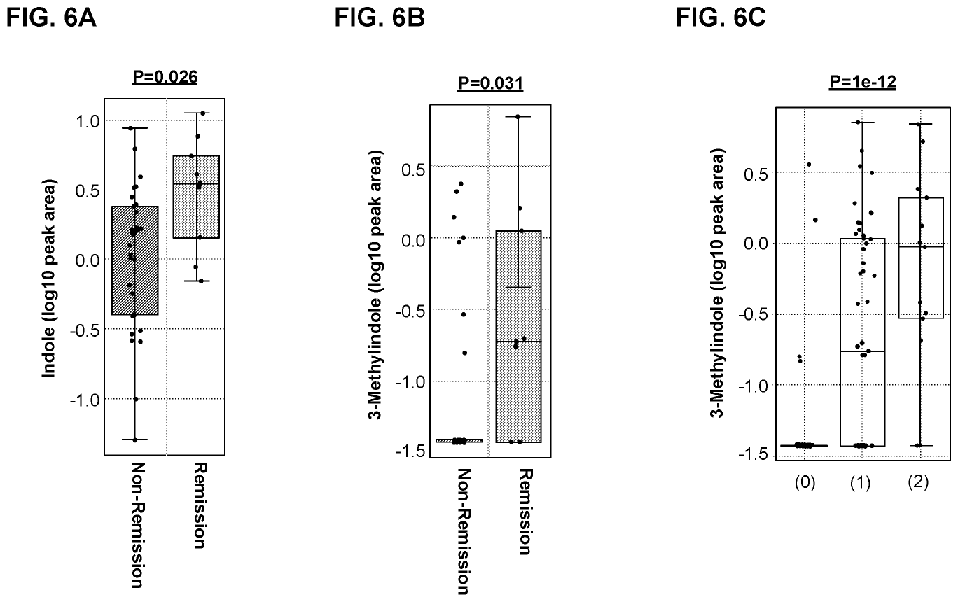

As shown in FIGS. 6A and 6B, ulcerative colitis patients who went into remission after HHSP administration had higher levels of both indole and 3-methylindole, suggesting a positive correlation between increased levels of these tryptophan metabolites and clinical remission. FIG. 6C explains the large variability seen for 3-methylindole (FIG. 6B). Increased tryptophan metabolite levels were associated with two bacterial species identified in HHSP compositions: Ruminococcus bromii and Eubacterium siraeum. Therefore, the variability in 3-methylindole levels seen in FIG. 6B may be due to some ulcerative colitis patients having zero, one, or both of these two bacterial species in their GI microbiome. For example, as shown in FIG. 6C, patients who had both bacterial species in their microbiome had a higher 3-methylindole level and higher rates of clinical remission compared to those who did not harbor these species. These data also indicate that in some aspects, inclusion of R. bromii and/or E. siraeum in a microbiome composition is advantageous e.g., for inducing and/or maintaining remission. Further, inclusion of one or both species is useful for increasing production of 3-methylindole in a treated subject.

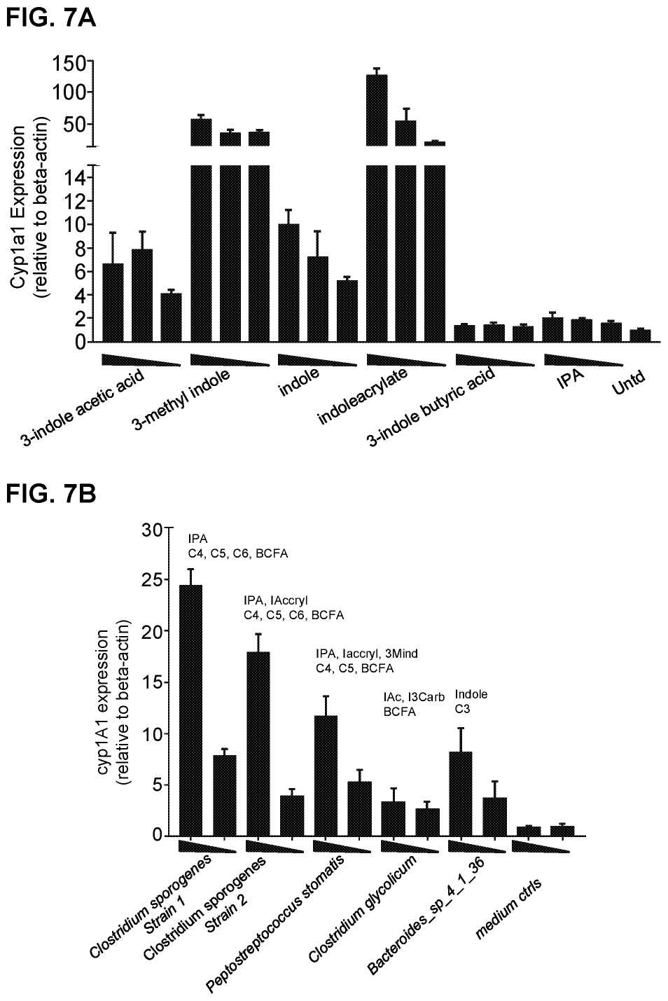

AhR activation is reportedly associated with strengthening of the intestinal epithelial barrier and mucosal homeostasis in the intestine by inducing broad changes in gene expression. As shown in FIG. 7A, indole and 3-methylindole, which were associated with clinical efficacy of a microbiome composition in ulcerative colitis patients as well as other related metabolites (e.g., 3-indole acetic acid and indoleacrylate) induced AhR-mediated cyp1a1 expression in intestinal epithelial organoids. An increase in Cyp1a1 expression is considered to be a specific measure of AhR-mediated gene expression. The increase in cyp1a1 expression also occurred when the epithelial organoids were treated with supernatants of bacteria known to produce the above metabolites. See FIG. 7B. In addition to tryptophan metabolites, the bacterial supernatants also contained a variety of SCFAs, MCFAs, and BCFAs and SCFAs are reported to enhance expression of AhR-responsive genes indicating that the combination of both classes of metabolites could enhance the protective effects of bacterial strains (Jin U. H., et al., Sci Rep 7(10):10163 (2017)).

Accordingly, these results indicate that one mechanism by which the bacteria associated with HHSP effect an improvement in UC is by restoring epithelial barrier integrity through the modulation of metabolites that induce AhR-mediated cyp1a1 expression.

These data indicate that a composition comprising bacteria that can increase levels of certain tryptophan metabolites, e.g., including but not limited to indole and/or 3-methylindole, are useful for treating UC.

### Example 6: Barrier Integrity Analysis

As reported above, certain bacteria associated with remission of UC can produce particular tryptophan metabolites and those metabolites are associated with a more robust intestinal epithelial barrier and mucosal homeostasis. Disruption of normal barrier function due to destruction of tight junctions between epithelial cells and apoptosis induced by chronic inflammation is an important factor in the pathogenesis of inflammatory bowel disease. Mucosal healing and re-establishment of barrier integrity are associated with an improvement of ulcerative colitis (e.g., clinical remission), as well as with an improved patient outcome (Lee S. H., Intest Res 13:11-18, 2015). This effect was further investigated using several bacterial species and additional metabolites in assays assessing restoration of barrier integrity.

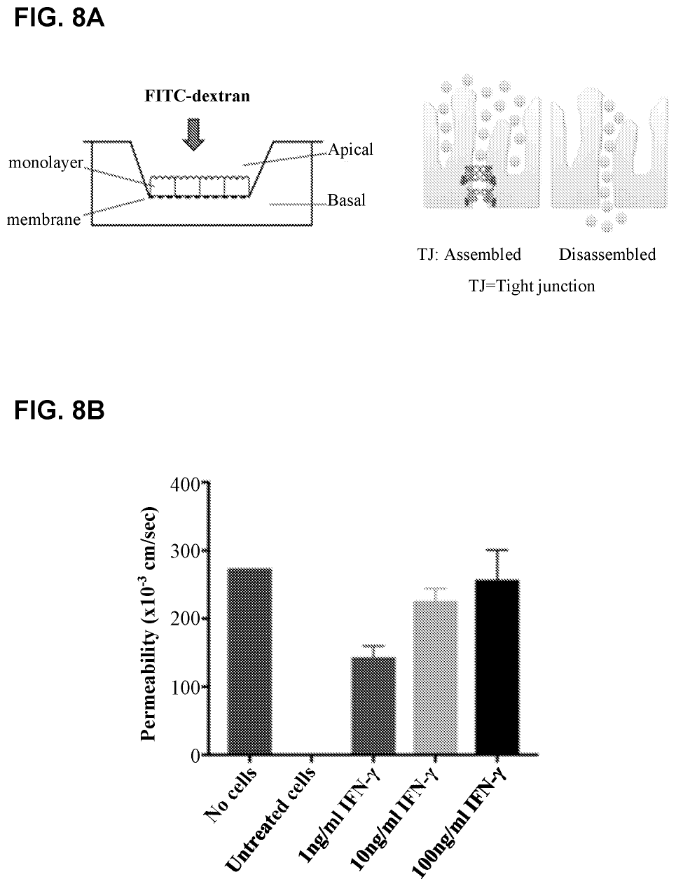

The assays were performed using a primary epithelial cell monolayer barrier integrity assay. As illustrated in FIG. 8A and FIG. 8B, the assay apparatus has an apical side and a basal side that are separated by a monolayer of epithelial cells on a permeable membrane. The addition of interferon-gamma (IFN-γ) disrupts the tight junctions of the epithelial monolayer and induces apoptosis of epithelial cells. The leakiness of the membrane can be assessed by adding FITC-dextran to the apical side of the apparatus and measuring how rapidly it can pass to the basolateral compartment. A leaky monolayer will allow FITC-dextran to the basal side of the apparatus more quickly than a monolayer with an intact monolayer.

Briefly, the barrier integrity assay was conducted as follows. Primary human colon organoid cultures established from isolated colon crypts were grown and expanded in Matrigel® (Corning) and 50% L-cell conditioned medium containing Wnt3a, R-spondin 3 and Noggin (L-WRN) as described by VanDussen et al. containing 10 uM Y-27632 and 10 uM SB43152 (Gut 64:911-920, 2015). Colon organoids were harvested and trypsinized into a suspension containing few cell clusters and seeded onto Matrigel coated transwell inserts (Corning) at a density of 100,000 cells per insert in 50% L-WRN medium supplemented with 10 μM Y-27632 (Millipore Sigma). Epithelial cell monolayers formed over 4-5 days in 50% L-WRN medium. These primarily stem cell population was differentiated into colonocytes by switching the culture medium to 5% L-WRN for 48 hours. After 24 hours of differentiation, specific SCFA or 5% bacterial culture supernatant treatments were added to apical interface in 100 μL of 5% L-WRN medium and 5-25 ng/ml INFγ (Peprotech), depending on the experiment, was added in 175 μL of 5% L-WRN medium to the basolateral interface and incubated for 48 hours at 37° C. After the 48-hour incubation, colonic epithelial monolayer permeability was assessed by adding 10 μL of 10 ng/ml FITC-Dextran (4 kDa, Sigma) to the apical interface, the organoids were incubated for 1 hour and then 100 μL of medium was collected from the basolateral compartment of each transwell and transferred to a 96 well plate for fluorescence detection.

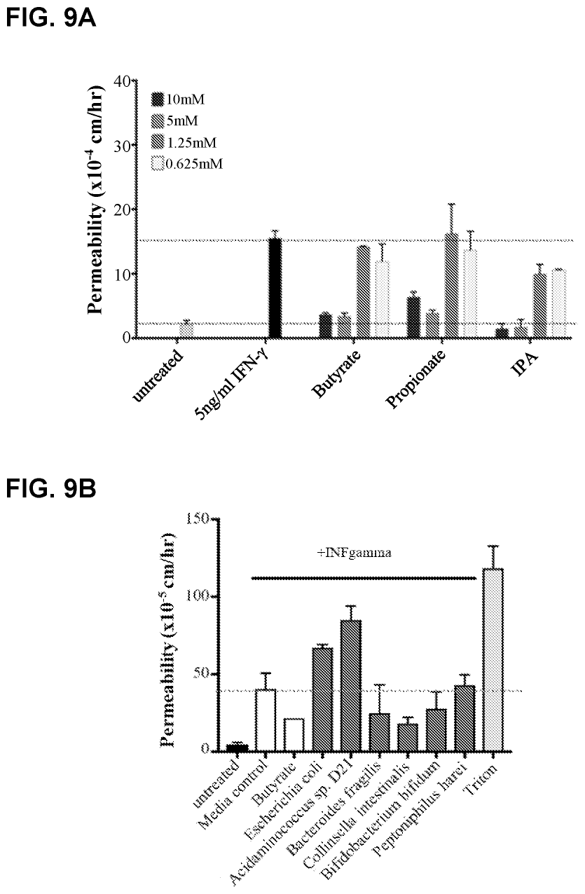

As shown in FIG. 9A, starting at a concentration of about 5 mM, the addition of short-chain fatty acids (butyrate and propionate) or a tryptophan metabolite (3-indolepropionic acid; IPA) restored barrier integrity under these conditions. FIG. 9B demonstrates that the addition of certain bacterial species reportedly associated with clinical remission (e.g., Collinsella intestinalis) can also restore barrier integrity. FIG. 9B also shows that certain bacteria (e.g., Escherichia coli and Acidaminococcus sp. D21) can have a deleterious effect on epithelial barrier integrity. This demonstrates that selection of bacteria for treating an IBD can be based on functional features.

In general, these data demonstrate that bacteria associated with restoration of barrier integrity and/or produce certain metabolites associated with restoration of barrier integrity can be useful for the treatment of ulcerative colitis. Accordingly, such bacteria are useful in bacterial compositions for treating conditions associated with impaired GI barrier integrity such as an IBD. These data also indicate that certain bacteria, Escherichia sp. and Acidaminococcus sp., may not be desirable for inclusion in a microbiome composition for use in treating a condition for which impaired barrier integrity is a feature.

### Example 7: Assessment of Anti-Inflammatory Effects in an Animal Model of Ulcerative Colitis

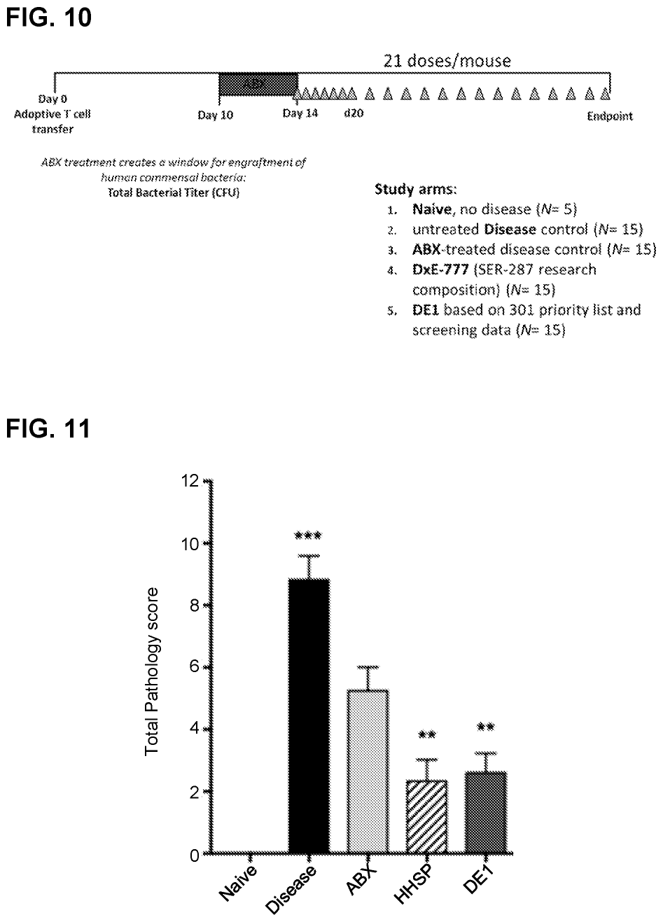

To further assess the effects of a microbiome composition, including a designed composition, on clinical remission, an animal model of ulcerative colitis was used. Briefly, naïve T cells (CD4+CD45RBhigh) obtained from the spleens C57Bl/6 mice (Using RAG IBD Cell Separation Protocol), were adoptively transferred into RAGn12 mice. Ten days later, the mice were treated with antibiotics orally for five days to deplete their natural intestinal microflora. Starting at day 14 post T cell transfer, some of the mice received a total of 21 doses of a spore composition (SP) or a designed composition (DE1) using oral gavage. DE1 is a synthetic composition consisting of 14 bacterial species: Anaerotruncus colihominis, Blautia producta, Clostridium bolteae, Clostridium disporicum, Clostridium ghonii, Clostridium glycolicum, Clostridium innocuum, Clostridium lactatifermentans, Clostridium viride, Eubacterium sp. WAL 14571, Lachnospiraceae bacterium 3 1 57FA, Lachnospiraceae bacterium oral taxon F15, Lactonifactor longoviformis, and Ruminococcus lactaris. In all, the different experimental groups included the following: (i) naïve animals (no disease, i.e., no T cell transfer; n=5); (ii) untreated disease control (T cell transfer only; n=15); (iii) antibiotic-treated disease control (T cell transfer+antibiotic treatment only; n=15); (iv) HHSP treated (T cell transfer+antibiotic treatment+HHSP treatment; n=15); and (v) DE1 treated (T cell transfer+antibiotic treatment+SP treatment; n=15). FIG. 10 provides a schematic of the protocol.

As shown in FIG. 11, animals that received either an HHSP or DE1 had a significantly reduced pathology score compared to the untreated disease control animals and antibiotic only treated disease control animals. The pathology score was based on the summation of 4 individual parameters; inflammation, gland loss, erosion, and hyperplasia (scored 0-5, 0=normal, 5=severe). Nanostring gene expression data were generated using the nCounter Mouse Immunology Panel with isolated total RNA from mouse colon. RNA was isolated from colon tissue stored in RNAlater (ThermoFisher) at −80° C. using a Qiagen RNeasy Plus Mini Kit per the manufacturers protocol. cDNA was then generated from mouse total-RNA using Invitrogen™ SuperScript™ III First-Strand Synthesis System for subsequent RT-qPCR analysis.

These data demonstrate that a composition comprising spore-forming bacteria derived from feces of a healthy donor or a subset of spore-former species can be effective for treating UC.

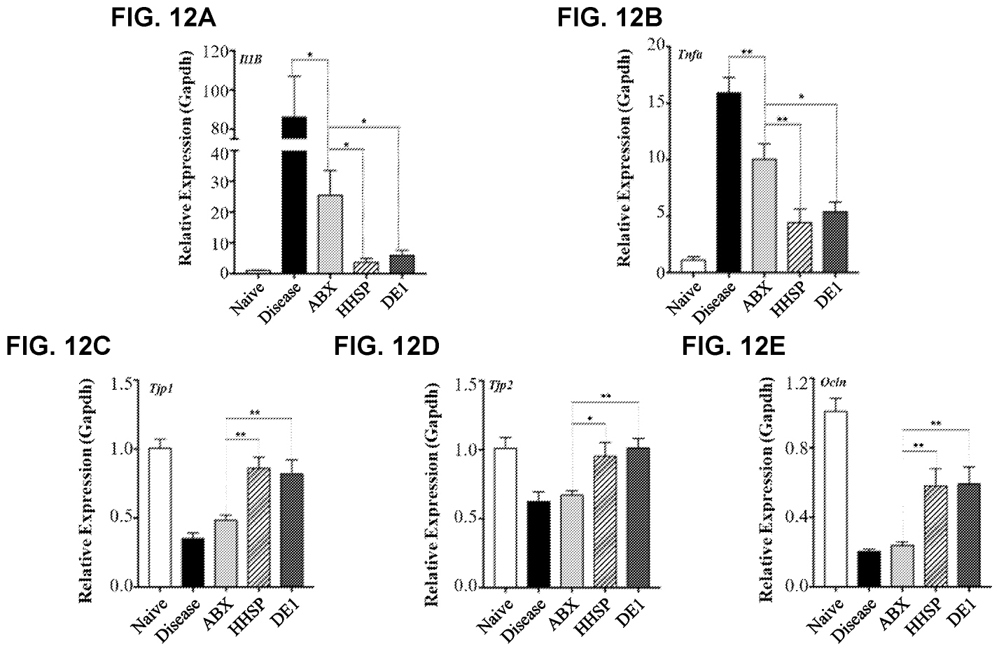

The NanoString gene expression profiles of colon samples from the mice indicated differences in the expression of several genes among the different groups. The following genes were downregulated in animals treated with an HHSP compared to the disease control animals: (i) T cell activation (e.g., Ctla4, Il18r1, Cxcl10/11, Lilrb3/4, Ifng, Nos2), (ii) proinflammatory cytokines (e.g., Tnf, Il1b, Ifng), and (iii) innate immune cell recruitment or activation (e.g., Cxcl1, Cxcl3, Ccl2, Cxcr6, Ltb, Cybb). The following genes were upregulated in animals treated with the HHSP compared to the disease control animals: (i) inhibition of inflammation (e.g., C4 bp, Zeb1, Cd109), and (ii) adhesion molecules (e.g., Ncam1, Cd34 36, Fn1, Cdh5, Tjp1, Tjp2, and Ocln). The decrease in the expression level of the proinflammatory cytokine genes Illb (FIG. 12A), Tnfa (FIG. 12B), and the increase in the expression of the adhesion molecule genes Tjp1 (FIG. 12C), Tjp2 (FIG. 12D), and OcIn (FIG. 12E) were further confirmed by qPCR and/or ELISA. RT-qPCR based gene expression data was generated using Applied Biosystems™ TaqMan™ Fast Advanced Master Mix on Applied Biosystems QuantStudio 7 Flex System.

Without committing to any specific theory, the above data suggest that such bacteria can treat ulcerative colitis through multiple pathways, such as by altering the patient's microbiota, modulating the production of various biological molecules (e.g., fecal calprotectin, secondary bile acids, tryptophan metabolites, short-chain and medium-chain fatty acids, sphingolipids, and kynurenine). These metabolites and other products of bacterial metabolism can globally regulate the expression of different immune genes in the colon, e.g., in the GI lamina propria, reducing inflammation and its associated histopathology.

### Example 8: Assessment of SCFA Production by HDAC Inhibition Assay

Short-chain fatty acids (SCFAs) have been described as playing a role in regulating host immunity. Studies have described altered patterns of SCFA in patients of different gastrointestinal diseases, e.g., colitis, and administration of butyrate and propionate have been reported to have therapeutic effects in a colitis animal model. Both in vitro and in vivo, SCFAs have been shown to inhibit histone deacetylate (HDAC) activity, which can then, in turn, regulate many aspects of an immune response (e.g., induction of FoxP3+ regulatory T cells). Therefore, bacteria that can produce SCFAs can be useful for the treatment of IBD (e.g., UC) patients.

Given that the type and level of SCFA production in fermentations with fecal slurries depends on the carbon source used (Yang et al., Anaerobe 23:74-81 (2013)), HDAC inhibition was evaluated in supernatants of bacterial strains grown in a variety of carbon (C) sources including mono-, di-, polysaccharides, and porcine mucine. For these experiments, 600 μL cultures in peptone/yeast extract medium (PY) alone or supplemented with 0.5% of one of seven C sources (glucose, fucose, sucrose, starch, pectin, FOS/inulin, or mucin) were inoculated in 96 deep-well plates and grown anaerobically for 4 days. Microbial cells were pelleted by centrifugation, and supernatants were used for the HDAC inhibition assay (HDAC-Glo I/II assay kit, Promega) and HeLa nuclear extract (Promega) as the source of HDAC enzymes. Assays were performed with 15 μL supernatant, 10 μL 1M Tris pH 8, 75 μL of assay buffer containing diluted HeLa nuclear extract which were preincubated for 15 minutes prior to the addition of developing reagent. Luminescence was measured after 20 minutes. Under these conditions, a sterile supernatant spiked with 15 mM butyrate resulted in 65-75% HDAC inhibition.

As shown in FIG. 13, a number of bacterial strains were associated with the ability to inhibit HDAC activity. The bacteria were grouped into one of seven phenotypic clusters (represented as 0-6 in FIG. 13) based on their ability to inhibit HDAC activity when grown in different nutrient sources (termed herein “HDAC clusters”). For example, Cluster 0 corresponds to strains that were able to inhibit HDAC when grown on fucose (a sugar found as a component of mucin glycoproteins) but not on other substrates. These strains utilized fucose as a substrate for propionate production, but not amino acids present in the basal media or other simple and complex carbohydrates added in other conditions. Phenotypic Cluster 5 corresponds to strains that inhibited HDAC when grown only in the presence of simple sugars or starch. Phenotypic Cluster 4 corresponds to strains that inhibited HDAC in all conditions but their activity did not increase by the addition of sugars or polysaccharides. Thus, while many bacterial strains had the capacity for HDAC inhibition, they were able to express that capacity only in the presence of certain substrates (e.g., fucose, mucin, or starch).

The above data indicate that to maximize the SCFA production in vivo, it can be useful to include in a bacterial composition for the treatment of an inflammatory disease (e.g., ulcerative colitis) at least one representative bacteria from each of the phenotypic clusters. The DE1 composition described above in Example 7 is an example of such a composition (i.e., includes at least one representative per HDAC cluster.) In some aspects, the bacteria of a microbiome composition are, collectively, capable of utilizing at least 2, 3, 4, 5, 6, or 7 of these C sources.

### Example 9: Anti-Inflammatory Activity with Intestinal Epithelial Cells

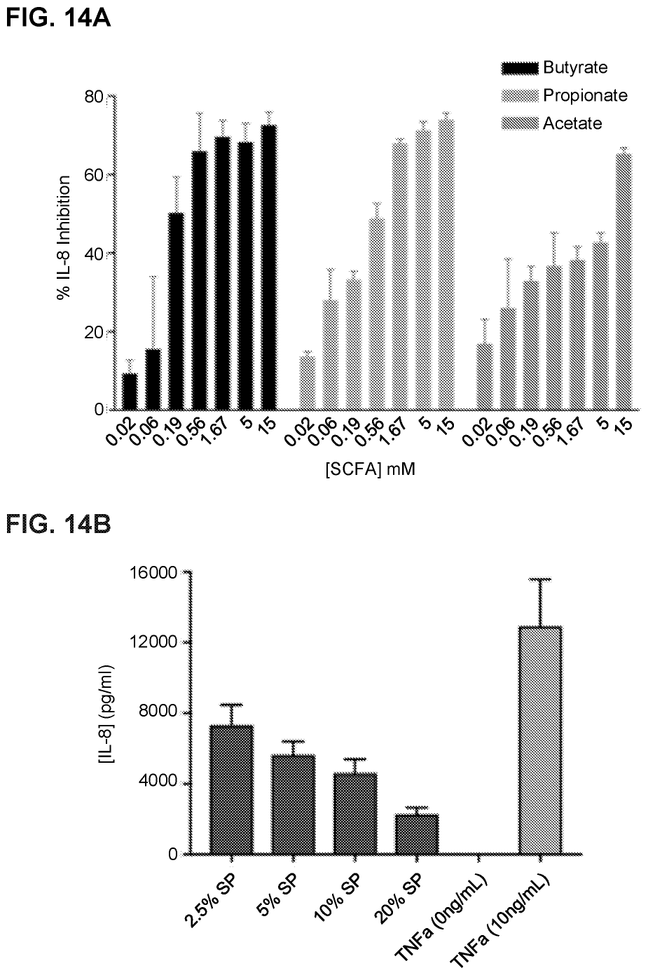

IL-8 level is generally elevated in the inflamed intestinal mucosa of UC patients. Accordingly, the ability to suppress IL-8 induction in intestinal epithelial cells is a relevant readout for identifying bacterial species that can modulate the anti-inflammatory innate immune response in UC patients. Briefly, HT29 cells (an epithelial cell line derived from a colorectal carcinoma), cultured in McCoys Medium supplemented with 10% FBS, GlutaMAX and Pen/Strep were plated at a density of 50 k cells/well in 96-well format and allowed to grow for 5 days until fully confluent. Culture medium was changed every two days. On day 5, cells were pre-treated for 1 hour with a bacterial metabolite (butyrate, propionate, or acetate; FIG. 14A) or with bacterial supernatants (10% in cell culture medium; FIG. 14B) before exposure to 1.25 ng/ml recombinant human TNF-α (Peprotech). Cells were incubated for 4 hours. Culture supernatants were collected and assayed for human IL-8 protein by ELISA (R&D systems) or AlphaLISA (Perkin Elmer). IL-8 levels of test samples were normalized to inflammatory controls that were 10% blank bacterial culture medium pre-treated samples that were exposed to the 1.25 ng/ml TNF-α. To measure the pro-inflammatory capacity of individual bacterial strains, human IL-8 concentrations were measured in cell culture supernatants treated with 10% bacterial supernatant in the absence of TNF-α stimulation.

As shown in FIG. 14A, treating the IECs with any of the short-chain fatty acids tested (i.e., butyrate, propionate, or acetate) resulted in reduced levels of TNF-α-dependent IL-8 secretion. Importantly, supernatants of an HHSP grown in vitro were also able to inhibit IL-8 secretion by IECs in a dose-dependent manner (see FIG. 14B), demonstrating the ability of a microbiome composition to reduce inflammation, e.g., in an IBD such as ulcerative colitis.

Because bacteria can also induce IL-8 directly through toll-like receptor (TLR) activation, a pro-inflammatory assay was designed to identify bacterial strains having this ability (i.e., bacteria capable of TNF-α-independent IL-8 activation). Such strains could be pro-inflammatory in vivo, therefore exacerbating inflammation in UC patients. Accordingly, it can be undesirable to include in a microbiome composition a bacterial strain that can exhibit this activity.

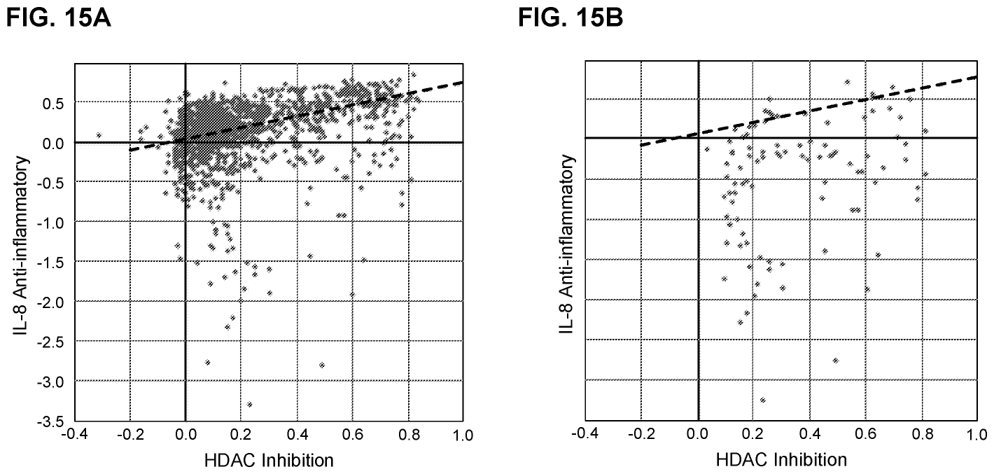

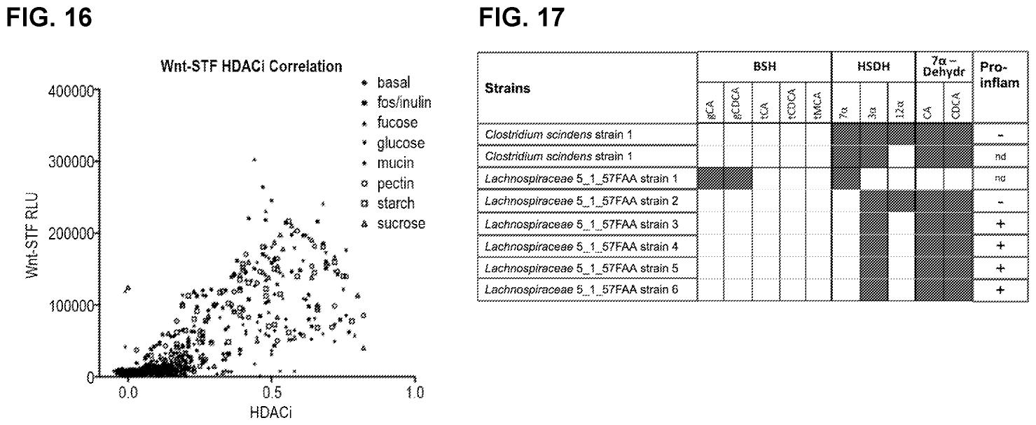

As shown in FIGS. 15A and 15B, many of the supernatants (each circle represents an individual supernatant) exhibited the ability to modulate (e.g., decrease) TNF-α-dependent IL-8 secretion (y-axis), and the anti-inflammatory activity generally correlated with inhibition of HDAC activity of the supernatants (x-axis). However, some of the supernatants had no anti-inflammatory activity in IECs despite having HDAC inhibitory activity, or even resulted in additional IL-8 production over that induced by TNF-α (i.e., these were points, where IL-8 anti-inflammatory activity, on the y-axis, did not correlate with HDAC inhibition, on the x-axis). The majority of these outliers were supernatants with activity in the pro-inflammatory assay (light gray); that is, these strains resulted in IL-8 secretion, which can lessen or even outweigh the anti-inflammatory effects of their inhibition of HDAC activity. In addition, strain-level variability was observed in the pro-inflammatory properties of closely related strains, indicating that bile acid activities and pro-inflammatory properties are not always conserved among different strains of the same species (at least among the Lachnospiraceae species) (FIG. 17). Similar results were observed for Wnt activity (FIG. 16).

These results underscore the fact that anti-inflammatory activity is not an inherent property of bacterial strains that produce SCFAs and inhibit HDAC, but rather that strains need to be tested, e.g., directly in cell-based assays to identify those with pro-inflammatory activity of their own. These data demonstrate that when constructing a microbiome composition, although closely related bacteria (e.g., species or OTUs) may typically share functional features leading to, for example, pro-inflammatory or anti-inflammatory activities, it can be advantageous to assay the specific strain to be used in a composition as well as the entire composition to define the appropriate set of functions for immune modulation.

### Example 10: Determination of SCFA and Tryptophan Metabolite Profiles in Single Strain Supernatants

As described supra, certain tryptophan (Trp) metabolites were associated with remission in patients treated with an HHSP. Accordingly, Applicant tested various bacterial species for the presence of SCFA or tryptophan metabolites in their supernatants. The presence of the tryptophan metabolites was determined using a colorimetric assay for detection of indolic compounds (Indole Reagent, Anaerob Systems). Indole produces a light blue color in this assay, while other Trp metabolites produce purple color. The presence of SCFAs were tested using the HDAC assay (described supra). Supernatants of selected strains that were identified as producers of Trp metabolites by the colorimetric indole assay, and/or producers of SCFAs by the HDAC assay were further analyzed by GC-MS to identify the specific metabolites produced.

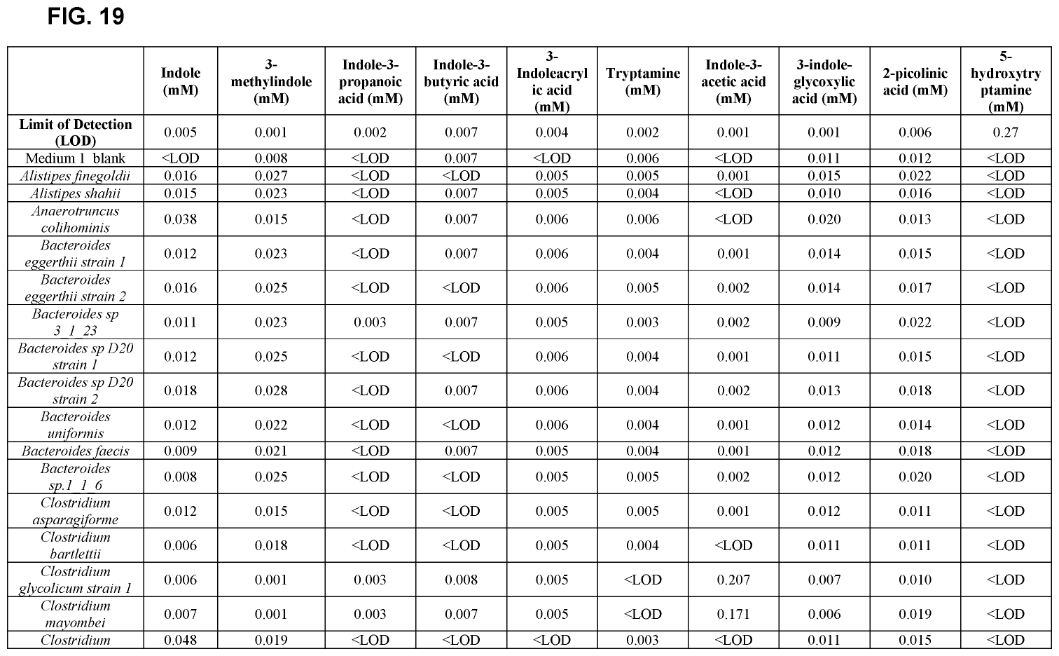

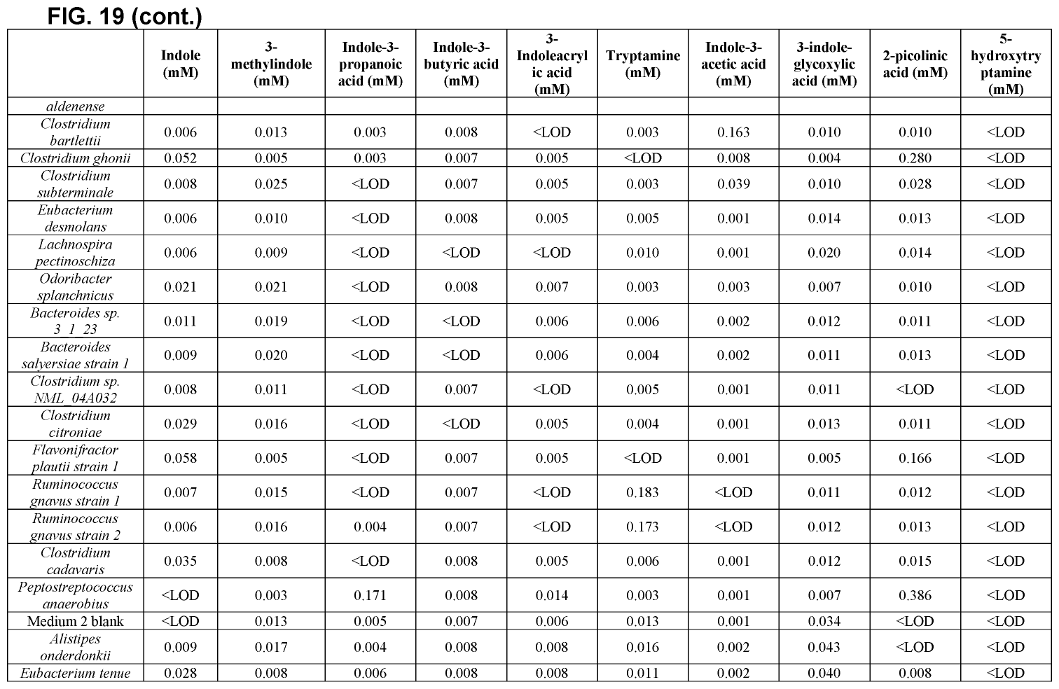

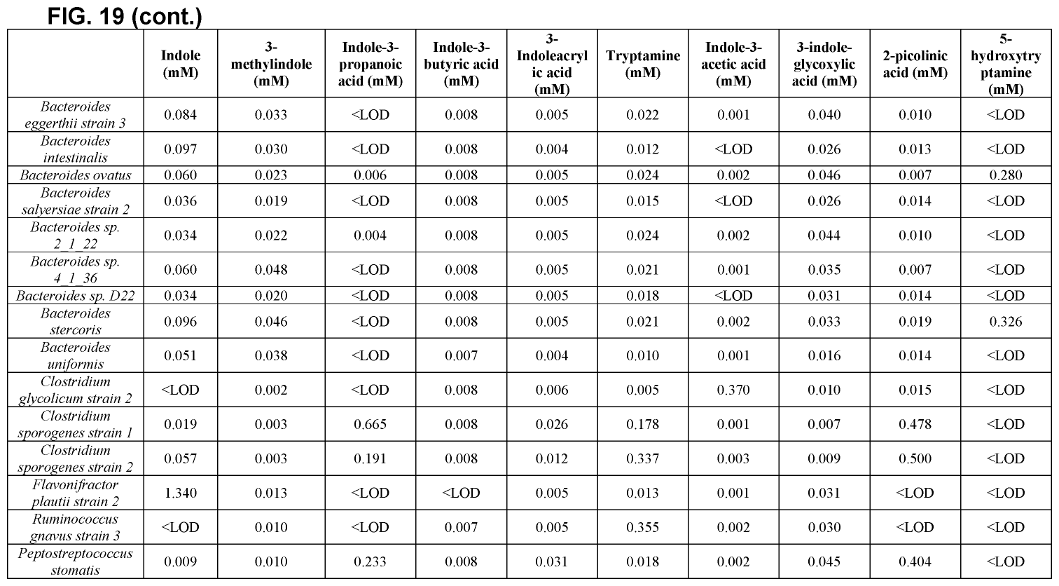

The results of the SCFA analysis are shown in FIG. 18 and the results of the Trp metabolites are shown in FIG. 19. Many bacterial supernatants contained one or more of the SCFAs generally associated in the literature with anti-inflammatory activity (butyrate and propionate) (see FIG. 18).

In addition, several bacterial species produced branched chain fatty acids, 2-methyl-propanoate, 3-methyl-butanoate, and 3-methyl-pentanoate, which are produced by bacterial fermentation of branched amino acids and have been shown to have HDAC inhibitory activity.

Several species were identified as producers of medium chain fatty acids (MCFAs), e.g., valerate and hexanoate, both of which were surprisingly correlated with efficacy in the metabolomic clinical data and are therefore species producing these are candidates for use in UC treatment. Valerate producing species included Anaerotruncus colihominis, Clostridium sporogenes, Flavonifractor plautii, Peptostreptococcus anaerobius, and Peptostreptococcus stomatis. Hexanoate producing strains include Anaerotruncus colihominis, Clostridium sporogenes, Flavonfractor plautii, Clostridium glycolicum, Clostridium innocuum, and Roseburia intestinalis.

Collectively, the above data indicate that the functional attributes of bacteria can be utilized to identify bacterial species that can be used to treat a disease and target multiple host pathways, such as ulcerative colitis. Summary of the phenotypic profile of different bacterial strains disclosed herein are provided in Table 4, below.

### Example 11: Catalase Activity

The inflammatory conditions associated with a disease or disorder disclosed herein (e.g., IBD) result in a high abundance of reactive oxygen species (ROS) that are toxic for many commensal organisms. For example, intestinal epithelial cells of UC and Crohn's disease patients can express high levels of DouxA which releases hydrogen peroxide into the lumen. Additional ROS can be released by activated macrophages. Some bacteria have ROS detoxyfing enzymes such as catalase and superoxide dismutase that allow them to survive under inflammatory conditions and thus, could be particularly well adapted to engraft in UC patients.

Cultures of a large number of bacterial symbionts were screened for catalase activity by addition of 5 ul of 30% solution of hydrogen peroxide. Catalse activity was detected by the appearance of oxygen bubbles in the cultures. Only 19 strains out of ˜400 strains tested were positive for catalase activity indicating that this is a rare function among the screened species. Non-limiting examples of catalase positive species included Bacteroides sp. 1 1 6, Bacteroides sp. 1 1 30, Bacteroides ovatus, Bacteroides intestinalis, Bacteroides faecis, Bacteroides salyersiae, Bacteroides eggerthii, Eggerthella lenta, Lachnospiraceae bacterium 5 157FAA, Clostridium lavalense, Ruminococcus gnavus, and Clostridium hathewayi. Inclusion of one or more of these species in a bacterial composition (e.g., those disclosed herein) could be beneficial for the survival of the administered bacterial composition in a patient suffering from a disease or disorder disclosed herein (e.g., UC and Crohn's disease).

### Example 12: Wnt Pathway Activation by Bacterial Supernatants

The cells of the intestinal epithelium are constantly replenished in order to maintain tissue homeostasis. Tissue renewal is driven by an active intestinal stem cell compartment that is dependent on Wnt pathway activation. Intestinal stem cells are exquisitely sensitive to Wnt due to the specific expression of Lgr5. Lgr5 forms a R-spondin co-receptor complex with ZNRF3, a membrane E3 ubiquitin ligase and Wnt pathway negative-feedback regulator that targets the Wnt receptor for removal from the cell surface. In the presence of R-spondin, Lgr5+ intestinal stem cells maintain elevated levels of the Wnt receptor, Frizzled, on the cell surface enabling sustained pathway activation (Clevers et al. Science. 2014). R-Spondin has been shown to protect the intestinal epithelium after injury by promoting intestinal stem cell driven tissue recovery (Takashima et al., The Journal of Experimental Medicine. 2011).

To assess whether amplification of Wnt pathway activation in intestinal stem cells by commensal bacteria could contribute to fortifying the epithelial barrier and tissue homeostasis, a Wnt pathway reporter cell line (HEK 293 STF (ATCC CRL-3249)) was utilized. The cell line was used evaluate the ability of bacterial culture supernatants and metabolites to activate the reporter in a similar manner to R-spondin. Addition of Wnt pathway stimulator compounds, such as Wnt3a protein or R-Spondin, to cultured HEK 293 STF cells leads to the production of luciferase that can be measured by luminescence detection. To measure the ability of bacterial supernatants to enhance Wnt pathway activation, HEK 293 STF cells cultured in DMEM medium supplemented with 10% FBS, GlutaMAX and Pen/Strep were plated at a density of 50 k cells per well in 96 well format and allowed to grow for 3 days until fully confluent. Culture medium was changed every other day. On day 3, cells were treated with 10% bacterial supernatant in Wnt3a conditioned medium (produced from L-Wnt3a cells ATCC CRL-2647) and incubated overnight. Wnt3a conditioned medium supplement with 250 ng/ml recombinant human R-spondin (R&D systems Cat #4645) was used as a positive control for enhanced Wnt pathway activation. After treatment incubation, Bright-Glo luciferase detection reagent (Promega) was added to all wells and incubated for 20 minutes at room temperature. Luminescence was measured using a Perkin Elmer Envision multi-mode plate reader. Supernatants from DEs grown in vitro differentially activate the HEK 293 STF reporter when added to Wnt3a conditioned medium. As seen in FIG. 16, bacterial supernatants were able to enhance Wnt pathway expression and there was a positive correlation between HDAC inhibition and Wnt activation. These results demonstrate that the inclusion of bacterial species capable of enhancing Wnt pathway activation in designing a bacterial composition could be beneficial in treating diseases characterized by epithelial damage, such as those disclosed herein (e.g., UC and graft-versus-host disease).

### Example 13: Designing Bacterial Compositions and Screening for Functional Properties

In designing the bacterial compositions of the present disclosure, the compositions were constructed to have one or more of the following features: (1) capable of engrafting (long-term and/or transient) one or more species when administered to a subject; (2) capable of having anti-inflammatory activity (e.g., inhibiting TNF-α-driven IL-8 secretion in epithelial cells in vitro, and/or ability to downmodulate expression of inflammatory genes (e.g., CXCL1, CXCL2, CXCL3, CXCL11, ICAM1)); (3) not capable of inducing pro-inflammatory activity (e.g., does not induce IL-8 production by IECs); (4) capable of producing secondary bile acids (e.g., 7α-dehydroxylase and bile salt hydrolase activity); (5) not capable of producing ursodeoxycholic acid (e.g., 7p-hydroxysteroid dehydrogenase activity) (6) capable of producing tryptophan metabolites (e.g., indole, 3-methyl indole, indolepropionic acid); (7) capable of producing medium-chain (e.g., valerate and hexanoate) and/or short-chain fatty acids (e.g., butyrate and propionate); (8) capable of inhibiting HDAC activity when grown with at least one carbon source; (9) including species belonging to one or more HDAC clusters; (10) capable of restoring epithelial integrity, as determined by a primary epithelial cell monolayer barrier integrity assay; (11) having bacterial species that are capable of being associated with clinical remission of an inflammatory bowel disease; (12) lacking bacterial species that are capable of being associated with non-remission of an inflammatory bowel disease; (13) capable of expressing catalase activity; (14) capable of having alpha-fucosidase activity; (15) capable of inducing Wnt activation; and (16) not capable of activating a toll-like receptor pathway, e.g., toll-like receptor 5 (TLR5) and/or toll-like receptor 4 (TLR4); (17) capable of inducing anti-inflammatory cytokine expression in macrophages; (18) not capable of inducing pro-inflammatory cytokine expression in macrophages. This was accomplished by including one or more bacterial species with the above features in the different designed compositions.

In total, fifty-six (56) different designed compositions were constructed (DE1-DE56). Many of them were screened for functional properties exhibited when grown as a bacterial community in vitro as follows. The designed bacterial compositions were mixed in equal ratios at ˜1-5×10⁷ colony forming units (CFU)/ml of vegetative bacteria and ˜1×10⁴-1×10⁵ CFU/ml of spore forming bacteria (when relevant) and frozen in 15% glycerol. For cultivation, the bacterial compositions were thawed, the glycerol was removed and the mix germinated in 0.5% BHI/Oxgall for 1 hour at room temperature when they contained spore preparations. Compositions containing vegetative bacteria did not undergo germination. The germinant was then washed out and the cultures diluted to a final concentration of 5×10⁷ cfu/ml and plated as biological replicates in a synthetically derived, fecal culture medium 4 (FCM4), that supports growth of many anaerobic gut bacteria. In experiments where secondary bile acid production by bacterial communities was assayed, FCM4 was supplemented with conjugated bile acids (glycocholic acid, taurocholic acid, glycochenodeoxycholic acid and taurochenodeoxycholic acid) at a final concentration of 50 uM. Bacterial cultures were incubated anaerobically at 37° C. for 7 days, after which their biomass was measured by absorbance of 100 μL culture at 600 nm. The remaining culture was centrifuged at 4000 rpm, the supernatants passed through a 0.2 uM filter and used in biochemical and cell-based assays. HDAC inhibition assays, pro-inflammatory assay in IECs, anti-inflammatory assay in IECs, epithelial integrity assay, macrophage signaling assay, and Wnt activation assay, determination of SCFAs, MCFAs, and tryptophan metabolites were performed as described in the previous examples.

For determination of bile acid metabolites, 100 μL of bacterial cell-free supernatant was then extracted with an equal volume of acetonitrile and filtered through a 0.2 μm filter, generating samples for LC-MS analysis. Bile acids were separated using an Agilent 1260 HPLC equipped with a Microsolv bidentate C18 column preceded by a 0.2 μm pre-column filter. Separation was achieved using a water and acetonitrile gradient with 0.1% formic acid at a flow rate of 0.4 ml/minute. Samples were injected at a volume of 5 μL. The HPLC system was coupled to a Bruker Compass™ qTOF mass spectrometer calibrated to a mass range of 50 to 1700 m/z using the Agilent low-mass tuning mix. Each run was additionally calibrated to a reference mass solution injected at the beginning of each run. Bile acids were detected in negative mode and identified by unique m/z and retention times compared to known pure standards. Area under the peak was determined using Bruker data analysis software. Metabolites were quantified using calibration curves generated from pure standards, ranging in concentration from 0.001 μM to 100 μM.

Supernatants from the DEs were also assayed for their ability to activate TLR4 and TLR5 pathways. Toll-like receptors (TLRs) are pattern recognition receptors (PRR) that bind to pathogen-associated molecular patterns (PAMP) such as bacterial cell wall components, i.e. peptidoglycans, lipopolysaccharides, surface proteins, etc. TLR4 and TLR5 receptors are known to bind to antigens and induce a pro-inflammatory response. TLR4 binds to lipopolysaccharide (LPS) which is present in gram-negative bacteria while TLR5 binds to flagellin (FLA), found in motile bacteria. We predict that designed bacterial compositions that exclude gram-negative and IL-8 inducing bacterial strains should not activate TLR4 or TLR5. We utilized a TLR receptor reporter cell lines, HEK-Blue hTLR4 (Invivogen, cat #hkb-htlr4), hTLR5 (Invivogen, cat #hkb-htlr5) to evaluate the ability of bacterial culture supernatants and metabolites to activate the TLR4 and TLR5 reporters. HEK-Blue Null1 (Invivogen, cat #hkb-null1) cells were included as a control reporter cell line for TLR receptor endogenously expressed in the parental cell line HEK 293 that allowed measurement of background HEK-Blue signal. HEK-Blue TLR reporter cell lines are co-transfected with a plasmid designed to overexpress a given TLR receptor and a Secreted Alkaline Phosphatase (SEAP) gene under the control of NF-kB and AP-1 promoters (Invivogen). Activation of the given TLR reporter in leads to secretion of SEAP in solution which is measured by absorbance (655 nm). To measure TLR4 and TLR5 activation by the bacterial supernatants, HEK-Blue hTLR4, hTLR5 and HEK-Blue Null1 cells cultured in DMEM medium supplemented with 10% FBS, GlutaMAX and Pen/Strep were plated at a density of 50,000 cells/well in 96 well format and allowed to reach 100% confluency after 5-7 days in culture. Culture medium was replaced every other day. Once the wells were 100% confluent, the cells were treated with 10% bacterial supernatant in cell culture medium and incubated overnight. For HEK-Blue hTLR4 reporter assay positive control we used cell culture medium supplemented with 100 ng/ml LPS-EK (Invivogen cat #tlr1-peklps) and 10% FCM4+ media. For HEK-Blue hTLR5 reporter assay positive control we used cell culture medium supplemented with 60 ng/ml of FLA-BS (invivogen cat #tlr1-pbsfla) and 10% FCM4+ media. Each TLR reporter cell line had a Null plate with same treatment and respective positive control. After treatment incubation overnight, HEK-Blue Detection Media (Invivogen, cat #hb-det3) was added to all wells and incubated for 2 hours at 37° C., 5% CO2. SEAP secretion was measured as absorbance (655 nm) using a Spectramax plate reader.

Bacterial composition supernatants were also evaluated for their capacity to modulate gene expression in primary human colonic organoids as follows. Primary human colon organoid cultures established from isolated colon crypts were grown and expanded in Matrigel® (Corning) and 50% L-cell conditioned medium containing Wnt3a, R-spondin 3 and Noggin (L-WRN) as described by VanDussen et al. (Gut 64:911-920, 2015). Colon organoids were grown in 24-well plates for 5 days in 50% L-WRN medium. After 5 days of mini-gut structure formation in 50% L-WRN medium, organoid culture medium was switched to 5% L-WRN medium to induce differentiation of the organoids. After 24 hours in 5% L-WRN medium, organoids were treated with 10% DE supernatants in fresh 5% L-WRN medium supplemented with the inflammatory cytokine, e.g., 12.5 ng/ml human TNFa (Peperotech) or 10 ng/ml IFN-γ [symbol] . Control conditions include organoids treated with 5% L-WRN+10% bacterial culture medium and 5% L-WRN+10% bacterial culture medium +12.ng/ml human TNFa or IFN-γ. Organoids were incubated in treatment conditions overnight and then collected in Qiagen RLT buffer for RNA analysis. Sample lysates were either purified into RNA using Qiagen RNeasy mini prep kit or lysates were assayed directly on the Nanostring nCounter platform. In some aspects, purified RNA was used to prepare amplified cDNA libraries that were sequenced using an Illumina NovaSeq 6000 instrument.

Table 6 summarizes the number of strains possessing several of these properties in the exemplary designed compositions disclosed herein. Table 6 describes the number of strains present in consortia: a) with HDAC inhibition phenotypes (rows HDAC cluster 0, HDAC cluster 1, HDAC cluster 2, HDAC cluster 3, HDAC cluster 4, HDAC cluster 5, HDAC cluster 6), b) that produce short-chain and medium-chain fatty acids (rows Propanoic acid, Butanoic acid, Pentanoic acid, Hexanoic acid), c) that produce tryptophan metabolites (rows Indole, 3-methyl indole, 3-indolacrylic acid), d) that have bile acid metabolic activity (rows BSH gCA [for bile salt hydrolase activity on glycocholic acid], BSH tCA [for bile salt hydrolase activity on taurocholic acid], BSH gCDCA [for bile salt hydrolase activity on glycochenodeoxycholic acid], BSH tCDCA [for bile salt hydrolase activity on taurochenodeoxycholic acid], 7aD CA [for 7α-dehydroxylase activity on cholic acid], 7aD CDCA [for 7α-dehydroxylase activity on chenodeoxycholic acid], 7bHSDH UDCA [for 7β-hydroxysteroid dehydrogenase activity on CDCA]), e) that express catalase activity (row Catalase), f) that have fucosidase activity (row a-L-Fucosidase), g) that induce IL-8 (row IL8 Inflammatory), h) that are long-term engrafters (row LTE) or transient engrafters (row TE); i) that are associated with clinical remission (row Remission Associated) or non-remission (row Non-remission Associated).

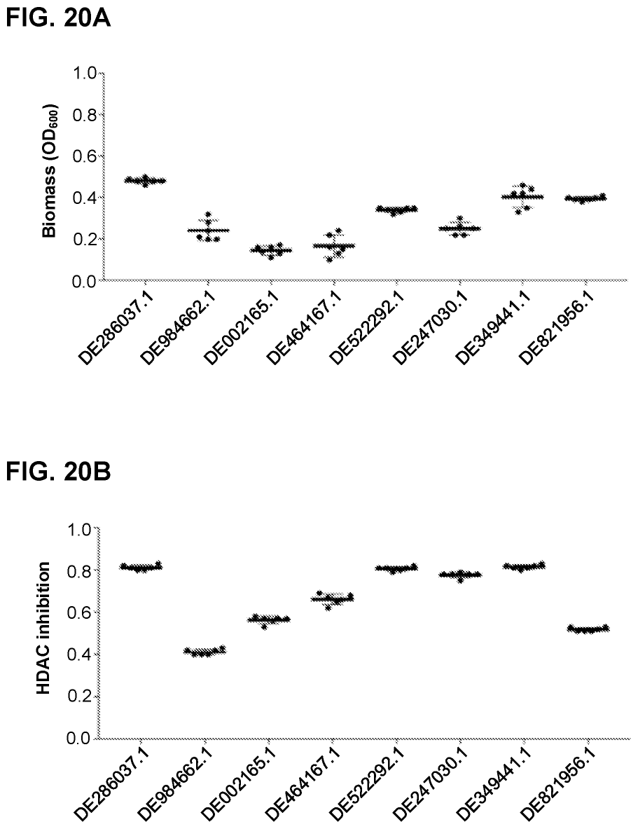

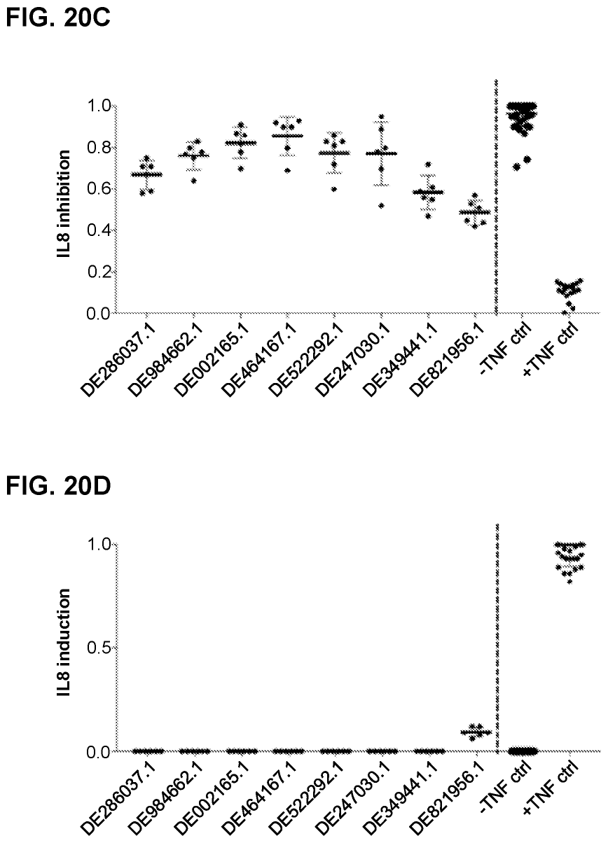

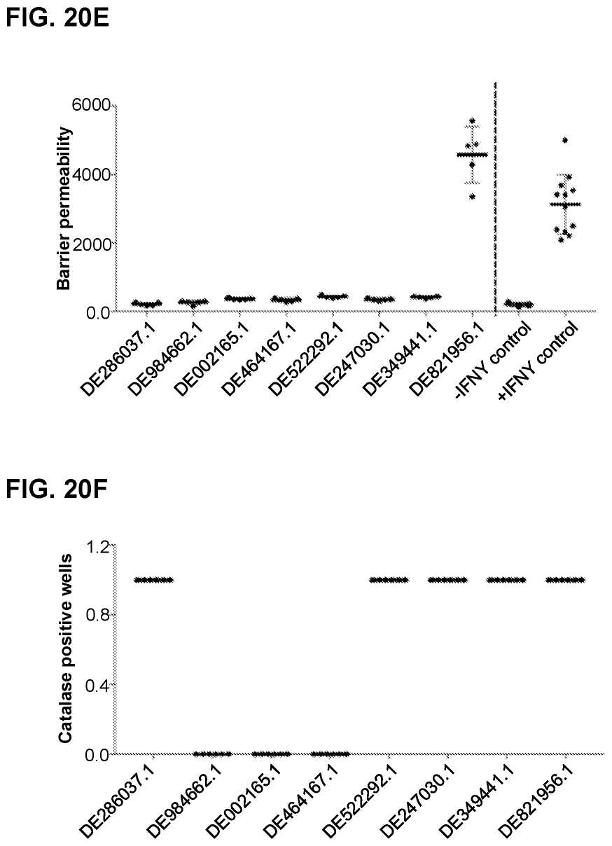

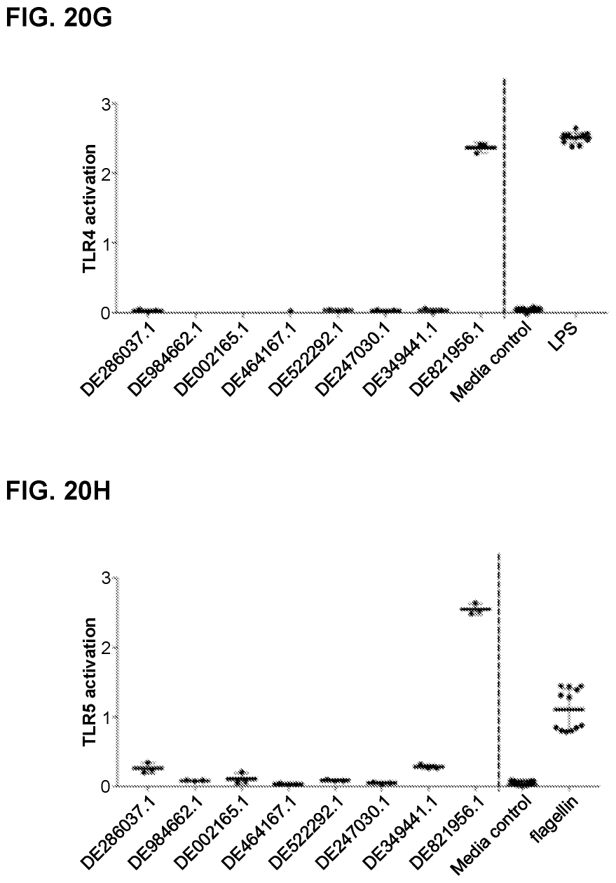

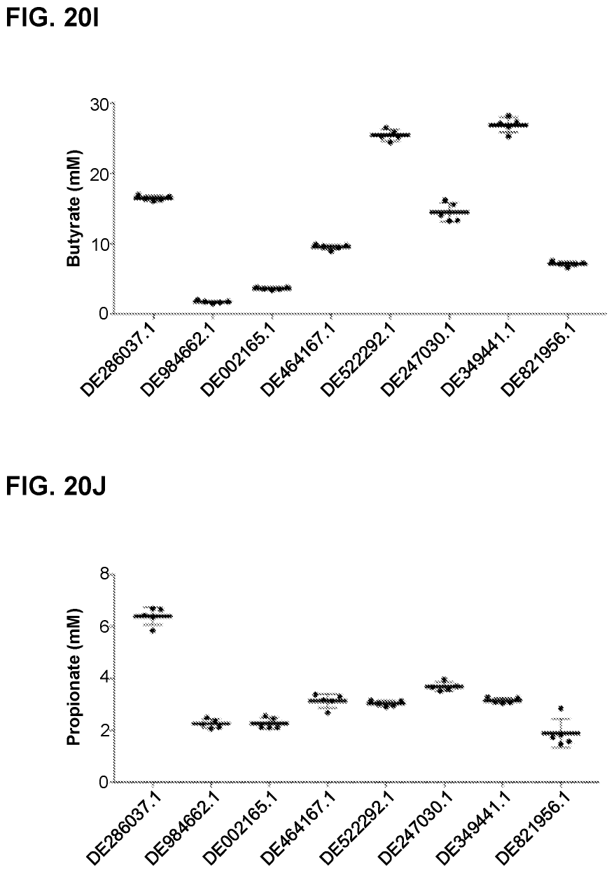

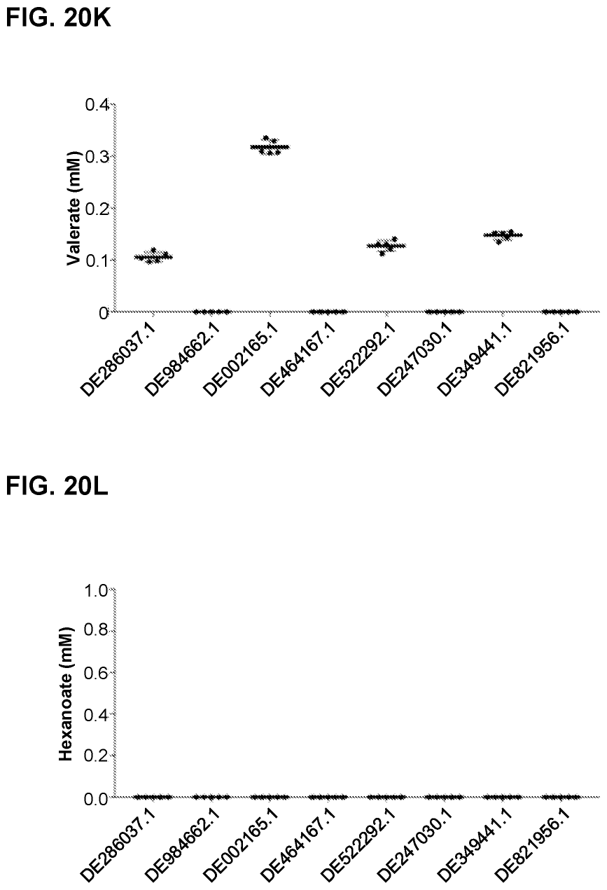

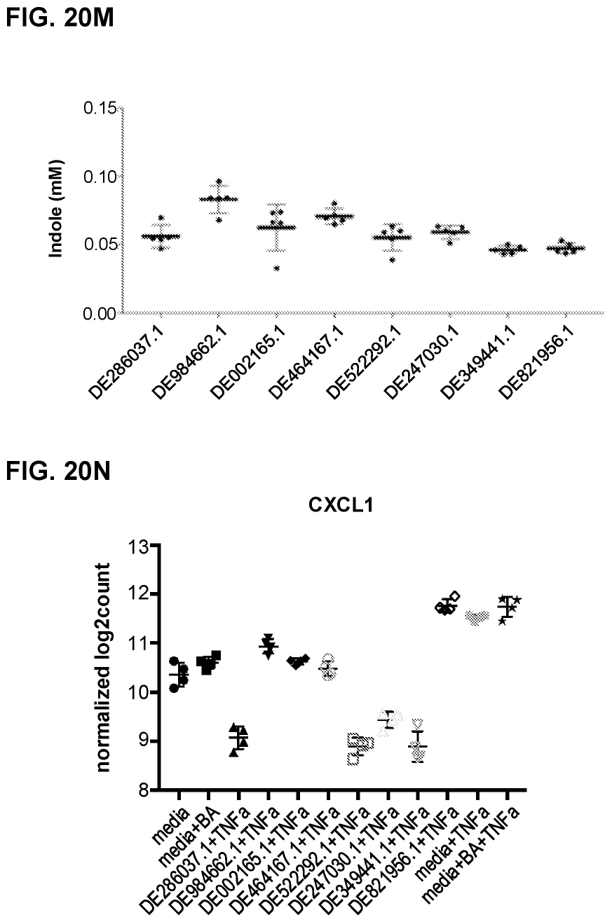

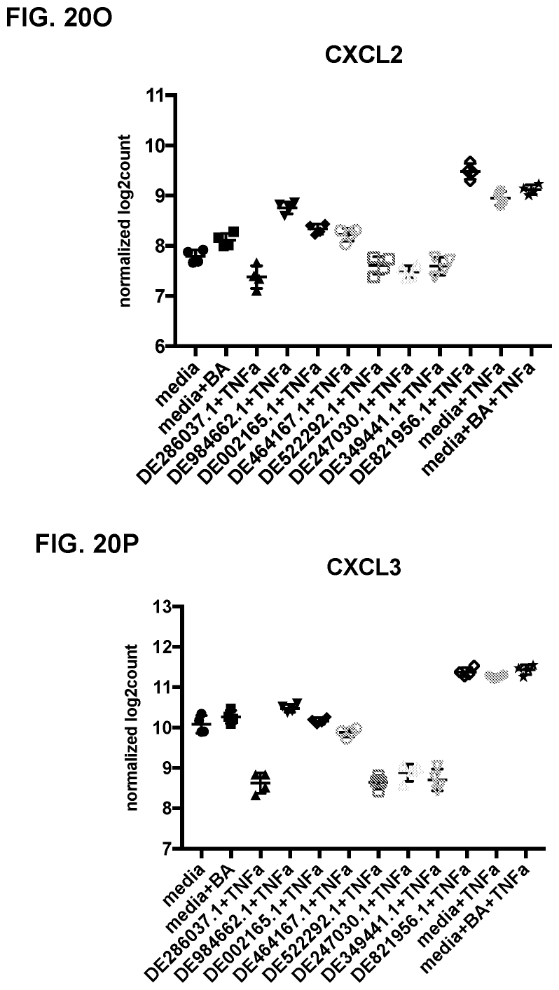

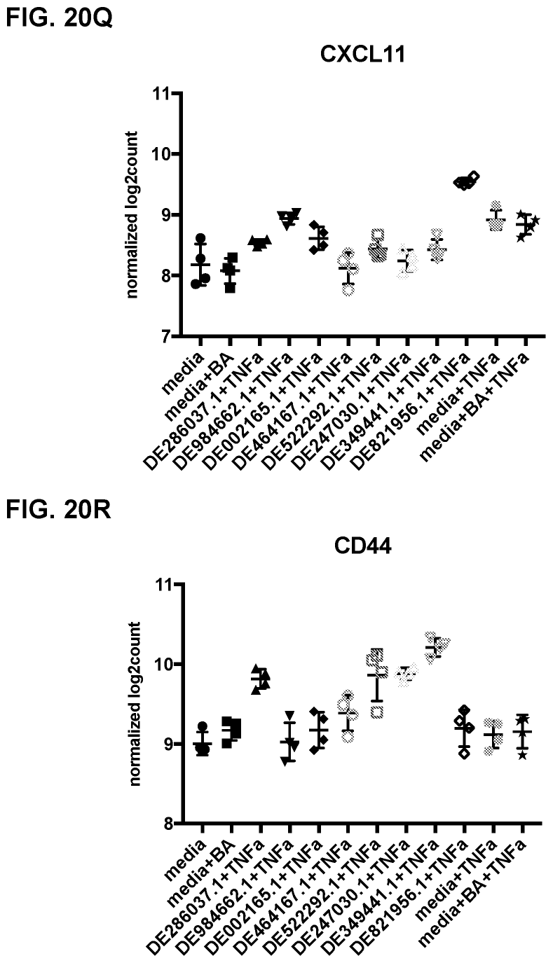

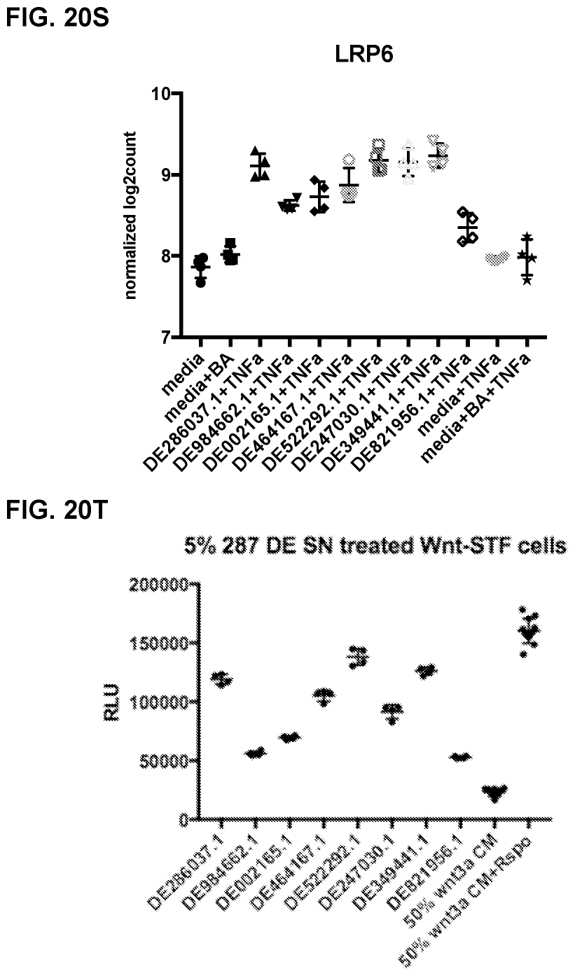

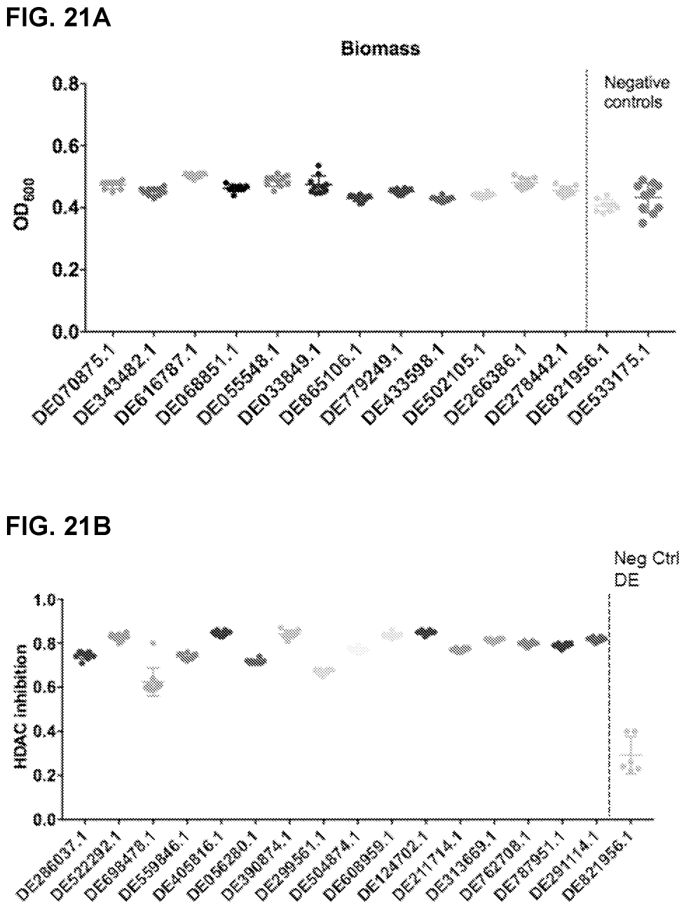

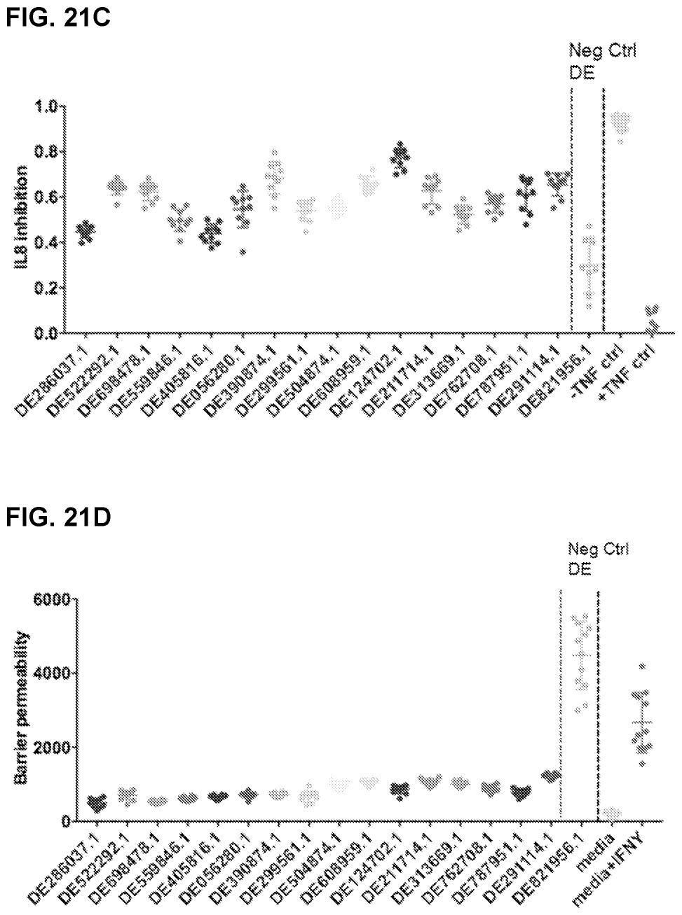

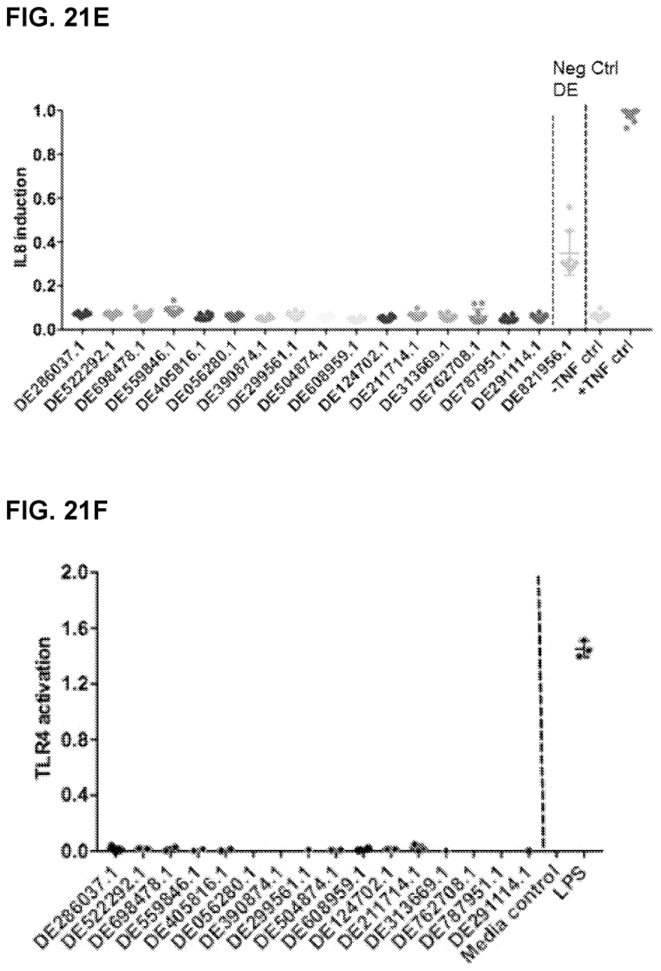

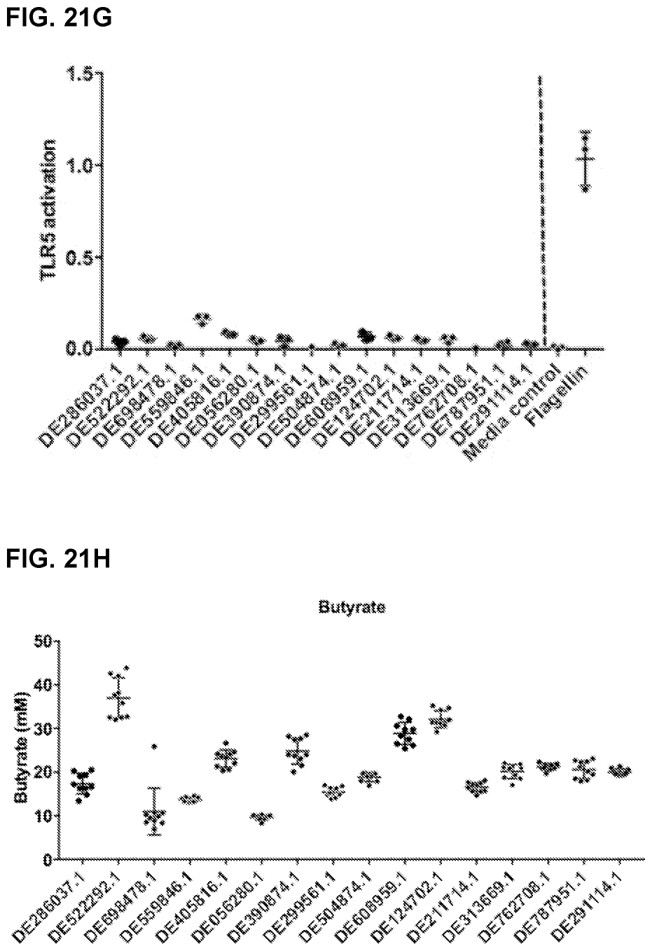

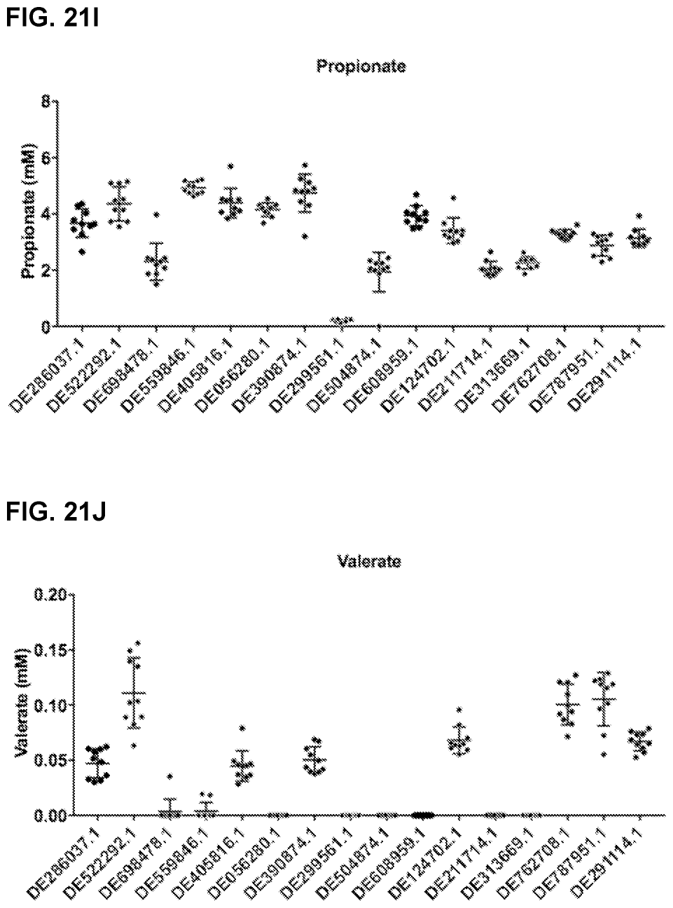

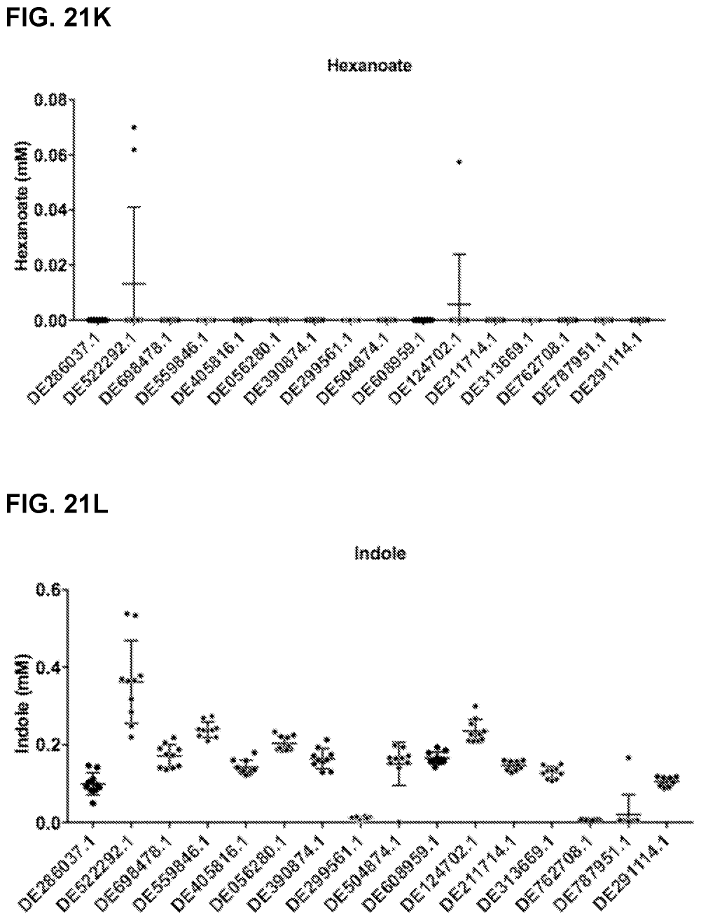

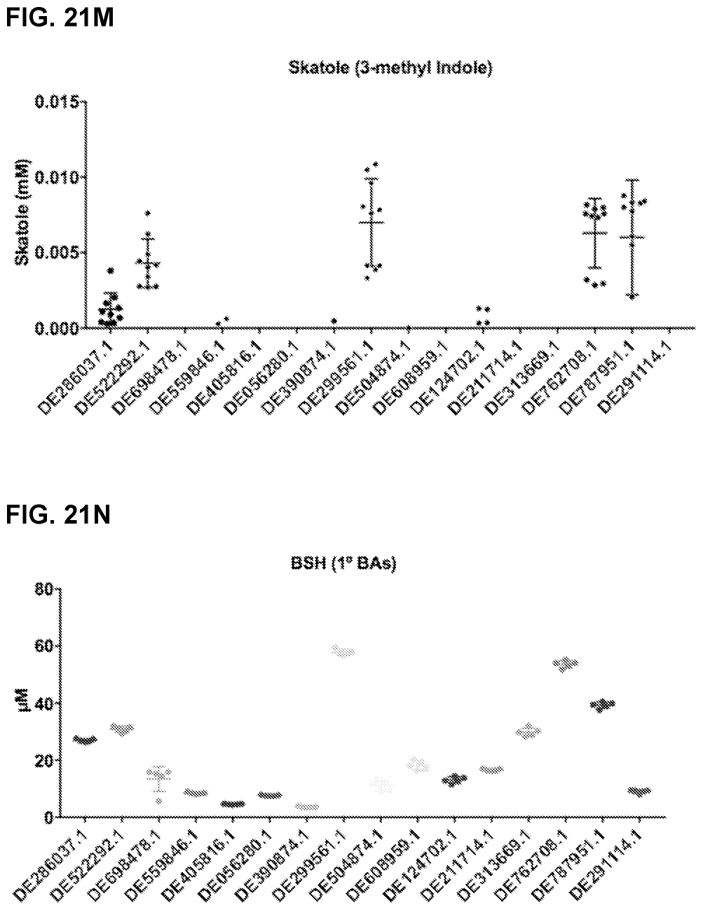

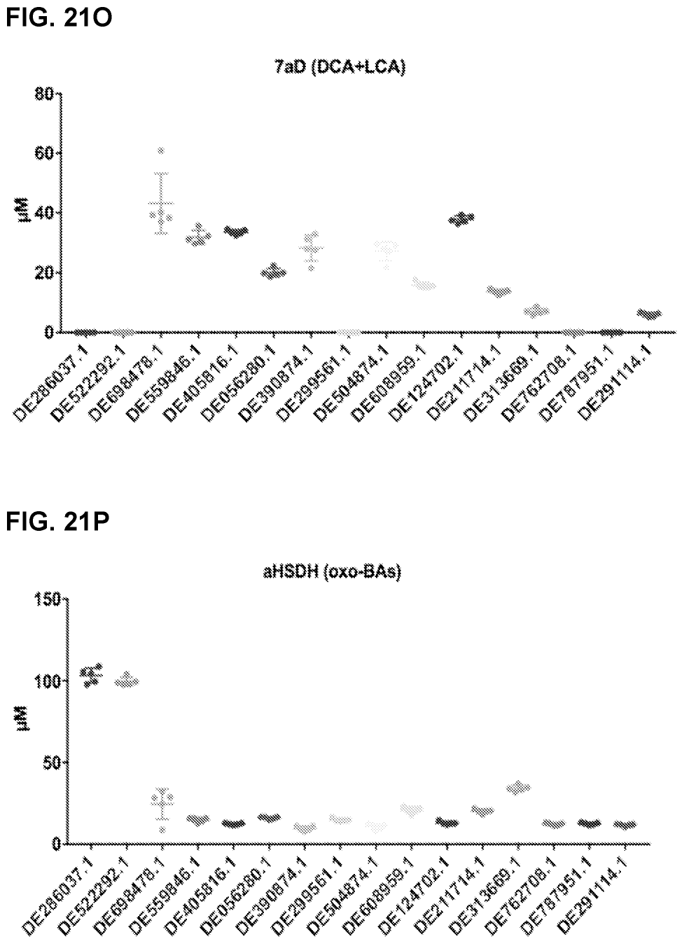

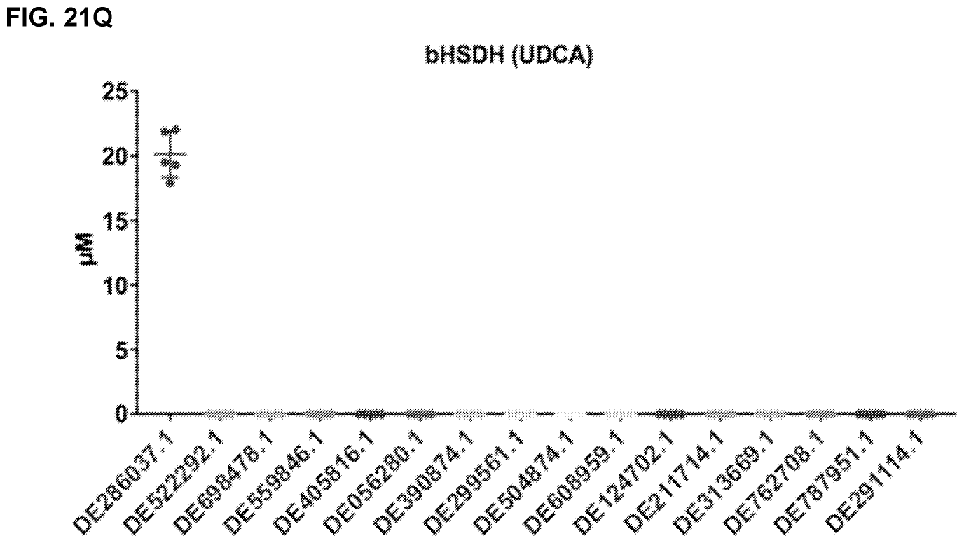

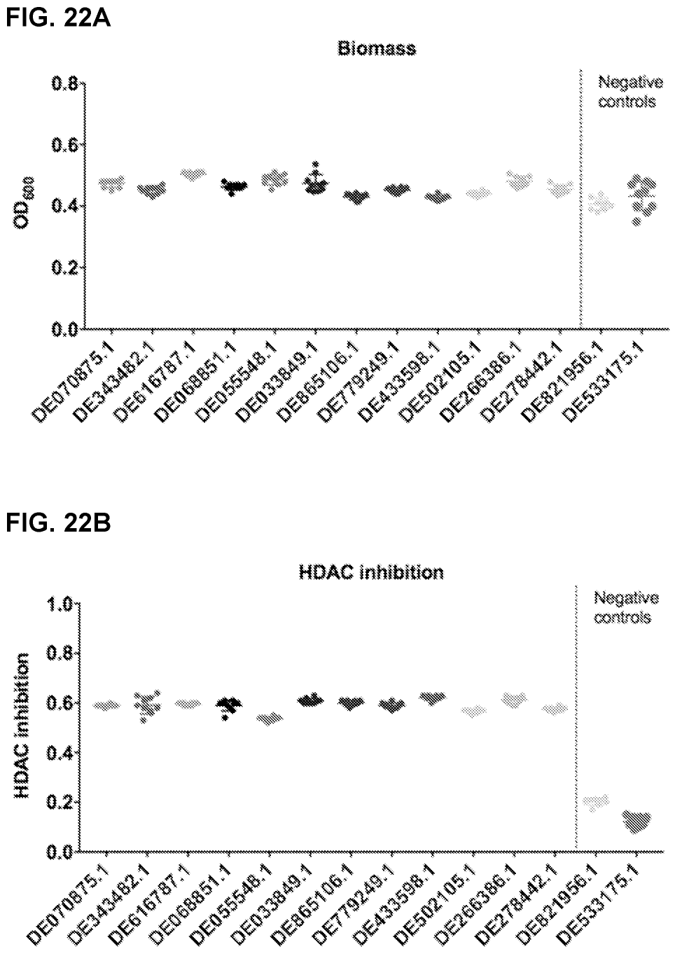

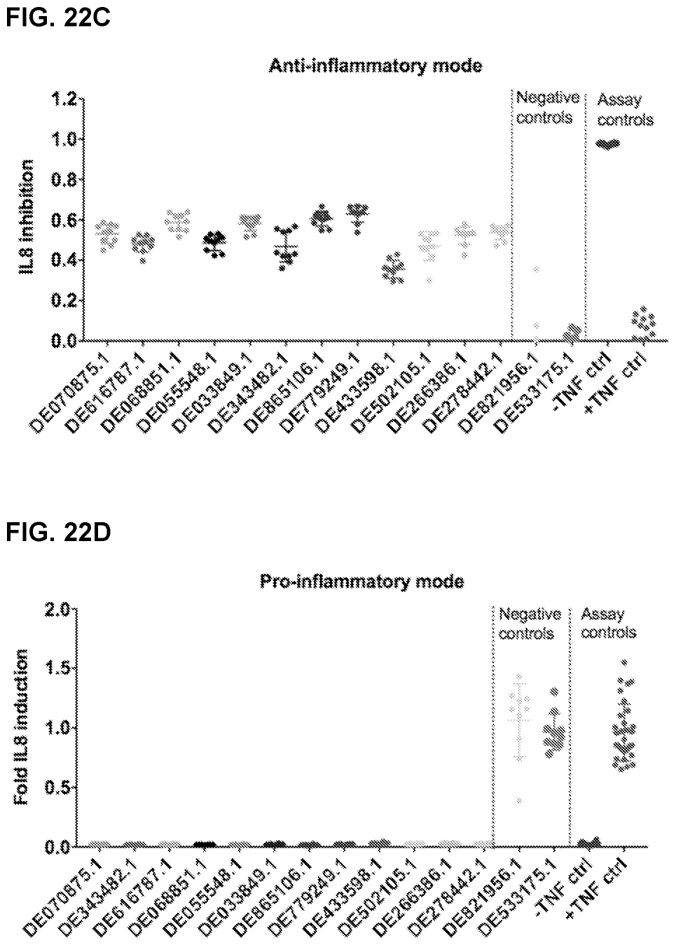

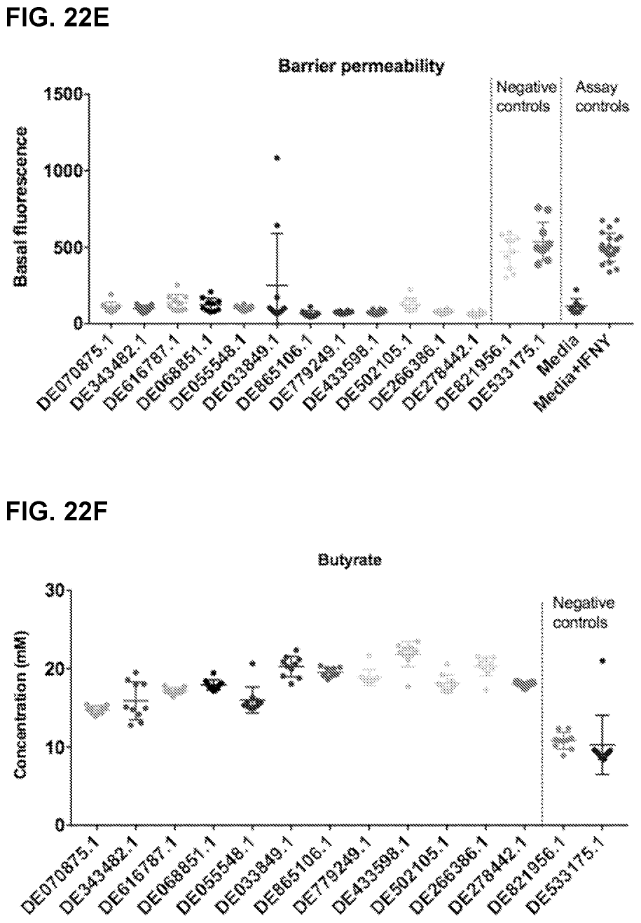

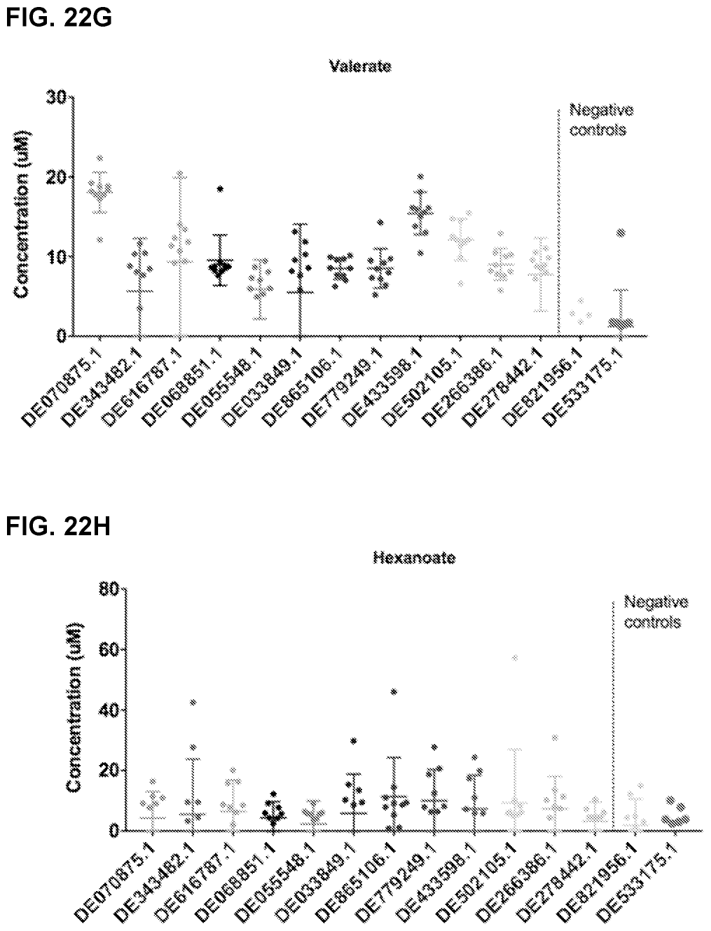

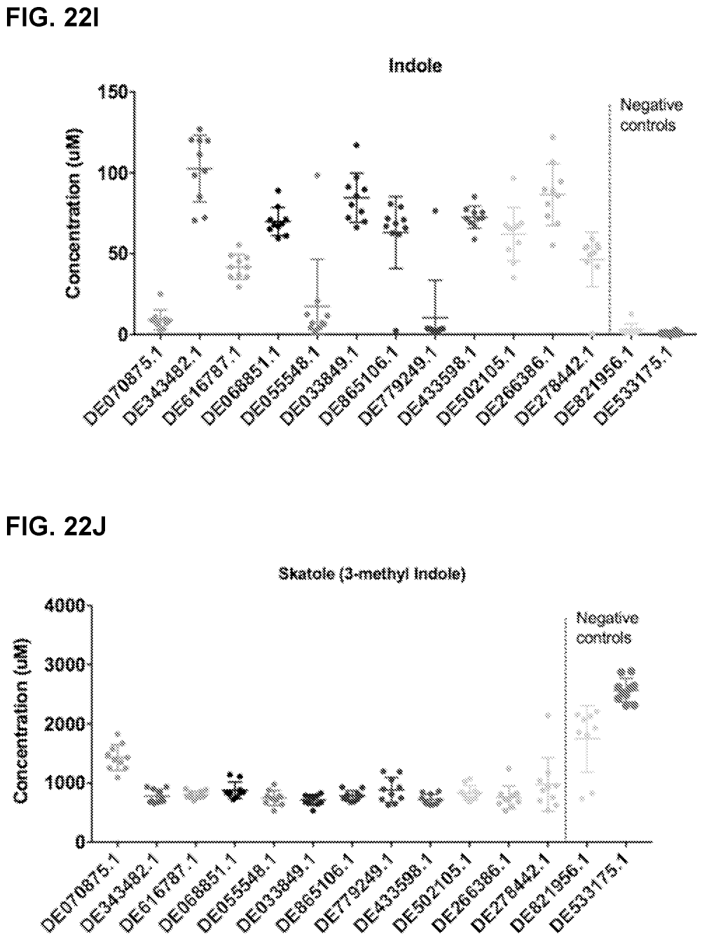

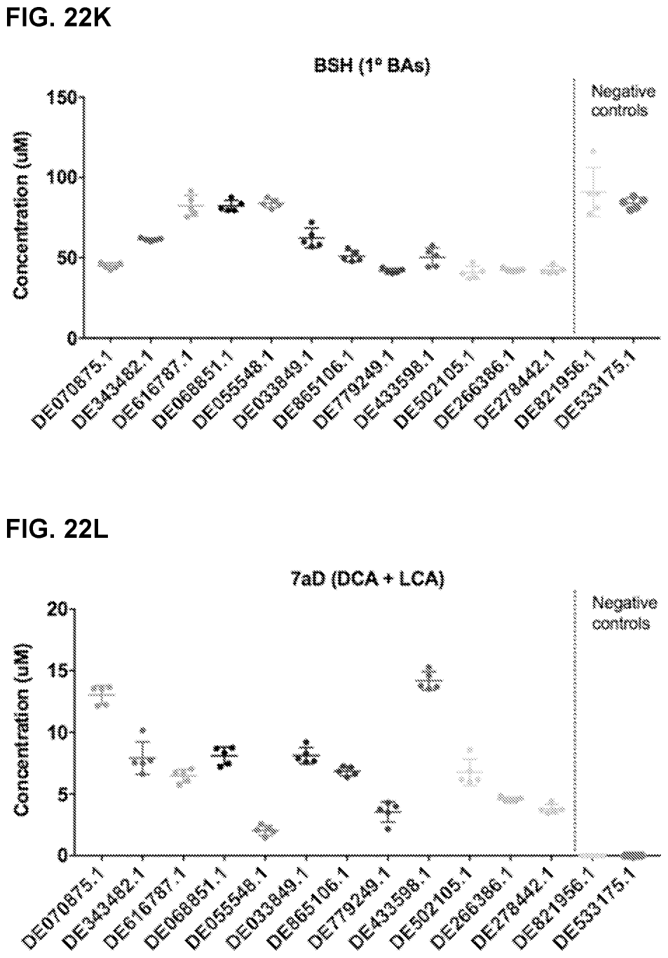

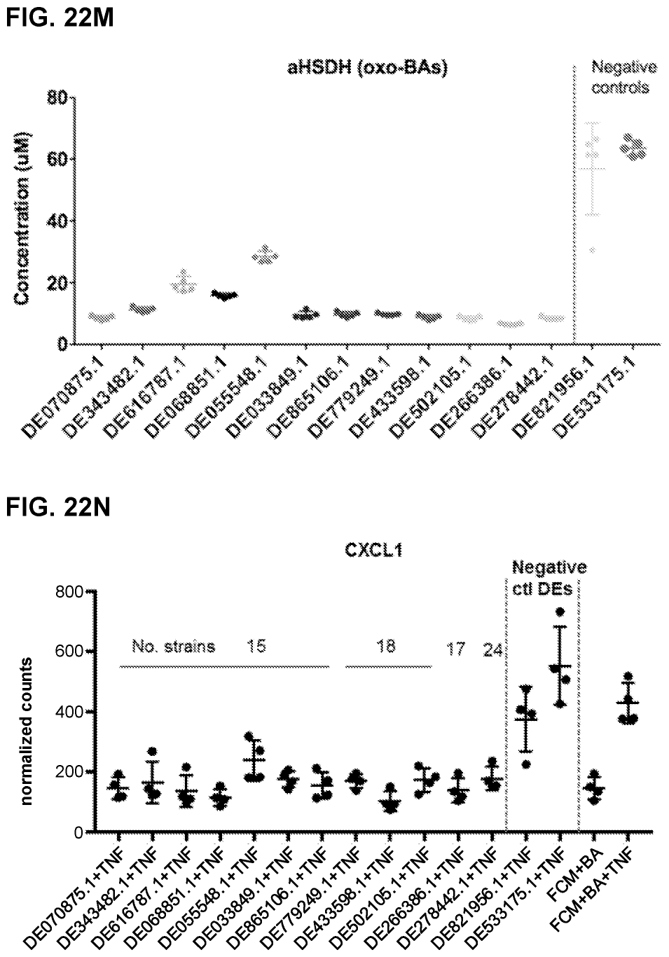

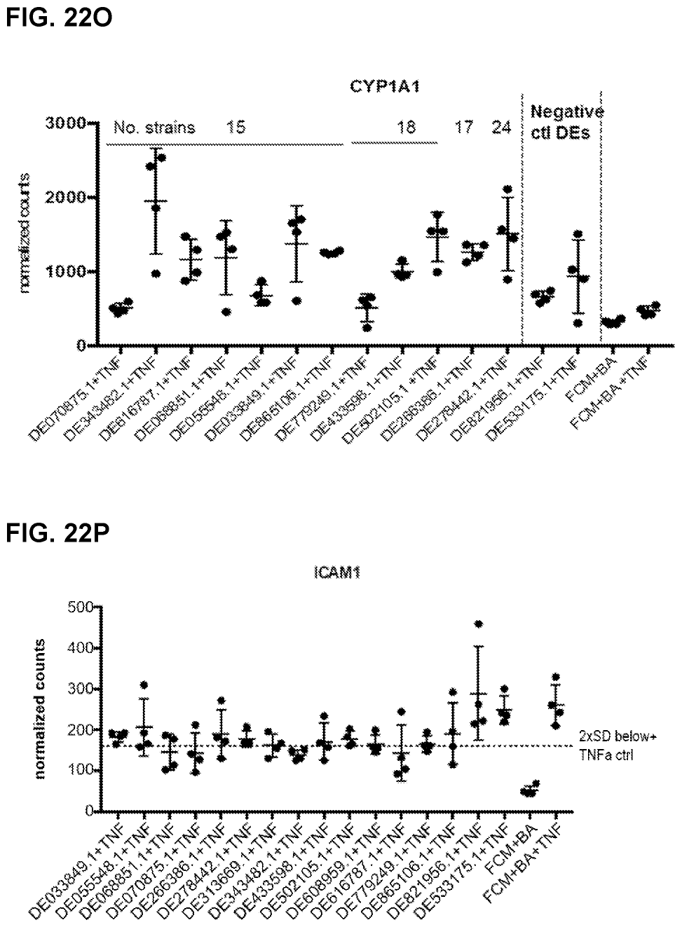

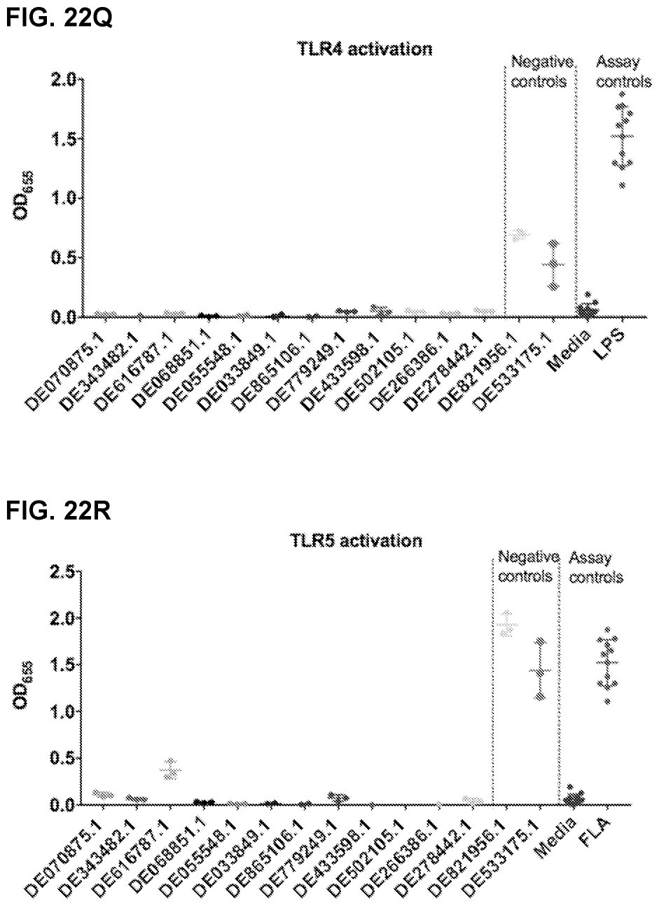

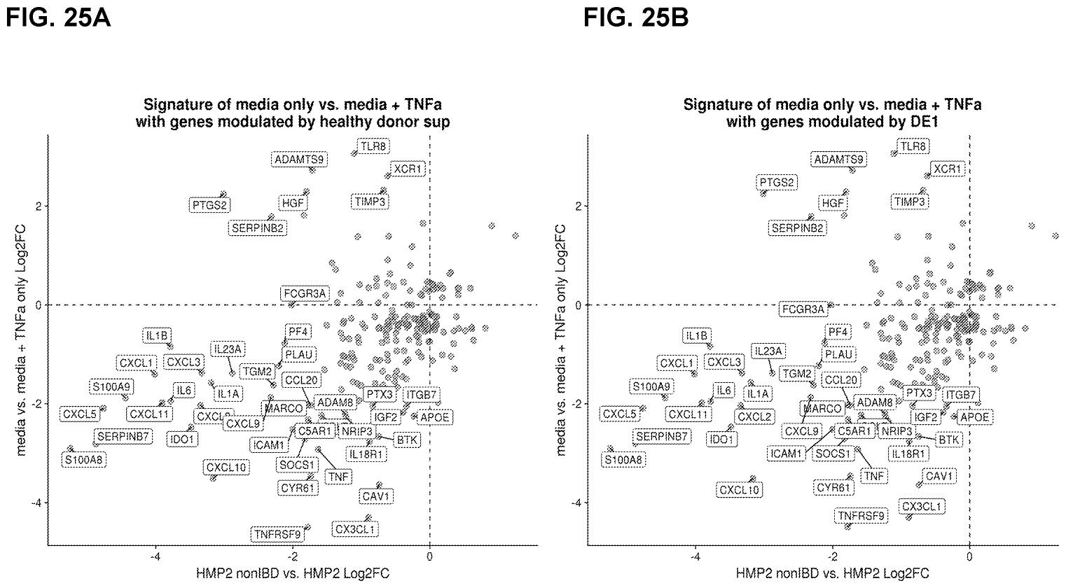

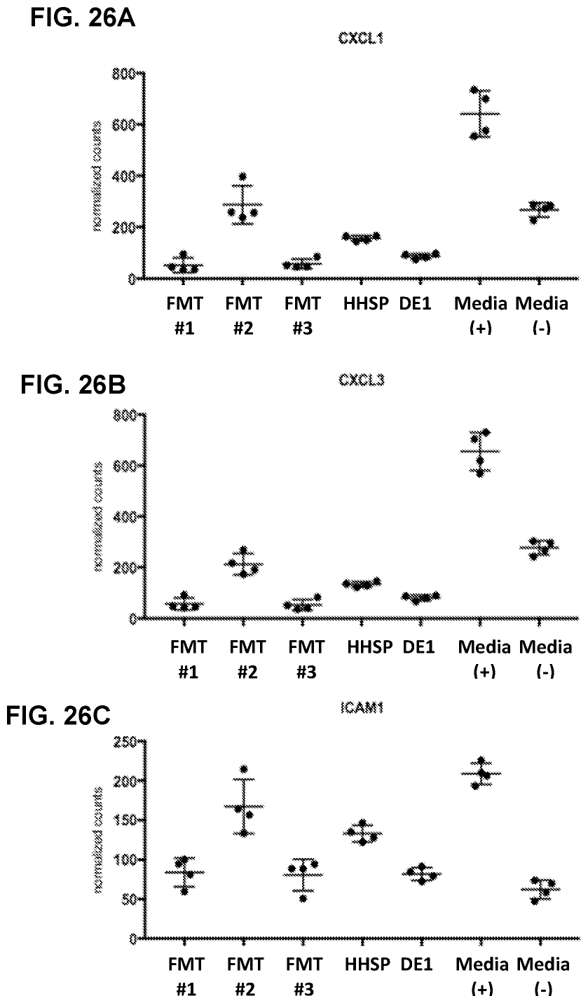

FIGS. 31, 32, 33, and 34 identify the bacterial species included in the different designed compositions. Depending on their bacterial species make-up, the designed bacterial compositions exhibited varying functional activity—see, e.g., FIGS. 20B, 21B, 23A, and 25B (inhibition of HDAC activity); FIGS. 20C, 21C, 22C, 23L, 23M, 23N, 23O, and 23P (anti-inflammatory activity); FIGS. 20D, 21E, 22D, and 23Q (pro-inflammatory activity); FIGS. 20E, 21D, 22E, 23K (restoration of epithelial integrity); FIGS. 20I-20L, 21H-21K, 22F-22H, and 23B-23F (short-chain and medium-chain fatty acid production); FIGS. 20M, 21L, 21M, 221, 22J, 23G, and 23H (tryptophan metabolite production); FIGS. 21N-21P, 22K-22M, and 59A-59C (secondary bile acid production); FIGS. 20N-20Q, 22N, and 22P (regulation of genes associated with inflammatory response); FIGS. 20R-20T (regulation of genes associated with Wnt activation); and FIGS. 20G, 20H, 21F, 21G, 22Q, and 22R (activation of a toll-like receptor pathway). And, as shown in FIGS. 26A and 26B, many of the designed compositions disclosed herein were similar or better at producing indole and butanoic acid (metabolites associated with anti-inflammatory responses) compared to FMT and even certain healthy human spore product (DXE).

From the fifty-six (56) different designed compositions constructed (DE1-DE56), most were designed to have beneficial properties for UC, while two (DE9 and DE38) were designed to include deleterious properties, such as the inclusion of strains with strong pro-inflammatory activity in the IEC assay to test the importance of excluding such strains from therapeutic compositions. The results presented here clearly showed that the two negative control compositions (DE9 and DE38), despite having HDAC inhibitory activity, failed to suppress TNFalpha-driven IL8 production, stimulated IL8 on their own, and failed to suppress the disruption of epithelial primary monolayers caused by interferon gamma. In addition, the negative control compositions were positive in the TLR4 and TLR5 activation assay (FIGS. 20G and 20H), failed to suppress TNFa-driven expression of pro-inflammatory genes in colonic organoids (FIG. 20C). In contrast, all the other 36 compositions tested did not exhibit any of these deleterious functions, demonstrating the importance of excluding IL8-inducing strains from compositions as described in this example.

Moreover, while all the bacterial compositions were designed to include species with HDAC inhibitory activity, compositions with lower number of such strains, or less coverage of the different HDAC clusters described herein, (e.g., DE984662.1 (DE3) and DE698478.1 (DE10)) resulted in decreased overall HDAC inhibitory activity, even after cultures had reached saturation. This result highlights the importance of including high representation of HDAC inhibitory strains and clusters to allow for maximum utilization of nutrients for production of SCFAs and HDAC inhibition.

Many of the therapeutic compositions described herein were designed for anti-inflammatory activity based on the single strain activity in the IEC assay but the effect of supernatants was also evaluated in a primary colonic organoid described above to explore the width of the anti-inflammatory activity and evaluate the modulation of additional disease-relevant pathways. Transcriptional analysis of colon organoids treated with TNFa revealed that pro-inflammatory cytokines relevant to ulcerative colitis (more highly expressed in UC in HMP2), such as CXCL1, CXCL2, CXCL3, and CXCL11, were also induced in vitro. Moreover, these levels of these transcripts in TNFa treated colon organoids were reduced in the presence of DEs with the highest levels of HDAC inhibition (FIGS. 20H, 20I, 20J, and 20K) underscoring the importance of designing compositions for maximum HDAC inhibition capacity as described here. Interestingly, DE8, which was designed to be an ineffective DE, did not lead to any decrease in abundance of TNFa induced transcripts, validating the exclusion of IL8-inducing strains from designed compositions. In addition, Wnt pathway target genes, CD44 and LRP6, were shown to have increased expression in response to DEs that most strongly activated the HEK 293 STF Wnt pathway reporter cell assay (FIG. 20R, FIG. 20S and FIG. 20T). These data suggest that Wnt activating consortia can contribute to supporting Wnt pathway driven intestinal epithelium homeostasis to facilitate repair of mucosal injuries associated with diseases or disorders disclosed herein (e.g., IBD).

Additionally, IFN-γ is a potent immunomodulatory Th1 cytokine, found elevated in UC patients and secreted by activated immune cells in the lamina propria. See, e.g., Olsen et al., Cytokine 56(3): 633-640 (2011). In order to evaluate the ability of bacterial compositions to reverse inflammatory pathways induced by IFN-γ, human primary organoids were incubated with 5% bacterial supernatants in the presence or absence of 10 ng/ml IFN-γ overnight. Following overnight incubation, the treatments were aspirated from each well and the organoid droplets were incubated for 15 minutes with 150 μl RLT buffer (Qiagen RNeasy kit)+β-Mercaptoethanol (β-ME) and then stored as cell lysates at −80° C. until processing for transcriptomics analysis. For an initial analyses of gene expression changes associated with inflammation, lysates were thawed and hybridized for 18 hours at 65° C. with the capture and reporter tags of the Nanostring Human Autoimmune panel (770 gene targets including reference genes) (NanoString Technologies, Inc.). The Nanostring prep station was used to purify probe-bound RNA and was loaded onto a cartridge. The Nanostring digital analyzer was then used to directly count transcripts. The results from the counts were then analyzed using the NSolver software Advanced analysis.

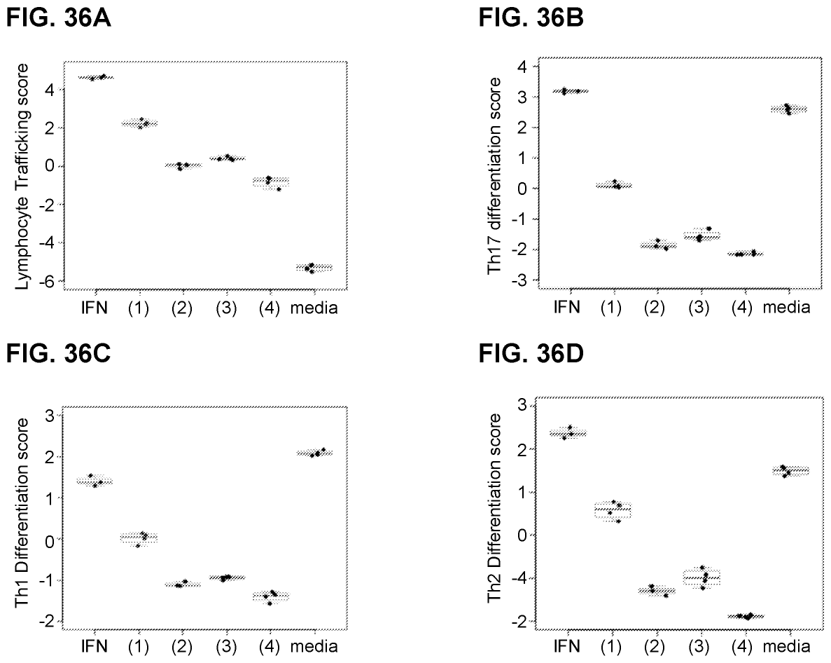

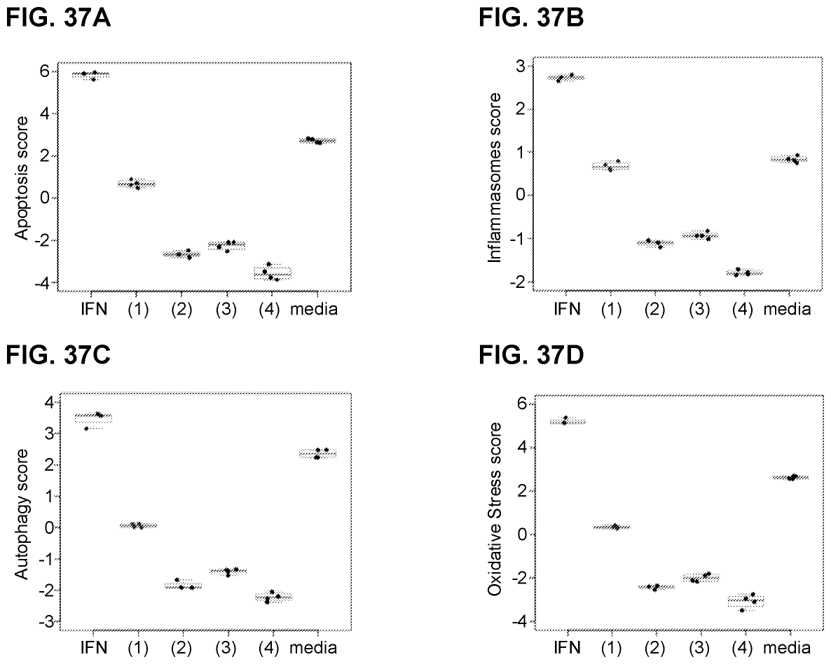

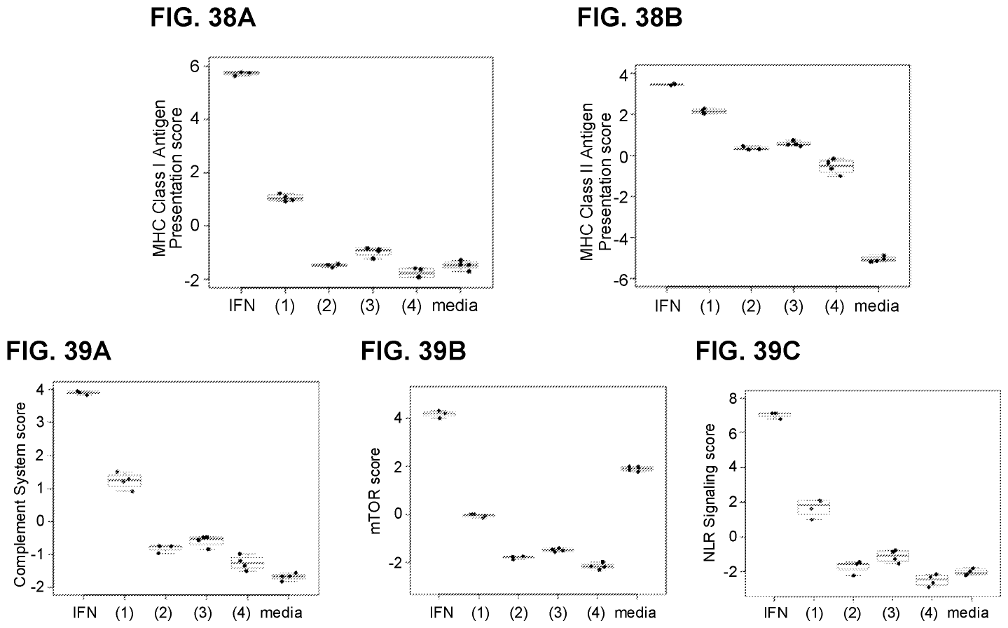

As observed above with the TNFa treated colonic organoids, some of the designed bacterial compositions disclosed herein were also able to downmodulate many of the gene pathways that are induced by IFN-γ as measured in the colonic organoids. Examples of such genes included those associated with inflammatory chemokine signaling (FIG. 35A), NF-κB signaling (FIG. 35B), TNF family signaling (FIG. 35C), type I interferon signaling (FIG. 35D), type II interferon signaling (FIG. 35E), TLR signaling (FIG. 35F), lymphocyte trafficking (FIG. 36A), Th17 cell differentiation (FIG. 36B), Th1 cell differentiation (FIG. 36C), Th2 cell differentiation (FIG. 36D), apoptosis (FIG. 37A), inflammasomes (FIG. 37B), autophagy (FIG. 37C), oxidative stress (FIG. 37D), MHC class I and II antigen presentation (FIGS. 38A and 38B, respectively), complement system (FIG. 39A), mTOR (FIG. 39B), and nod-like receptor signaling (FIG. 39C).

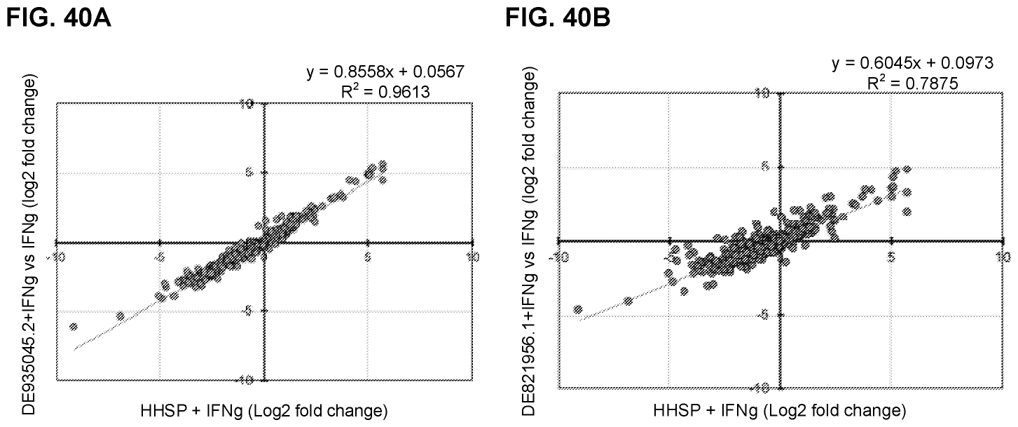

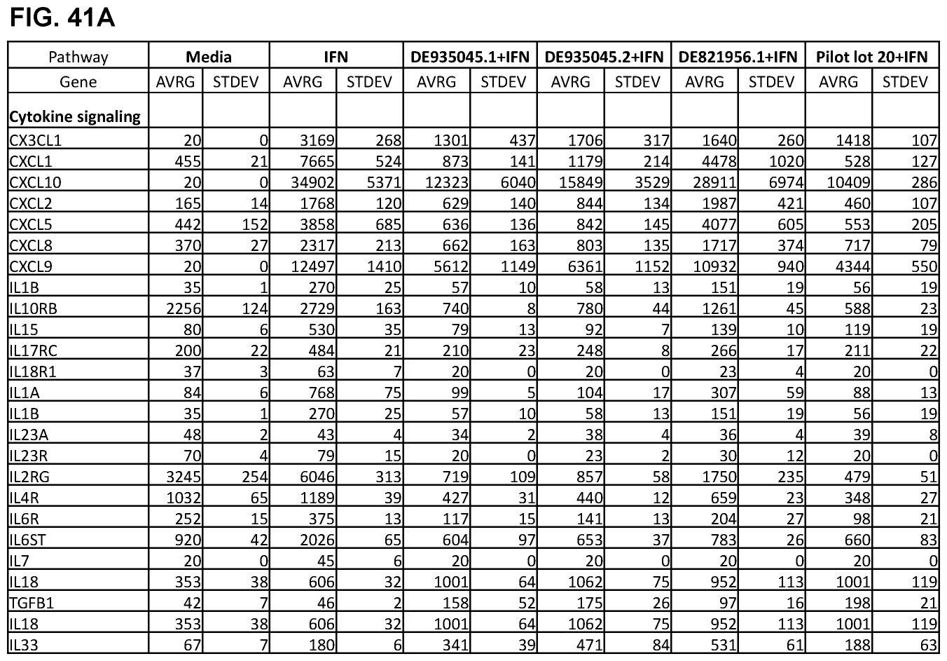

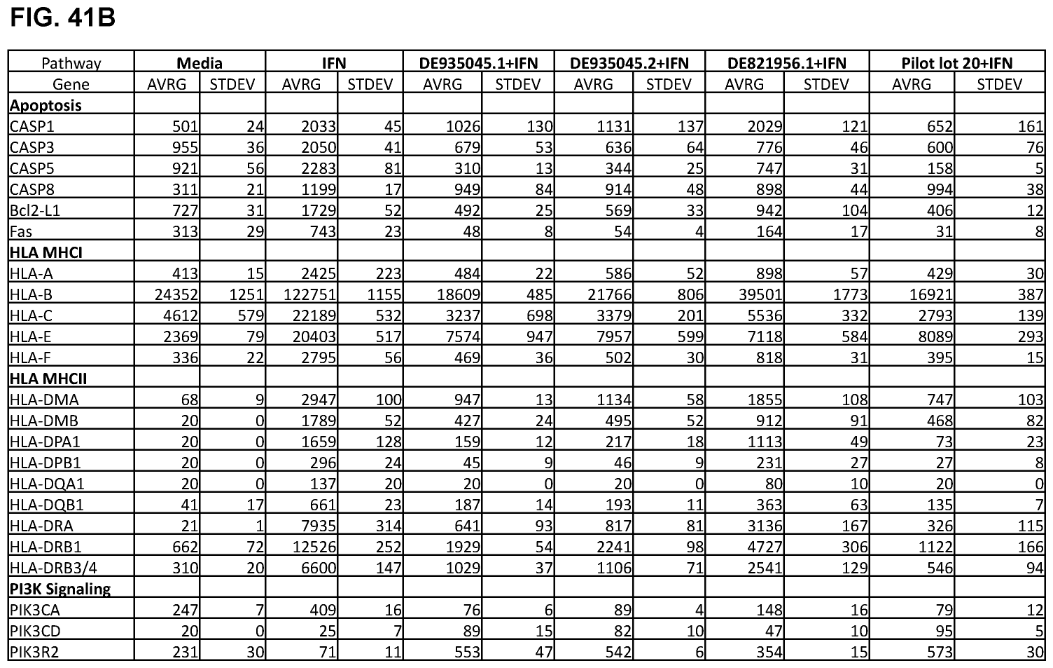

At the individual gene level, some of the designed compositions disclosed herein were able to induce gene expression changes similar to that observed with HHSP in the colonic organoids challenged with IFN-γ (see, e.g., FIGS. 41A and 41). For example, the gene expression profile observed with the DE935045.1 (DE37) and DE935045.2 (DE39) compositions closely mirrored that observed with HHSP (FIG. 40A). With the DE821956.1 (DE9) composition, which was specifically constructed to be pro-inflammatory, there was some correlation with the gene expression profile observed with HHSP, the correlation was not as strong overall and multiple genes in the inflammation gene panel used were not successfully modulated (FIG. 40B). These results suggest that bacterial compositions can be specifically designed to recapitulate many of the properties of complex bacterial consortia.

In support of the anti-inflammatory nature of many of the designed compositions disclosed herein, some of the tested compositions were able to decrease the expression of pro-inflammatory cytokine genes (e.g., IL1B and IL15), while increasing the expression of certain cytokine genes that are thought to be associated with anti-inflammation and/or mucosal wound healing (e.g., TGFB1, IL18, and IL33) (see FIG. 41A). In addition, these compositions were able to downregulate expression of genes involved in cellular apoptosis (e.g., caspases Casp1, Casp3, Casp5, Casp8, Fas, and Bcl2) as well as MHC antigen presenting markers, all of which are induced by IFN-γ (FIG. 41). See, e.g., McEntee et al., Front Immunol 10:1266 (2019).

To confirm the results described above using Nanostring, gene expression changes were also evaluated using RNASeq using an Illumina NovaSeq 6000 instrument. Pathway enrichment analysis was performed on differential gene expression data using the R package for fast pre-ranked gene set enrichment analysis (fgsea v 1.10.1). A total of 330 KEGG pathways (annotations downloaded on 2019 Jan. 24) were tested for each DE gene list. Gene ranking was determined by the t-statistic of differential expression, encapsulating both fold change and significance into a single test statistic. A total of 100,000 permutations were run for each gene set to create a reliable background distribution to calculate a robust enrichment score to assess significance of each pathway tested.

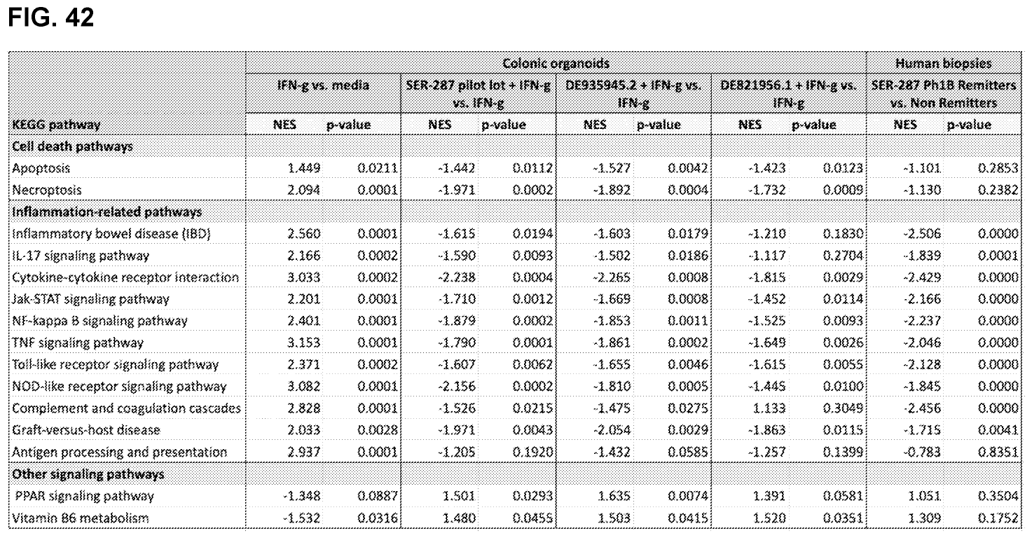

As shown in FIG. 42, treatment of the colonic organoids with IFN-γ also induced the activation of many inflammatory pathways in the KEGG databse, including Inflammatory bowel disease, cytokine-cytokine receptor interaction, IL17, JAK-STAT, NFKb, TNF, Toll-like receptor, and NOD-like receptor signaling pathways, Complement and coagulation cascades, Graft vs Host Disease, and Antigen processing and presentation. Additional pathways modulated by IFNg included apoptosis and necroptosis, PPAR signaling, and vitamin B6 metabolism. As observed with the Nanostring data, co-treatment of the colonic organids with either DE935045.2 (DE39) or HHSP, reversed the IFN-γ-induced gene signature. The effect was less apparent with the pro-inflammatory DE821956.1 (DE9) composition. Importantly, the changes in pathway expression achieved by co-treatment of colonic organoids with bacterial supernatants and IFN-γ overlapped with pathways differentially expressed between Remitters and Non Remitters in colonic biopsies of UC patients in a HHSP-based clinical trial of mild to moderate UC (NCT02618187). These results demonstrate that certain designed bacterial compositions disclosed herein can modulate host gene expression similarly to a natural complete spore community (HHSP) and induce gene expression changes that correlate with remission in a clinical setting.

As described herein, some of the designed bacterial compositions were constructed to exhibit little to no inflammatory activity (e.g., DE935045.1 (DE37) and DE935045.2 (DE39)). The anti-inflammatory properties of such designed bacterial compositions were also tested by assessing their effect on macrophage function. Specifically, viability and anti-inflammatory and pro-inflammatory cytokine expression and production were assessed in human macrophages treated with a designed bacterial composition described herein. Human macrophages were derived from the THP-1 monocytic cell line (ATCC). THP-1 monocytes were grown in RPMI (Gibco) supplemented with 10% FBS, Pen/Strep, and sodium pyruvate. Cells were differentiated into macrophages by incubation with 25 ug/mL phorbol 12-myristate-13-acetate (PMA, Peprotech) for 24 hours. Cells were grown in 96 well tissue culture treated sterile microtiter plates (Corning) with 100,000 cells seeded per well. Macrophage differentiation was confirmed by quantifying attachment to the tissue culture growth plate (cellular adhesion assay) and expression of macrophage cell surface markers (determined by flow cytometry). The differentiation medium was then replaced with fresh medium, and cells were rested for 24 hours to return the cells to a basal signaling state. Following rest, differentiated macrophages were stimulated with 1% bacterial culture supernatant, a multiplicity of infection (MOI) of 20 bacterial cells (counted via flow cytometry) per macrophage, or a combination of 1% supernatant and MOI20 bacterial cells. 1% supernatant examined the effect of bacterial metabolites and <0.2 um filterable bacterial cell surface products on macrophage signaling, and the use of whole bacterial cells examined the contribution of the bacterial cell surface (and its inherent stimulatory molecules) to macrophage signaling. The combination thereof examined macrophage sensing of both constituents (bacterial metabolites and products as well as surface molecules). As innate immune cells, macrophages are tuned to sense microbial cells and their products.

After 24 hours of stimulation, culture supernatants were collected for cytokine measurements (Luminex). The cells were harvested for determination of viability or were used to generate cellular lysates for transcriptional analyses. Cell viability was measured via luminescence in an assay that directly measures the presence of cellular ATP (a marker of cell health; CellTiterGlo 2.0, Promega). Assay performance of CellTiterGlo was controlled via an ATP standard curve, and cellular viability was normalized to the respective medium alone (non-stimulated) wells. Quantitation of cytokine production was performed with a ThermoFisher multi-plexed Luminex panel with commercial standards. All analyte standard curves were quality controlled in xPONENT (custom Luminex software), and cytokines were detected above the limit of quantitation of each respective analyte. Transcriptional changes were assessed via NanoString (human myeloid 2.0 panel) with analogous hybridization and sample prep conditions across cellular treatments. Raw probe counts were normalized using nSolver (NanoString software) with analogous background correction and data normalization across samples. The internal negative and positive controls (commercially provided by NanoString in each panel) all passed quality control across the samples. Data were plotted in GraphPad Prism 8.4.3.

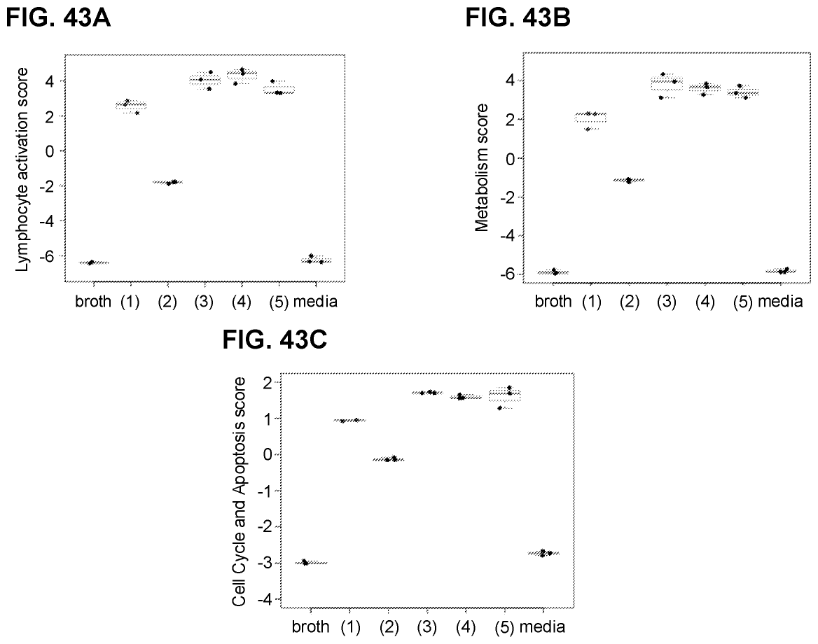

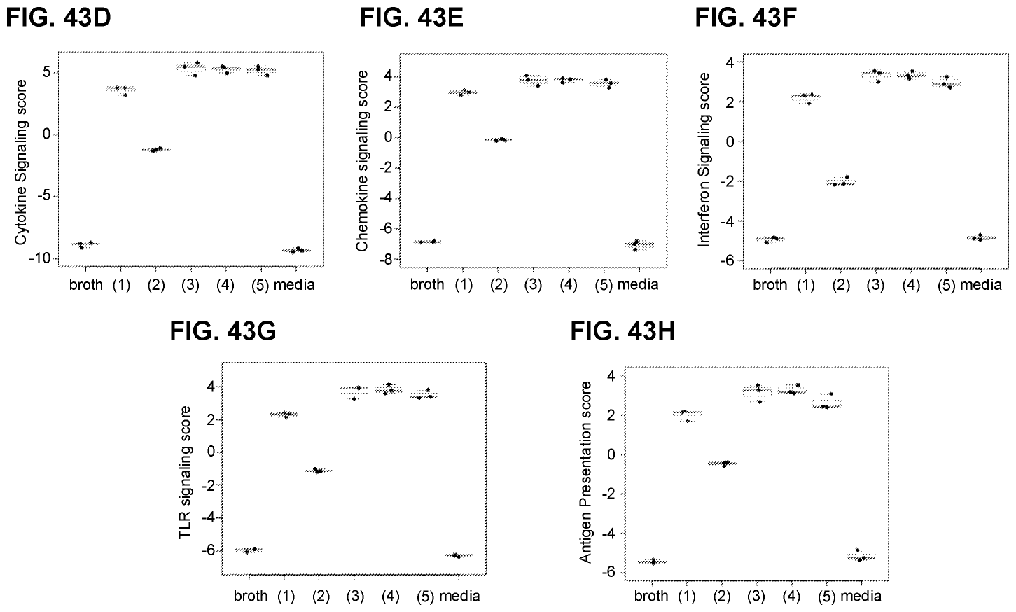

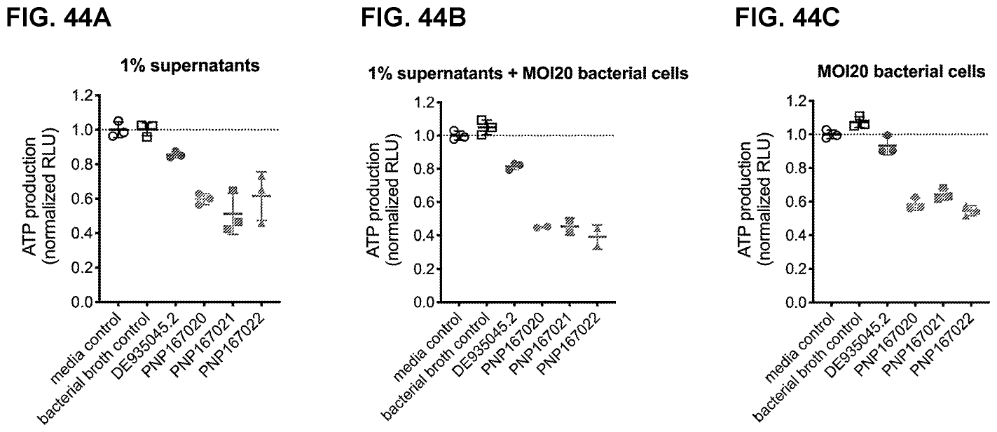

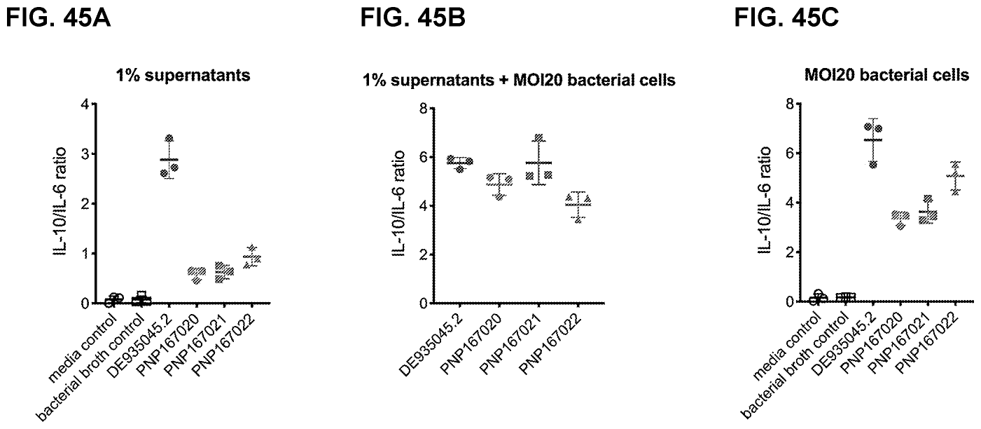

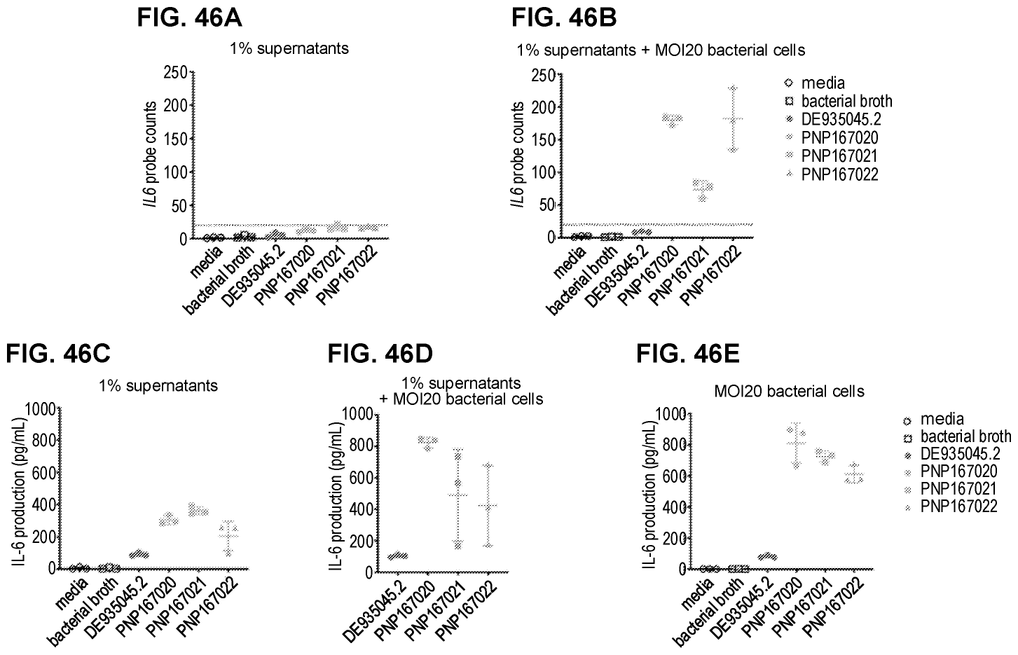

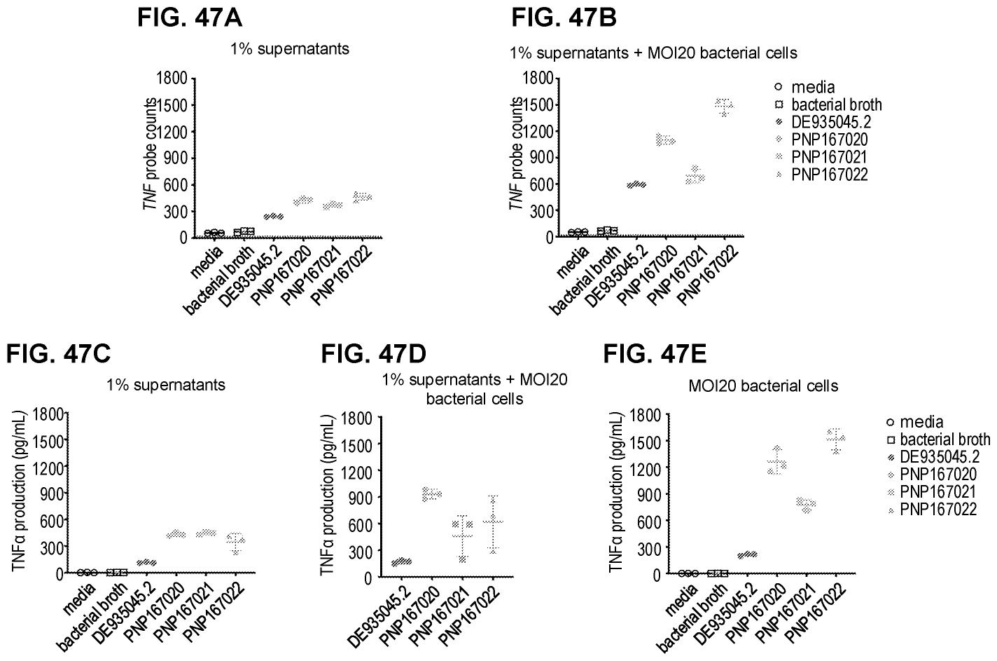

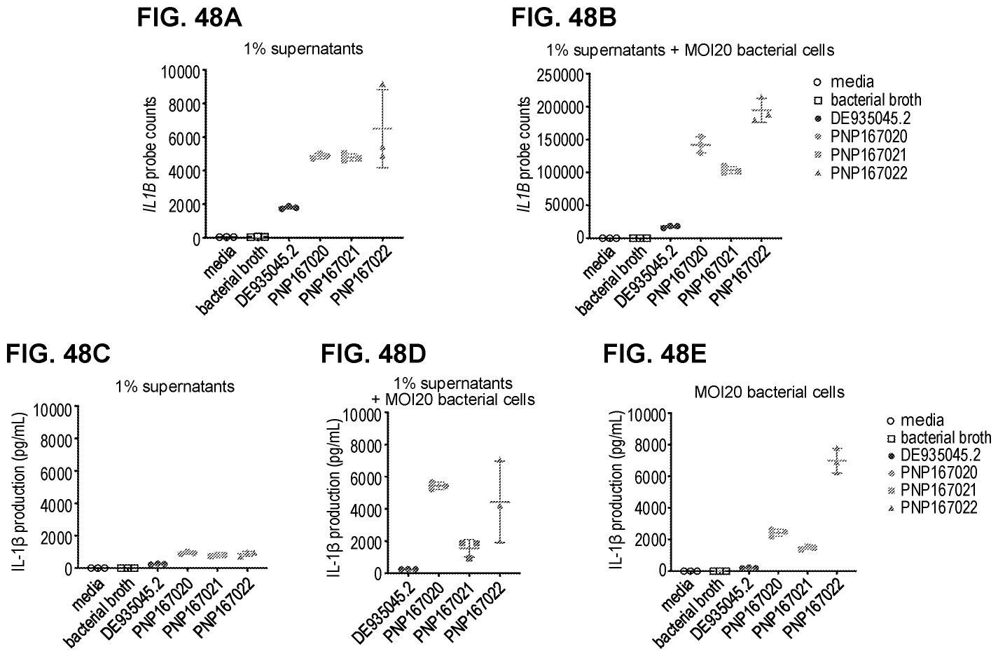

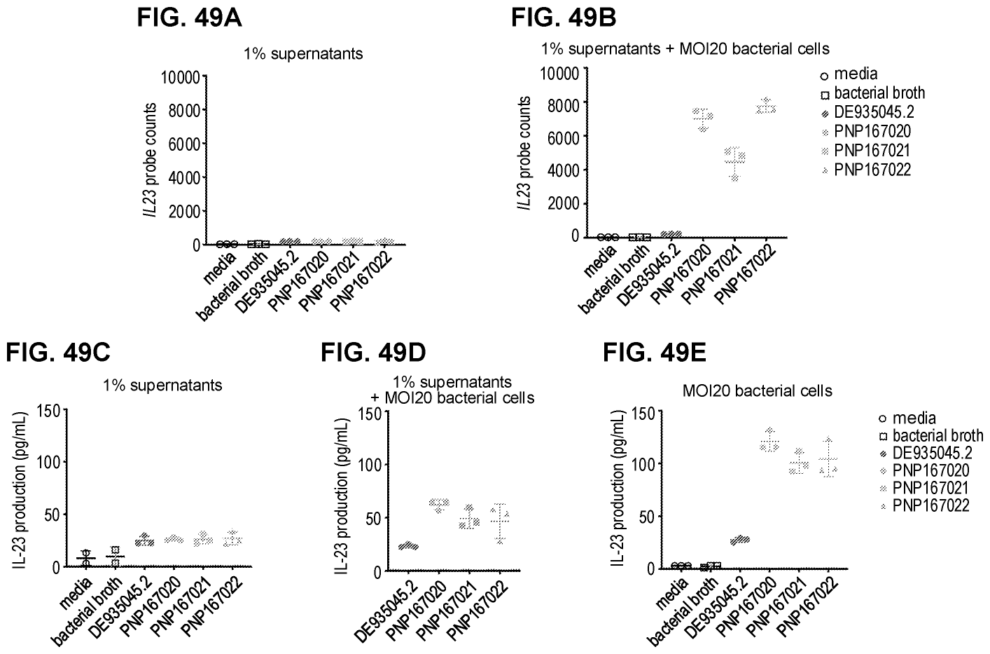

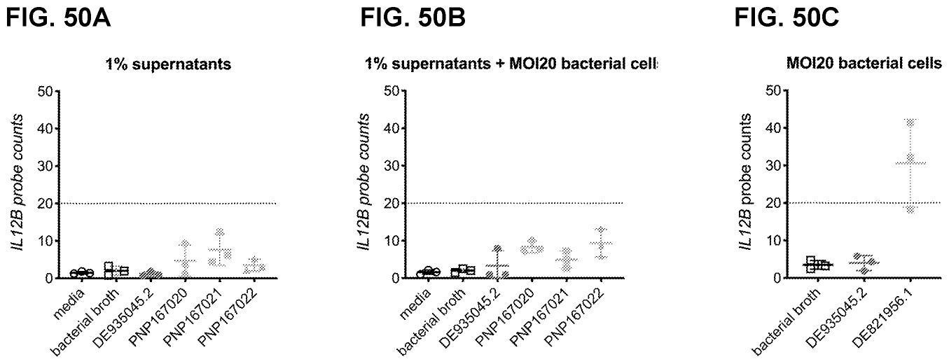

As shown in FIGS. 44A-44C, macrophages treated with the DE821956.1 composition (DE9), which was designed to exhibit strong inflammatory properties, produced significantly lower amounts of ATP compared to the other groups, highlighting the negative effect that inflammation has on macrophage viability. Additionally, treatment with three HSSPs, natural community pilot lots derived from healthy donors (PNP167020, PNP167021 and PNP167022), also significantly decreased macrophage viability. In contrast, macrophages treated with the DE935045.2 (DE39) composition exhibited the robust viability across the different treatment groups of supernatants, supernatants plus bacterial cells, or bacterial cells. As explained elsewhere in the present disclosure, the DE935045.2 (DE39) composition was specifically designed to exhibit minimal inflammatory properties and to avoid inclusion of any bacterial strains that could induce inflammation. In support, the macrophages treated with the DE935045.2 (DE39) composition also produced a greater anti-inflammatory IL-10 skewed IL-10/IL-6 ratio (e.g., compared to macrophages treated with DE821956.1 (also referred to herein as “DE9”) or three natural healthy communities (see FIG. 45), while producing little to no inflammatory cytokines, such as IL-6 (see FIGS. 46A-46E), TNF-α (see FIGS. 47A-47E), IL-10 (see FIGS. 48A-48E), IL-23 (see FIGS. 49A-49E), and IL-12 (see FIGS. 50A-50C). The decrease in the inflammatory cytokines expression after DE935045.2 (DE39) treatment compared to that elicited by HHSP was apparent both at the gene and protein levels. Similar effects were observed when the expression of other genes associated with macrophage function (see FIGS. 43A-43H). These data consistently show that a composition designed to be anti-inflammatory while avoiding inclusion of strains with inflammatory properties can induce more robust anti-inflammatory properties in human macrophages than complex communities derived from healthy human donors. Additionally, expression of genes under the control of the Ahr pathway which is involved in barrier protection and immunomodulation were also evaluated in the human organoid system. As seen in, e.g., FIG. 20O, designed compositions were able to induce expression of CyplA1 gene which encodes an enzyme of the cytochrome P450 superfamily in the AhR pathway. Importantly, the ability to induce CyplA1 was directly correlated to the abundance of indole, and described AhR agonist, in the supernatants and, in contrast with Wnt and anti-inflammatory activities, is not proportional to SCFAs and HDAC inhibition indicating that the design compositions successfully affect host responses by more than one mechanism of action.

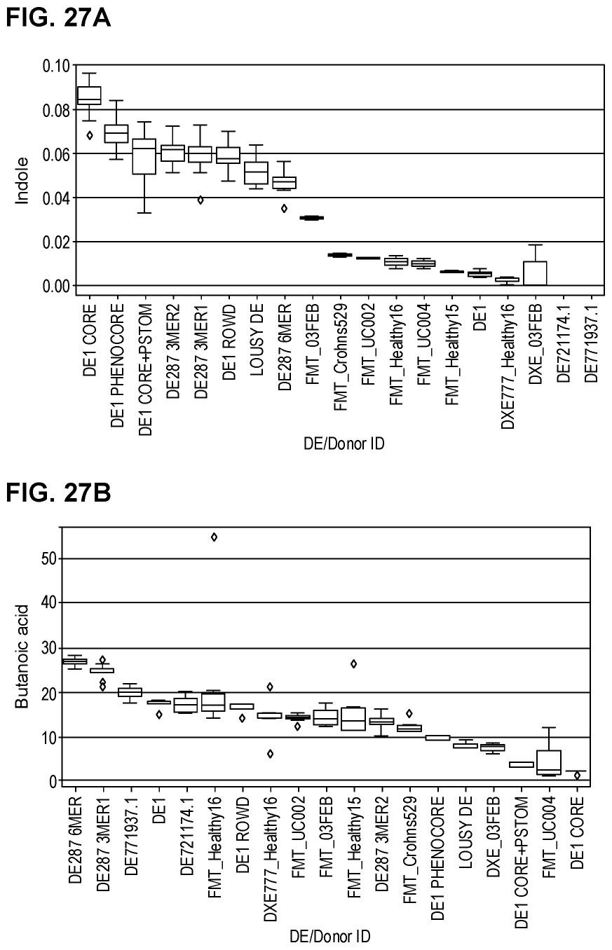

Finally, as can be seen in FIGS. 26A to 26C, 27A, and 27B, designed compositions described herein had similar (if not better) properties as an FMT and spore fraction (HHSP) of a healthy donor: HDAC inhibition, anti-inflammatory activity and SCFA production. Importantly, the analysis of gene expression in colonic organoids showed that there was very significant overlap between the gene expression signature of a TNFalpha treated organoid and the gene expression in biopsies of UC subjects, and that both the HHSP and composition supernatants can reverse a significant part of that signature including several inflammation related genes, such as Cxcl1, Cxcl2 and ICAM1. These results indicate that compositions designed by the criteria describe here recapitulate many features of complex natural products and have the potential to modulate host gene expression to restore intestinal health.

These results demonstrate that bacterial compositions can be designed to have specific functional features. Such ability suggests that depending on the pathways involved, different compositions can be designed to treat a wide range of diseases and/or disorders. The results also show that compared to much more complex products (e.g., FMT and spore-prep compositions), the designed compositions disclosed herein are superior at producing certain metabolites that can be important in treating certain inflammatory diseases.

Collectively, the results disclosed herein show that combining data on functional features of strains and bacterial consortia with data on which species will engraft in human subjects (Table 5) ensures that the consortia will express these functional features when administered to human subjects. Importantly, the results further demonstrate that while many strains could be selected that may possess one or more of the desired functional features disclosed herein, such species will not necessarily engraft when administered to human subjects. Therefore, such species would not likely be of therapeutic value since they would not be able to express these functional features and have the desired effect when administered to patients. The bacterial compositions disclosed herein comprise one or more bacteria that not only allow the composition to exert the different functional features disclosed herein, but are also capable of engrafting when administered to human subjects.

Furthermore, combining data on functional features of strains with their association with clinical remission in human subjects (Table 3) ensures that the consortia will express functional features with therapeutic benefit while not promoting non-remission through other mechanisms.

Data across these consortia furthermore show that, for example: 1) consortia containing multiple (e.g., 5, 7, 10, 15, 18) HDAC inhibiting strains, sometimes coming from distinct HDAC clusters, have stronger HDAC inhibition than those with few HDAC inhibiting strains (e.g., 2, 3, 4, 5), 2) unlike HDAC, consortia affect certain other functional targets equally despite if there is only one or a few strains possessing that function, 3) exclusion of pro-inflammatory strains results in better repair of the intestinal epithelial barrier, 4) these designed compositions have the same effect as donor-derived HHSP or fecal microbial transplant on the host expression of a wide range of genes associated with ulcerative colitis, 5) compositions designed to affect the levels of several distinct molecules (e.g. short-chain fatty acids and tryptophan metabolites) can modulate diverse disease-relevant pathways and have multiple mechanisms of action (reduction of pro-inflammatory cytokine expression and increase in Wnt pathway expression, or increase expression of AhR pathway, respectively).

### Example 14: Analysis of the Effect of Designed Compositions in Treating Colitis in an IL-10−/− Animal Model

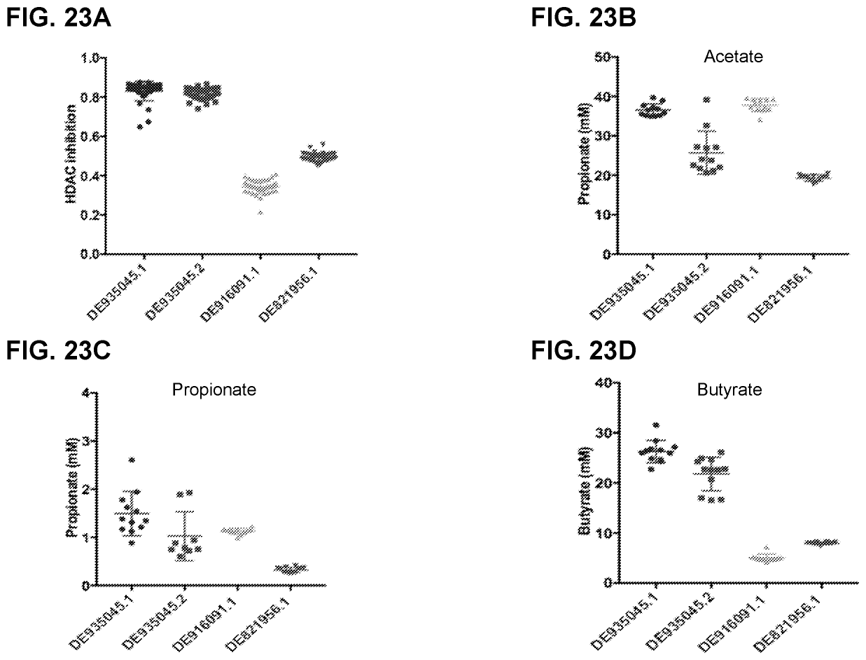

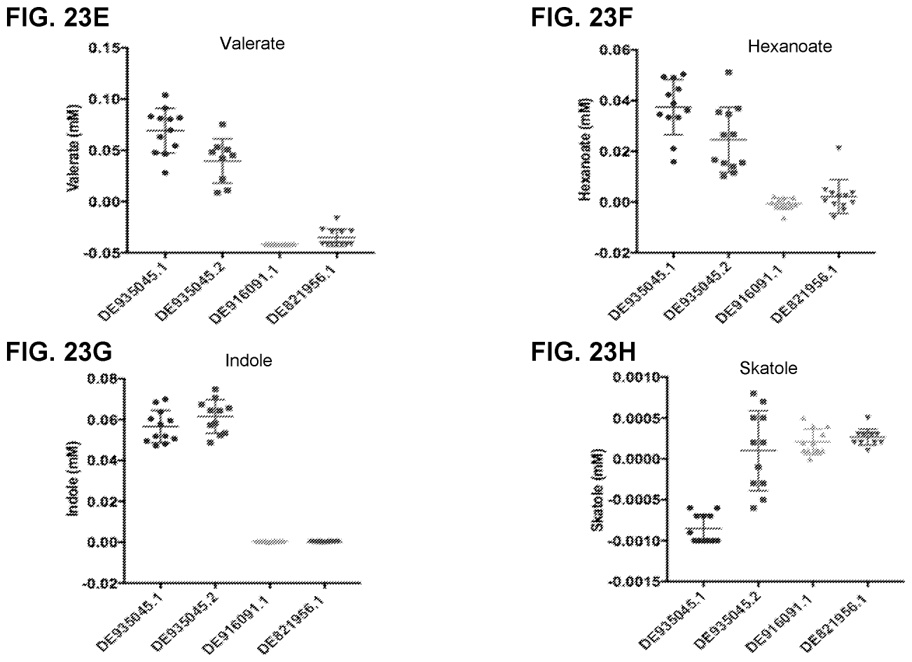

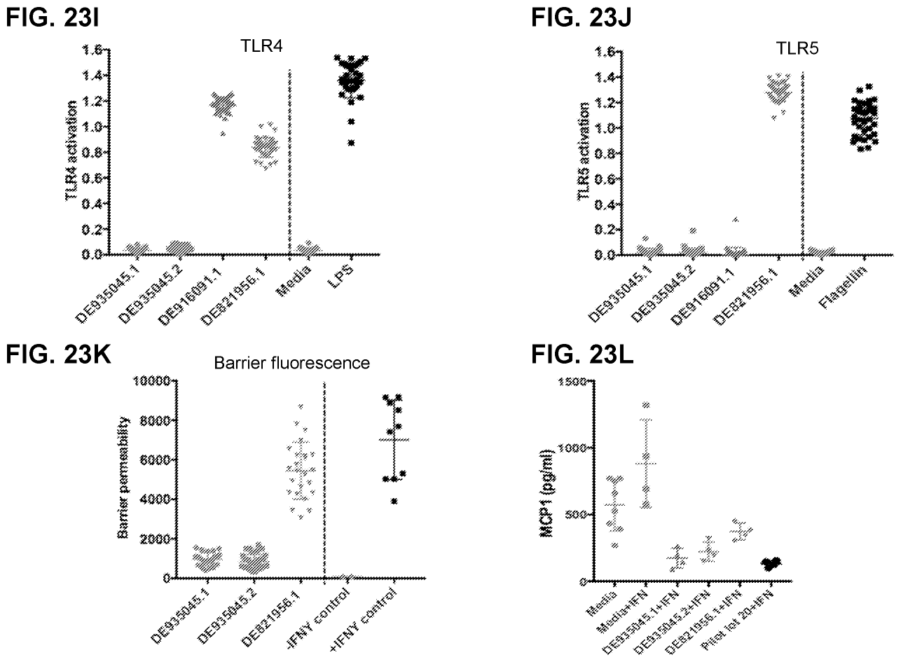

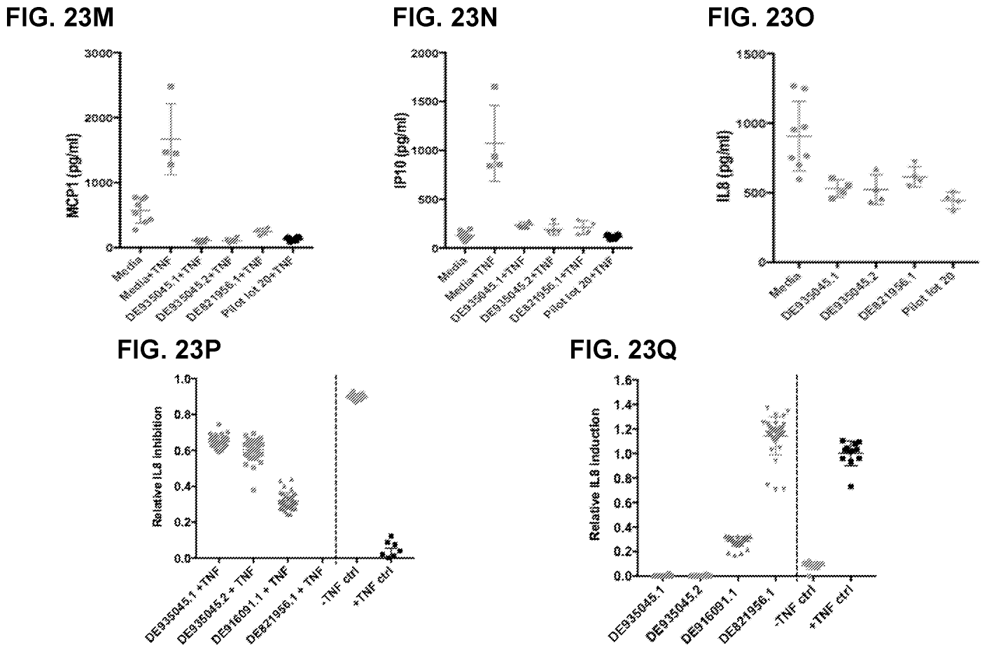

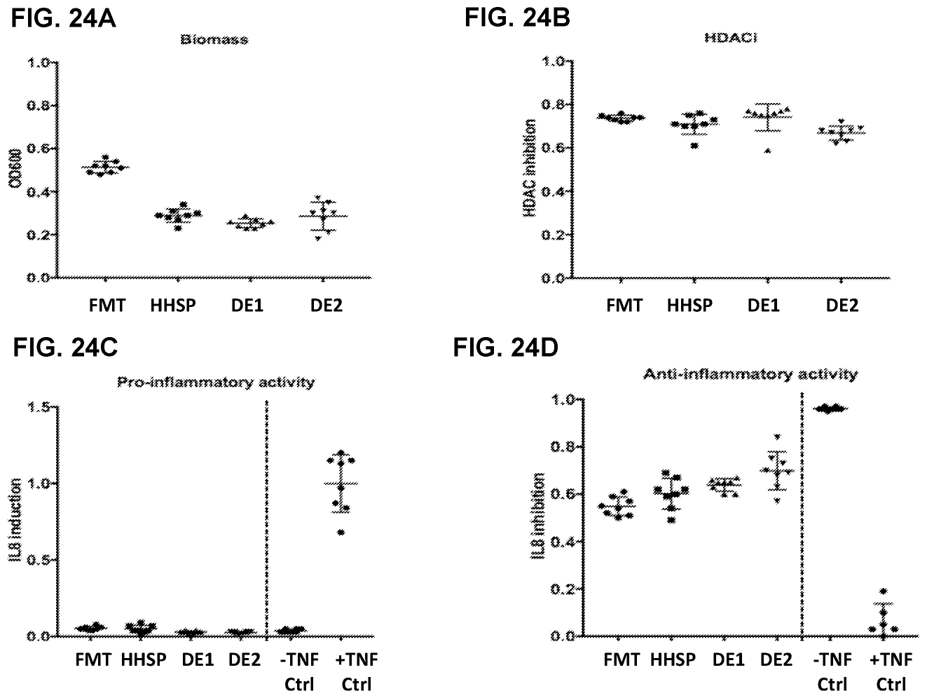

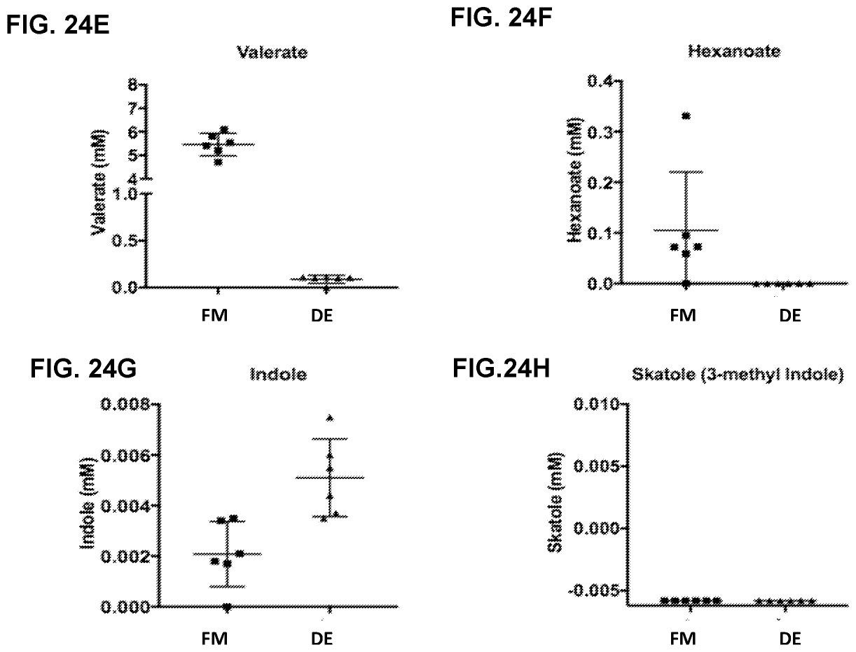

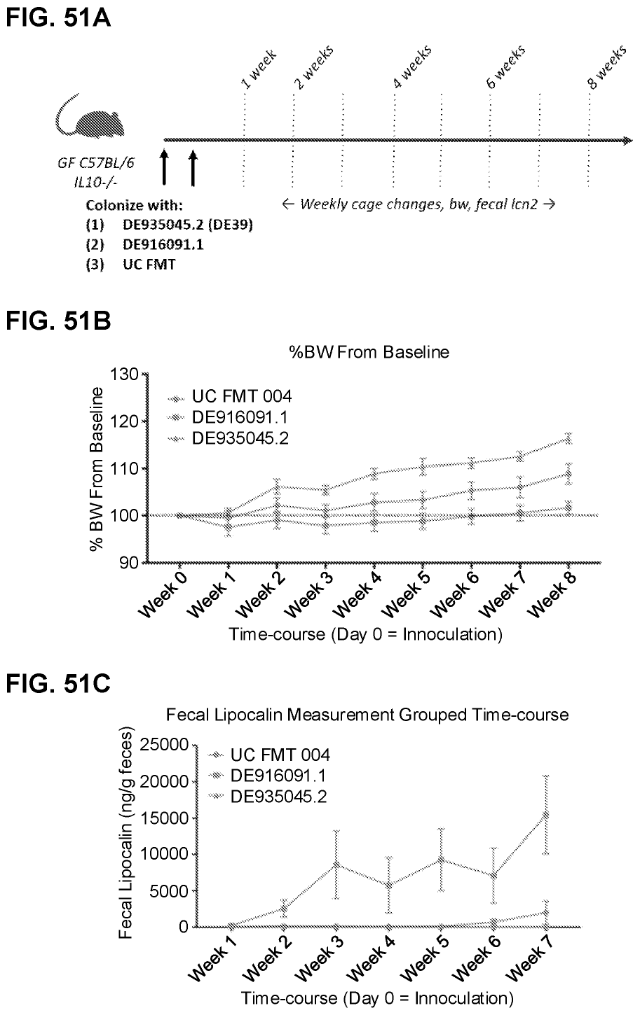

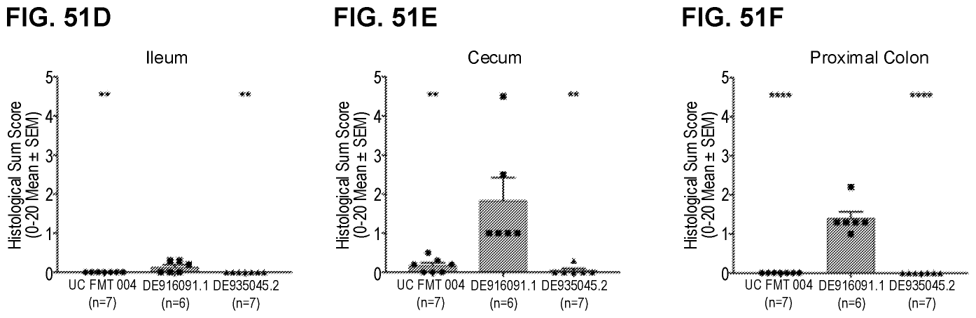

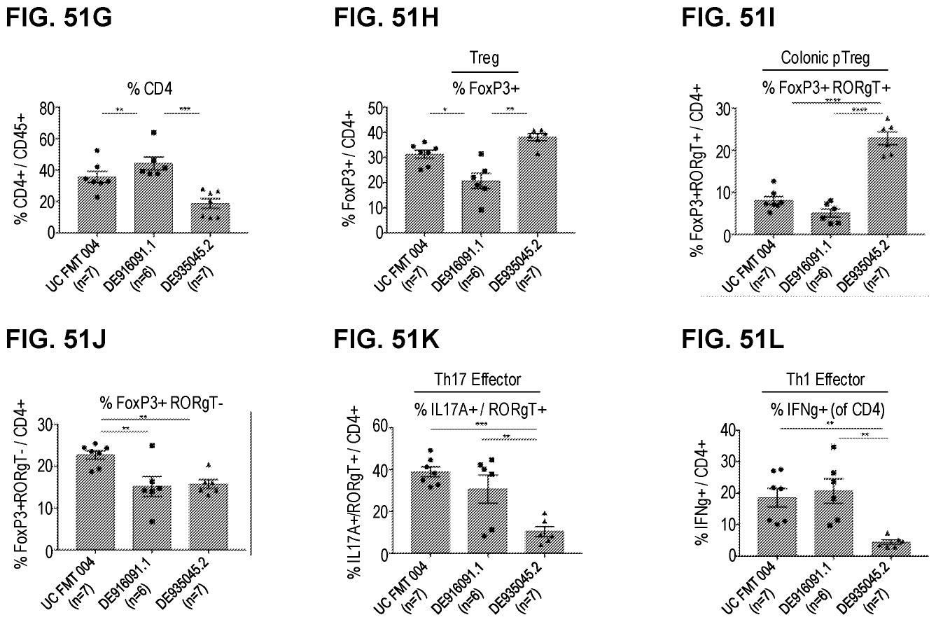

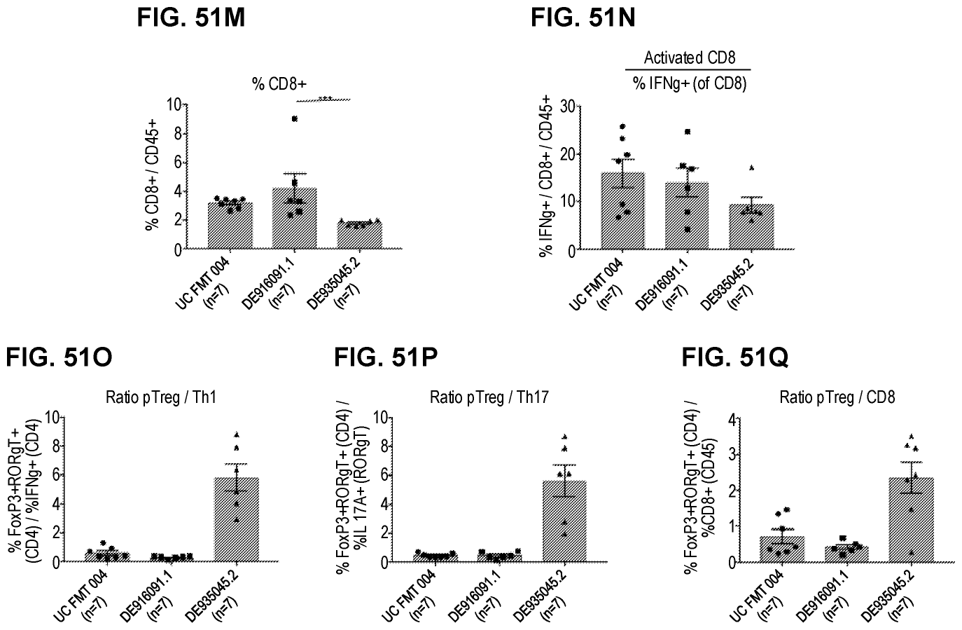

Next, the ability of different designed compositions described herein to exert therapeutic effects in vivo was assessed using an IL-10 knockout (KO) mouse model, which is used as a model for colitis. See, e.g., Scheinin et al., Clin Exp Immunol 133(1): 38-43 (July 2003). Briefly, as shown in FIG. 51A, germ-free IL-10 KO animals were colonized with either DE935045.2 (DE39) or DE916091.1 (IgA+) bacterial compositions. As described herein, DE935045.2 was specifically constructed to exhibit various properties that would be useful in treating UC (e.g., capable of exerting anti-inflammatory activity). In contrast, DE916091.1, a composition integrated by IgA-binding strains isolated from UC patients, was designed to be pro-inflammatory and shown to induce TL8 (FIG. 23Q) and TLR4 (FIG. 23 I) expression. As a further control, some of the animals were colonized using stool from UC patients. Then, both body weight and fecal lipocalin levels were measured weekly. At 8 weeks post-colonization, the animals were sacrificed for further analysis.

As shown in FIGS. 51B and 51C, compared to the other groups, IL-10 KO mice colonized with DE935045.2 had improved body weight and no detectable levels of fecal lipocalin in their fecal samples, suggesting that these animals did not suffer from colitis. In contrast, animals colonized with DE916091.1 had reduced body weight (compared to other groups) and high levels of fecal lipocalin in their fecal samples. The DE916091.1 colonized animals also had significantly higher histological score (measurement of inflammatory damage), particularly within the cecum and the proximal colon, confirming the onset of colitis in these animals (FIGS. 51D-51F). In contrast, the intestinal tissues of animals colonized with DE935045.2 had no significant inflammatory damage. And, as shown in FIGS. 51G-51Q, animals colonized with DE935045.2 generally had a greater number of regulatory T cells (Tregs) (including colonic peripheral Tregs) with reduced number of effector CD4+ T cells (both Th17 and Th1 cells) and effector CD8+ T cells.

### Example 15: Analysis of the Effect of Designed Compositions in a DSS-Induced Colitis Animal Model

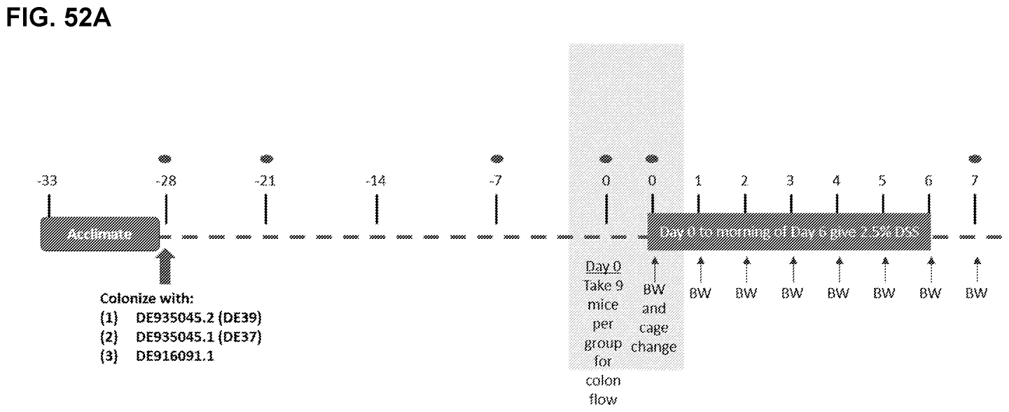

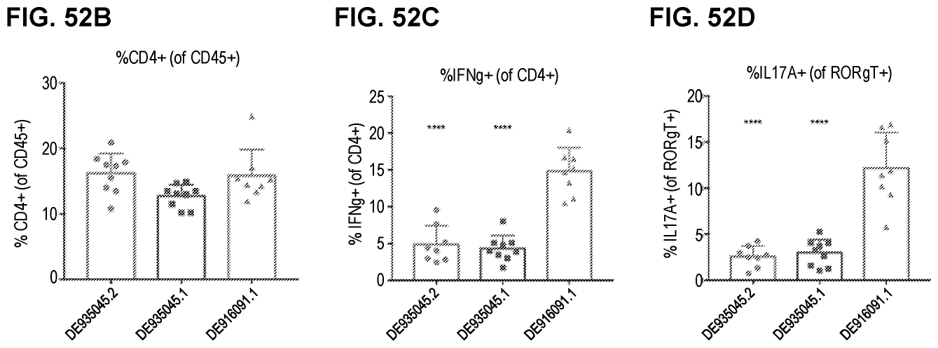

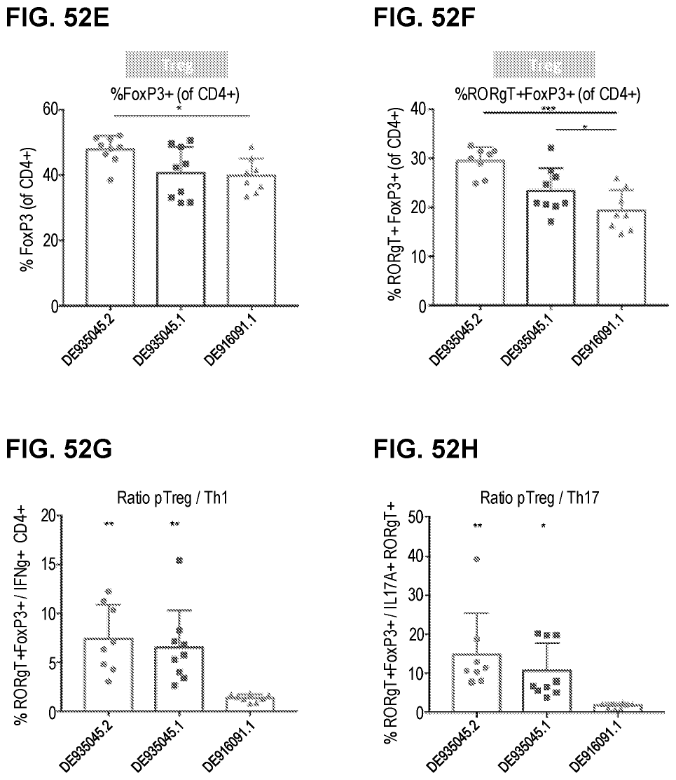

To confirm the results observed in Example 14, the therapeutic efficacy of the designed bacterial compositions was also assessed in a DSS-induced colitis animal model. Briefly, as shown in FIG. 52A, germ-free C57BL/6 mice were colonized with one of the following bacterial compositions: (i) DE935045.2 (DE39); (ii) DE935045.1 (DE37); or (iii) DE916091.1 (IgA+). Similar to DE935045.2, as described herein, DE935045.1 was also designed to exert minimal inflammatory activity. Then, at 4 weeks post colonization (i.e., day 0), some of the animals were sacrificed and sera, fecal pellets, colon, and cecal contents were collected for analysis. The remaining animals were given 2.5% DSS for six days in their drinking water to induce colitis. On day 7, the DSS-treated animals were also sacrificed for further analysis.

As shown in FIGS. 52B-52H, similar to the results observed with the IL-10 KO animal model, colonization of the animals with either the DE935045.2 or the DE935045.1 composition resulted in a significantly greater number of Tregs (including colonic peripheral Tregs) and decreased number of effector cells (e.g., Th1 and Th17 cells).

Collectively, the results described in Examples 14 and 15 confirm that bacterial compositions can be designed to exert specific properties (e.g., capable of inducing anti-inflammatory activity) and that such compositions can have therapeutic effects in vivo. The various properties associated with the DE935045.2 composition can be useful in treating inflammatory disorders, such as UC.

### Example 16: Analysis of the Effect of Designed Compositions on Anti-Tumor Responses to Immune Checkpoint Inhibitors

To assess whether the designed compositions disclosed herein could also be useful in treating cancers, a MC38 tumor model was used. Briefly, approximately three weeks prior to tumor inoculation, the DE286037.1 (DE1) composition was administered to the animals. DE1 was administered once, on week −3, at a dose of 10⁷ per strain; 3 weeks of colonization were allowed before tumor cell inoculation on day 0. Then, the MC38 tumor cells were transplanted into the animals (via subcutaneous administration). Anti-PD-1 antibody was administered to the animals at days 7, 10, 13, and 16 post tumor inoculation. Control animals received a control isotype antibody instead. Tumor volume was measured at days 8, 10, 13, 15, and 17 post tumor inoculation. At day 17, the animals were sacrificed and the percentages of tumor infiltrating CD8 T cells and regulatory T cells were determined in the tumors of the animals.

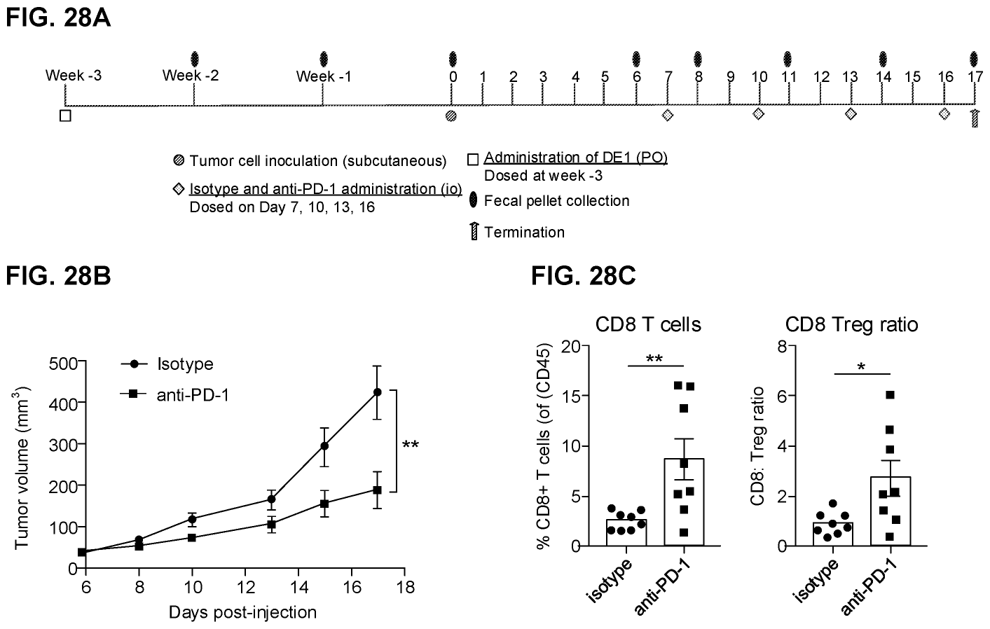

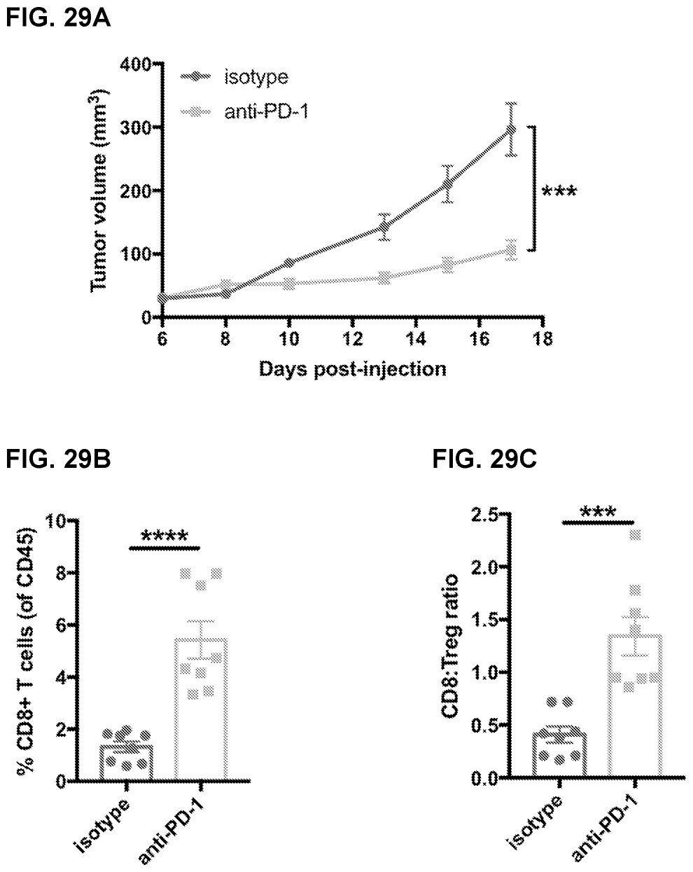

Suprisingly, as shown in FIG. 28B, animals that received both the DE1 (DE286037.1) composition and the anti-PD-1 antibody had greater reduction in tumor volume, compared to the control animals. The increased reduction in tumor volume was apparent as early as days 8-10 post tumor inoculation. The improved effect on tumor volume was associated with increased percentage of CD8 T cells in the tumors, resulting in increased CD8 T cell:Treg ratio (FIG. 28C). Similar results were observed with the DE2 (DE924221.1) composition in combination with anti-PD-1 antibody (FIGS. 29A, 29B, and 29C).

Next, to confirm the anti-tumor effects of the DE1 composition described above, a BP tumor model was used. The tumor was a melanoma derived from a Braf/pTEN knockout mouse. Briefly, the DE1 composition was administered to the animals, and then, approximately three weeks later, the animals were subcutaneously inoculated with the BP tumor cells. Anti-PD-L1 antibody or a control isotype antibody was administered to the animals at days 5, 8, 11, and 14 post tumor inoculation. Tumor volume was measured at days 8, 10, 12, and 15 post tumor inoculation. At day 15, animals were sacrificed, and the tumors analyzed.

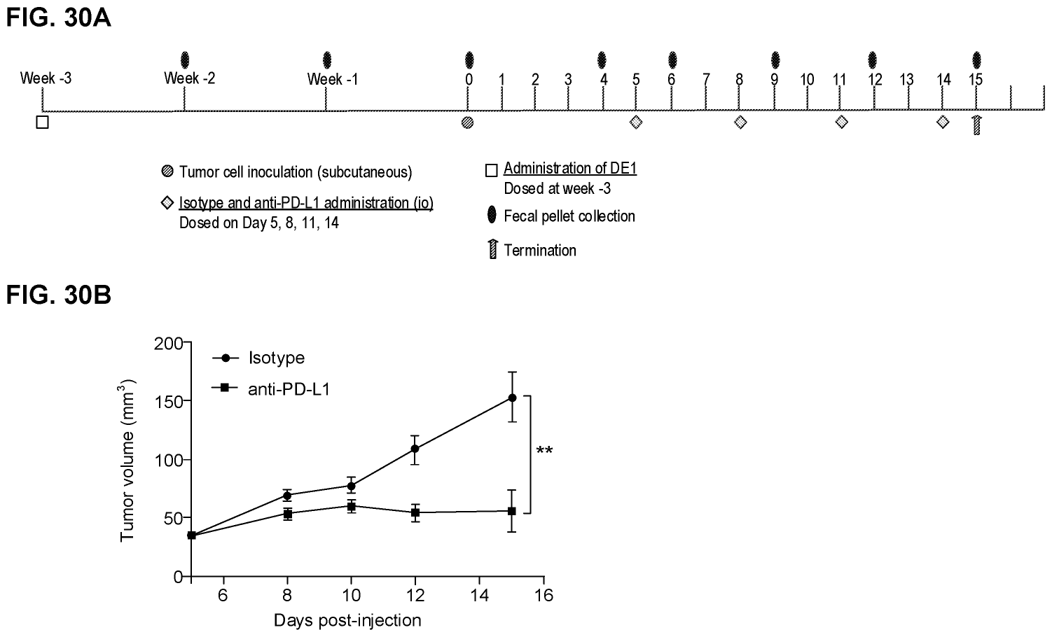

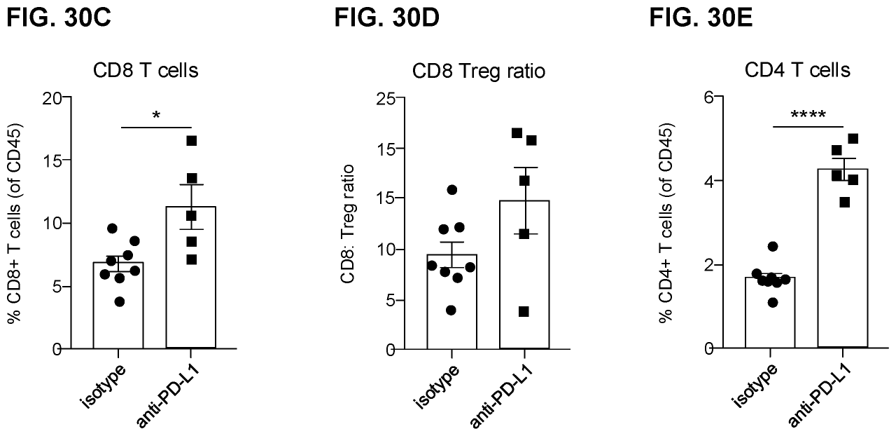

In agreement with the earlier data, animals that received the anti-PD-L1 antibody in combination with the DE286037.1 (DE1) composition had increased reduction in tumor volume, compared to the control group (FIG. 30B). Again, the animals treated with the combination of anti-PD-L1 antibody and DE1 had greater percentage of CD8 T cells in their tumors, resulting in increased CD8 T cell:Treg ratio (FIGS. 30C and 30D). The tumors also had greater percentage of CD4 T cells, compared to the control animals (FIG. 30E).

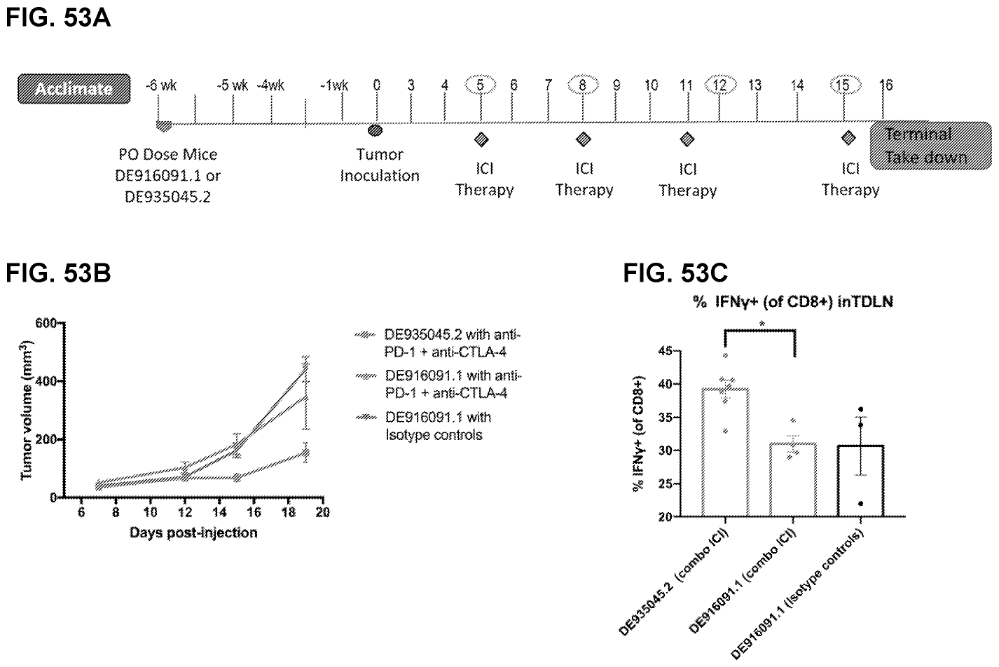

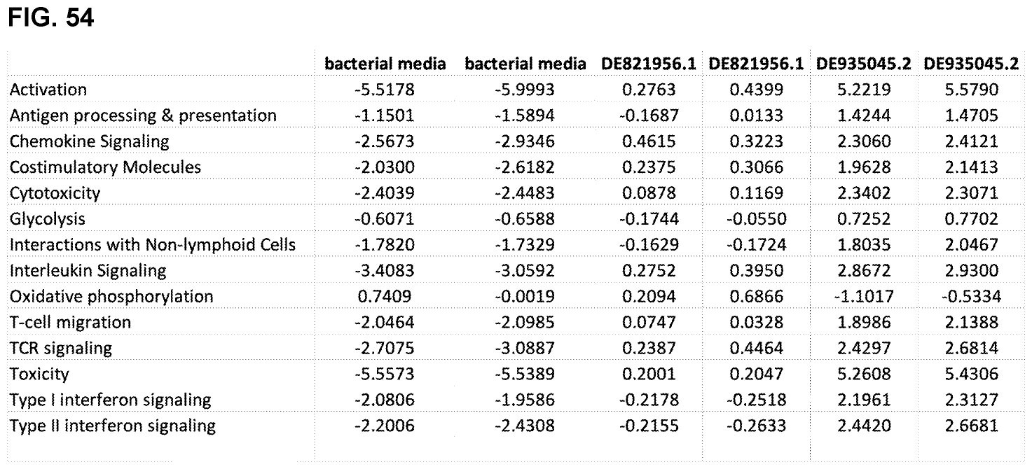

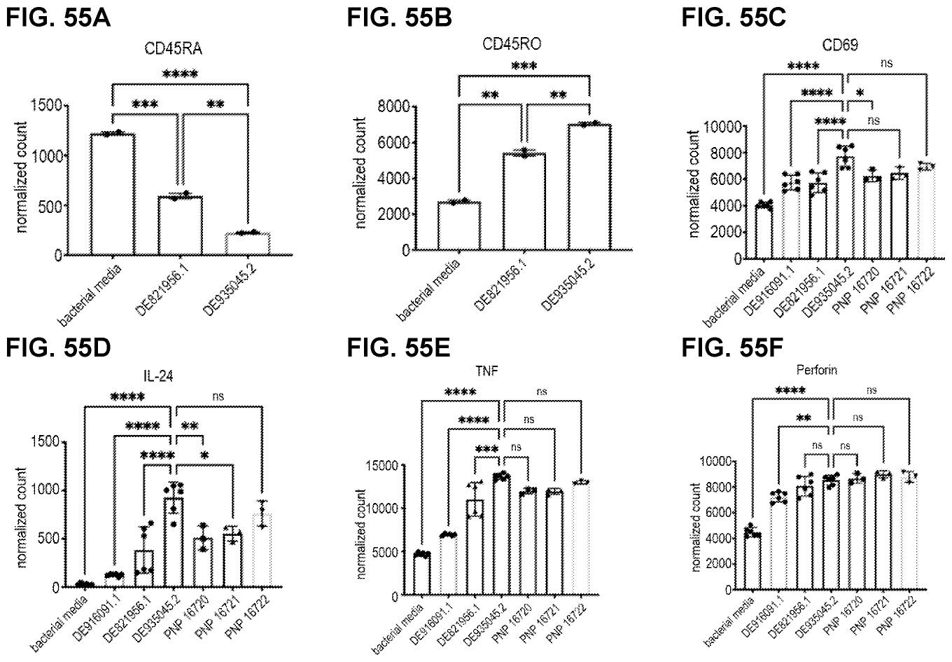

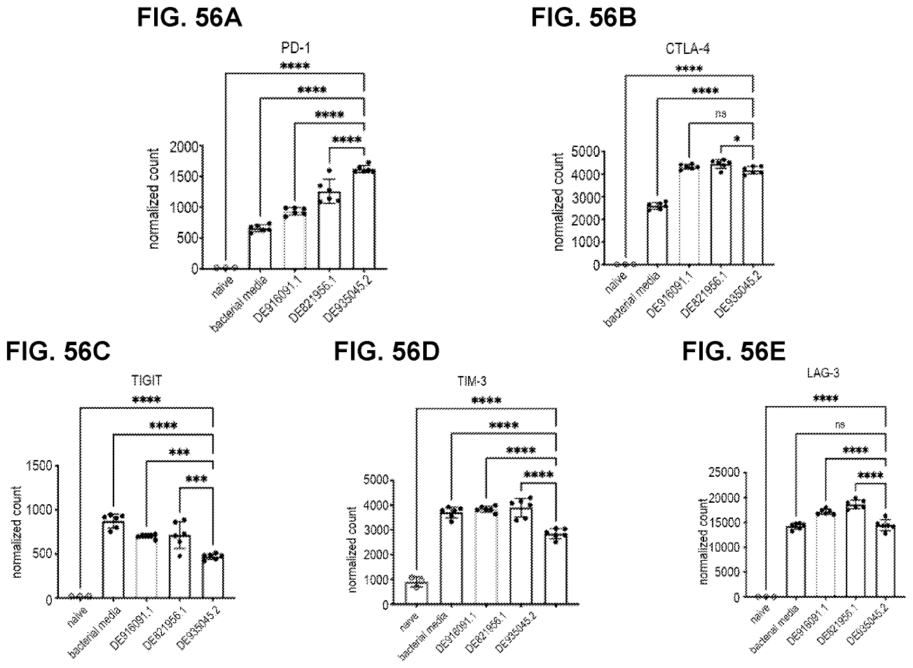

To further assess the anti-tumor effects described-above, the effect of the designed bacterial compositions on the efficacy of the combination of anti-PD-1 antibody and anti-CTLA-4 antibody treatment in the MC38 animal tumor model was next assessed. As shown in FIG. 53A, C57BL/6 germ-free mice were colonized with either DE935045.2 (DE39) or DE916091.1. Then, 6 weeks later, the mice were inoculated with the MC38 cells (5×10⁵) via subcutaneous administration. At weeks 5, 8, 11, and 15 post tumor inoculation, the mice were treated with either isotype antibodies or a combination of anti-PD1 and anti-CTLA-4 antibodies (200 μg/mL; i.p. administration). Then, at weeks 5, 8, 12, and 15, tumor volume was assessed in the animals.

As shown in FIG. 53B, animals that were earlier colonized with the pro-inflammatory DE916091.1 composition failed to control tumor growth regardless of whether the animals received the combination treatment or the isotype control antibodies. In contrast, mice that were colonized with the DE935045.2 (DE39) composition and then subsequently treated with a combination of the anti-PD-1 and anti-CTLA-4 antibodies exhibited reduced tumor size. In agreement with the tumor volume data, animals colonized with DE935045.2 (DE39) and then subsequently treated with the combination immune checkpoint inhibitory therapy had the greatest number of tumor-specific CD8+ T cells (as determined by IFN-γ expression) in the draining lymph nodes, suggesting an enhanced T cell immune response.

Collectively, the above data demonstrate that when administered in combination with an immune checkpoint inhibitor, some of the designed bacterial compositions disclosed herein (e.g., DE286037.1 (DE1), DE924221.1 (DE2), and DE935045.2 (DE39) compositions) can be useful in treating certain cancers. As described supra, and cancer immunotherapy generally aims to increase host pro-inflammatory responses targeting cancer cells. Therefore, it was not reasonably expected that a bacterial composition designed to have anti-inflammatory properties (e.g., DE1, DE2, and DE39) would be effective for enhancing anti-tumor response and that a pro-inflammatory composition (DE916091.1) would not. These results further highlights that a bacterial composition can be designed to target multiple immune pathways, and thereby, treat wide range of diseases, including both inflammatory diseases and cancers.

### Example 17: Effect of Designed Bacterials Compositions on Anti-Tumor Immunity

To further understand the anti-tumor effects described above in Example 16, the ability of the designed bacterial compositions to modulate human T cell function in vitro was also assessed. Briefly, primary human CD8 T cells were thawed at 37° C. for 24 hours and activated with beads conjugated to α-CD3 and α-CD28 antibodies at 37° C. for 2 days. The cells were then treated at 37° C. for 24 hours with supernatants from one of the following: (1) bacterial media, (2) DE916091.1, (3) DE821956.1 (DE9), (4) DE935045.2 (DE39), (5) HHSP #1, (6) HHSP #2, and (7) HHSP #3. Naïve T cells (i.e., not stimulated with either the α-CD3 and α-CD28 beads or the bacterial compositions) were used as control. Then, the expression of various genes associated with T cell function was assessed either by a Nanostring gene expression or multiplex panel. For IFN-γ, intracellular protein was also quantified by both flow cytometry and a Luminex assay.

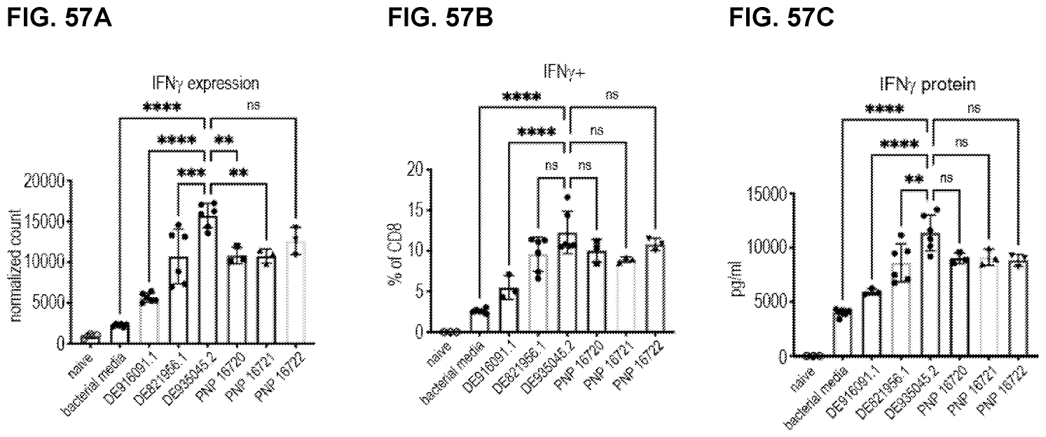

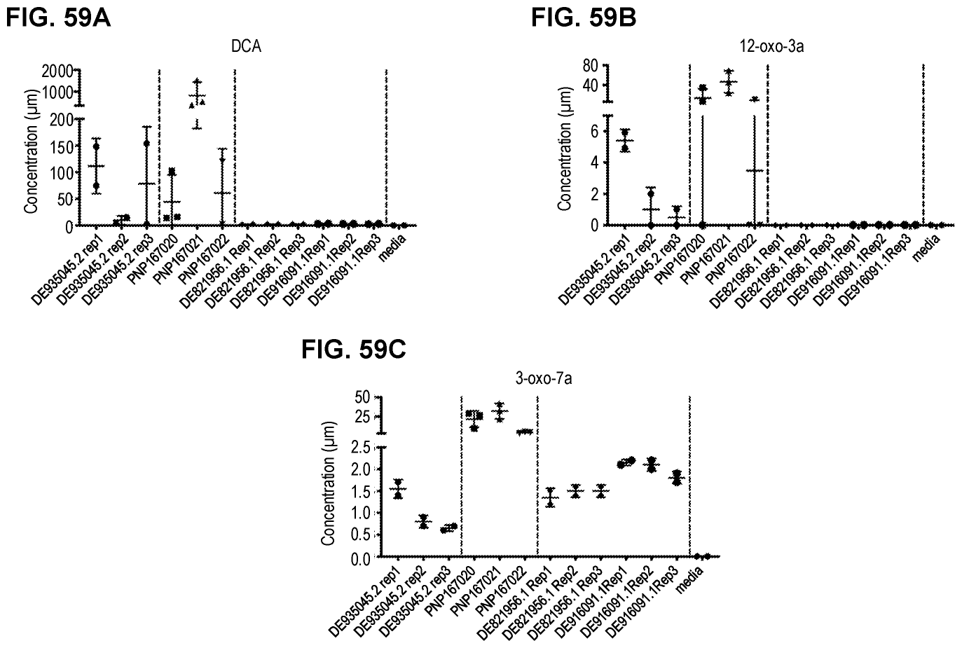

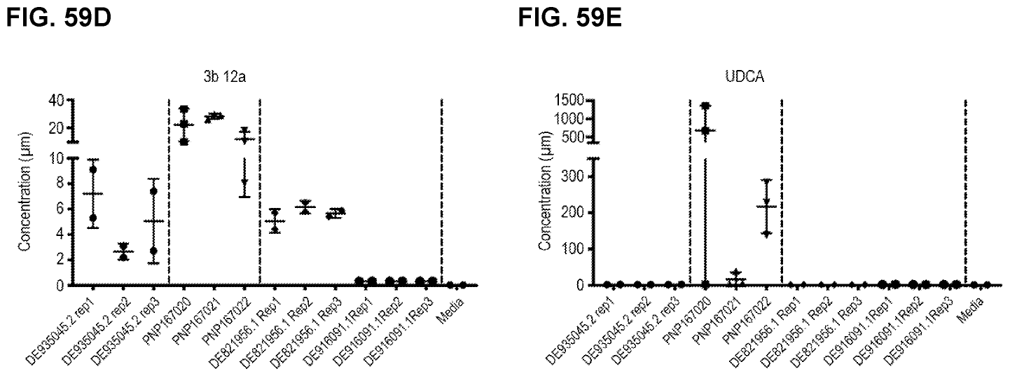

As shown in FIGS. 57A-57C, compared to the other treatment groups, T cells cultured with DE935045.2 composition (DE39) were much more activated, as evidenced by greater reduction in CD45RA gene (expressed on naïve T cells and downregulated upon activation) expression and much increased expression of CD45RO and CD69 genes (activation markers). In agreement with the enhanced activated phenotype, the T cells cultured with DE935045.2 (DE39) were also more functional, as they exhibited greater expression of several genes associated with cytotoxic T cell function (IL-24, TNF-α, perforin, and IFN-γ) (FIGS. 57D-57F; and FIGS. 60A-60C). The T cells were also associated with reduced expression of genes associated with exhaustion (e.g., TIGIT, TIM-3 and LAG-3) (FIGS. 59C-59E), further demonstrating the positive effect that the DE935045.2 composition (DE39) had on T cell activation.

Next, to assess whether the above positive effects can be associated with the enhanced anti-tumor effects observed in Example 16, an in vitro CD8+ T cell cytotoxicity assay was developed. Briefly, primary human CD8+ T cells were added to a 96-well plate and activated using beads conjugated to anti-CD28 and anti-CD3 antibodies. Then, the activated CD8+ T cells were co-cultured for 24 hours with HT29 cells (human colon cancer cell line) in the presence or absence of the bacterial compositions, and the ability of the activated CD8+ T cells to kill the HT29 cells was assessed by flow cytometry.

As shown in FIG. 61, compared to the other groups, activated T cells cultured in the presence of the DE935045.2 composition (DE39) exhibited increased ability to kill the tumor cells. In contrast, activated T cells incubated with supernatants of pro-inflammatory compositions, DE821956.1 (DE9) and DE916091.1 (IgA-plus), did not show enhanced tumor cell killing. Collectively, the above data further demonstrate that bacterial compositions can be designed to exhibit certain properties which can be useful in the treatment of various inflammatory disorders as well as for the treatment of cancer. This conclusion, which is also supported by the tumor model data in previous example, is unexpected as the prevailing literature points to induction of inflammation as the mechanism of action for microbiome effect in oncology. Not to be bound by any one theory, the above data also suggest that the bacterial species present in the DE935045.2 composition (DE39) may also help enhance CD8 T cell activity independent of the immune checkpoint inhibitors resulting in greater anti-tumor efficacy.

TABLE 4
Phenotypic Summary

| | | | | Anti-inflammatory | Pro-Inflammatory |
|---|---|---|---|---|---|
| | | | | in any C | in any C- |
| | HDAC | | Trp | source (>50% | source (>25% |
| | Inhibition in | HDAC | metabolite | reduction on | increase in |
| | any C source | cluster | positive in | IL8 compared | IL8 relative to |
| Species | (25/18% cutoff) | assignment | any C source | to TNFa control) | medium control) |
| Akkermansia muciniphila | 0 | 1 | 0 | 0 | 0 |
| Alistipes finepoldii | 0 | 1 | 1 | 0 | 0 |
| Alistipes onderdonkii | 1 | 1 | 1 | 1 | 0 |
| Alistipes shahii | 1 | 1 | 0 | 0 | 0 |
| Anaerotruncus colihominis | 0 | 1 | 0 | 0 | 0 |
| Anaerotruncus colihominis | 1 | 4 | 1 | 0 | 0 |
| Bacteroides caccae str. 1 | 0 | 1 | 0 | 0 | 0 |
| Bacteroides caccae str. 2 | 1 | 1 | 0 | 0 | 0 |
| Bacteroides caccae str. 3 | 0 | 1 | 0 | 0 | 0 |
| Bacteroides dorei | 0 | 1 | 0 | 0 | 0 |
| Bacteroides epperthii str. 1 | 0 | 1 | 1 | 0 | 0 |
| Bacteroides epperthii str. 2 | 0 | 1 | 1 | 0 | 0 |
| Bacteroides epperthii str. 3 | 1 | 3 | 1 | 0 | 0 |
| Bacteroides faecis | 1 | 3 | 1 | 0 | 0 |
| Bacteroides intestinalis | 1 | 3 | 1 | 0 | 0 |
| Bacteroides nordii | 0 | 1 | 0 | 0 | 0 |
| Bacteroides ovatus str. 1 | 1 | 3 | 1 | 0 | 0 |
| Bacteroides ovatus str. 2 | 0 | 1 | 0 | 1 | 0 |
| Bacteroides salyersiae str. 1 | 0 | 1 | 1 | 0 | 0 |
| Bacteroides salyersiae str. 2 | 1 | 3 | 1 | 0 | 0 |
| Bacteroides sp 1 1 30 | 1 | 0 | 0 | 0 | 0 |
| Bacteroides sp 1 1 6 | 1 | 1 | 1 | 0 | 0 |
| Bacteroides sp 2 1 22 | 0 | 1 | 1 | 0 | 0 |
| Bacteroides sp 3 1 23 str. 1 | 0 | 1 | 1 | 0 | 0 |
| Bacteroides sp 3 1 23 str. 2 | 0 | 1 | 1 | 0 | 0 |
| Bacteroides sp 4 1 36 | 1 | 2 | 1 | 0 | 0 |
| Bacteroides sp D20 str. 1 | 1 | 3 | 1 | 0 | 0 |
| Bacteroides sp D20 str. 2 | 1 | 0 | 1 | 1 | 0 |
| Bacteroides sp D22 | 1 | 3 | 1 | 0 | 0 |
| Bacteroides stercoris | 1 | 4 | 1 | 0 | 0 |
| Bacteroides uniformis str. 1 | 1 | 3 | 1 | 1 | 0 |
| Bacteroides uniformis str. 2 | 1 | 2 | 1 | 1 | 0 |
| Bacteroides vulpatus str. 1 | 1 | 2 | 0 | 0 | 0 |
| Bacteroides vulpatus str. 2 | 1 | 2 | 0 | 0 | 0 |
| Bifidobacterium adolescentis | 0 | 1 | 0 | 0 | 0 |
| Bifidobacterium catenulatum | 0 | 1 | 0 | n.d. | n.d. |
| Bifidobacterium lonpum str. 1 | 0 | 1 | 0 | 1 | 0 |
| Bifidobacterium lonpum str. 2 | 0 | 1 | 0 | 0 | 0 |
| Bifidobacterium lonpum str. 4 | 0 | 1 | 0 | n.d. | n.d. |
| Bifidobacterium lonpum str. 5 | 0 | 1 | 0 | 0 | 0 |
| Bifidobacterium | 0 | 1 | 0 | 0 | 0 |
| pseudocatenulatum str. 1 | | | | | |
| Bifidobacterium | 0 | 1 | 0 | n.d. | n.d. |
| pseudocatenulatum str. 2 | | | | | |
| Blautia coccoides str. 1 | 0 | 1 | 0 | 0 | 0 |
| Blautia coccoides str. 2 | 1 | 3 | 0 | 1 | 0 |
| Blautia glucerasei | 0 | 1 | 0 | 0 | 0 |
| Blautia producta str. 1 | 1 | 1 | 0 | 1 | 0 |
| Blautia producta str. 2 | 0 | 1 | 0 | 0 | 1 |
| Blautia producta str. 3 | 0 | 1 | 0 | n.d. | n.d. |
| Blautia producta str. 4 | 0 | 1 | 0 | 0 | 1 |
| Blautia producta str. 5 | 0 | 1 | 0 | 1 | 0 |
| Blautia producta str. 6 | 0 | 1 | 0 | 0 | 0 |
| Blautia schinkii str. 1 | 0 | 1 | 0 | 0 | 0 |
| Blautia schinkii str. 2 | 0 | 1 | 0 | 0 | 0 |
| Blautia sp M25 | 1 | 0 | 0 | 0 | 0 |
| Blautia wexlerae | 1 | 0 | 0 | 0 | 0 |
| Butyrivibrio crossotus | 1 | 4 | 0 | 1 | 0 |
| Clostridiaceae bacterium END 2 | 0 | 1 | 0 | 1 | 0 |
| Clostridiales sp SSC 2 | 1 | 4 | 0 | 1 | 0 |
| Clostridium aldenense | 1 | 1 | 1 | 1 | 0 |
| Clostridium asparagiforme | 0 | 1 | 1 | 0 | 0 |
| Clostridium bartlettii str. 1 | 1 | 2 | 1 | 1 | 0 |
| Clostridium bartlettii str. 2 | 0 | 1 | 1 | 1 | 0 |
| Clostridium bolteae str. 1 | 1 | 3 | 0 | n.d. | n.d. |
| Clostridium bolteae str. 2 | 1 | 1 | 0 | 0 | 1 |
| Clostridium bolteae str. 3 | 0 | 1 | 0 | 0 | 0 |
| Clostridium butyricum str. 1 | 1 | 4 | 0 | 0 | 1 |
| Clostridium butyricum str. 2 | 1 | 4 | 0 | 1 | 1 |
| Clostridium butyricum str. 2 | 1 | 4 | 0 | 1 | 1 |
| Clostridium citroniae | 1 | 4 | 1 | 1 | 1 |
| Clostridium clostridioforme | 1 | 6 | 0 | 0 | 1 |
| Clostridium disporicum | 1 | 0 | 0 | n.d. | n.d. |
| Clostridium ghonii | 1 | 4 | 1 | 1 | 0 |
| Clostridium glycolicum str. 1 | 1 | 2 | 1 | n.d. | n.d. |
| Clostridium glycolicum str. 2 | 1 | 2 | 0 | 0 | 0 |
| Clostridium hathewayi str. 1 | 0 | 1 | 0 | 0 | 0 |
| Clostridium hathewayi str. 2 | 0 | 1 | 0 | 0 | 0 |
| Clostridium hathewayi str. 3 | 0 | 1 | 0 | 0 | 0 |
| Clostridium hylemonae | 0 | 1 | 0 | n.d. | n.d. |
| Clostridium innocuum | 1 | 6 | 0 | n.d. | n.d. |
| Clostridium lactatifermentans | 0 | 1 | 0 | 0 | 0 |
| Clostridium lavalense | 0 | 1 | 1 | 0 | 0 |
| Clostridium leptum | 0 | 1 | 0 | 0 | 0 |
| Clostridium mayombei | 1 | 2 | 1 | 0 | 0 |
| Clostridium nexile | 0 | 1 | 0 | 0 | 0 |
| Clostridium oroticum str. 1 | 1 | 0 | 0 | 0 | 0 |
| Clostridium oroticum str. 2 | 1 | 0 | 0 | n.d. | n.d. |
| Clostridium scindens | 0 | 1 | 0 | 0 | 0 |
| Clostridium sp 7 2 43FAA | 1 | 5 | 0 | 0 | 1 |
| Clostridium sp NML 04A032 | 1 | 4 | 1 | 1 | 0 |
| Clostridium spiroforme str. 1 | 0 | 1 | 0 | 0 | 0 |
| Clostridium spiroforme str. 2 | 0 | 1 | 0 | 0 | 0 |
| Clostridium sporopenes str. 1 | 1 | 4 | 1 | 1 | 0 |
| Clostridium sporopenes str. 2 | 1 | 4 | 1 | 1 | 0 |
| Clostridium straminisolvens | 0 | 1 | 0 | 0 | 0 |
| Clostridium subterminale | 1 | 4 | 1 | 1 | 0 |
| Clostridium symbiosum str. 1 | 1 | 4 | 0 | 1 | 0 |
| Clostridium symbiosum str. 2 | 1 | 4 | 0 | 0 | 0 |
| Clostridium tertium | 1 | 5 | 0 | 0 | 1 |
| Clostridium tyrobutyricum | 1 | 6 | 0 | 1 | 1 |
| Clostridium viride str. 1 | 1 | 4 | 0 | 1 | 0 |
| Clostridium viride str. 2 | 1 | 4 | 0 | 1 | 0 |
| Coprobacillus sp D7 str. 1 | 0 | 1 | 0 | 0 | 0 |
| Coprobacillus sp D7 str. 2 | 0 | 1 | 0 | 0 | 0 |
| Coprococcus comes | 1 | 4 | 0 | 1 | 0 |
| Coprococcus eutactus str. 1 | 1 | 1 | 0 | 0 | 0 |
| Coprococcus eutactus str. 2 | 1 | 6 | 0 | 1 | 0 |
| Coriobacteriaceae sp 7 10 1 b | 0 | 1 | 0 | 0 | 0 |
| Dorea formicipenerans str. 1 | 0 | 1 | 0 | 0 | 0 |
| Dorea formicipenerans str. 2 | 0 | 1 | 0 | 0 | 0 |
| Dorea formicipenerans str. 3 | 0 | 1 | 0 | 0 | 0 |
| Dorea formicipenerans str. 4 | 0 | 1 | 0 | 0 | 0 |
| Dorea lonpicatena str. 1 | 0 | 1 | 0 | 0 | 0 |
| Dorea lonpicatena str. 2 | 0 | 1 | 0 | 0 | 0 |
| Dorea lonpicatena str. 3 | 0 | 1 | 0 | 0 | 0 |
| Epperthella lenta str. 1 | 0 | 1 | 0 | 0 | 0 |
| Epperthella lenta str. 2 | 0 | 1 | 0 | 0 | 0 |
| Epperthella lenta str. 3 | 0 | 1 | 0 | 0 | 0 |
| Epperthella sp 1 3 56FAA | 0 | 1 | 0 | 0 | 0 |
| Erysipelotrichaceae bacterium | 1 | 4 | 0 | 1 | 0 |
| 3 1 53 str. 1 | | | | | |
| Erysipelotrichaceae bacterium | 0 | 1 | 0 | 0 | 0 |
| 3 1 53 str. 2 | | | | | |
| Erysipelotrichaceae bacterium | 1 | 6 | 0 | 0 | 0 |
| 5 2 54FAA | | | | | |
| Eubacterium contortum str. 1 | 1 | 0 | 0 | 0 | 0 |
| Eubacterium contortum str. 2 | 1 | 0 | 0 | n.d. | n.d. |
| Eubacterium desmolans | 1 | 5 | 0 | 1 | 0 |
| Eubacterium dolichum | 1 | 6 | 0 | 0 | 0 |
| Eubacterium hallii | 1 | 0 | 0 | 0 | 0 |
| Eubacterium limosum | 1 | 6 | 0 | 1 | 0 |
| Eubacterium rectale str. 1 | 1 | 5 | 0 | 0 | 1 |
| Eubacterium rectale str. 2 | 1 | 5 | 0 | 0 | 1 |
| Eubacterium siraeum | 0 | 1 | 0 | 0 | 0 |
| Eubacterium sp WAL 14571 | 1 | 4 | 0 | 1 | 0 |
| str. 1 | | | | | |
| Eubacterium sp WAL 14571 | 1 | 4 | 0 | 1 | 0 |
| str. 1 | | | | | |
| Eubacterium tenue | 1 | 3 | 1 | 0 | 0 |
| Eubacterium ventriosum | 0 | 1 | 0 | 0 | 0 |
| Faecalibacterium prausnitzii | 1 | 6 | 0 | 0 | 0 |
| str. 1 | | | | | |
| Faecalibacterium prausnitzii | 1 | 3 | 0 | 0 | 0 |
| str. 2 | | | | | |
| Faecalibacterium prausnitzii | 1 | 3 | 0 | 1 | 0 |
| str. 3 | | | | | |
| Faecalibacterium prausnitzii | 1 | 3 | 0 | 0 | 0 |
| str. 4 | | | | | |
| Faecalibacterium prausnitzii | 0 | 1 | 0 | 0 | 0 |
| str. 5 | | | | | |
| Faecalibacterium prausnitzii | 1 | 6 | 0 | 0 | 1 |
| str. 6 | | | | | |
| Faecalibacterium prausnitzii | 1 | 1 | 0 | 0 | 0 |
| str. 7 | | | | | |
| Flavonifractor plautii str. 1 | 1 | 4 | 1 | 1 | 1 |
| Flavonifractor plautii str. 2 | 1 | 4 | 1 | n.d. | n.d. |
| Gemmiger formicilis str. 1 | 1 | 6 | 0 | 1 | 0 |
| Gemmiger formicilis str. 2 | 1 | 6 | 0 | 0 | 0 |
| Gemmiger formicilis str. 3 | 1 | 6 | 0 | 1 | 0 |
| Hydrogenoanaerobacterium | 0 | 1 | 0 | 0 | 0 |
| saccharovorans | | | | | |
| Lachnospira pectinoschiza | 0 | 1 | 0 | 0 | 0 |
| Lachnospiraceae bacterium 1 | 0 | 1 | 0 | 0 | 0 |
| 4 56FAA | | | | | |
| Lachnospiraceae bacterium 2 | 1 | 0 | 0 | 1 | 0 |
| 1 58FAA | | | | | |
| Lachnospiraceae bacterium 3 | 1 | 4 | 0 | 1 | 0 |
| 1 57FAA str. 1 | | | | | |
| Lachnospiraceae bacterium 3 | 1 | 4 | 0 | 1 | 0 |
| 1 57FAA str. 2 | | | | | |
| Lachnospiraceae bacterium 5 | 0 | 1 | 0 | 0 | 1 |
| 1 57FAA str. 1 | | | | | |
| Lachnospiraceae bacterium 5 | 0 | 1 | 0 | 0 | 1 |
| 1 57FAA str. 2 | | | | | |
| Lachnospiraceae bacterium 5 | 0 | 1 | 0 | 0 | 0 |
| 1 57FAA str. 3 | | | | | |
| Lachnospiraceae bacterium 5 | 0 | 1 | 0 | 0 | 1 |
| 1 57FAA str. 4 | | | | | |
| Lachnospiraceae bacterium 5 | 0 | 1 | 0 | 0 | 1 |
| 1 57FAA str. 5 | | | | | |
| Lachnospiraceae bacterium 6 | 0 | 1 | 0 | 0 | 0 |
| 1 63FAA | | | | | |
| Lachnospiraceae bacterium | 0 | 1 | 0 | 0 | 0 |
| oral taxon F15 str. 1 | | | | | |
| Lachnospiraceae bacterium | 1 | 0 | 0 | 0 | 0 |
| oral taxon F15 str. 2 | | | | | |
| Lachnospiraceae sp 10972 | 1 | 0 | 0 | n.d. | n.d. |
| Lachnospiraceae sp 11041 | 0 | 1 | 0 | n.d. | n.d. |
| Lactobacillus gasseri | 0 | 1 | 0 | 0 | 0 |
| Lactonifactor longoviformis | 0 | 1 | 0 | 0 | 0 |
| Odoribacter splanchnicus | 1 | 4 | 1 | 1 | 0 |
| Oscillibacter valericigenes | 1 | 4 | 0 | 1 | 0 |
| Parabacteroides distasonis | 1 | 3 | 0 | 1 | 0 |
| Roseburia faecalis | 1 | 1 | 0 | 0 | 0 |
| Roseburia hominis str. 1 | 1 | 5 | 0 | 1 | 1 |
| Roseburia hominis str. 2 | 1 | 6 | 0 | 1 | 0 |
| Roseburia intestinalis str. 1 | 1 | 5 | 0 | 0 | 0 |
| Roseburia intestinalis str. 2 | 1 | 5 | 0 | 0 | 1 |
| Roseburia intestinalis str. 3 | 1 | 5 | 0 | 1 | 1 |
| Roseburia intestinalis str. 4 | 1 | 5 | 0 | 1 | 0 |
| Roseburia inulinivorans | 1 | 1 | 0 | 0 | 1 |
| Ruminococcaceae bacterium D16 | 1 | 4 | 0 | 0 | 0 |
| Ruminococcus albus | 0 | 1 | 0 | 0 | 0 |
| Ruminococcus bromii str. 1 | 0 | 1 | 0 | 0 | 0 |
| Ruminococcus bromii str. 2 | 0 | 1 | 0 | 0 | 0 |
| Ruminococcus bromii str. 3 | 0 | 1 | 0 | 0 | 0 |
| Ruminococcus gnavus | 0 | 1 | 1 | 0 | 0 |
| Ruminococcus hansenii | 0 | 1 | 0 | 0 | 0 |
| Ruminococcus lactaris str. 1 | 0 | 1 | 0 | 0 | 0 |
| Ruminococcus lactaris str. 2 | 0 | 1 | 0 | 1 | 0 |
| Ruminococcus obeum str. 1 | 1 | 0 | 0 | 0 | 0 |
| Ruminococcus obeum str. 2 | 1 | 0 | 0 | 1 | 0 |
| Ruminococcus obeum str. 3 | 1 | 0 | 0 | 1 | 0 |
| Ruminococcus obeum str. 4 | 1 | 0 | 0 | 0 | 0 |
| Ruminococcus obeum str. 5 | 1 | 0 | 0 | 1 | 0 |
| Ruminococcus sp 5 1 39BFAA | 1 | 0 | 0 | 1 | 0 |
| Ruminococcus sp K-1 | 1 | 0 | 0 | 0 | 0 |
| Ruminococcus torques str. 1 | 0 | 1 | 0 | 0 | 0 |
| Ruminococcus torques str. 2 | 0 | 1 | 0 | 0 | 0 |
| Subdoligranulum variabile | 1 | 6 | 0 | 1 | 0 |
| Turicibacter sanguinis str. 1 | 0 | 1 | 0 | n.d. | n.d. |
| Turicibacter sanguinis str. 2 | 0 | 1 | 0 | 0 | 0 |

TABLE 5
Engraftment Summary

| | | Long-Term |
|---|---|---|
| | SEQ ID NO | Engrafter (LTE) |
| | for 16S | or Transient |
| Species | Sequence | Engrafter (TE) |
| Acetivibrio unclassified | 258 | LTE |
| Anaerostipes hadrus | 363 | LTE |
| Anaerostipes unclassified | 229 | LTE |
| Anaerotruncus colihominis str. 1 | 230 | TE |
| Anaerotruncus colihominis str. 2 | 232 | TE |
| Anaerotruncus unclassified | 231 | TE |
| Blautia hydrogenotrophica | 238 | LTE |
| Blautia obeum str. 1 | 389 | LTE |
| Blautia obeum str. 2 | 390 | LTE |
| Blautia producta | 239 | TE |
| Blautia unclassified str. 1 | 233 | LTE |
| Blautia unclassified str. 2 | 236 | LTE |
| Blautia unclassified str. 3 | 391 | LTE |
| Blautia wexlerae str. 1 | 240 | LTE |
| Blautia wexlerae str. 2 | 241 | LTE |
| Blautia wexlerae str. 3 | 242 | LTE |
| Blautia wexlerae str. 4 | 243 | LTE |
| Blautia wexlerae str. 5 | 244 | LTE |
| Blautia wexlerae str. 6 | 245 | LTE |
| Blautia wexlerae str. 7 | 246 | LTE |
| Blautia wexlerae str. 8 | 247 | LTE |
| Butyricicoccus unclassified str. 1 | 251 | LTE |
| Butyricicoccus unclassified str. 2 | 259 | LTE |
| Butyricicoccus unclassified str. 3 | 313 | TE |
| Clostridiales unclassified str. 1 | 234 | LTE |
| Clostridiales unclassified str. 2 | 235 | LTE |
| Clostridiales unclassified str. 3 | 302 | TE |
| Clostridium aldenense | 263 | TE |
| Clostridium bolteae str. 1 | 270 | TE |
| Clostridium bolteae str. 2 | 272 | TE |
| Clostridium bolteae str. 3 | 273 | TE |
| Clostridium bolteae str. 4 | 274 | TE |
| Clostridium citroniae | 271 | TE |
| Clostridium innocuum str. 1 | 278 | TE |
| Clostridium innocuum str. 2 | 279 | TE |
| Clostridium innocuum str. 3 | 280 | TE |
| Clostridium innocuum str. 4 | 281 | TE |
| Clostridium innocuum str. 5 | 282 | TE |
| Clostridium innocuum str. 6 | 308 | TE |
| Clostridium innocuum str. 7 | 310 | TE |
| Clostridium innocuum str. 8 | 311 | TE |
| Clostridium innocuum str. 9 | 312 | TE |
| Clostridium lavalense str. 1 | 264 | TE |
| Clostridium lavalense str. 2 | 283 | TE |
| Clostridium leptum str. 1 | 284 | LTE |
| Clostridium leptum str. 2 | 285 | LTE |
| Clostridium paraputrificum | 286 | TE |
| Clostridium perfringens | 287 | TE |
| Clostridium saudiense | 275 | LTE |
| Clostridium scindens | 362 | TE |
| Clostridium subterminale | 290 | TE |
| Clostridium symbiosum | 291 | TE |
| Clostridium unclassified | 237 | LTE |
| Coprobacillus unclassified | 250 | LTE |
| Coprococcus comes | 293 | LTE |
| Coprococcus unclassified | 292 | LTE |
| Dielma fastidiosa | 248 | LTE |
| Dorea formicigenerans str. 1 | 294 | LTE |
| Dorea formicigenerans str. 2 | 295 | LTE |
| Dorea formicigenerans str. 3 | 296 | LTE |
| Dorea formicigenerans str. 4 | 297 | LTE |
| Dorea formicigenerans str. 5 | 298 | LTE |
| Dorea formicigenerans str. 6 | 299 | LTE |
| Dorea longicatena str. 1 | 300 | LTE |
| Dorea longicatena str. 2 | 301 | LTE |
| Eisenbergiella tayi str. 1 | 359 | LTE |
| Eisenbergiella tayi str. 2 | 360 | LTE |
| Eisenbergiella tayi str. 3 | 361 | LTE |
| Erysipelatoclostridium ramosum | 288 | TE |
| Eubacterium eligens str. 1 | 318 | LTE |
| Eubacterium eligens str. 2 | 319 | LTE |
| Eubacterium eligens str. 3 | 320 | LTE |
| Eubacterium eligens str. 4 | 321 | LTE |
| Eubacterium eligens str. 5 | 322 | LTE |
| Eubacterium hallii | 323 | LTE |
| Eubacterium rectale str. 1 | 325 | LTE |
| Eubacterium rectale str. 2 | 326 | LTE |
| Eubacterium rectale str. 3 | 327 | LTE |
| Eubacterium rectale str. 4 | 328 | LTE |
| Eubacterium rectale str. 5 | 329 | LTE |
| Eubacterium siraeum str. 1 | 330 | LTE |
| Eubacterium siraeum str. 2 | 331 | LTE |
| Eubacterium siraeum str. 3 | 332 | LTE |
| Eubacterium siraeum str. 4 | 333 | LTE |
| Eubacterium ventriosum | 339 | LTE |
| Faecalibacterium prausnitzii str. 1 | 340 | LTE |
| Faecalibacterium prausnitzii str. 2 | 341 | LTE |
| Faecalibacterium prausnitzii str. 3 | 342 | LTE |
| Faecalibacterium prausnitzii str. 4 | 343 | LTE |
| Faecalibacterium prausnitzii str. 5 | 344 | LTE |
| Faecalibacterium prausnitzii str. 6 | 345 | LTE |
| Faecalicatena contorta str. 1 | 314 | TE |
| Faecalicatena contorta str. 2 | 315 | TE |
| Faecalicatena contorta str. 3 | 316 | TE |
| Faecalicatena contorta str. 4 | 317 | TE |
| Firmicutes unclassified str. 1 | 303 | TE |
| Firmicutes unclassified str. 2 | 304 | TE |
| Firmicutes unclassified str. 3 | 305 | TE |
| Firmicutes unclassified str. 4 | 306 | TE |
| Firmicutes unclassified str. 5 | 307 | TE |
| Firmicutes unclassified str. 6 | 309 | TE |
| Flavonifractor plautii str. 1 | 348 | LTE |
| Flavonifractor plautii str. 2 | 364 | LTE |
| Fusicatenibacter saccharivorans | 349 | LTE |
| Gemmiger formicilis | 350 | LTE |
| Holdemania filiformis | 352 | LTE |
| Hungatella effluvii str. 1 | 276 | TE |
| Hungatella effluvii str. 2 | 277 | TE |
| Intestinibacter bartlettii str. 1 | 265 | LTE |
| Intestinibacter bartlettii str. 2 | 266 | LTE |
| Intestinibacter bartlettii str. 3 | 267 | LTE |
| Intestinibacter bartlettii str. 4 | 268 | LTE |
| Intestinibacter bartlettii str. 5 | 269 | LTE |
| Intestinimonas butyriciproducens | 353 | LTE |
| Lachnoclostridium pacaense | 249 | TE |
| Lachnospiraceae unclassified str. 1 | 228 | LTE |
| Lachnospiraceae unclassified str. 2 | 252 | LTE |
| Lachnospiraceae unclassified str. 3 | 253 | LTE |
| Lachnospiraceae unclassified str. 4 | 254 | LTE |
| Lachnospiraceae unclassified str. 5 | 255 | LTE |
| Lachnospiraceae unclassified str. 6 | 256 | LTE |
| Lachnospiraceae unclassified str. 7 | 260 | LTE |
| Lachnospiraceae unclassified str. 8 | 289 | LTE |
| Lachnospiraceae unclassified str. 9 | 354 | LTE |
| Lachnospiraceae unclassified str. 10 | 381 | LTE |
| Lactobacillus rogosae | 355 | LTE |
| Lactonifactor unclassified | 366 | TE |
| Longicatena caecimuris str. 1 | 334 | LTE |
| Longicatena caecimuris str. 2 | 335 | LTE |
| Longicatena caecimuris str. 3 | 336 | LTE |
| Longicatena caecimuris str. 4 | 337 | LTE |
| Longicatena caecimuris str. 5 | 338 | LTE |
| Oscillibacter unclassified | 367 | LTE |
| Robinsoniella unclassified | 257 | LTE |
| Roseburia faecis | 368 | LTE |
| Roseburia hominis str. 1 | 369 | LTE |
| Roseburia hominis str. 2 | 370 | LTE |
| Roseburia hominis str. 3 | 371 | LTE |
| Roseburia hominis str. 4 | 372 | LTE |
| Roseburia inulinivorans | 374 | LTE |
| Roseburia unclassified str. 1 | 324 | LTE |
| Roseburia unclassified str. 2 | 373 | LTE |
| Roseburia unclassified str. 3 | 375 | LTE |
| Ruminococcaceae unclassified str. 1 | 261 | LTE |
| Ruminococcaceae unclassified str. 2 | 262 | TE |
| Ruminococcaceae unclassified str. 3 | 346 | LTE |
| Ruminococcaceae unclassified str. 4 | 347 | LTE |
| Ruminococcaceae unclassified str. 5 | 376 | LTE |
| Ruminococcus bromii | 383 | LTE |
| Ruminococcus gnavus str. 1 | 357 | LTE |
| Ruminococcus gnavus str. 2 | 384 | LTE |
| Ruminococcus gnavus str. 3 | 385 | LTE |
| Ruminococcus gnavus str. 4 | 386 | LTE |
| Ruminococcus gnavus str. 5 | 387 | LTE |
| Ruminococcus gnavus str. 6 | 388 | LTE |
| Ruminococcus torques str. 1 | 356 | LTE |
| Ruminococcus torques str. 2 | 358 | LTE |
| Ruminococcus torques str. 3 | 365 | LTE |
| Ruminococcus torques str. 4 | 392 | LTE |
| Ruminococcus torques str. 5 | 393 | LTE |
| Ruminococcus torques str. 6 | 394 | LTE |
| Ruminococcus torques str. 7 | 395 | LTE |
| Ruminococcus torques str. 8 | 396 | LTE |
| Ruminococcus torques str. 9 | 397 | LTE |
| Ruminococcus unclassified str. 1 | 377 | TE |
| Ruminococcus unclassified str. 2 | 378 | TE |
| Ruminococcus unclassified str. 3 | 379 | TE |
| Ruminococcus unclassified str. 4 | 380 | TE |
| Ruminococcus unclassified str. 5 | 382 | LTE |
| Ruthenibacterium lactatiformans | 398 | TE |
| Subdoligranulum unclassified | 351 | LTE |

TABLE 6
Designed Bacterial Compositions (DE1 and DE3-DE12) Properties

| | DE | | | | | |
|---|---|---|---|---|---|---|
| | DE286037.1 | DE984662.1 | DE002165.1 | DE464167.1 | DE522292.1 | DE247030.1 |
| | (DE1) | (DE3) | (DE4) | (DE5) | (DE6) | (DE7) |
| | Alias | | | | | |
| | | | de1 | | | |
| | | | core | de1 | | |
| | | de1 | plus | pheno | de | de |
| | de1 | core | pstrep | core | 2873mer1 | 2873mer2 |
| HDAC cluster 0 | 2 | 1 | 1 | 1 | 1 | 1 |
| HDAC cluster 1 | 4 | 0 | 1 | 0 | 3 | 4 |
| HDAC cluster 2 | 1 | 0 | 0 | 0 | 0 | 0 |
| HDAC cluster 3 | 1 | 0 | 0 | 0 | 0 | 0 |
| HDAC cluster 4 | 5 | 2 | 2 | 3 | 8 | 5 |
| HDAC cluster 5 | 0 | 0 | 0 | 0 | 0 | 0 |
| HDAC cluster 6 | 1 | 0 | 0 | 0 | 0 | 2 |
| HDAC inhibition | 11 | 3 | 4 | 4 | 10 | 9 |
| Propanoic acid | 5 | 0 | 1 | 3 | 2 | 3 |
| Butanoic acid | 5 | 1 | 2 | 2 | 6 | 4 |
| Pentanoic acid | 1 | 1 | 2 | 0 | 2 | 1 |
| Hexanoic acid | 2 | 1 | 1 | 0 | 3 | 2 |
| Indole | 1 | 1 | 1 | 3 | 1 | 1 |
| 3-methylindole | 2 | 0 | 1 | 1 | 4 | 3 |
| 3-indoleacrylic acid | 0 | 0 | 1 | 0 | 0 | 0 |
| BSH gCA | 13 | 2 | 3 | 3 | 11 | 11 |
| BSH tCA | 11 | 1 | 1 | 2 | 10 | 10 |
| BSH gCDCA | 9 | 1 | 1 | 2 | 8 | 8 |
| BSH tCDCA | 10 | 1 | 1 | 2 | 9 | 9 |
| 7aD CA | 6 | 2 | 2 | 1 | 5 | 5 |
| 7aD CDCA | 0 | 0 | 0 | 0 | 0 | 0 |
| 7bHSDH UDCA | 1 | 0 | 1 | 0 | 0 | 0 |
| Catalase | 0 | 0 | 0 | 0 | 1 | 0 |
| a-L-Fucosidase | 3 | 1 | 1 | 0 | 2 | 2 |
| IL8 Inflammatory | 0 | 0 | 0 | 0 | 0 | 0 |
| LTE | 1 | 0 | 0 | 3 | 1 | 4 |
| TE | 7 | 2 | 2 | 1 | 8 | 5 |
| Remission Associated | 2 | 0 | 0 | 0 | 2 | 1 |
| NonRemission Associated | 1 | 1 | 1 | 1 | 1 | 1 |

| | DE | | | |
|---|---|---|---|---|
| | DE349441.1 | DE698478.1 | DE559846.1 | DE405816.1 |
| | (DE8) | (DE10) | (DE11) | (DE12) |
| | Alias | | | |
| | | | | de |
| | de | new | de1 | 2873mer1 |
| | 2876mer | core | modNewCore | modNewCore |
| HDAC cluster 0 | 1 | 1 | 1 | 1 |
| HDAC cluster 1 | 2 | 2 | 4 | 4 |
| HDAC cluster 2 | 0 | 0 | 0 | 0 |
| HDAC cluster 3 | 0 | 0 | 1 | 0 |
| HDAC cluster 4 | 7 | 1 | 5 | 8 |
| HDAC cluster 5 | 0 | 0 | 0 | 0 |
| HDAC cluster 6 | 2 | 0 | 1 | 0 |
| HDAC inhibition | 11 | 3 | 10 | 11 |
| Propanoic acid | 2 | 1 | 3 | 2 |
| Butanoic acid | 6 | 1 | 4 | 6 |
| Pentanoic acid | 2 | 0 | 1 | 2 |
| Hexanoic acid | 2 | 0 | 1 | 2 |
| Indole | 1 | 2 | 3 | 3 |
| 3-methylindole | 5 | 1 | 2 | 4 |
| 3-indoleacrylic acid | 0 | 0 | 0 | 0 |
| BSH gCA | 11 | 4 | 12 | 13 |
| BSH tCA | 10 | 3 | 9 | 11 |
| BSH gCDCA | 9 | 3 | 7 | 9 |
| BSH tCDCA | 9 | 3 | 8 | 10 |
| 7aD CA | 4 | 1 | 4 | 4 |
| 7aD CDCA | 0 | 1 | 1 | 1 |
| 7bHSDH UDCA | 0 | 0 | 0 | 0 |
| Catalase | 1 | 1 | 1 | 2 |
| a-L-Fucosidase | 1 | 1 | 1 | 1 |
| IL8 Inflammatory | 0 | 0 | 0 | 0 |
| LTE | 4 | 1 | 2 | 2 |
| TE | 7 | 3 | 10 | 11 |
| Remission Associated | 3 | 0 | 2 | 2 |
| NonRemission Associated | 1 | 1 | 2 | 2 |

TABLE 7
Designed Bacterial Compositions (DE13-DE19 and DE21-DE23) Properties

| | DE | | | | |
|---|---|---|---|---|---|
| | DE056280.1 | DE390874.1 | DE299561.1 | DE504874.1 | DE124702.1 |
| | (DE13) | (DE14) | (DE15) | (DE16) | (DE18) |
| | Alias | | | | |
| | | | | | t1 eff |
| | | | | t1 eff | s287 |
| | de | de | t1 eff | s287 | isolate |
| | 2873mer2 | 2876mer | s287 | isolate | plusCore |
| | modNewCore | modNewCore | isolate | plusCore | allTryp |
| HDAC cluster 0 | 1 | 1 | 1 | 2 | 2 |
| HDAC cluster 1 | 4 | 3 | 2 | 4 | 5 |
| HDAC cluster 2 | 0 | 0 | 0 | 0 | 1 |
| HDAC cluster 3 | 0 | 0 | 0 | 0 | 0 |
| HDAC cluster 4 | 5 | 8 | 2 | 3 | 6 |
| HDAC cluster 5 | 0 | 0 | 0 | 0 | 0 |
| HDAC cluster 6 | 2 | 2 | 1 | 1 | 1 |
| HDAC inhibition | 10 | 13 | 4 | 7 | 11 |
| Propanoic acid | 3 | 3 | 1 | 2 | 3 |
| Butanoic acid | 4 | 7 | 3 | 4 | 6 |
| Pentanoic acid | 1 | 2 | 0 | 0 | 1 |
| Hexanoic acid | 1 | 2 | 0 | 0 | 1 |
| Indole | 3 | 3 | 0 | 2 | 4 |
| 3-methylindole | 2 | 5 | 3 | 4 | 5 |
| 3-indoleacrylic acid | 0 | 0 | 0 | 0 | 0 |
| BSH gCA | 12 | 14 | 5 | 9 | 14 |
| BSH tCA | 10 | 12 | 5 | 8 | 12 |
| BSH gCDCA | 8 | 11 | 5 | 8 | 12 |
| BSH tCDCA | 9 | 11 | 5 | 8 | 12 |
| 7aD CA | 4 | 4 | 0 | 1 | 2 |
| 7aD CDCA | 1 | 1 | 0 | 1 | 1 |
| 7bHSDH UDCA | 0 | 0 | 0 | 0 | 0 |
| Catalase | 1 | 2 | 1 | 2 | 2 |
| a-L-Fucosidase | 1 | 1 | 1 | 2 | 2 |
| IL8 Inflammatory | 0 | 0 | 0 | 0 | 0 |
| LTE | 4 | 4 | 4 | 5 | 6 |
| TE | 8 | 10 | 2 | 5 | 9 |
| Remission Associated | 0 | 2 | 4 | 4 | 4 |
| NonRemission Associated | 2 | 2 | 0 | 1 | 2 |

| | DE | | | | |
|---|---|---|---|---|---|
| | DE211714.1 | DE762708.1 | DE787951.1 | DE291114.1 | DE608959.1 |
| | (DE19) | (DE21) | (DE22) | (DE23) | (DE17) |
| | Alias | | | | |
| | t1 eff | | | max | t1 eff |
| | s287 | | max | eff | s287 |
| | isolate | max | eff | s287 | isolate |
| | plusCore | spp | s287 | isolate | plusCore |
| | maxHDAC0 | eff | isolate | plusCore | sppCluster1 |
| HDAC cluster 0 | 4 | 1 | 1 | 2 | 2 |
| HDAC cluster 1 | 4 | 5 | 3 | 5 | 5 |
| HDAC cluster 2 | 0 | 0 | 0 | 0 | 0 |
| HDAC cluster 3 | 0 | 1 | 0 | 0 | 0 |
| HDAC cluster 4 | 3 | 3 | 3 | 4 | 4 |
| HDAC cluster 5 | 0 | 1 | 1 | 1 | 1 |
| HDAC cluster 6 | 1 | 4 | 3 | 3 | 2 |
| HDAC inhibition | 9 | 10 | 8 | 11 | 10 |
| Propanoic acid | 2 | 4 | 3 | 4 | 4 |
| Butanoic acid | 4 | 6 | 5 | 6 | 5 |
| Pentanoic acid | 0 | 1 | 1 | 1 | 0 |
| Hexanoic acid | 0 | 1 | 1 | 1 | 0 |
| Indole | 2 | 1 | 0 | 2 | 2 |
| 3-methylindole | 4 | 6 | 5 | 6 | 5 |
| 3-indoleacrylic acid | 0 | 0 | 0 | 0 | 0 |
| BSH gCA | 11 | 14 | 10 | 14 | 13 |
| BSH tCA | 8 | 12 | 10 | 13 | 12 |
| BSH gCDCA | 8 | 11 | 9 | 12 | 10 |
| BSH tCDCA | 8 | 12 | 10 | 13 | 11 |
| 7aD CA | 1 | 0 | 0 | 1 | 2 |
| 7aD CDCA | 1 | 0 | 0 | 1 | 1 |
| 7bHSDH UDCA | 0 | 0 | 0 | 0 | 0 |
| Catalase | 2 | 1 | 1 | 2 | 2 |
| a-L-Fucosidase | 3 | 1 | 1 | 2 | 2 |
| IL8 Inflammatory | 0 | 0 | 0 | 0 | 0 |
| LTE | 5 | 7 | 7 | 8 | 8 |
| TE | 5 | 7 | 3 | 6 | 5 |
| Remission Associated | 4 | 7 | 5 | 5 | 4 |
| NonRemission Associated | 1 | 0 | 0 | 1 | 1 |

TABLE 8
Designed Bacterial Compositions (DE20, DE24-DE30, DE32 and DE33) Properties

| | DE | | | | | |
|---|---|---|---|---|---|---|
| | DE313669.1 | DE070875.1 | DE343482.1 | DE616787.1 | DE068851.1 | DE055548.1 |
| | (DE20) | (DE24) | (DE26) | (DE25) | (DE30) | (DE28) |
| | Alias | | | | | |
| | t1 eff | | | | | |
| | s287 | | 15mer | 15mer | | 15mer |
| | isolate | 15mer | wRedundancy | wRedundancy | 15mer | wRedundancy |
| | plusCore | wRedundancy | max3veg | max3veg | wRedundancy | max5veg |
| | maxHDACDiv | distant | maxSpore | maxPropionate | maxT1Eff | maxSkatol |
| HDAC cluster 0 | 2 | 2 | 2 | 2 | 2 | 2 |
| HDAC cluster 1 | 4 | 3 | 2 | 3 | 5 | 4 |
| HDAC cluster 2 | 1 | 1 | 1 | 1 | 0 | 1 |
| HDAC cluster 3 | 1 | 1 | 1 | 1 | 1 | 0 |
| HDAC cluster 4 | 3 | 6 | 7 | 6 | 4 | 6 |
| HDAC cluster 5 | 2 | 0 | 0 | 0 | 0 | 0 |
| HDAC cluster 6 | 2 | 2 | 2 | 2 | 3 | 2 |
| HDAC inhibition | 12 | 12 | 14 | 13 | 11 | 13 |
| Propanoic acid | 6 | 7 | 5 | 7 | 4 | 5 |
| Butanoic acid | 6 | 7 | 8 | 8 | 7 | 9 |
| Pentanoic acid | 0 | 2 | 2 | 2 | 2 | 2 |
| Hexanoic acid | 0 | 2 | 2 | 2 | 2 | 2 |
| Indole | 2 | 4 | 3 | 4 | 3 | 4 |
| 3-methylindole | 6 | 5 | 7 | 6 | 6 | 8 |
| 3-indoleacrylic acid | 0 | 0 | 0 | 0 | 0 | 0 |
| BSH gCA | 14 | 13 | 13 | 13 | 13 | 12 |
| BSH tCA | 13 | 9 | 10 | 10 | 11 | 9 |
| BSH gCDCA | 12 | 9 | 10 | 10 | 11 | 9 |
| BSH tCDCA | 13 | 9 | 10 | 10 | 11 | 9 |
| 7aD CA | 1 | 1 | 1 | 1 | 1 | 1 |
| 7aD CDCA | 1 | 1 | 1 | 1 | 1 | 1 |
| 7bHSDH UDCA | 0 | 0 | 0 | 0 | 0 | 0 |
| Catalase | 2 | 1 | 2 | 2 | 1 | 1 |
| a-L-Fucosidase | 2 | 1 | 1 | 0 | 1 | 0 |
| IL8 Inflammatory | 0 | 0 | 0 | 0 | 0 | 0 |
| LTE | 7 | 9 | 7 | 7 | 9 | 8 |
| TE | 7 | 6 | 8 | 8 | 6 | 7 |
| Remission Associated | 5 | 4 | 5 | 4 | 8 | 5 |
| NonRemission Associated | 1 | 0 | 0 | 0 | 0 | 0 |

| | DE | | | |
|---|---|---|---|---|
| | DE033849.1 | DE865106.1 | DE779249.1 | DE433598.1 |
| | (DE27) | (DE29) | (DE32) | (DE33) |
| | Alias | | | |
| | 15mer | 15mer | | 18mer |
| | wRedundancy | wRedundancy | 18mer | wRedundnancy |
| | max3veg | max5veg | wRedundancy | max5veg |
| | maxT1Eff | maxT1Eff | maxT1Eff | maxT1Eff |
| HDAC cluster 0 | 2 | 2 | 2 | 2 |
| HDAC cluster 1 | 2 | 4 | 5 | 5 |
| HDAC cluster 2 | 1 | 0 | 1 | 0 |
| HDAC cluster 3 | 1 | 1 | 1 | 1 |
| HDAC cluster 4 | 6 | 5 | 6 | 7 |
| HDAC cluster 5 | 0 | 0 | 0 | 0 |
| HDAC cluster 6 | 3 | 3 | 3 | 3 |
| HDAC inhibition | 14 | 12 | 13 | 14 |
| Propanoic acid | 6 | 4 | 6 | 5 |
| Butanoic acid | 8 | 7 | 9 | 8 |
| Pentanoic acid | 2 | 2 | 2 | 2 |
| Hexanoic acid | 2 | 2 | 2 | 2 |
| Indole | 4 | 3 | 3 | 5 |
| 3-methylindole | 6 | 7 | 8 | 6 |
| 3-indoleacrylic acid | 0 | 0 | 0 | 0 |
| BSH gCA | 13 | 13 | 16 | 16 |
| BSH tCA | 10 | 11 | 13 | 12 |
| BSH gCDCA | 10 | 11 | 13 | 12 |
| BSH tCDCA | 10 | 11 | 13 | 12 |
| 7aD CA | 1 | 1 | 1 | 1 |
| 7aD CDCA | 1 | 1 | 1 | 1 |
| 7bHSDH UDCA | 0 | 0 | 0 | 0 |
| Catalase | 1 | 1 | 1 | 1 |
| a-L-Fucosidase | 0 | 0 | 1 | 2 |
| IL8 Inflammatory | 0 | 0 | 0 | 0 |
| LTE | 8 | 8 | 12 | 10 |
| TE | 7 | 7 | 6 | 8 |
| Remission Associated | 5 | 7 | 9 | 7 |
| NonRemission Associated | 0 | 0 | 0 | 0 |

TABLE 9
Designed Bacterial Compositions (DE2, DE9, DE31, and DE34-DE38) Properties

| | DE | | | | | | | |
|---|---|---|---|---|---|---|---|---|
| | DE502105.1 | DE266386.1 | DE278442.1 | DE821956.1 | DE533175.1 | DE346897.1 | DE935045.1 | DE924221.1 |
| | (DE31) | (DE34) | (DE35) | (DE9) | (DE38) | (DE36) | (DE37) | (DE2) |
| | Alias | | | | | | | |
| | 18mer | | | | | | | |
| | wRedundancy | common | mega | lousy | lousier | 23mer | 18mer | |
| | distant | 20mer | 24mer | de | de | swaps | swaps | de2 |
| HDAC | 2 | 2 | 2 | 1 | 1 | 2 | 2 | 2 |
| cluster 0 | | | | | | | | |
| HDAC | 6 | 4 | 9 | 3 | 3 | 7 | 4 | 2 |
| cluster 1 | | | | | | | | |
| HDAC | 1 | 1 | 1 | 0 | 0 | 1 | 0 | 2 |
| cluster 2 | | | | | | | | |
| HDAC | 1 | 1 | 1 | 0 | 0 | 1 | 1 | 3 |
| cluster 3 | | | | | | | | |
| HDAC | 6 | 6 | 8 | 1 | 1 | 8 | 7 | 3 |
| cluster 4 | | | | | | | | |
| HDAC | 0 | 0 | 0 | 1 | 2 | 0 | 0 | 0 |
| cluster 5 | | | | | | | | |
| HDAC | 2 | 3 | 3 | 0 | 1 | 4 | 4 | 1 |
| cluster 6 | | | | | | | | |
| HDAC | 14 | 14 | 17 | 3 | 5 | 18 | 15 | 12 |
| inhibition | | | | | | | | |
| Propanoic | 7 | 6 | 7 | 0 | 0 | 7 | 6 | 6 |
| acid | | | | | | | | |
| Butanoic | 9 | 8 | 11 | 0 | 0 | 12 | 9 | 3 |
| acid | | | | | | | | |
| Pentanoic | 2 | 2 | 2 | 0 | 0 | 1 | 1 | 1 |
| acid | | | | | | | | |
| Hexanoic | 2 | 2 | 2 | 0 | 0 | 1 | 1 | 1 |
| acid | | | | | | | | |
| Indole | 5 | 4 | 5 | 0 | 0 | 5 | 5 | 9 |
| 3-methylindole | 8 | 6 | 10 | 0 | 0 | 10 | 6 | 1 |
| 3-indoleacrylic | 0 | 0 | 0 | 0 | 0 | 0 | 0 | 0 |
| acid | | | | | | | | |
| BSH gCA | 15 | 15 | 21 | 1 | 1 | 20 | 16 | 7 |
| BSH tCA | 12 | 12 | 17 | 1 | 1 | 14 | 10 | 5 |
| BSH gCDCA | 12 | 12 | 17 | 1 | 1 | 15 | 11 | 6 |
| BSH tCDCA | 12 | 12 | 17 | 1 | 1 | 15 | 11 | 6 |
| 7aD CA | 1 | 1 | 1 | 0 | 0 | 1 | 1 | 0 |
| 7aD CDCA | 1 | 1 | 1 | 0 | 0 | 1 | 1 | 0 |
| 7bHSDH UDCA | 0 | 0 | 0 | 0 | 0 | 0 | 0 | 1 |
| Catalase | 1 | 1 | 2 | 0 | 0 | 2 | 1 | 1 |
| a-L- | 0 | 1 | 2 | 0 | 0 | 1 | 1 | 3 |
| Fucosidase | | | | | | | | |
| IL8 | 0 | 0 | 0 | 3 | 5 | 0 | 0 | 0 |
| Inflammatory | | | | | | | | |
| LTE | 9 | 10 | 14 | 1 | 1 | 14 | 11 | 2 |
| TE | 9 | 7 | 10 | 1 | 2 | 9 | 7 | 2 |
| Remission | 6 | 7 | 9 | 1 | 1 | 9 | 7 | 0 |
| Associated | | | | | | | | |
| NonRennission | 0 | 0 | 0 | 0 | 0 | 0 | 0 | 1 |
| Associated | | | | | | | | |

1. A composition comprising a first bacterial species and a second bacterial species,
wherein the first bacterial species and the second bacterial species are different,
wherein the first bacterial species comprises a 16S rDNA sequence that is at least 97% identical to a 16S rDNA sequence set forth in SEQ ID NO: 215, SEQ ID NO: 112, SEQ ID NO: 113, SEQ ID NO: 114, SEQ ID NO: 115, SEQ ID NO: 116, SEQ ID NO: 188, SEQ ID NO: 212, SEQ ID NO: 160, SEQ ID NO: 186, SEQ ID NO: 203, SEQ ID NO: 104, SEQ ID NO: 208, SEQ ID NO: 189, SEQ ID NO: 187, SEQ ID NO: 207, SEQ ID NO: 190, SEQ ID NO: 191, SEQ ID NO: 211, SEQ ID NO: 209, SEQ ID NO: 110, SEQ ID NO: 150, SEQ ID NO: 175, SEQ ID NO: 158, SEQ ID NO: 210, or SEQ ID NO: 106, and
wherein the second bacterial species exhibits a feature selected from: (i) capable of engrafting when administered to a subject, (ii) capable of having anti-inflammatory activity, (iii) not capable of inducing pro-inflammatory activity, (iv) capable of producing a secondary bile acid, (v) capable of producing a tryptophan metabolite, (vi) capable of restoring epithelial integrity as determined by a primary epithelial cell monolayer barrier integrity assay, (vii) capable of being associated with remission of an inflammatory bowel disease, (viii) capable of producing a short-chain fatty acid, (ix) capable of inhibiting a HDAC activity, (x) capable of producing a medium-chain fatty acid, (xi) capable of expressing catalase activity, (xii) capable of having alpha-fucosidase activity, (xiii) capable of inducing Wnt activation, (xiv) capable of producing a B vitamin, (xv) capable of modulating host metabolism of endocannabinoid, (xvi) capable of producing a polyamine and/or modulating a host metabolism of a polyamine, (xvii) capable of reducing fecal levels of a sphingolipid, (xviii) capable of modulating host production of kynurenine, (xix) capable of reducing fecal calprotectin level, (xx) not capable of activating a toll-like receptor pathway, (xxi) capable of activating a toll-like receptor pathway, (xxii) not capable of producing ursodeoxycholic acid, (xxiii) capable of not being associated with clinical non-remission of an inflammatory bowel disease, (xxiv) capable of inhibiting apoptosis of intestinal epithelial cells, (xxv) capable of inducing an anti-inflammatory IL-10-skewed IL-10/IL-6 cytokine ratio in macrophages, (xxvi) capable of not inducing pro-inflammatory IL-6, TNFa, IL-1b, IL-23 or IL-12 production or gene expression in macrophages, (xxvii) capable of downmodulating one or more genes induced in IFN-γ treated colonic organoids (xxix) capable of producing IL-18, (xxx) capable of inducing the activation of antigen presenting cells, (xxxi) capable of reducing the expression of one or more inhibitory receptors on T cells, (xxxii) capable of increasing expression of one or more genes/proteins associated with T cell activation and/or function, (xxxiii) capable of enhancing the ability of CD8+ T cells to kill tumor cells, (xxxiv) capable of enhancing the efficacy of an immune checkpoint inhibitor therapy, (xxxv) capable of reducing colonic inflammation, (xxxvi) capable of promoting the recruitment of CD8+ T cells to tumors, and (xxxvii) any combination thereof.

2. The composition of claim 1,
which further comprises one or more additional bacterial species, wherein the additional bacterial species is different from the first bacterial species and the second bacterial species, and wherein the additional bacterial species has a 16S rDNA sequence that is at least 97% identical to a 16S rDNA sequence of any of the bacteria set forth in FIG. 31, FIG. 32, FIG. 33, or FIG. 34.

3-21. (canceled)

22. A composition comprising a bacterial population, wherein the bacterial population comprises two or more features selected from the group consisting of (i) capable of engrafting when administered to a subject, (ii) capable of having anti-inflammatory activity, (iii) not capable of inducing pro-inflammatory activity, (iv) capable of producing a secondary bile acid, (v) capable of producing a tryptophan metabolite, (vi) capable of restoring epithelial integrity as determined by a primary epithelial cell monolayer barrier integrity assay, (vii) capable of being associated with remission of an inflammatory bowel disease, (viii) capable of producing a short-chain fatty acid, (ix) capable of inhibiting a HDAC activity, (x) capable of producing a medium-chain fatty acid, (xi) capable of expressing catalase activity, (xii) capable of having alpha-fucosidase activity, (xiii) capable of inducing Wnt activation, (xiv) capable of producing a B vitamin, (xv) capable of modulating host metabolism of endocannabinoid, (xvi) capable of producing a polyamine and/or modulating a host metabolism of a polyamine, (xvii) capable of reducing fecal levels of a sphingolipid, (xviii) capable of modulating host production of kynurenine, (xix) capable of reducing fecal calprotectin level, (xx) not capable of activating a toll-like receptor pathway, (xxi) capable of activating a toll-like receptor pathway, (xxii) not capable of producing ursodeoxycholic acid, (xxiii) capable of not being associated with clinical non-remission of an inflammatory bowel disease, (xxiv) capable of inhibiting apoptosis of intestinal epithelial cells, (xxv) capable of inducing an anti-inflammatory IL-10-skewed IL-10/IL-6 cytokine ratio in macrophages, (xxvi) capable of not inducing pro-inflammatory IL-6, TNFa, IL-1b, IL-23 or IL-12 production or gene expression in macrophages, (xxvii) capable of downmodulating one or more genes induced in IFN-γ treated colonic organoids (xxix) capable of producing IL-18, (xxx) capable of inducing the activation of antigen presenting cells, (xxxi) capable of reducing the expression of one or more inhibitory receptors on T cells, (xxxii) capable of increasing expression of one or more genes/proteins associated with T cell activation and/or function (xxxiii) capable of enhancing the ability of CD8+ T cells to kill tumor cells, (xxxiv) capable of enhancing the efficacy of an immune checkpoint inhibitor therapy, (xxxv) capable of reducing colonic inflammation, (xxxvi) capable of promoting the recruitment of CD8+ T cells to tumors, and (xxxvii) any combination thereof.

23. (canceled)

24. (canceled)

25. The composition of claim 22, wherein the bacterial population comprises one or more bacteria having a 16S rDNA sequence that is at least 97% identical to a 16S rDNA sequence of any of the bacteria set forth in FIG. 31, FIG. 32, FIG. 33, or FIG. 34.

26. (canceled)

27. A composition comprising a bacterial population, comprising two or more bacteria, wherein the two or more bacteria comprises a long-term engrafter and a transient engrafter.

28. The composition of claim 27, wherein the bacterial population further comprises one or more bacteria, which has one or more features selected from the group consisting of: (i) capable of engrafting when administered to a subject, (ii) capable of having anti-inflammatory activity, (iii) not capable of inducing pro-inflammatory activity, (iv) capable of producing a secondary bile acid, (v) capable of producing a tryptophan metabolite, (vi) capable of restoring epithelial integrity as determined by a primary epithelial cell monolayer barrier integrity assay, (vii) capable of being associated with remission of an inflammatory bowel disease, (viii) capable of producing a short-chain fatty acid, (ix) capable of inhibiting a HDAC activity, (x) capable of producing a medium-chain fatty acid, (xi) capable of expressing catalase activity, (xii) capable of having alpha-fucosidase activity, (xiii) capable of inducing Wnt activation, (xiv) capable of producing a B vitamin, (xv) capable of modulating host metabolism of endocannabinoid, (xvi) capable of producing a polyamine and/or modulating host metabolism of polyamines, (xvii) capable of reducing fecal levels of a sphingolipid, (xviii) capable of modulating host production of kynurenine, (xix) capable of reducing fecal calprotectin level, (xx) not capable of activating a toll-like receptor pathway, (xxi) capable of activating a toll-like receptor pathway, (xxii) not capable of producing ursodeoxycholic acid, (xxiii) capable of not being associated with clinical non-remission of an inflammatory bowel disease, (xxiv) capable of inhibiting apoptosis of intestinal epithelial cells, (xxv) capable of inducing an anti-inflammatory IL-10-skewed IL-10/IL-6 cytokine ratio in macrophages, (xxvi) capable of not inducing pro-inflammatory IL-6, TNFa, IL-1b, TL-23 or IL-12 production or gene expression in macrophages, (xxvii) capable of downmodulating one or more genes induced in IFN-γ treated colonic organoids, (xxix) capable of producing IL-18, (xxx) capable of inducing the activation of antigen presenting cells, (xxxi) capable of reducing the expression of one or more inhibitory receptors on T cells, (xxxii) capable of increasing expression of one or more genes/proteins associated with T cell activation and/or function, (xxxiii) capable of enhancing the ability of CD8+ T cells to kill tumor cells, (xxxiv) capable of enhancing the efficacy of an immune checkpoint inhibitor therapy, (xxxv) capable of reducing colonic inflammation, (xxxvi) capable of promoting the recruitment of CD8+ T cells to tumors, and (xxxvii) any combination thereof.

29-55. (canceled)

56. The composition of claim 27, wherein: (i) the long-term engrafter has a 16S rDNA sequence that is at least 97% identical to a 16S rDNA sequence of a long-term engrafter provided in Table 5: (ii) the transient engrafter has a 16S rDNA sequence that is at least 97% identical to a 16S rDNA sequence of a transient engrafter provided in Table 5; or (iii) both (i) and (ii).

57-61. (canceled)

62. The composition of claim 25, wherein the a bacterial population comprises any of DE1, DE2, DE3, DE4, DE5, DE6, DE7, DE8, DE9, DE10, DE11, DE12, DE13, DE14, DE15, DE16, DE17, DE18, DE19, DE20, DE21, DE22, DE23, DE24, DE25, DE26, DE27, DE28, DE29, DE30, DE31, DE32, DE33, DE34, DE35, DE36, DE37, DE38, DE39, DE40, DE41, DE42, DE43, DE44, DE45, DE46, DE47, DE48, DE49, DE50, DE51, DE52, DE53, DE54, DE55, or DE56 set forth in FIG. 31, FIG. 32, FIG. 33, or FIG.

63-68. (canceled)

69. The composition of claim 1, further comprising an enteric polymer.

70. A pharmaceutical formulation comprising the composition of claim 1, and a pharmaceutically acceptable excipient.

71. The pharmaceutical formulation of claim 70, wherein the excipient is glycerol.

72. The pharmaceutical formulation of claim 70, wherein at least one of the bacterial species is lyophilized.

73. The pharmaceutical formulation of claim 70, wherein the composition is formulated for oral delivery.

74. A method of treating an inflammatory disease in a subject in need thereof, comprising administering to the subject the composition of claim 1.

75. (canceled)

76. The method of claim 74, wherein the inflammatory disease comprises an inflammatory bowel disease.

77-79. (canceled)

80. A method of modulating the level of a biological molecule in a subject in need thereof, comprising administering to the subject the composition of claim 1.

81. The method of claim 80, wherein the biological molecule comprises a fecal calprotectin, a secondary bile acid, a tryptophan metabolite, a short-chain fatty acid, a medium-chain fatty acid, a sphingolipid, a kynurenine, or any combination thereof.

82. (canceled)

83. (canceled)

84. The method of claim 81, wherein: (i) the secondary bile acid comprises deoxycholic acid (DCA), 3α 12-oxo-deoxycholic acid, 3β 12α-deoxycholic acid (3-isodeoxycholic acid), 7α 3-oxo-chenodeoxycholic acid, lithocholic acid (LCA), 3-oxo LCA, or any combination thereof; (ii) the tryptophan metabolite comprises indole, 3-methylindole, or both: (iii) the short-chain fatty acid is selected from formate, acetate, propionate, butyrate, isobutryate, valerate, isovalerate, or any combination thereof; or (iv) any combination of (i) to (iii).

85-90. (canceled)

91. A method of treating a cancer in a subject in need thereof, comprising administering to the subject the composition of claim 1.

92-96. (canceled)

97. A method of enhancing an immune response in a subject in need thereof, comprising administering to the subject the composition of claim 1.

98-107. (canceled)
